(19) United States
(12) Patent Application Publication
(10) Pub. No.: US 2025/0301905 A1
(43) Pub. Date: Sep. 25, 2025

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si (KR)

(72) Inventors: Hanill LEE, Suwon-si (KR); Hyung Sun KIM, Suwon-si (KR); Dalho HUH, Suwon-si (KR); Jiyun KWON, Suwon-si (KR); Wook KIM, Suwon-si (KR); Jonghoon KIM, Suwon-si (KR); Seungjae LEE, Suwon-si (KR); Jiah YOON, Suwon-si (KR); Seonyeong GWAK, Suwon-si (KR); Suheon IM, Suwon-si (KR); Jaehoon KIM, Suwon-si (KR); Dongkyu RYU, Suwon-si (KR); Jihun SHIN, Suwon-si (KR); Mijin LEE, Suwon-si (KR)

(21) Appl. No.: 19/052,441

(22) Filed: Feb. 13, 2025

(30) Foreign Application Priority Data
Mar. 22, 2024 (KR) 10-2024-0039903

(51) Int. Cl. H10K 85/60; H10K 50/12; H10K 50/15; H10K 50/16; H10K 85/40

(52) U.S. Cl. CPC H10K 85/6572; H10K 50/12; H10K 85/40; H10K 85/622; H10K 85/633; H10K 85/636; H10K 85/654; H10K 85/6574; H10K 85/6576; H10K 50/15; H10K 50/16

(57) ABSTRACT

A compound for an organic optoelectronic device, a composition for an organic optoelectronic device including the compound, an organic optoelectronic device including the compound or the composition for an organic optoelectronic device, and a display device including the organic optoelectronic device, the compound being represented by Chemical

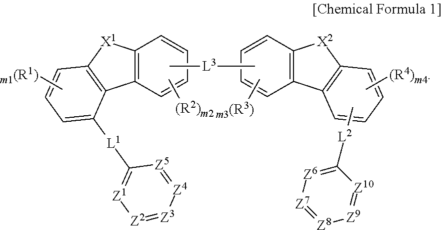

### CROSS-REFERENCE TO RELATED APPLICATION

This application claims priority to and the benefit of Korean Patent Application No. 10-2024-0039903 filed in the Korean Intellectual Property Office on Mar. 22, 2024, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device may include an organic photoelectric device, an organic light emitting diode, an organic solar cell, or an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode may be influenced by an organic material between electrodes.

### SUMMARY

The embodiments may be realized by providing a compound for an organic optoelectronic device, the compound being represented by Chemical Formula 1:

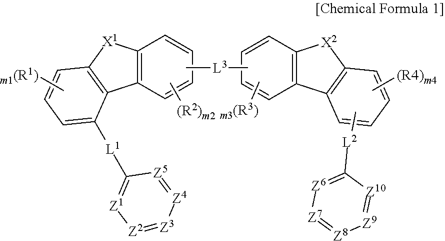

wherein, in Chemical Formula 1, X¹ and X² are each independently O, S, CR^{a}R^{b}, SiR^{c}R^{d}, or S(O)₂, Z¹ to Z¹⁰ are each independently N or CR^{e}, at least two of Z¹ to Z⁵ are N, at least two of Z⁶ to Z¹⁰ are N, R^{a}, R^{b}, R^{c}, R^{d}, R^{e}, and R¹ to R⁴ are each independently hydrogen, deuterium, a cyano group, a substituted or unsubstituted C1 to C20 alkyl group, a substituted or unsubstituted C6 to C20 aryl group, a substituted or unsubstituted C2 to C20 heterocyclic group, or a combination thereof, L¹ to L³ are each independently a single bond, a substituted or unsubstituted C6 to C20 arylene group or a substituted or unsubstituted C2 to C30 heterocyclic group, m1 to m4 are each independently an integer of 1 to 3, when m1 to m4 are 2 or more, each of R¹ to R⁴ is the same or different, and R¹ to R⁴ are each independently present or adjacent groups are linked to each other to form a substituted or unsubstituted aromatic monocyclic ring, a substituted or unsubstituted aromatic polycyclic ring, a substituted or unsubstituted heteroaromatic monocyclic ring, or a substituted or unsubstituted heteroaromatic polycyclic ring, and each R^{e} is the same or different, and each R^{e} is separately present or adjacent groups are linked to form a substituted or unsubstituted aromatic monocyclic ring, a substituted or unsubstituted aromatic polycyclic ring, a substituted or unsubstituted heteroaromatic monocyclic ring, or a substituted or unsubstituted heteroaromatic polycyclic ring.

The embodiments may be realized by providing a composition for an organic optoelectronic device, the composition including a first compound; and a second compound, wherein the first compound is the compound for an organic optoelectronic device according to an embodiment, and the second compound is represented by Chemical Formula 2, a combination of Chemical Formula 3 and Chemical Formula 4, or Chemical Formula 5:

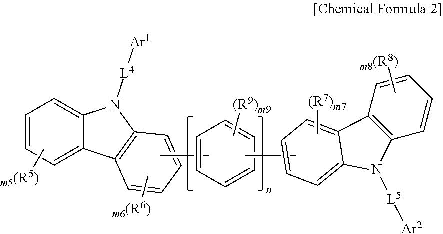

in Chemical Formula 2, R⁵ to R⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar¹ and Ar² are each independently a substituted or unsubstituted C6 to C20 aryl group or a substituted or unsubstituted C2 to C30 heterocyclic group, L⁴ and L⁵ are each independently a single bond or a substituted or unsubstituted C6 to C20 arylene group, m5, m8, and m9 are each independently an integer of 1 to 4, m6 and m7 are each independently an integer of 1 to 3, when m5 is 2, 3, or 4, each R⁵ is the same or different from each other, when m6 is 2 or 3, each R⁶ is the same or different from each other, when m7 is 2 or 3, each R⁷ is the same or different from each other, when m8 is 2, 3, or 4, each R⁸ is the same or different from each other, when m9 is 2, 3, or 4, each R⁹ is the same or different from each other, and n is an integer of 0 to 2;

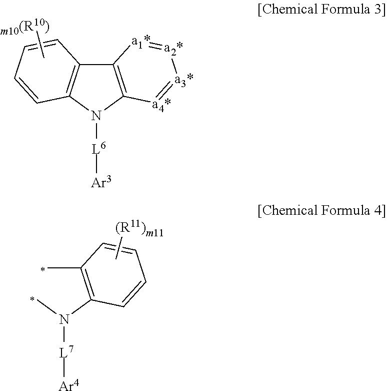

in Chemical Formula 3 and Chemical Formula 4, two adjacent ones of a1* to a4* of Chemical Formula 3, are each linking carbons linked at * of Chemical Formula 4, the remaining two of a1* to a4* of Chemical Formula 3, not linked at * of Chemical Formula 4, are independently C-L^{a}-R^{f}, L^{a}, L⁶, and L⁷ are each independently a single bond or a substituted or unsubstituted C6 to C20 arylene group, R^{f}, R¹⁰, and R¹¹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar³ and Ar⁴ are each independently a substituted or unsubstituted C6 to C20 aryl group or a substituted or unsubstituted C2 to C30 heterocyclic group, m10 and m11 are each independently an integer of 1 to 4, when m10 is 2, 3, or 4, each R¹⁰ is the same or different from each other, and when m11 is 2, 3, or 4, each R¹¹ is the same or different from each other;

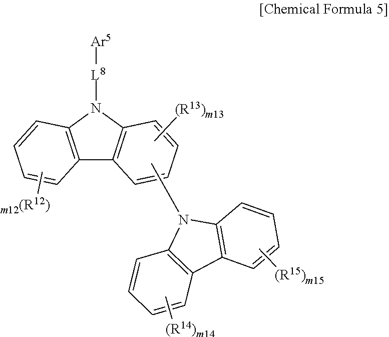

in Chemical Formula 5, L⁸ is a single bond or a substituted or unsubstituted C6 to C20 arylene group, R¹² to R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar⁵ is a substituted or unsubstituted C6 to C20 aryl group or a substituted or unsubstituted C2 to C30 heterocyclic group, m12, m14, and m15 are each independently an integer of 1 to 4, m13 is an integer of 1 to 3, when m12 is 2, 3, or 4, each R¹² is the same or different from each other, when m13 is 2 or 3, each R¹³ is the same or different from each other, when m14 is 2, 3, or 4, each R¹⁴ is the same or different from each other, and when m15 is 2, 3, or 4, each R¹⁵ is the same or different from each other.

The embodiments may be realized by providing, an organic optoelectronic device including an anode and a cathode facing each other, and at least one organic layer between the anode and the cathode, wherein the at least one organic layer includes the compound for an organic optoelectronic device according to an embodiment.

The embodiments may be realized by providing an organic optoelectronic device including an anode and a cathode facing each other, and at least one organic layer between the anode and the cathode, wherein the organic layer includes the composition for an organic optoelectronic device according to an embodiment.

The embodiments may be realized by providing a display device including the organic optoelectronic device according to an embodiment.

### BRIEF DESCRIPTION OF THE DRAWING

Features will become apparent to those of skill in the art by describing in detail exemplary embodiments with reference to the attached drawing in which:

the FIGURE is a cross-sectional view showing an organic light emitting diode according to some embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. As used herein, the term “or” is not necessarily an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present embodiments, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present embodiments, the “substituted” refers to replacement of at least one hydrogen of a substituent or a compound by deuterium, a C1 to C20 alkyl group, a C1 to C5 alkylsilyl group, a C6 to C20 aryl group, a C2 to C20 heteroaryl group, or a cyano group. In specific example of the present embodiments, the “substituted” refers to replacement of at least one hydrogen of a substituent or a compound by deuterium, a C1 to C5 alkyl group, a C1 to C5 alkylsilyl group, a C6 to C18 aryl group, a C2 to C18 heteroaryl group, or a cyano group. In specific example of the present embodiments, the “substituted” refers to replacement of at least one hydrogen of a substituent or a compound by deuterium, a cyano group, a methyl group, an ethyl group, an n-propyl group, an iso-propyl group, an n-butyl group, an iso-butyl group, a sec-butyl group, a tert-butyl group, a trimethylsilyl group, a phenyl group, a biphenyl group, a terphenyl group, or a naphthyl group.

“Unsubstituted” refers to non-replacement of a hydrogen atom by another substituent and remaining of the hydrogen atom.

As used herein, “hydrogen substitution (—H)” may include “deuterium substitution (-D)” or “tritium substitution (-T).” For example, any hydrogen in any compound described herein may be protium, deuterium, or tritium (e.g., based on natural or artificial substitution).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “a heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to some embodiments is described.

The compound for an organic optoelectronic device according to some embodiments may be represented by Chemical Formula 1.

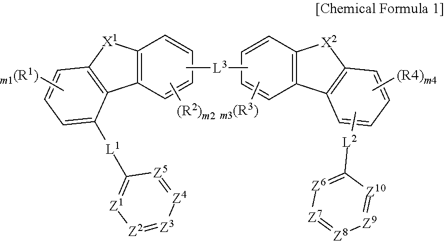

In Chemical Formula 1, X¹ and X² may each independently be or include, e.g., O, S, CR^{a}R^{b}, SiR^{c}R^{d}, or S(O)₂.

Z¹ to Z¹⁰ may each independently be or include, e.g., N or CR^{e}.

In an implementation, at least two of Z¹ to Z⁵ are N.

In an implementation, at least two of Z⁶ to Z¹⁰ are N.

R^{a}, R^{b}, R^{c}, R^{d}, R^{e}, and R¹ to R⁴ may each independently be or include, e.g., hydrogen, deuterium, a cyano group, a substituted or unsubstituted C1 to C20 alkyl group, a substituted or unsubstituted C6 to C20 aryl group, a substituted or unsubstituted C2 to C20 heterocyclic group, or a combination thereof.

L¹ to L³ may each independently be or include, e.g., a single bond, a substituted or unsubstituted C6 to C20 arylene group or a substituted or unsubstituted C2 to C30 heterocyclic group.

m1 to m4 may each independently be, e.g., an integer of 1 to 3.

In an implementation, m1 may be 2 to 4, and each R¹ may be the same or different from each other, and each R¹ may be separately present or adjacent groups may be linked to each other to form a substituted or unsubstituted aromatic monocyclic ring, a substituted or unsubstituted aromatic polycyclic ring, a substituted or unsubstituted heteroaromatic monocyclic ring, or a substituted or unsubstituted heteroaromatic polycyclic ring.

In an implementation, m2 may be 2 to 4, and each R² may be the same or different from each other, and each R² may be separately present or adjacent groups may be linked to each other to form a substituted or unsubstituted aromatic monocyclic ring, a substituted or unsubstituted aromatic polycyclic ring, a substituted or unsubstituted heteroaromatic monocyclic ring, or a substituted or unsubstituted heteroaromatic polycyclic ring.

In an implementation, m3 may be 2 to 4, and each R³ may be the same or different, and each R³ may be separately present or adjacent groups may be linked to each other to form a substituted or unsubstituted aromatic monocyclic ring, a substituted or unsubstituted aromatic polycyclic ring, a substituted or unsubstituted heteroaromatic monocyclic ring, or a substituted or unsubstituted heteroaromatic polycyclic ring.

In an implementation, m4 may be 2 to 4, and each R⁴ may be the same or different, and each R⁴ may be separately present or adjacent groups may be linked to each other to form a substituted or unsubstituted aromatic monocyclic ring, a substituted or unsubstituted aromatic polycyclic ring, a substituted or unsubstituted heteroaromatic monocyclic ring, or a substituted or unsubstituted heteroaromatic polycyclic ring.

In an implementation, each R^{e} may be the same or different, and each R^{e} may be separately present or adjacent groups may be linked to form a substituted or unsubstituted aromatic monocyclic ring, a substituted or unsubstituted aromatic polycyclic ring, a substituted or unsubstituted heteroaromatic monocyclic ring, or a substituted or unsubstituted heteroaromatic polycyclic ring.

The compound for an organic optoelectronic device represented by Chemical Formula 1 according to the present disclosure may have a high charge mobility by being linked to triazine at the 1st position of the fused ring including X¹.

In addition, by substituting the fused ring including X² with an additional triazine and thus by adding a triazine that can contact an anion of the device in an electromagnetic field, an energy received by one triazine may be lowered, degradation may be prevented, and the life-span of the device may be significantly improved.

Meanwhile, the lone pair of electrons of X¹ may easily hop, like the free electrons of a metal, in an electromagnetic field, and in particular, if triazine is substituted at the 1st position, steric hindrance may be greatly increased due to the substituent at the 9th position and this structural twist may serve to greatly lower the energy barrier that lone pairs of electrons may hop for. The role of lowering the attraction between atoms for lone pairs of electrons due to steric hindrance may facilitate charge transfer between molecules, the hopping speed due to an effect of delocalizing electrons by additionally linking the fused ring including X² may be controlled, and stability of the molecule may be increased to help solve the problem of degradation.

In summary, the significance of the present disclosure is to design a molecule with high stability and high charge mobility without reducing charge mobility. When two dibenzo derivatives (fused rings including X¹ or X²) are linked and each is linked to triazine, delocalization occurs in which electrons are shared with each other, and due to X¹ and X² having lone pairs of electrons, charge mobility may be significantly increased compared to the case where only the lone pair of X¹ is present.

In addition, by linking three different substituents around the triazine, the symmetry of the molecule may be broken, and the steric hindrance may be large, resulting in a low deposition temperature. Due to this effect, the deposition film may be improved, making it possible to develop a material with a much better life-span.

In an implementation, Chemical Formula 1 may be represented by one of Chemical Formulae 1-1 to Chemical Formula 1-4.

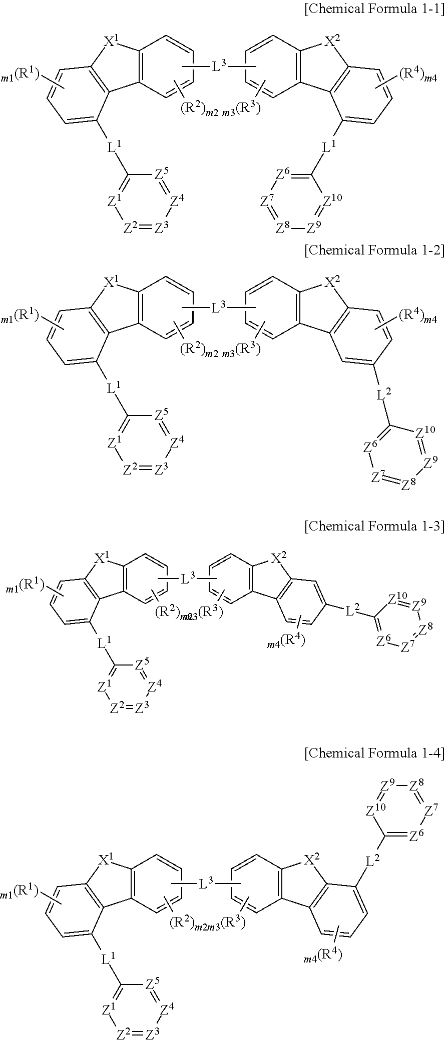

In Chemical Formula 1-1 to Chemical Formula 1-4, X¹ and X², Z¹ to Z¹⁰, R¹ to R⁴, L¹ to L³, and m1 to m4 may be defined the same as those of Chemical Formula 1.

In an implementation, Chemical Formula 1 may be represented by one of Chemical Formulae 1-5 to Chemical Formula 1-7.

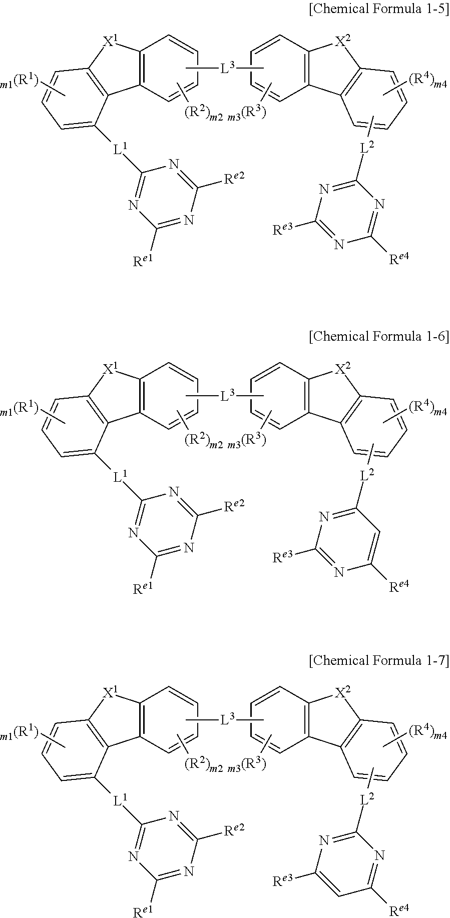

In Chemical Formula 1-5 to Chemical Formula 1-7, X¹ and X², R¹ to R⁴, L¹ to L³, and m1 to m4 may be defined the same as those of Chemical Formula 1 and R^{e1} to R^{e4} may each be defined the same as R^{e} of Chemical Formula 1.

In an implementation, X¹ and X² may each independently be O, S, or SiR^{c}R^{d} in which R^{c} and R^{d} may each independently be or include, e.g., hydrogen, deuterium, a cyano group, a substituted or unsubstituted C1 to C20 alkyl group, a substituted or unsubstituted C6 to C20 aryl group, or a combination thereof.

In an implementation, (X¹ and X²) may each be (O and O), (O and S), or (O and SiR^{c}R^{d}, wherein R^{c} and R^{d} may each independently be, e.g., hydrogen, deuterium, a cyano group, a substituted or unsubstituted C1 to C20 alkyl group, a substituted or unsubstituted C6 to C20 aryl group, or a combination thereof.

In an implementation, each of L¹ to L³ may be a single bond.

In an implementation, R^{a}, R^{b}, R^{c}, and R^{d} may each independently be, e.g., a substituted or unsubstituted C1 to C10 alkyl group, or a substituted or unsubstituted C6 to C12 aryl group.

In an implementation, R^{a}, R^{b}, R^{c}, and R^{d} may each independently be, e.g., a substituted or unsubstituted methyl group, a substituted or unsubstituted ethyl group, a substituted or unsubstituted propyl group, a substituted or unsubstituted butyl group, a substituted or unsubstituted phenyl group, a substituted or unsubstituted biphenyl group, or a substituted or unsubstituted naphthyl group.

In an implementation, R¹ to R⁴ may each independently be, e.g., hydrogen, deuterium, a cyano group, a substituted or unsubstituted C1 to C10 alkyl group, or a substituted or unsubstituted C6 to C12 aryl group.

In an implementation, R¹ to R⁴ may each independently be, e.g., hydrogen, deuterium, a cyano group, a substituted or unsubstituted methyl group, a substituted or unsubstituted ethyl group, a substituted or unsubstituted propyl group, a substituted or unsubstituted butyl group, a substituted or unsubstituted phenyl group, a substituted or unsubstituted biphenyl group, or a substituted or unsubstituted naphthyl group.

In an implementation, R^{e} and R^{e1} to R^{e4} may each independently be, e.g., hydrogen, deuterium, a cyano group, a substituted or unsubstituted C1 to C10 alkyl group, a substituted or unsubstituted C6 to C12 aryl group, or a substituted or unsubstituted C6 to C20 heterocyclic group.

In an implementation, R^{e} and R^{e1} to R^{e4} may each independently be, e.g., hydrogen, deuterium, a cyano group, a substituted or unsubstituted methyl group, a substituted or unsubstituted ethyl group, a substituted or unsubstituted propyl group, a substituted or unsubstituted butyl group, a substituted or unsubstituted phenyl group, a substituted or unsubstituted biphenyl group, a substituted or unsubstituted naphthyl group, or a substituted or unsubstituted carbazolyl group.

In an implementation, the compound represented by Chemical Formula 1 may be, e.g., a compound of Group 1.

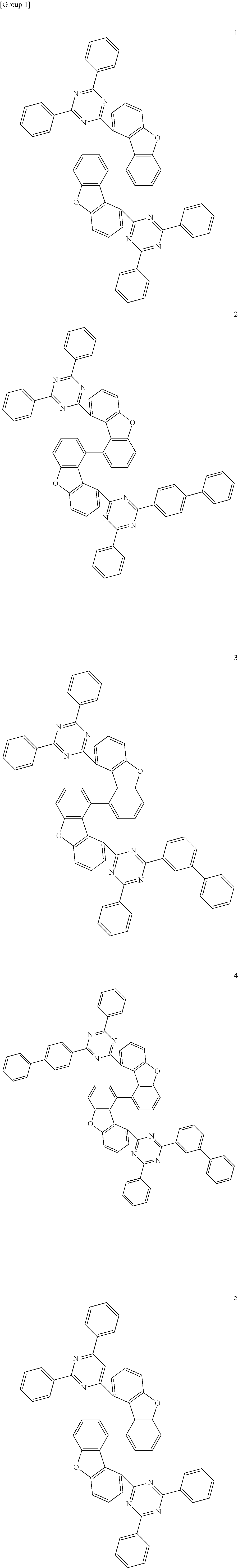

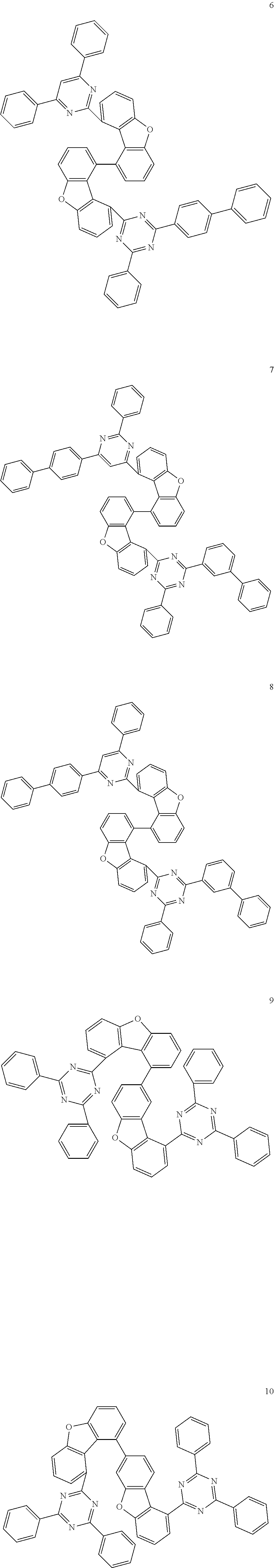

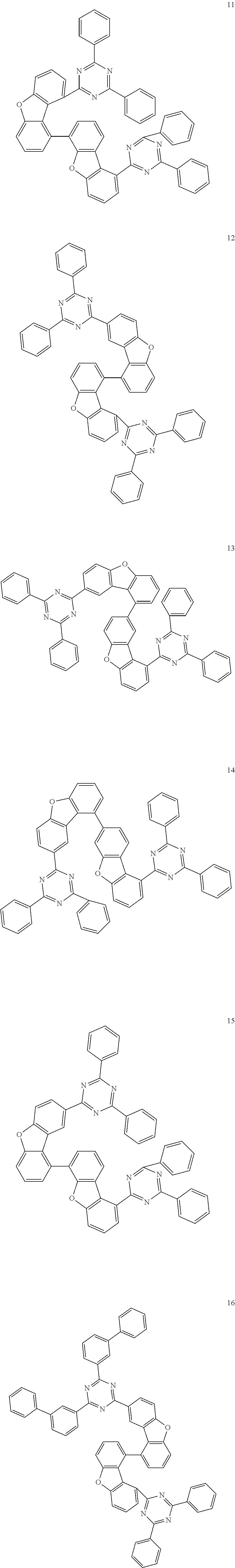

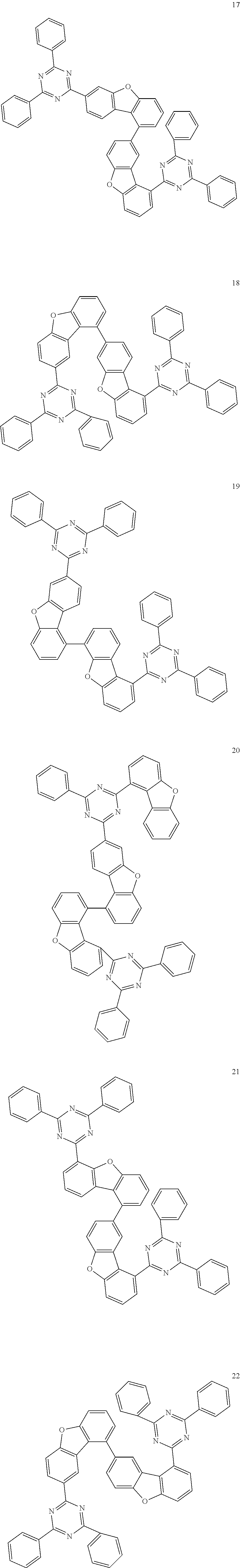

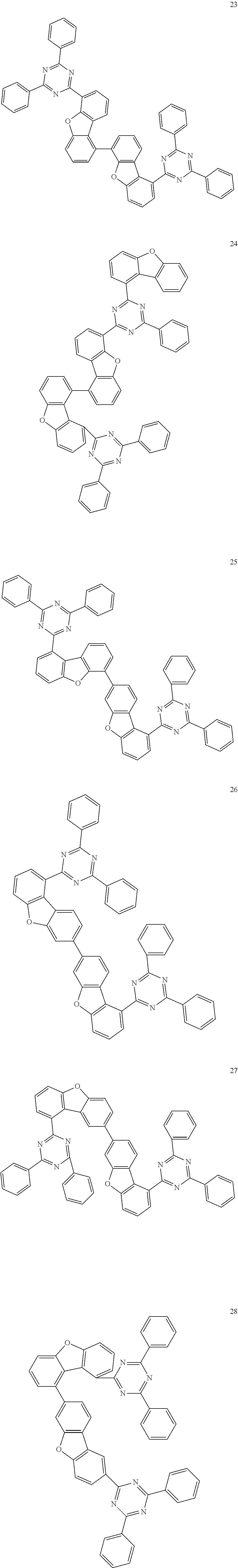

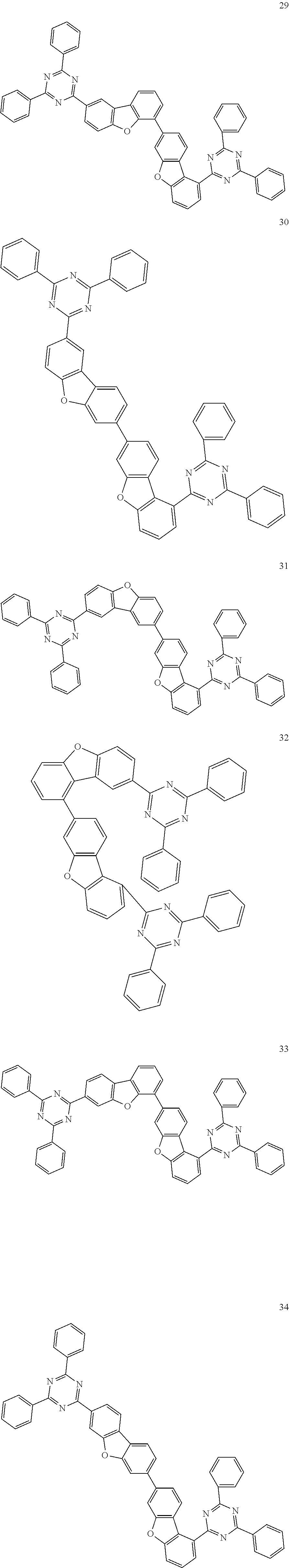

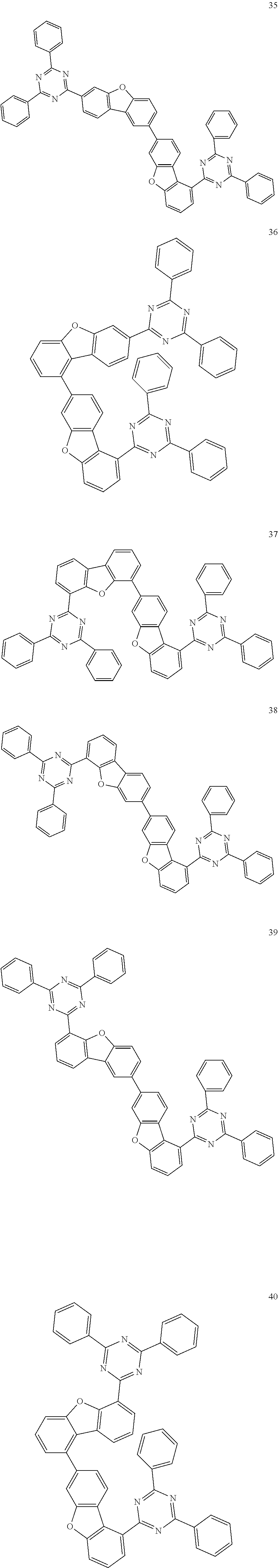

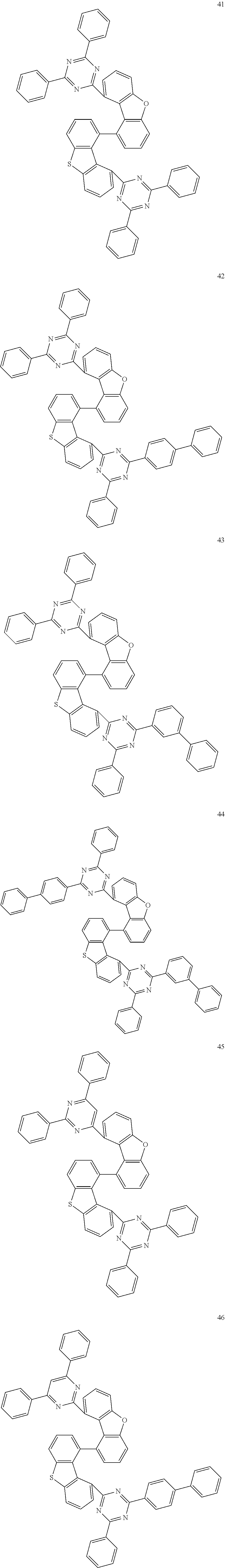

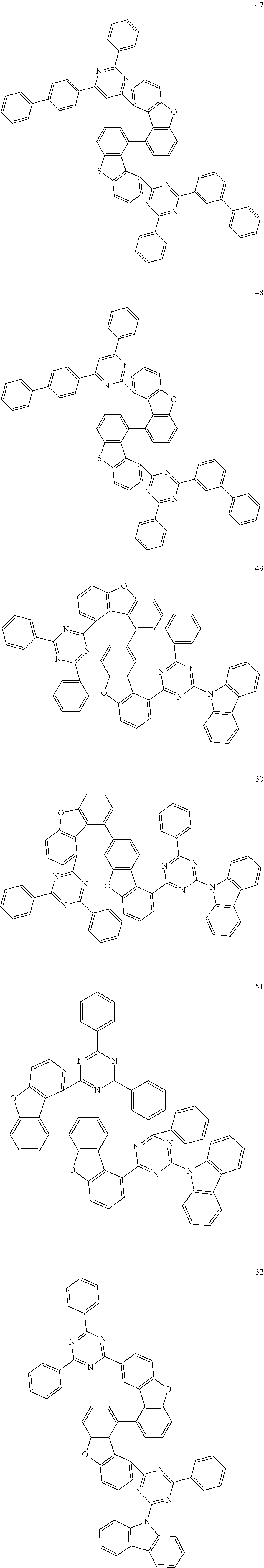

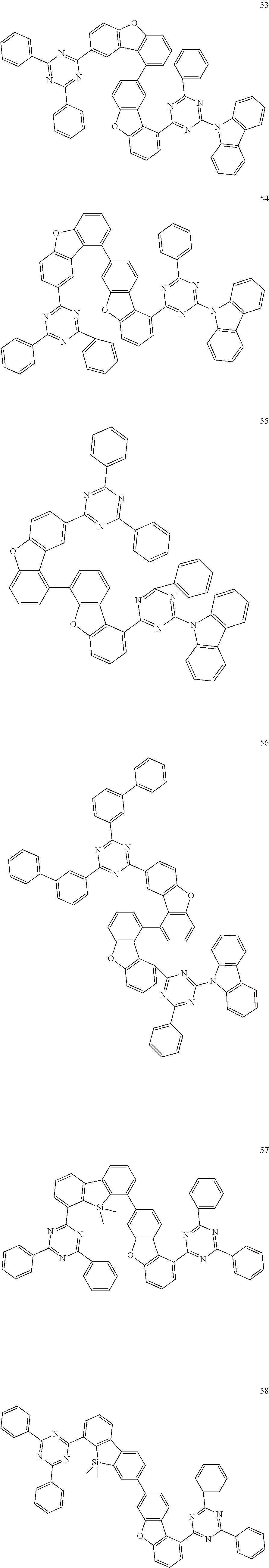

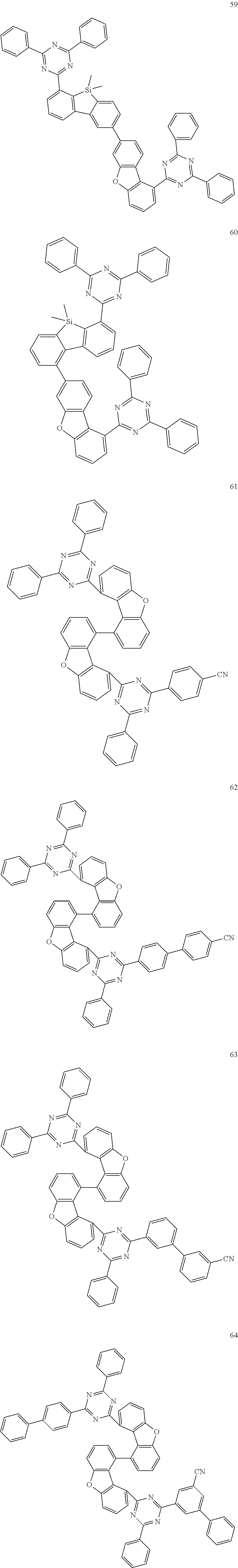

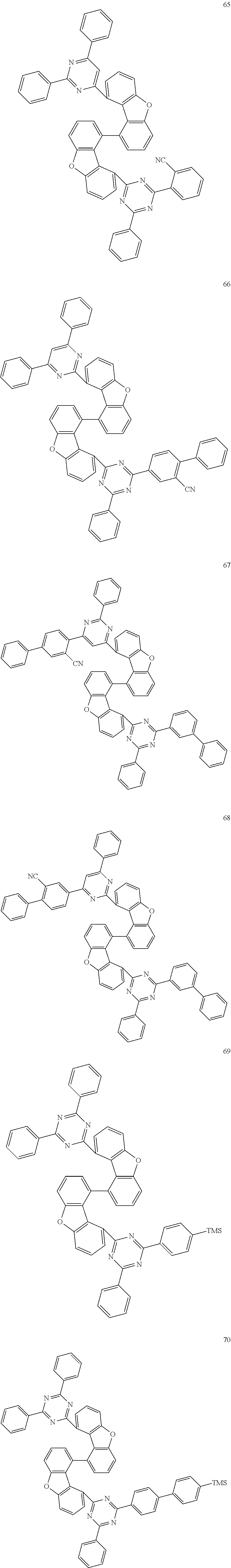

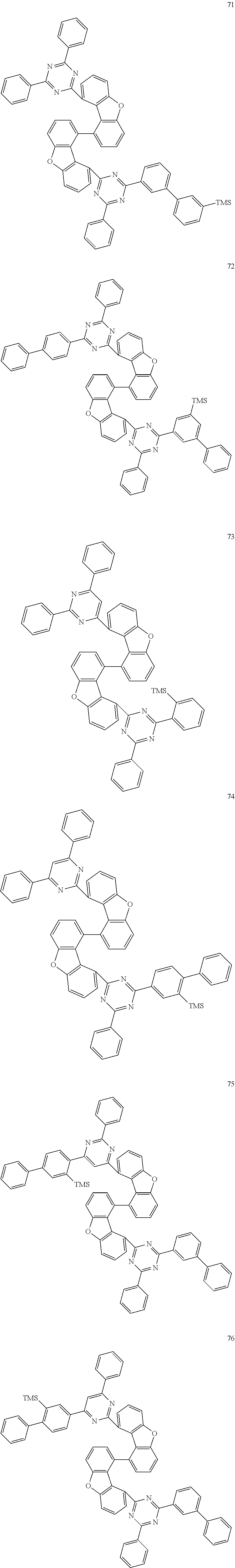

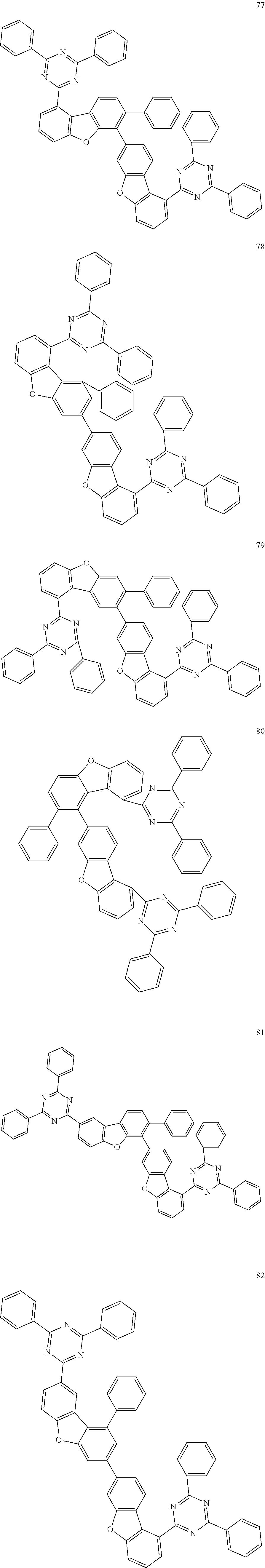

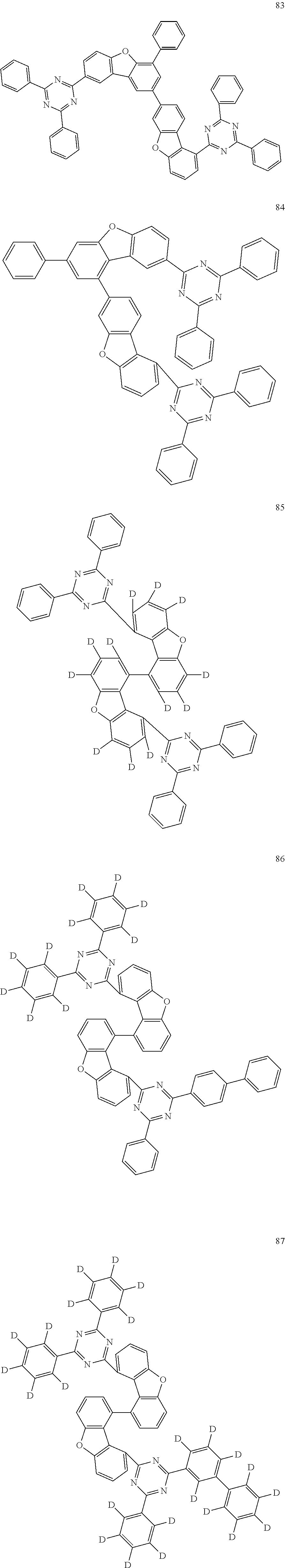

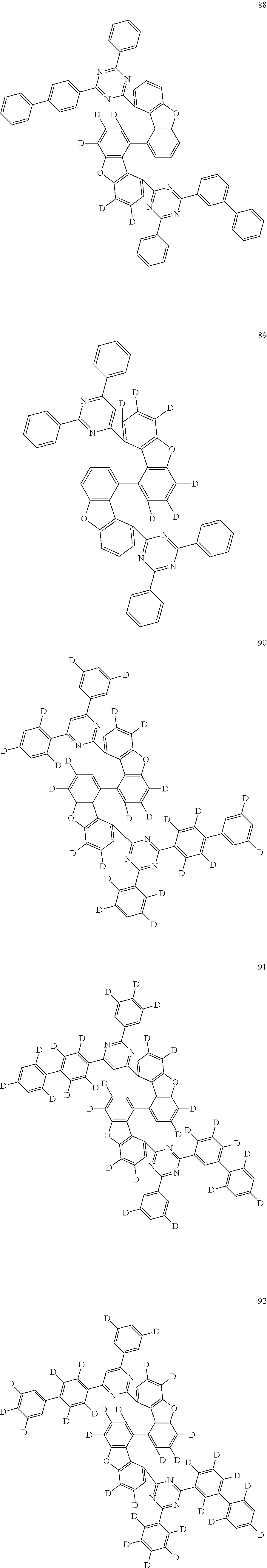

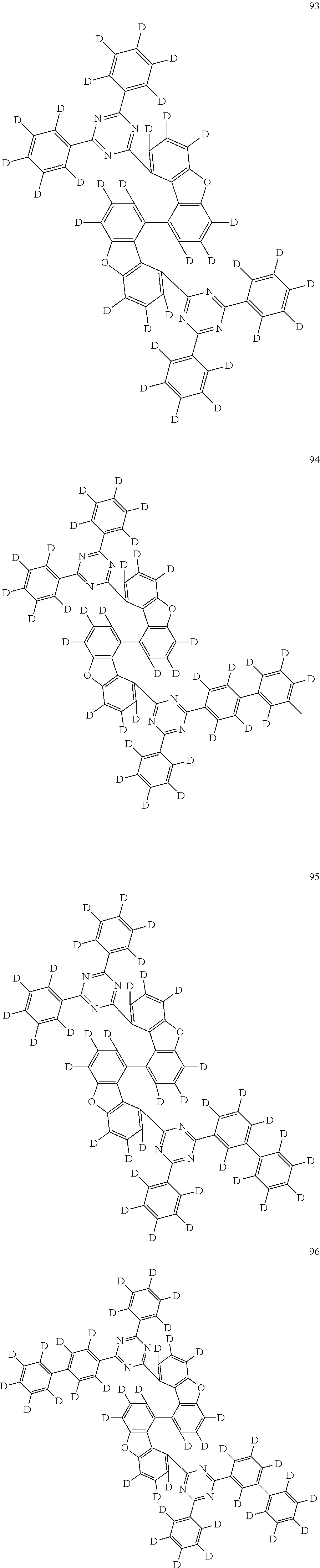

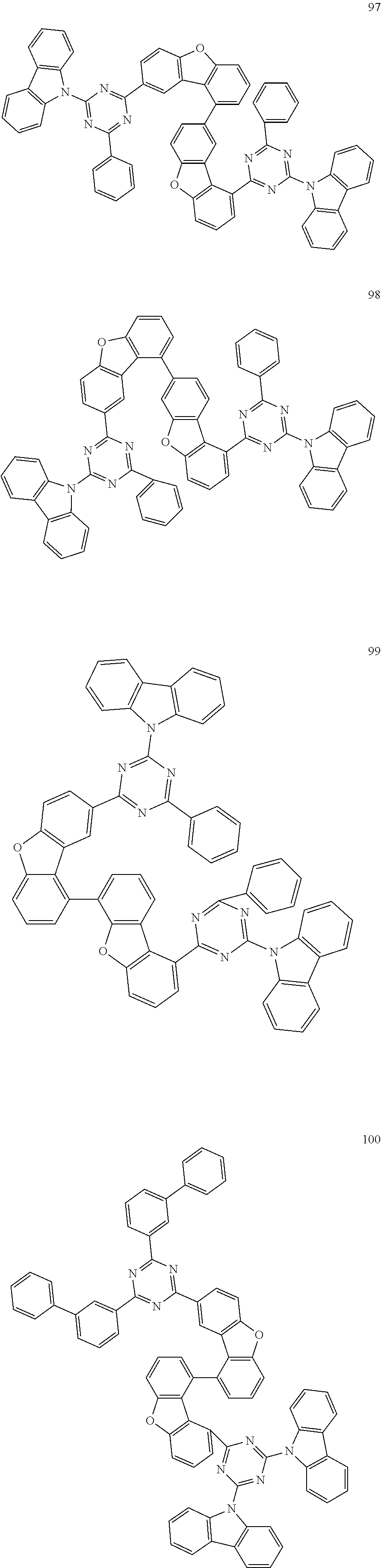

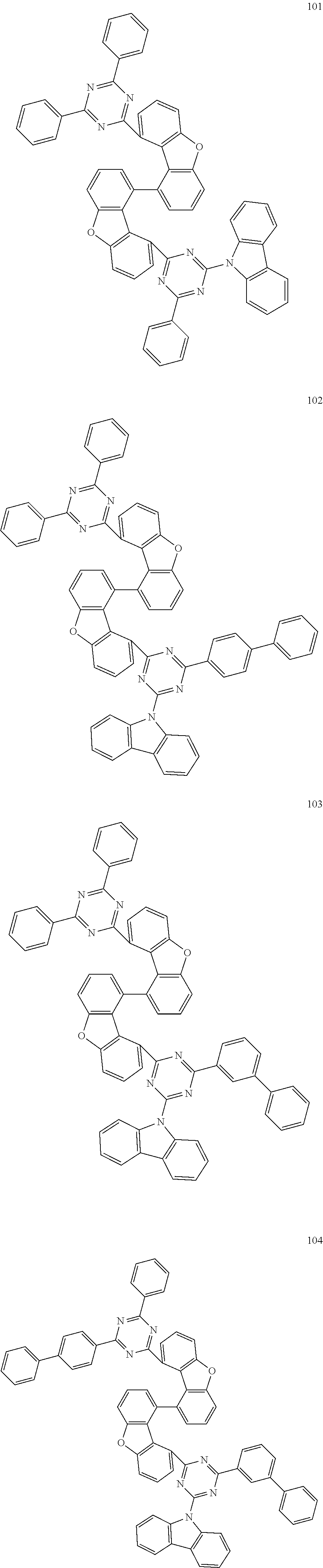

wherein D is deuterium, and TMS is —Si(CH₃)₃.

A composition for an organic optoelectronic device according to some embodiments may include a first compound, and a second compound. The first compound may be the aforementioned compound for an organic optoelectronic device and the second compound may be represented by Chemical Formula 2; a combination of Chemical Formula 3 and Chemical Formula 4; or Chemical Formula 5.

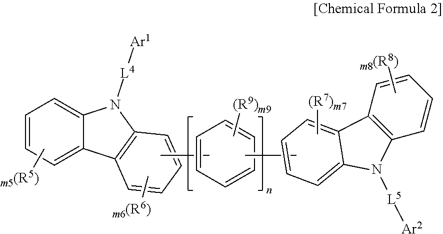

In Chemical Formula 2, R⁵ to R⁹ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar¹ and Ar² may each independently be or include, e.g., a substituted or unsubstituted C6 to C20 aryl group or a substituted or unsubstituted C2 to C30 heterocyclic group.

L⁴ and L⁵ may each independently be or include, e.g., a single bond or a substituted or unsubstituted C6 to C20 arylene group.

m5, m8, and m9 may each independently be, e.g., an integer of 1 to 4.

m6 and m7 may each independently be, e.g., an integer of 1 to 3.

In an implementation, m5 may be 2 to 4, and each R⁵ may be the same or different from each other.

In an implementation, m6 may be 2 or 3, and each R⁶ may be the same or different from each other.

In an implementation, m7 may be 2 or 3, and each R⁷ may be the same or different from each other.

In an implementation, m8 may be 2 to 4, and each R⁸ may be the same or different from each other.

In an implementation, m9 may be 2 to 4, and each R⁹ may be the same or different from each other.

n may be, e.g., an integer of 0 to 2.

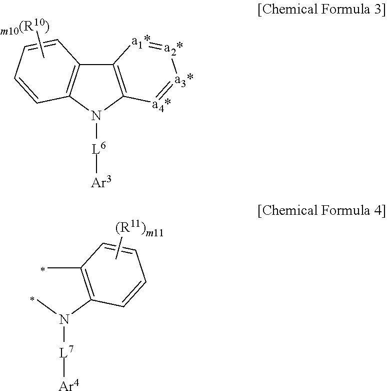

In Chemical Formula 3 and Chemical Formula 4, two adjacent ones of a1* to a4* of Chemical Formula 3 are linking carbons linked at * of Chemical Formula 4. The remaining two of a1* to a4* of Chemical Formula 3, not linked at * of Chemical Formula 4, may each independently be, e.g., C-L^{a}-R^{f}. As used herein, the term “linking carbon” refers to a shared carbon at which fused rings are linked.

L^{a}, L⁶, and L⁷ may each independently be or include, e.g., a single bond or a substituted or unsubstituted C6 to C20 arylene group.

R^{f}, R¹⁰, and R¹¹ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar³ and Ar⁴ may each independently be, e.g., a substituted or unsubstituted C6 to C20 aryl group or a substituted or unsubstituted C2 to C30 heterocyclic group.

m10 and m11 may each independently be, e.g., an integer of 1 to 4,

In an implementation, m10 may be 2 to 4, and each R¹⁰ may be the same or different from each other.

In an implementation, m11 may be 2 to 4, and each R¹¹ may be the same or different from each other.

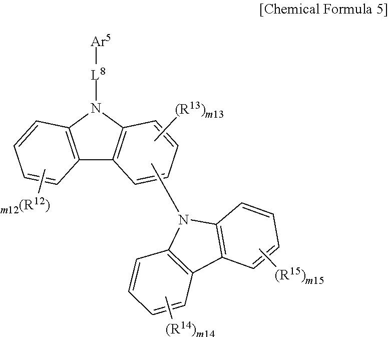

In Chemical Formula 5, L⁸ may be, e.g., a single bond or a substituted or unsubstituted C6 to C20 arylene group.

R¹² to R¹⁵ may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar⁵ may be, e.g., a substituted or unsubstituted C6 to C20 aryl group or a substituted or unsubstituted C2 to C30 heterocyclic group.

m12, m14 and m15 may each independently be, e.g., an integer of 1 to 4.

m13 may be, e.g., an integer of 1 to 3.

In an implementation, m12 may be 2 to 4, and each R¹² may be the same or different from each other.

In an implementation, m13 may be 2 or 3, and each R¹³ may be the same or different from each other.

In an implementation, m14 may be 2 to 4, and each R¹⁴ may be the same or different from each other.

In an implementation, m15 may be 2 to 4, and each R¹⁵ may be the same or different from each other.

The second compound may be included in the light emitting layer together with the first compound to improve luminous efficiency and life-span characteristics by increasing charge mobility and stability.

In an implementation, in Chemical Formula 2, Ar¹ and Ar² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, in Chemical Formula 2, L⁴ and L⁵ may each independently be, e.g., a single bond, a substituted or unsubstituted phenylene group, or a substituted or unsubstituted biphenylene group.

In an implementation, in Chemical Formula 2, R⁵ to R⁹ may each independently be, e.g., hydrogen, deuterium, or a substituted or unsubstituted C6 to C12 aryl group.

n may be 0 or 1.

In Chemical Formula 2, “substituted” may refer to replacement of at least one hydrogen by deuterium, a C1 to C4 alkyl group, a C6 to C18 aryl group, or a C2 to C30 heteroaryl group.

In an implementation, in Chemical Formula 2, Ar¹ and Ar² may each independently be, e.g., a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, Chemical Formula 2 may be represented by one of Chemical Formula 2-1 to Chemical Formula 2-15.

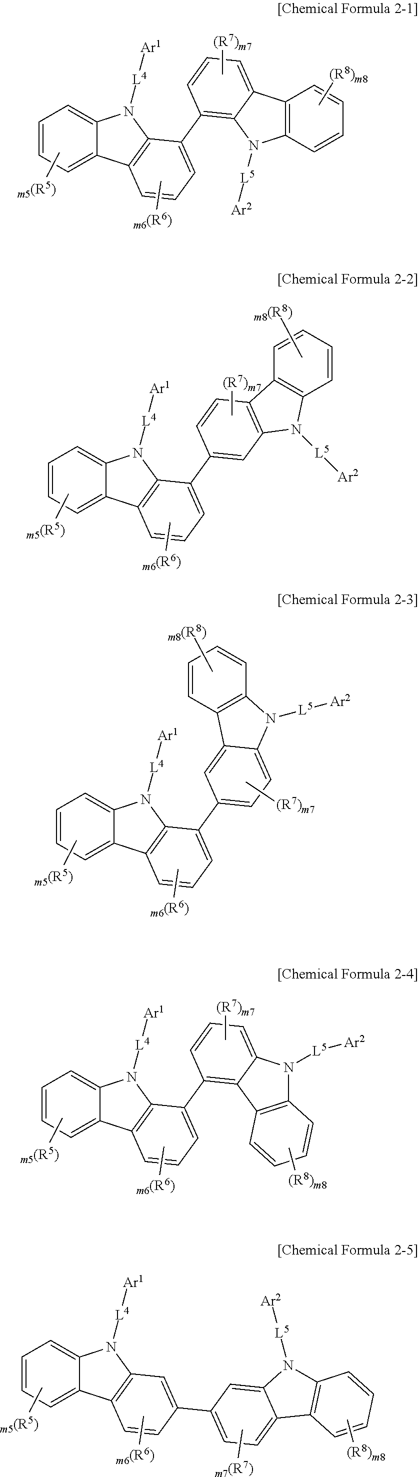

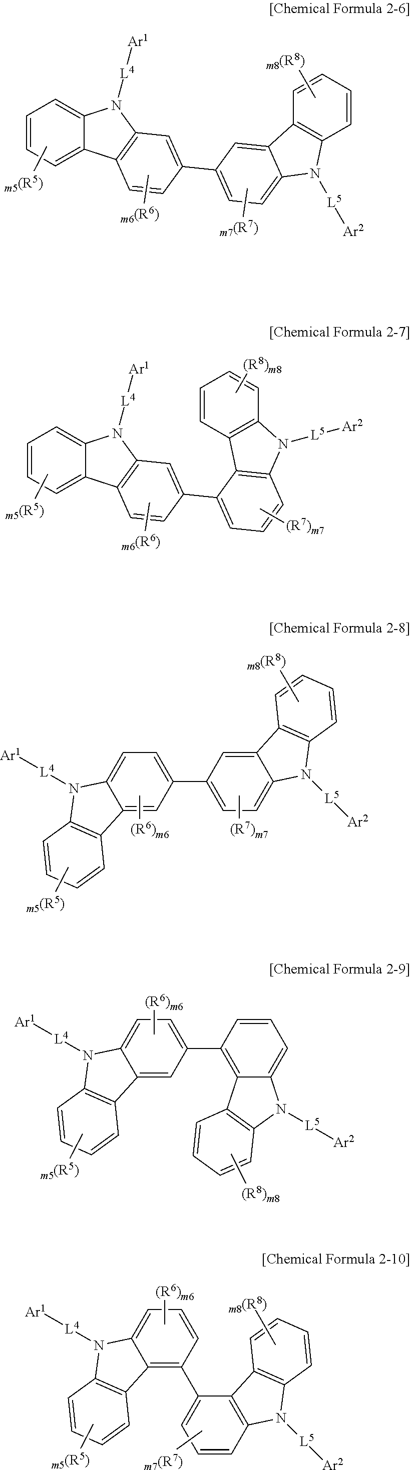

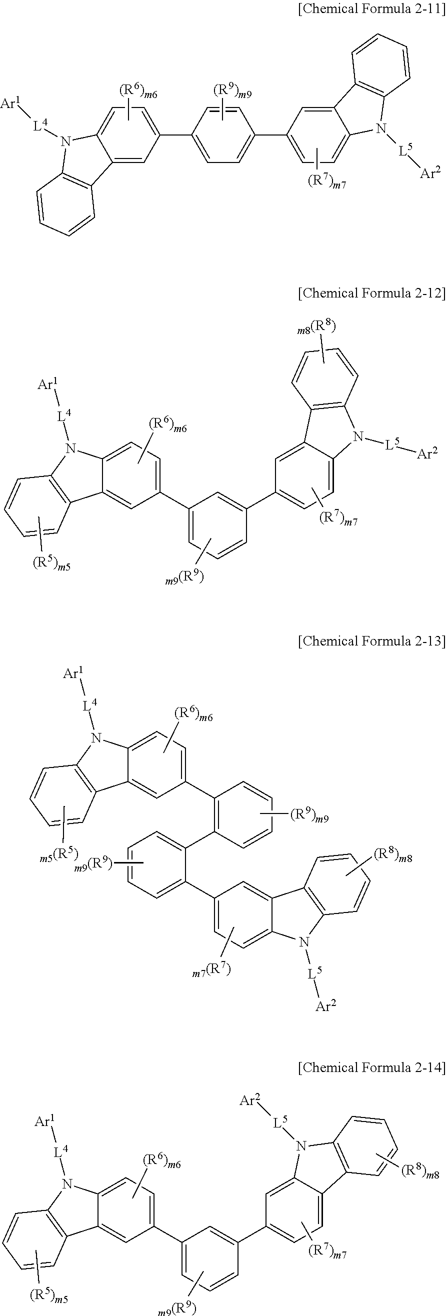

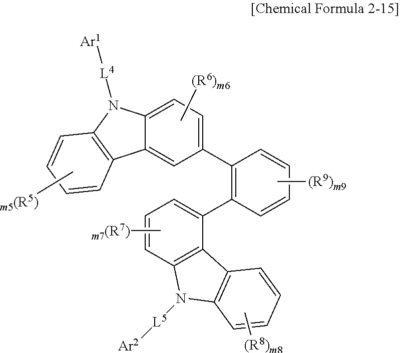

In Chemical Formulae 2-1 to Chemical Formula 2-15, R⁵ to R⁹ may each independently be, e.g., hydrogen, deuterium, or a substituted or unsubstituted C6 to C12 aryl group, and moieties -L⁴-Ar¹ and -L⁵-Ar² may each independently be, e.g., a moiety of Group I.

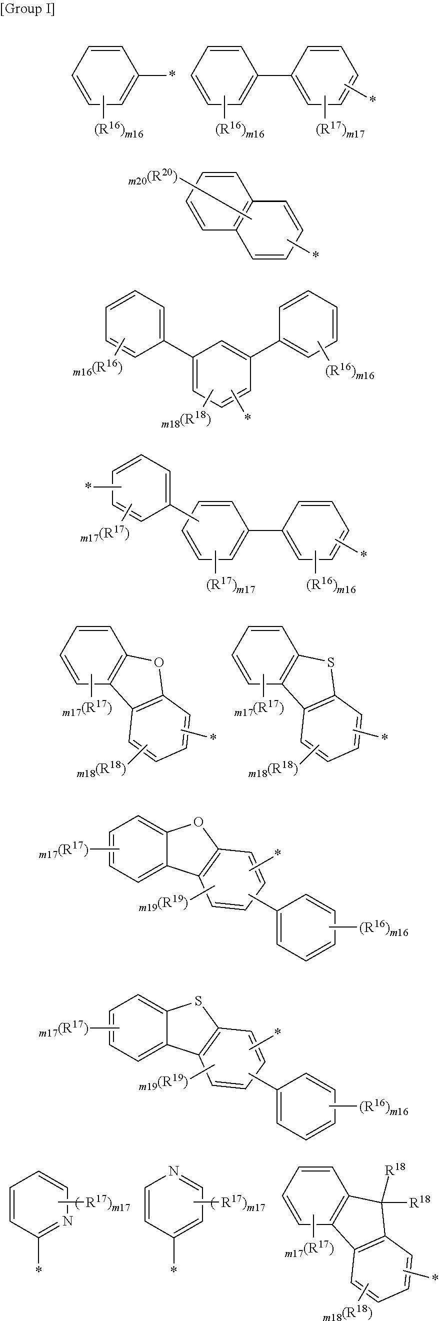

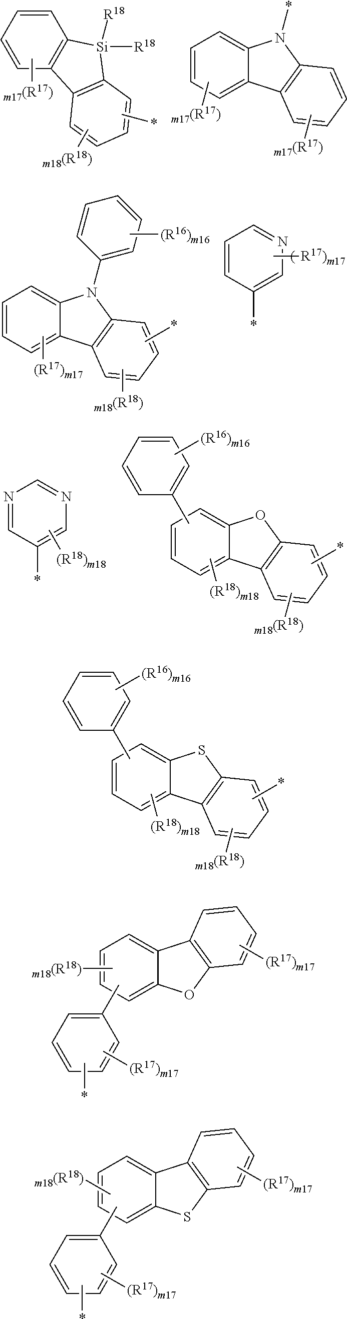

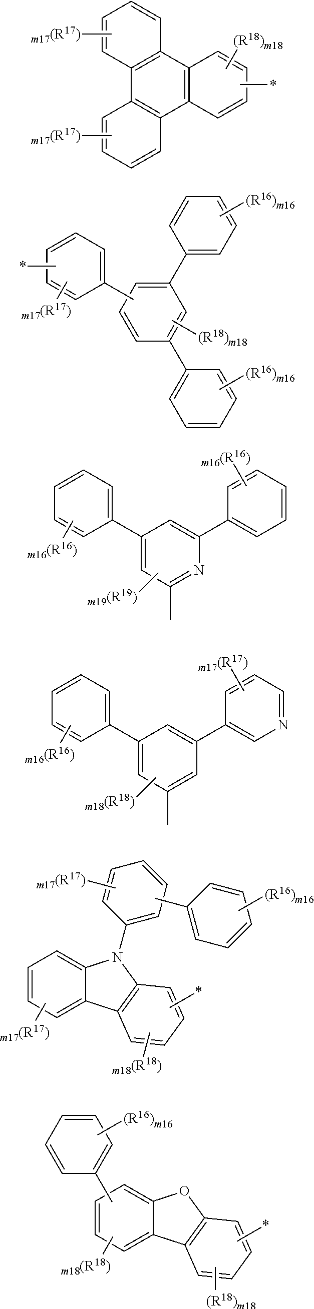

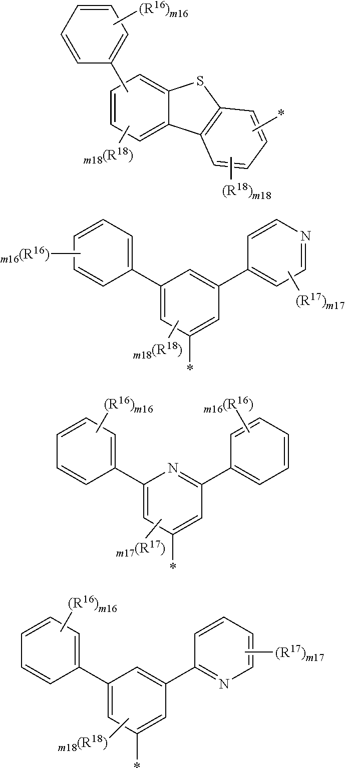

In Group I, R¹⁶ to R²⁰ may each independently be, e.g., hydrogen, deuterium, a cyano group, a substituted or unsubstituted C1 to C10 alkyl group, or a substituted or unsubstituted C6 to C12 aryl group.

m16 may be, e.g., an integer of 1 to 5.

m17 may be, e.g., an integer of 1 to 4.

m18 may be, e.g., an integer of 1 to 3.

m19 may be, e.g., an integer of 1 or 2.

m20 may be, e.g., an integer of 1 to 7.

* is a linking point.

In an implementation, m16 may be 2 to 5, and each R¹⁶ may be the same or different from each other.

In an implementation, m17 may be 2 to 4, and each R¹⁷ may be the same or different from each other.

In an implementation, m18 may be 2 or 3, and each R¹⁸ may be the same or different from each other.

In an implementation, m19 may be 2, and each R¹⁹ may be the same or different from each other.

In an implementation, m20 may be 2 to 7, and each R²⁰ may be the same or different from each other.

The combination of Chemical Formula 3 and Chemical Formula 4 may be represented, e.g., by one of Chemical Formula 3A, Chemical Formula 3B, Chemical Formula 3C, Chemical Formula 3D, and Chemical Formula 3E.

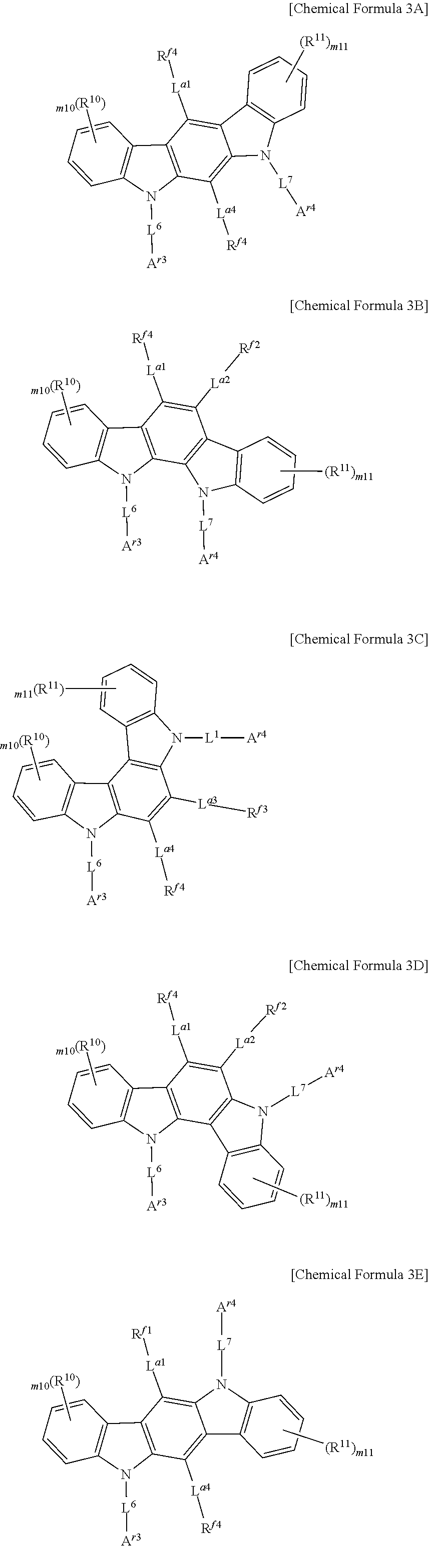

In Chemical Formula 3A to Chemical Formula 3E, L⁶, L⁷, Ar³, Ar⁴, R¹⁰, R¹¹, m10, and m11 may be defined the same as described above.

L^{a1} to L^{a4} may be defined the same as L⁶ and L⁷ described above.

R^{f1} to R^{f4} may be defined the same as R¹⁰ and R¹¹ described above.

In an implementation, in Chemical Formula 3 and Chemical Formula 4, Ar³ and Ar⁴ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

R^{f1} to R^{f4}, R¹⁰, and R¹¹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formulas 3 and 4, moieties -L⁶-Ar³ and -L⁷-Ar⁴ may each independently be a moiety of Group I.

In an implementation, R^{f1} to R^{f4}, R¹⁰, and R¹¹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{f1} to R^{f4}, R¹⁰ and R¹¹ may each independently be, e.g., hydrogen, deuterium, a cyano group, or a substituted or unsubstituted phenyl group.

In an implementation, R^{f1} to R^{f4}, R¹⁰ and R¹¹ may each independently be, e.g., hydrogen, deuterium or a substituted or unsubstituted phenyl group.

In an implementation, the second compound may be represented by Chemical Formula 2-8, and in Chemical Formula 2-8, Ar¹ and Ar² may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L⁴ and L⁵ may each independently be, e.g., a single bond, or a substituted or unsubstituted C6 to C20 arylene group, and R⁵ to R⁸ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 2-8, R⁵ to R⁸ may each independently be, e.g., hydrogen, deuterium, or a substituted or unsubstituted C6 to C12 aryl group, and moieties -L⁴-Ar¹ and -L⁵-Ar² may each independently be, e.g., a moiety of Group I.

In an implementation, the second compound may be represented by, e.g., Chemical Formula 3C, and in Chemical Formula 3C, L^{a3} and L^{a4} may each be, e.g., a single bond, L⁶ and L⁷ may each independently be, e.g., a single bond or a substituted or unsubstituted C6 to C12 arylene group, R¹⁰, R¹¹, R^{f3}, and R^{f4} may each be, e.g., hydrogen, deuterium or phenyl group, and Ar³ and Ar⁴ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 3C, L^{a3} and L^{a4} may be, e.g., a single bond, R¹⁰, R¹¹, R^{f3}, and R^{f4} may each independently be, e.g., hydrogen, deuterium, or a C6 to C12 aryl group, and moieties -L⁶-Ar³ and -L⁷-Ar⁴ may each independently be, e.g., a moiety of Group I.

Chemical Formula 5 may be, e.g., represented by one of Chemical Formula 5-1 to Chemical Formula 5-4.

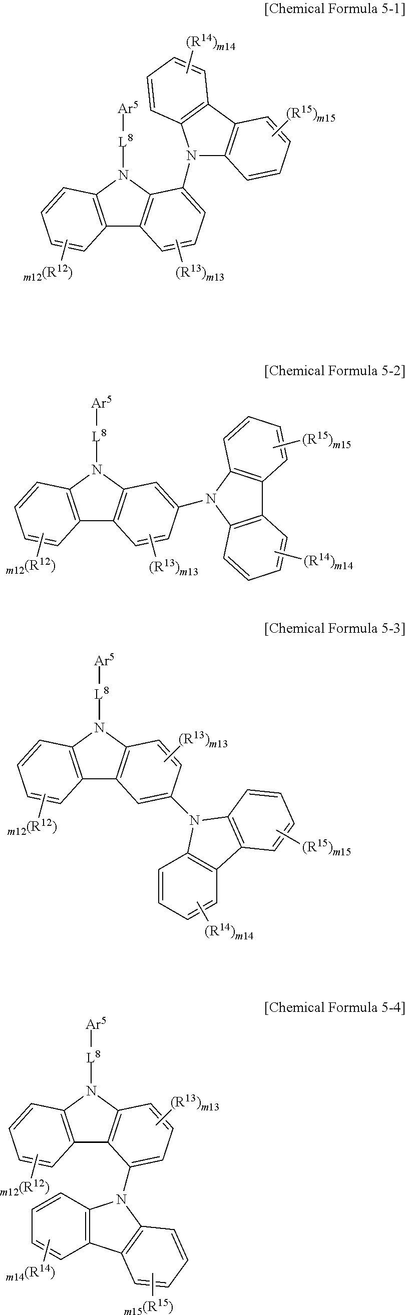

In Chemical Formula 5-1 to Chemical Formula 5-4, L⁸, Ar⁵, R¹² to R¹⁵, and m12 to m15 may be defined the same as described above.

In an implementation, in Chemical Formula 5, Ar⁵ may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

R¹² to R¹⁵ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 5, moiety-L⁸-Ar⁵ may be a moiety of Group I.

In an implementation, R¹² to R¹⁵ may each independently be, e.g., hydrogen, deuterium, a cyano group, or a substituted or unsubstituted phenyl group.

In an implementation, the second compound for an organic optoelectronic device may be, e.g., a compound of Group 2.

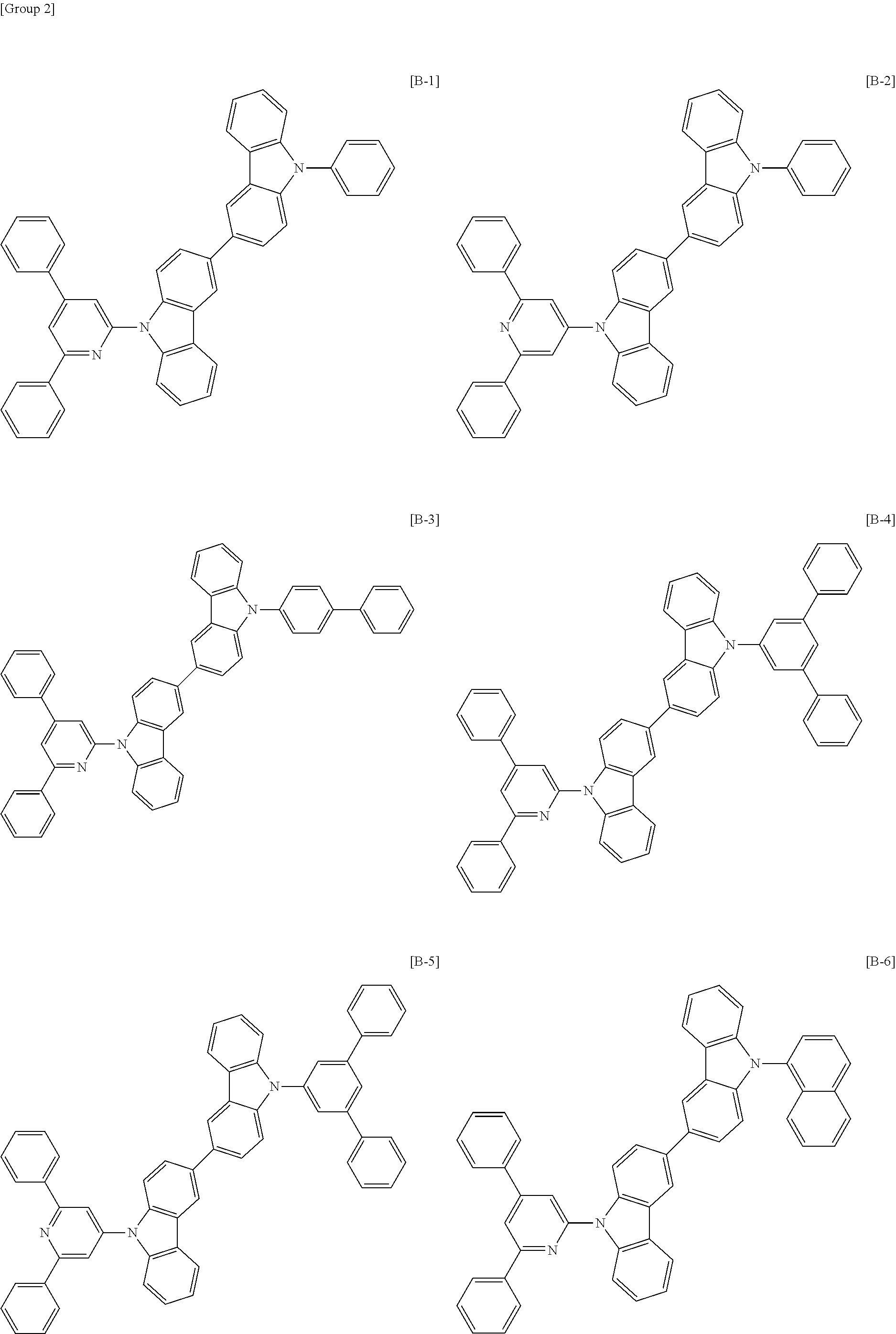

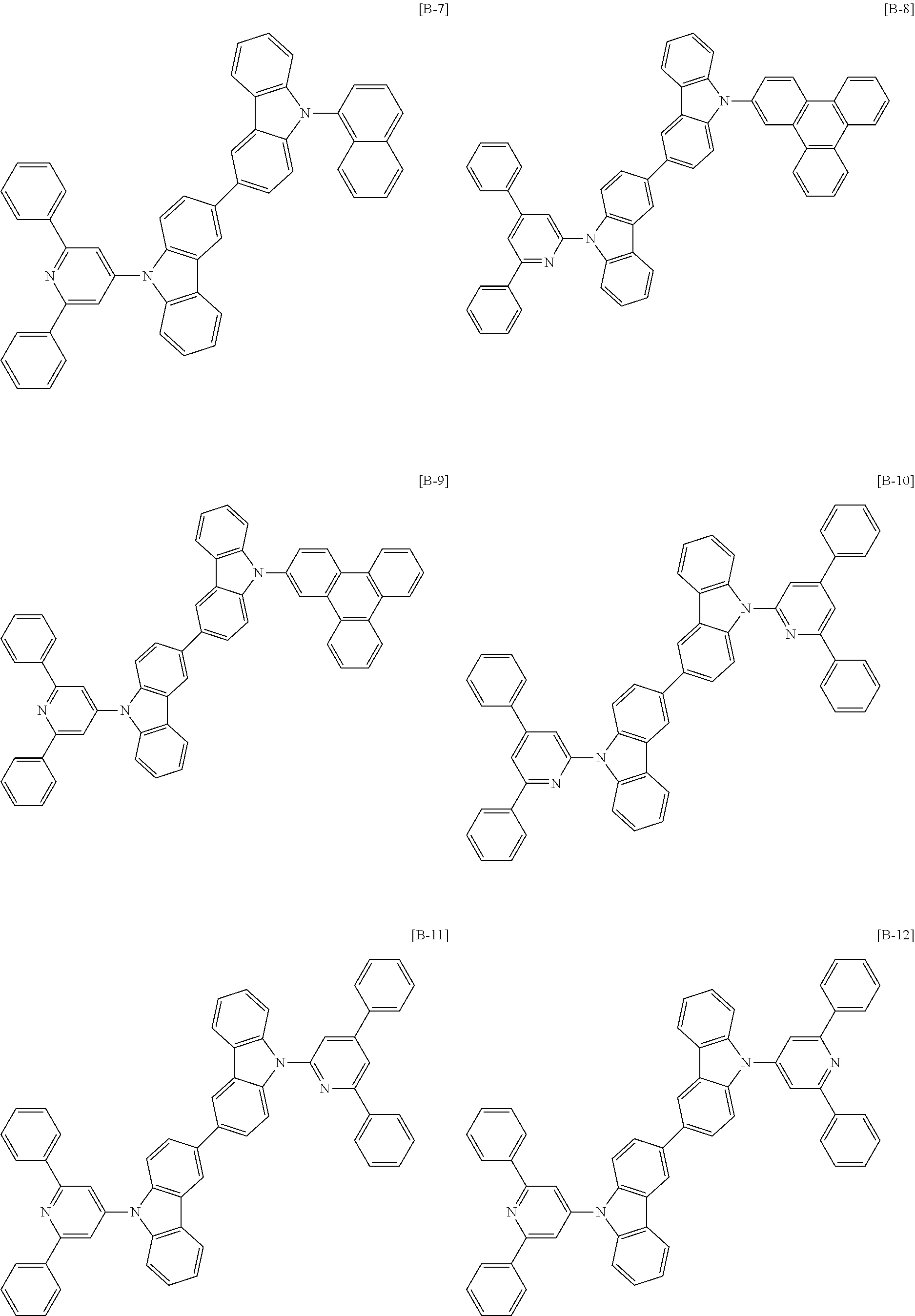

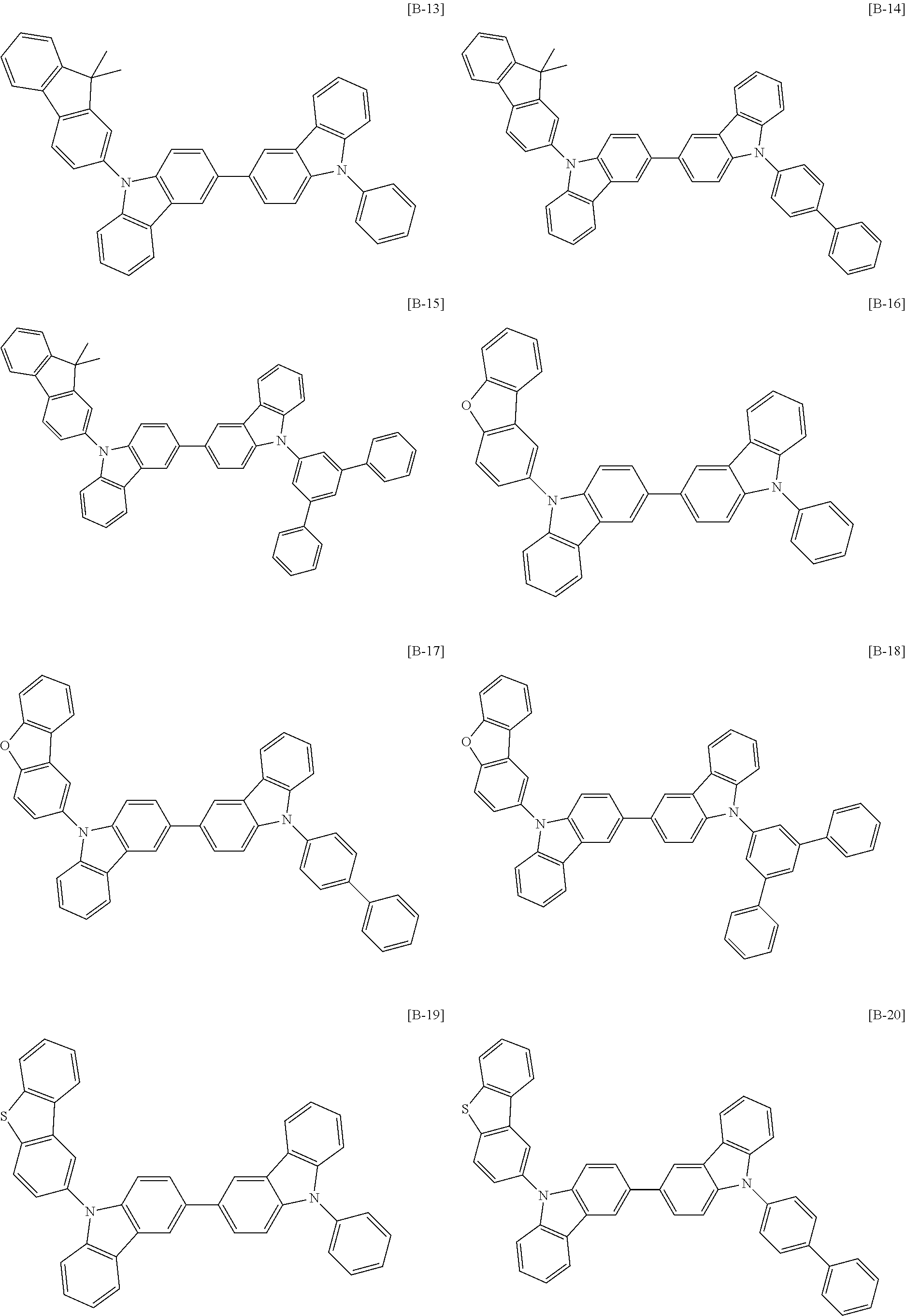

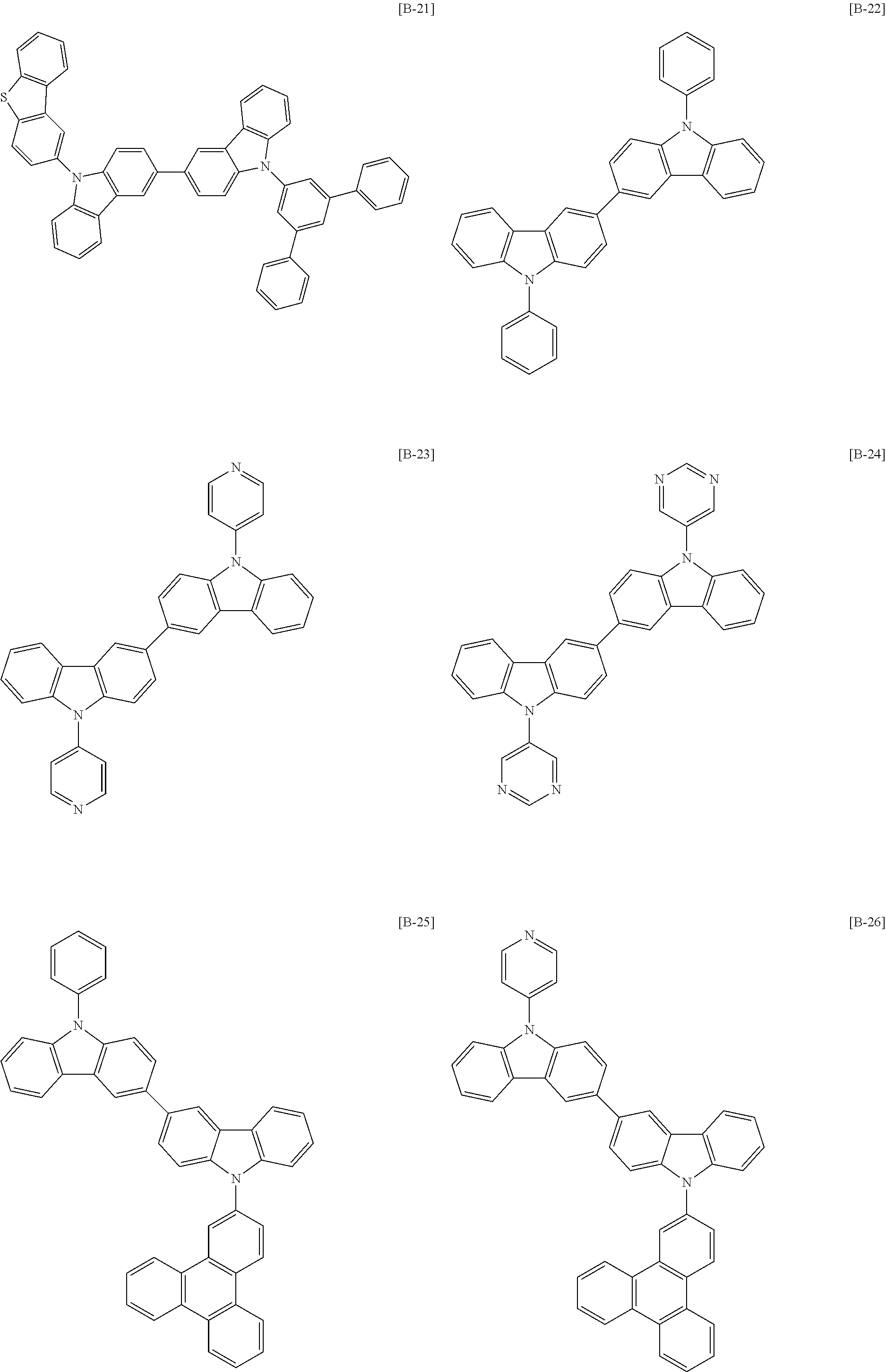 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21]

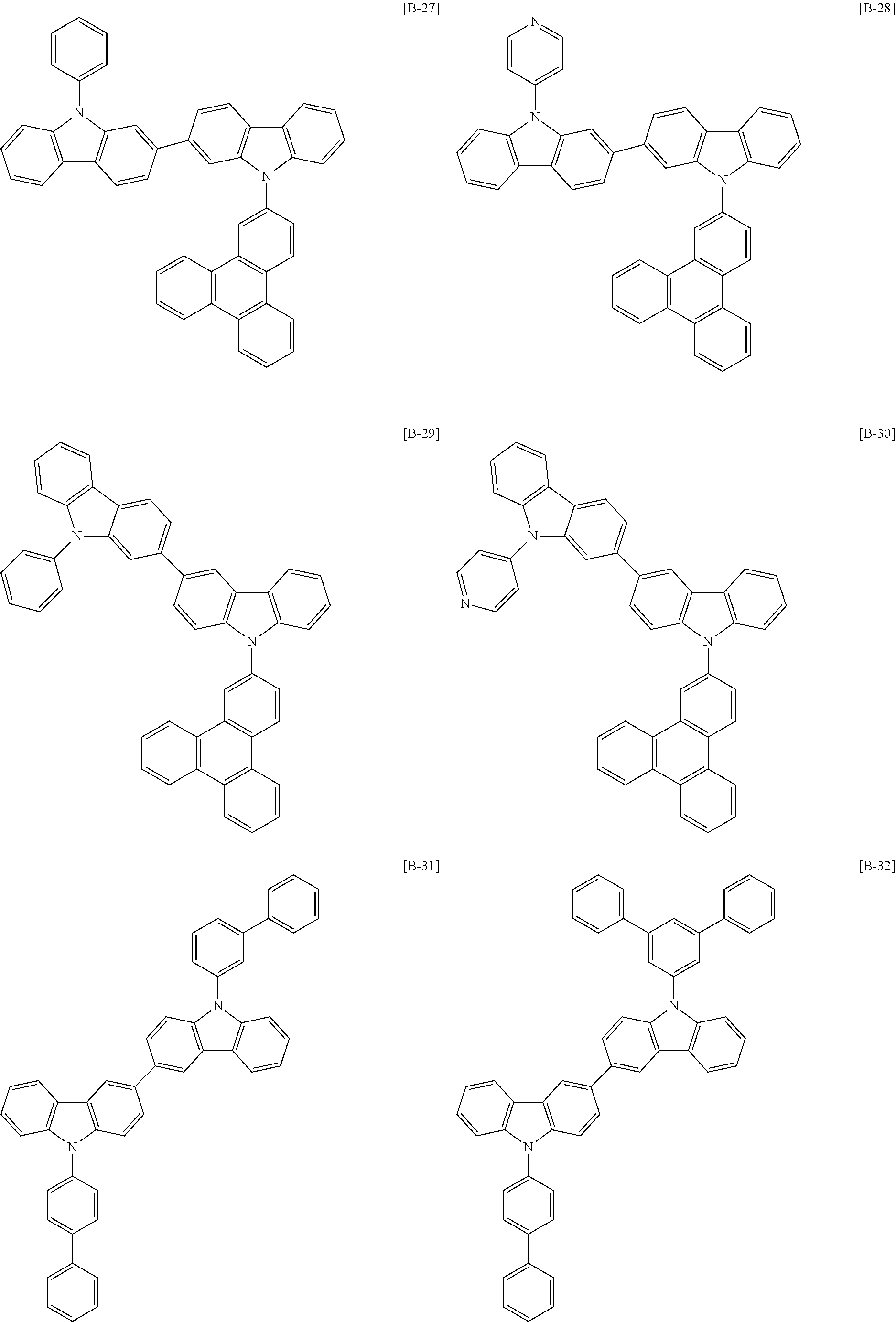

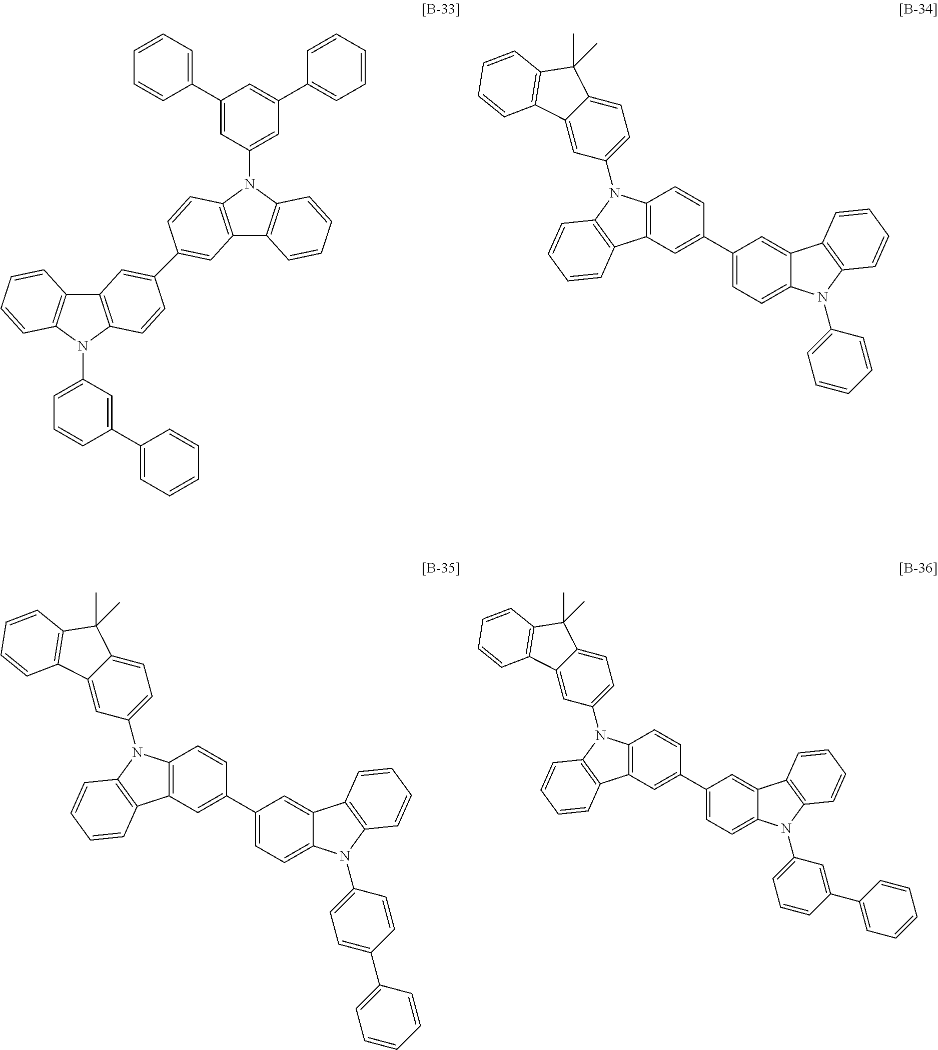 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21 ; CC1(C)c2ccccc2-c2cc(-n3c4ccccc4c4cc(-c5ccc6c(c5)c5ccccc5n6-c5cccc(-c6ccccc6)c5)ccc43)ccc21]

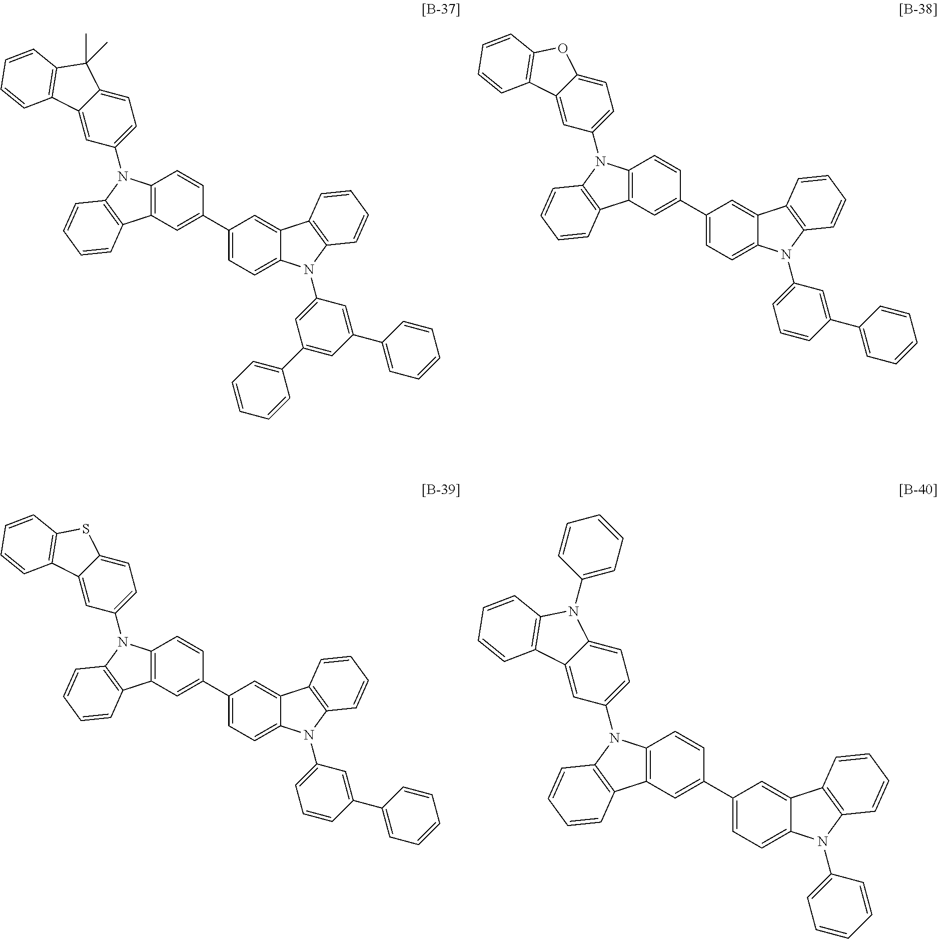 [SMILES: CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1]

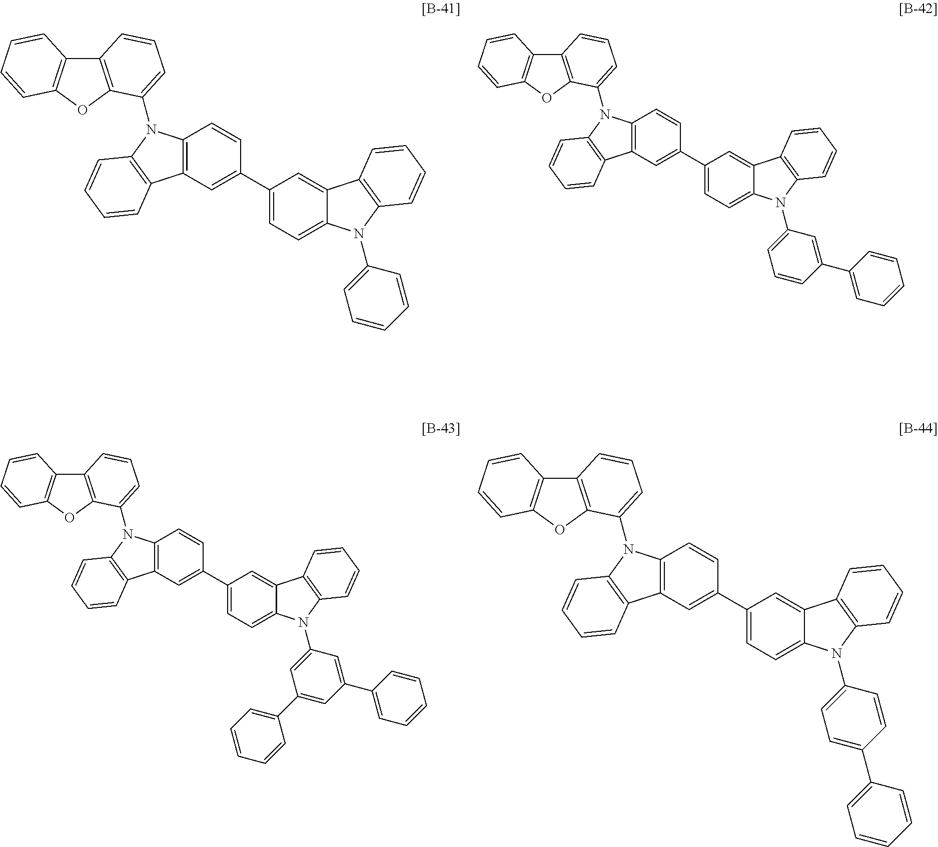 [SMILES: c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5cccc6c5oc5ccccc56)ccc43)cc2)cc1]

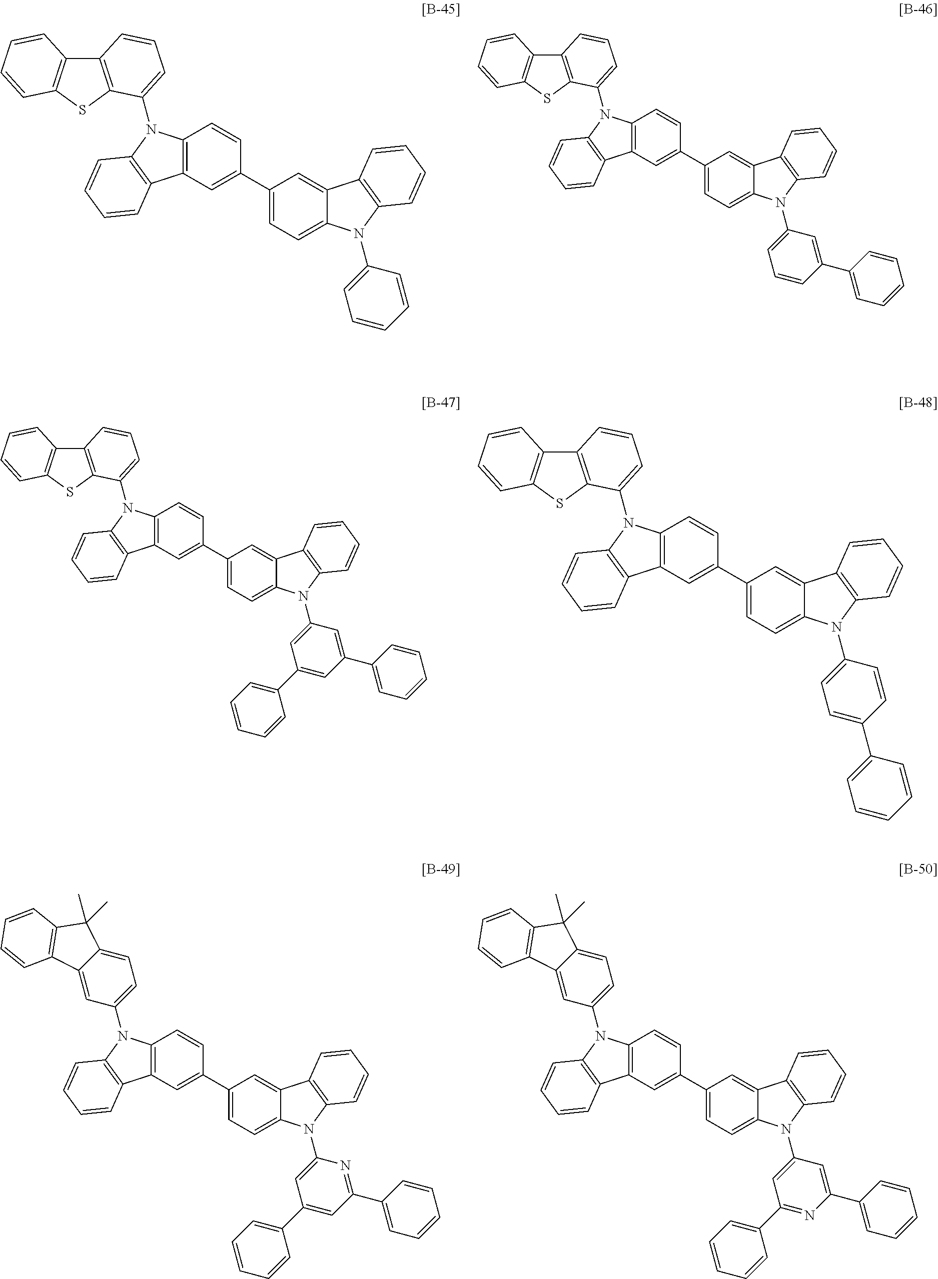

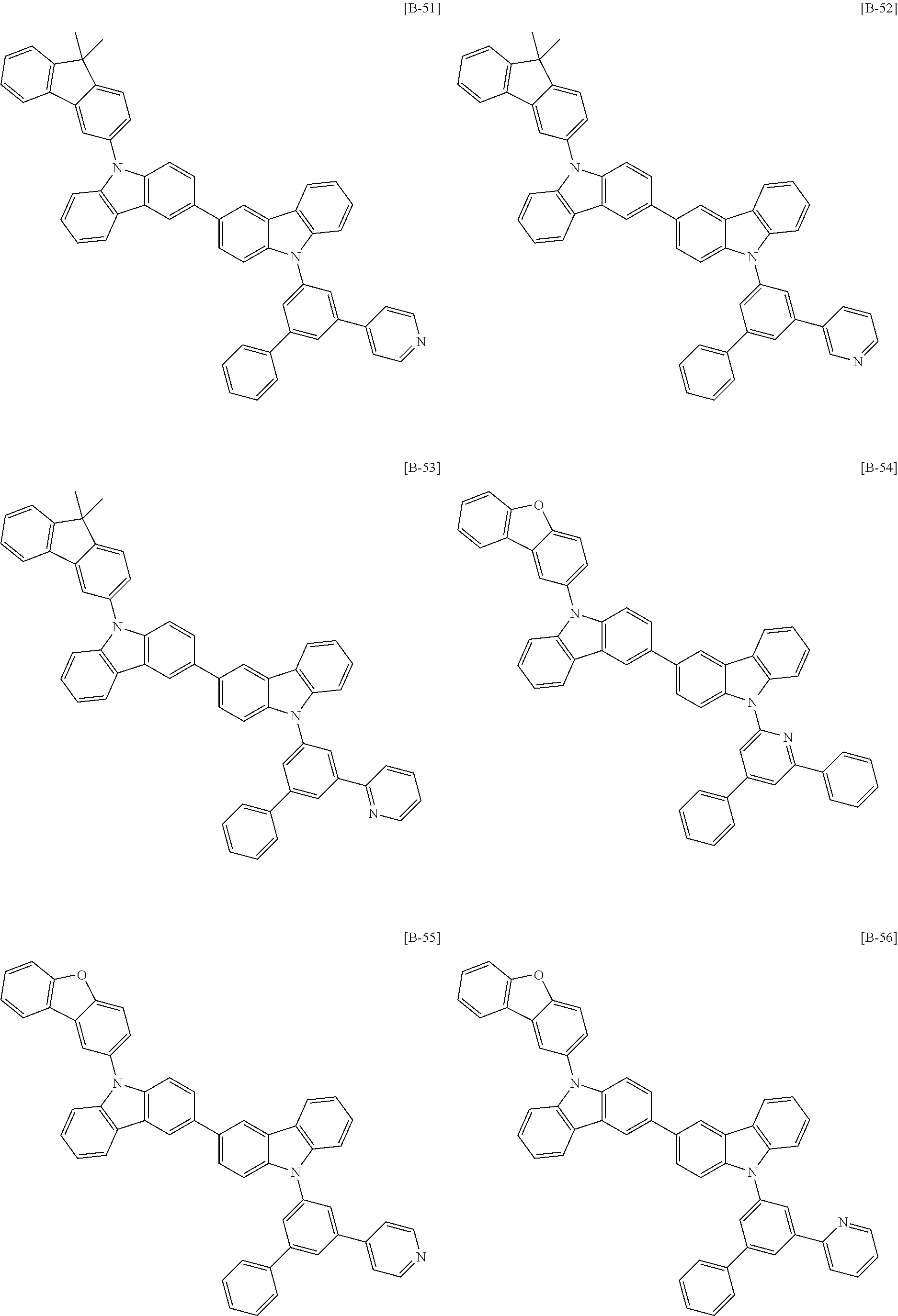

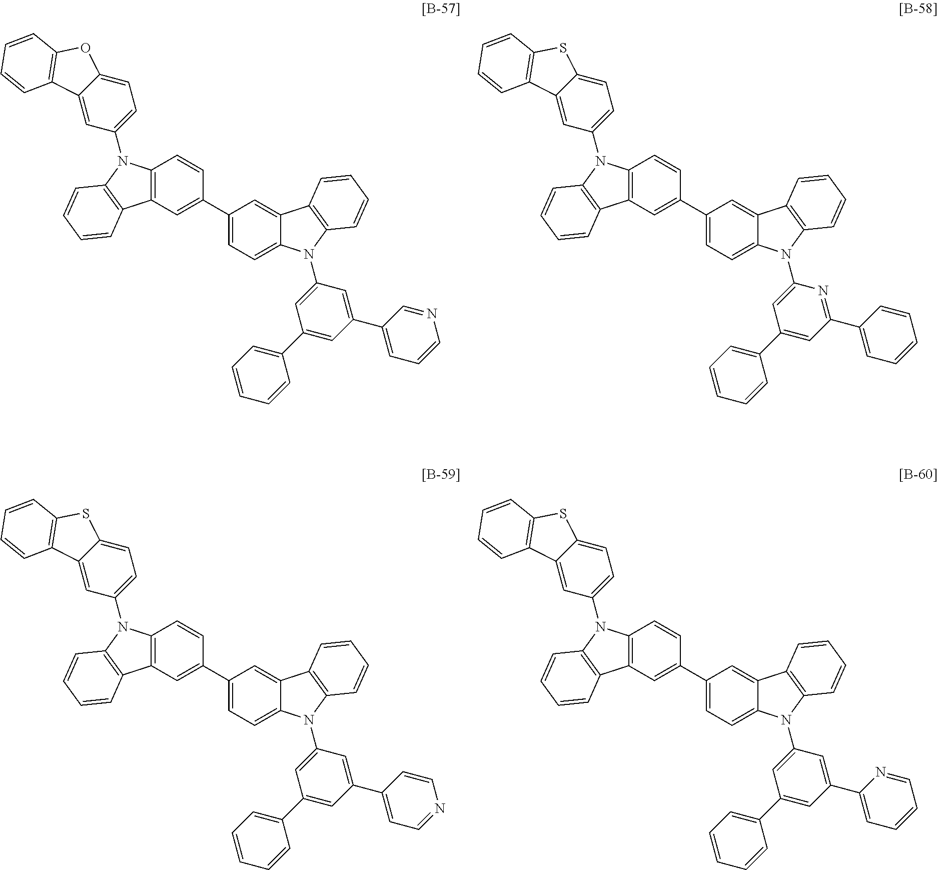 [SMILES: c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

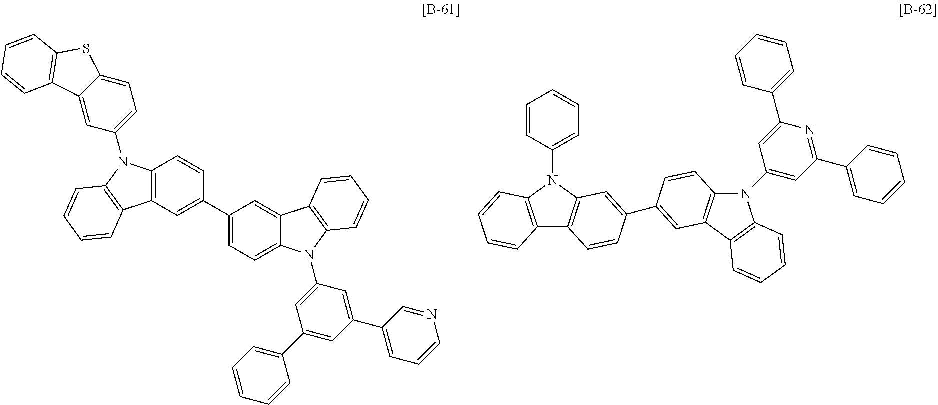 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1]

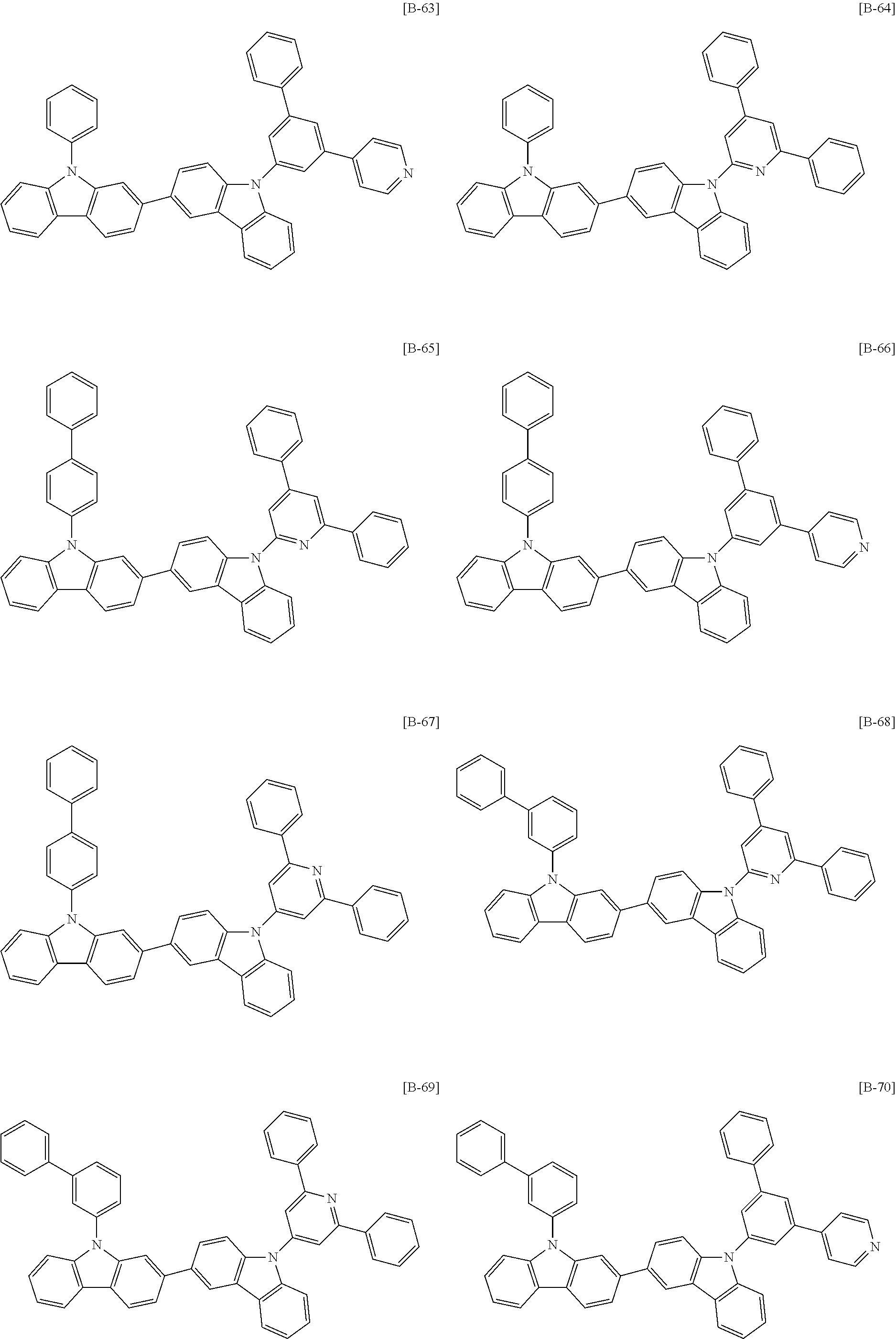

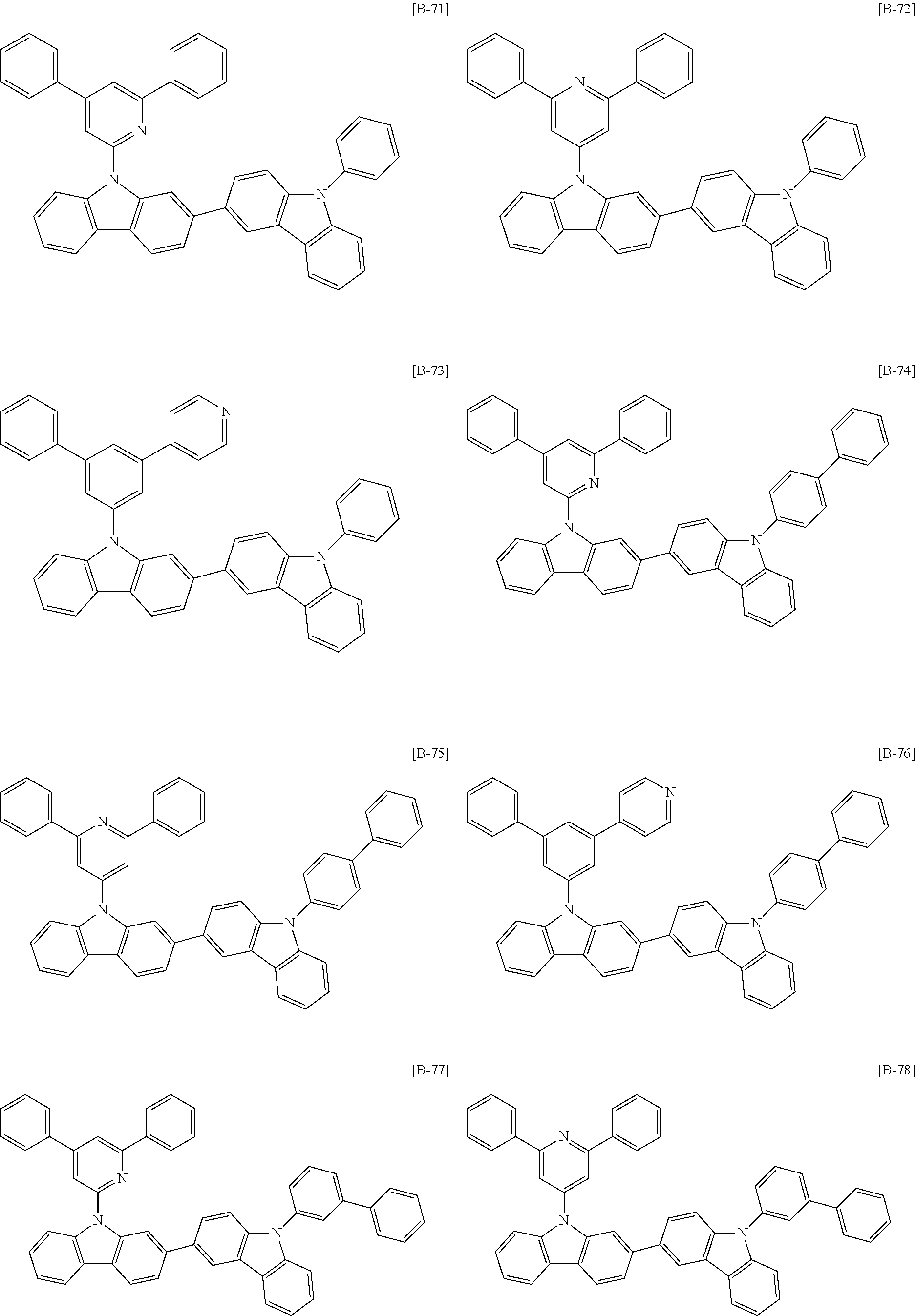

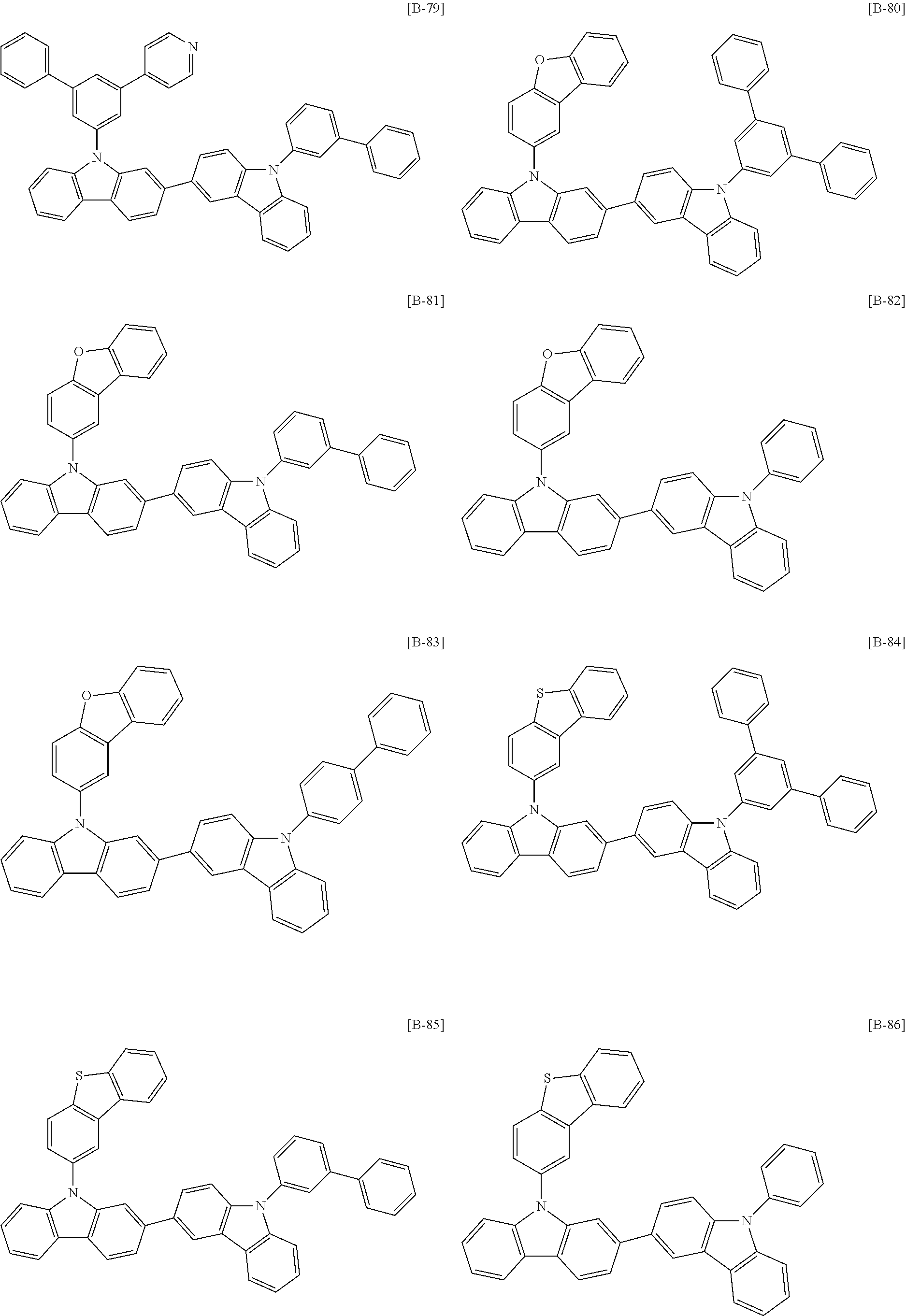

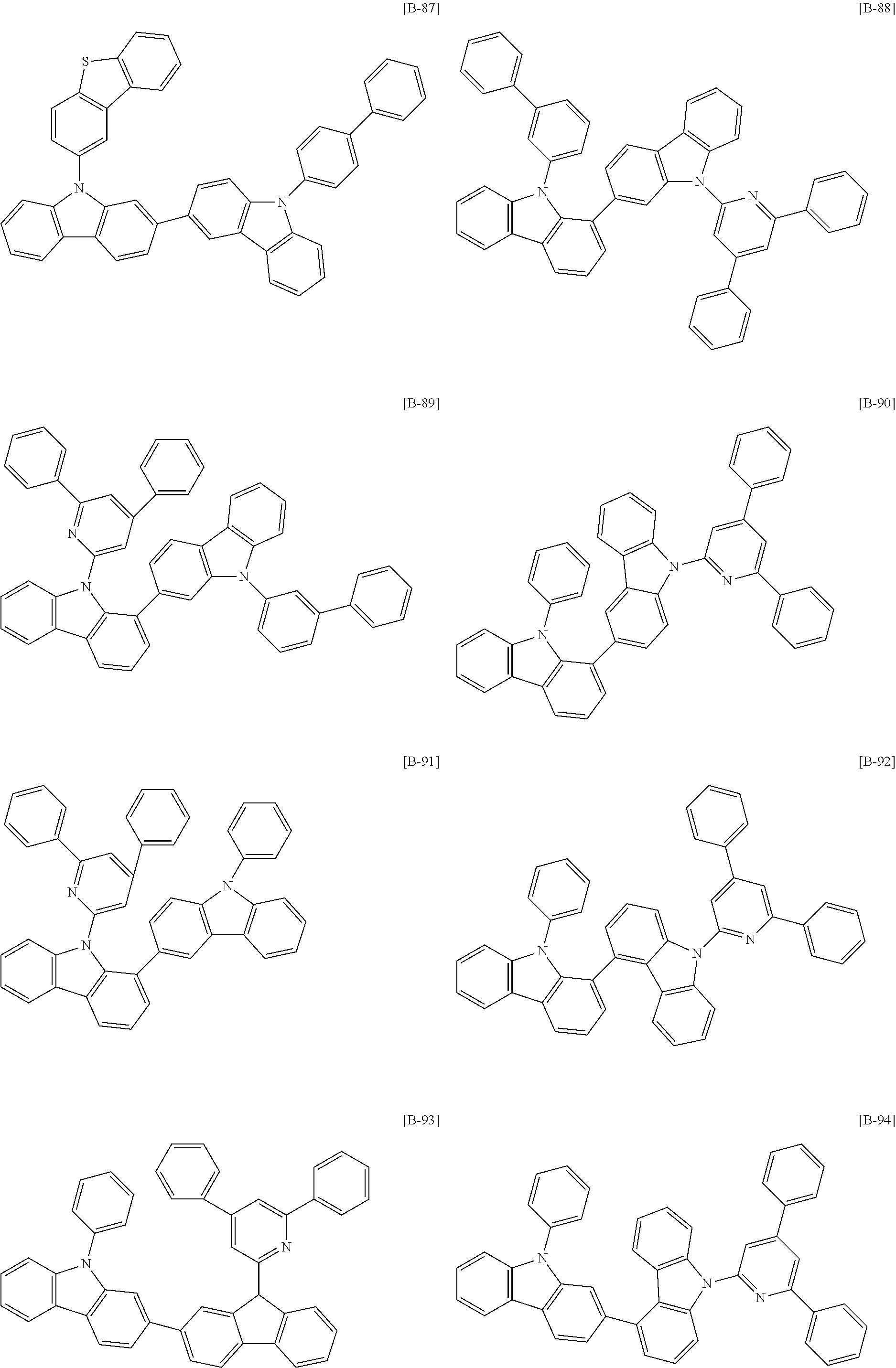

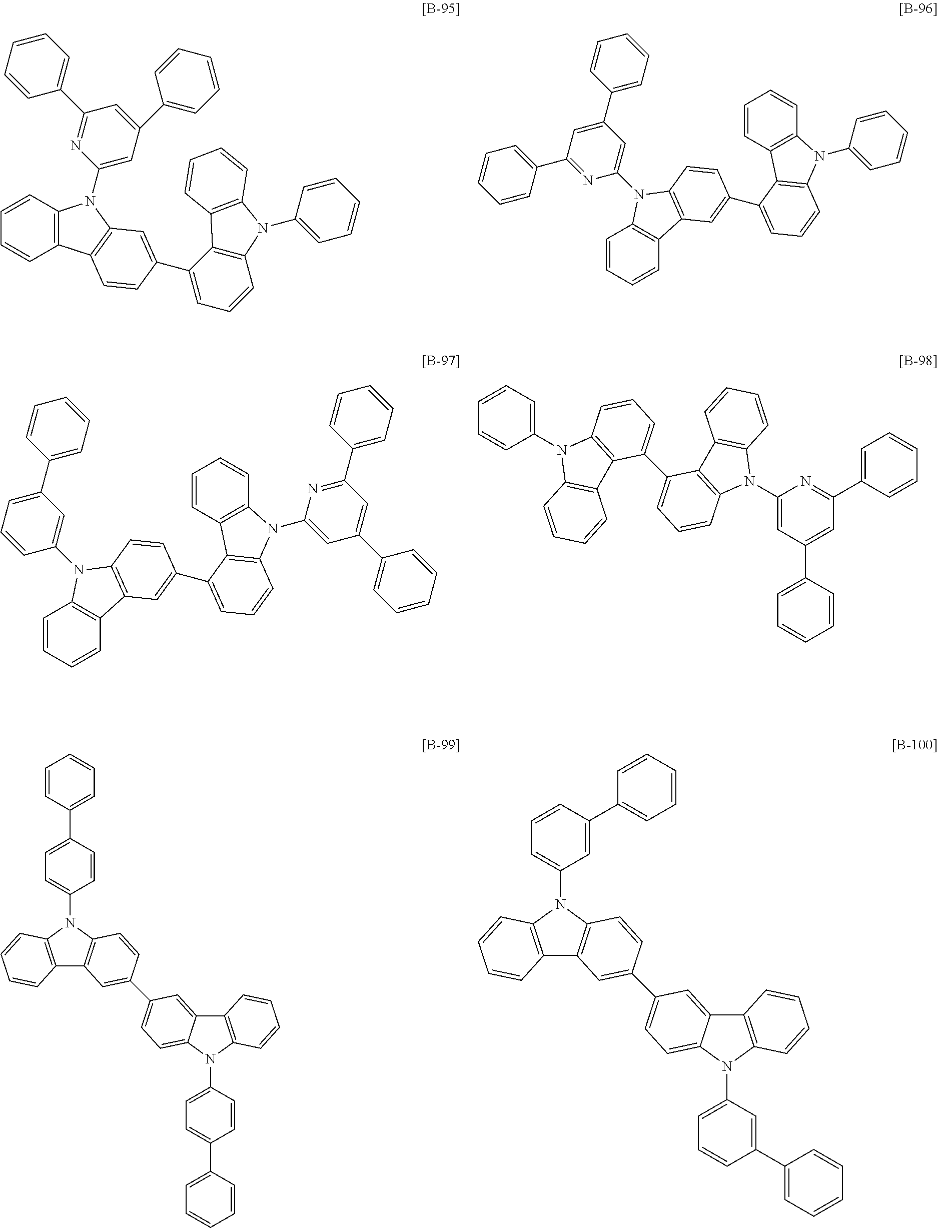

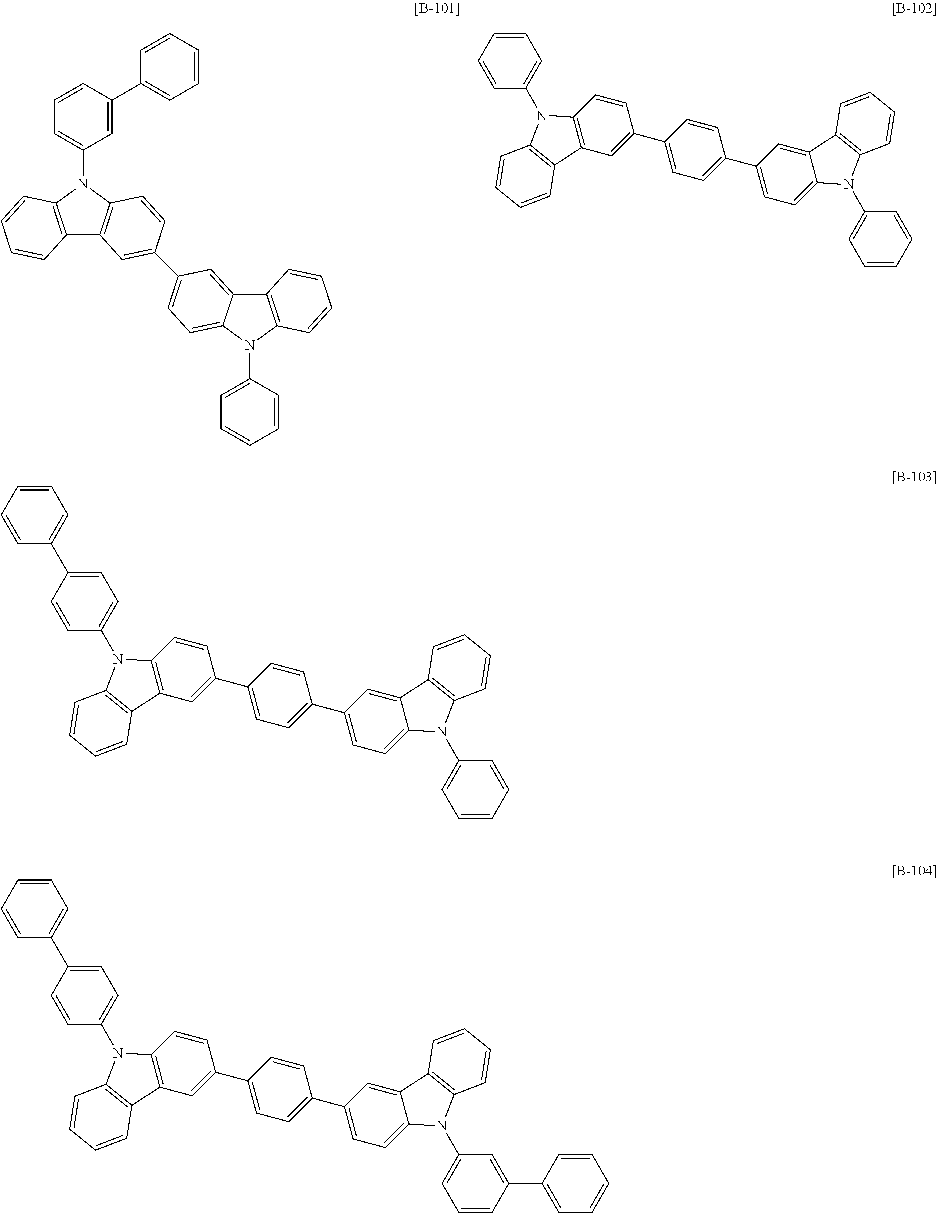 [SMILES: c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1]

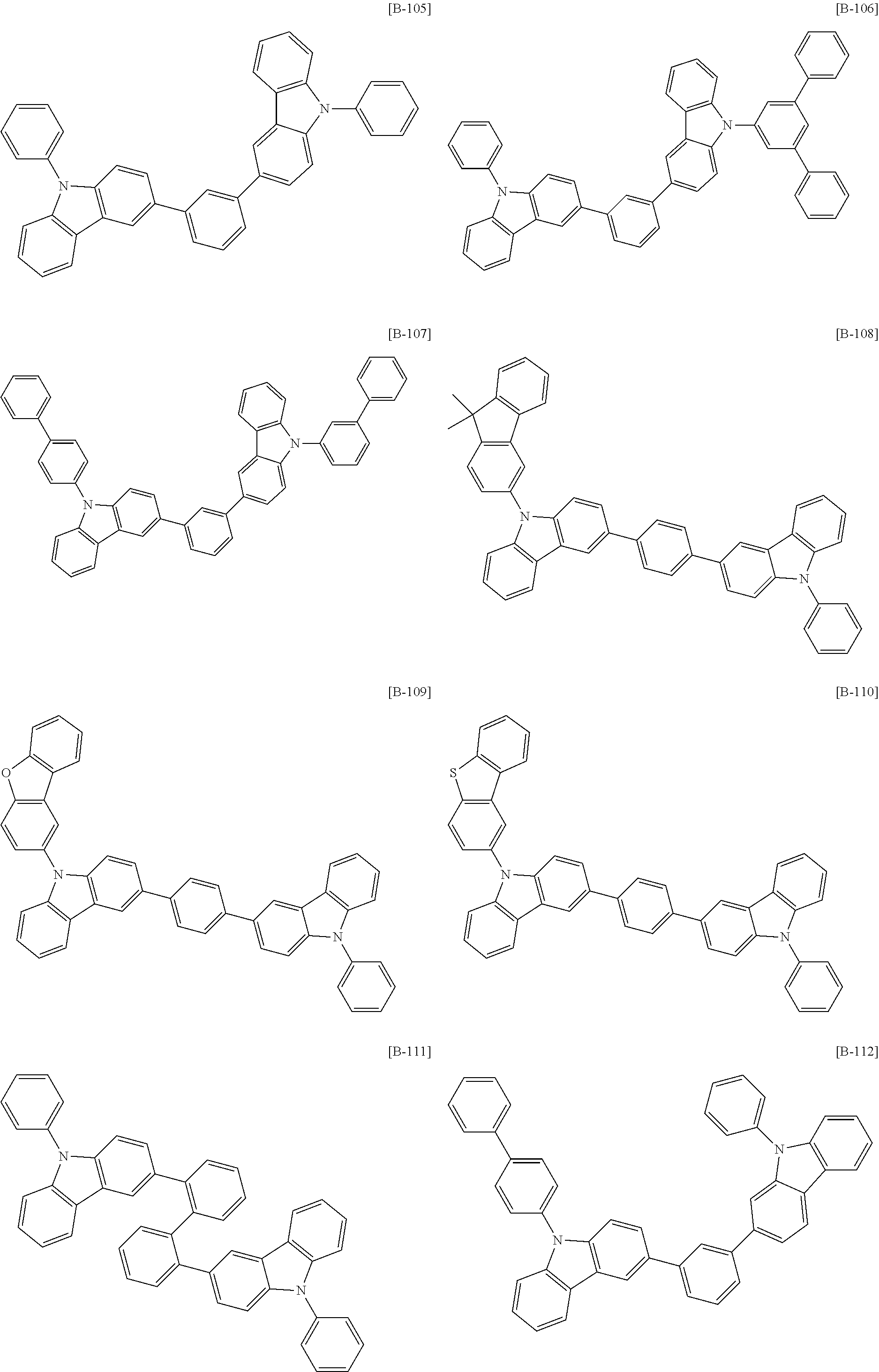

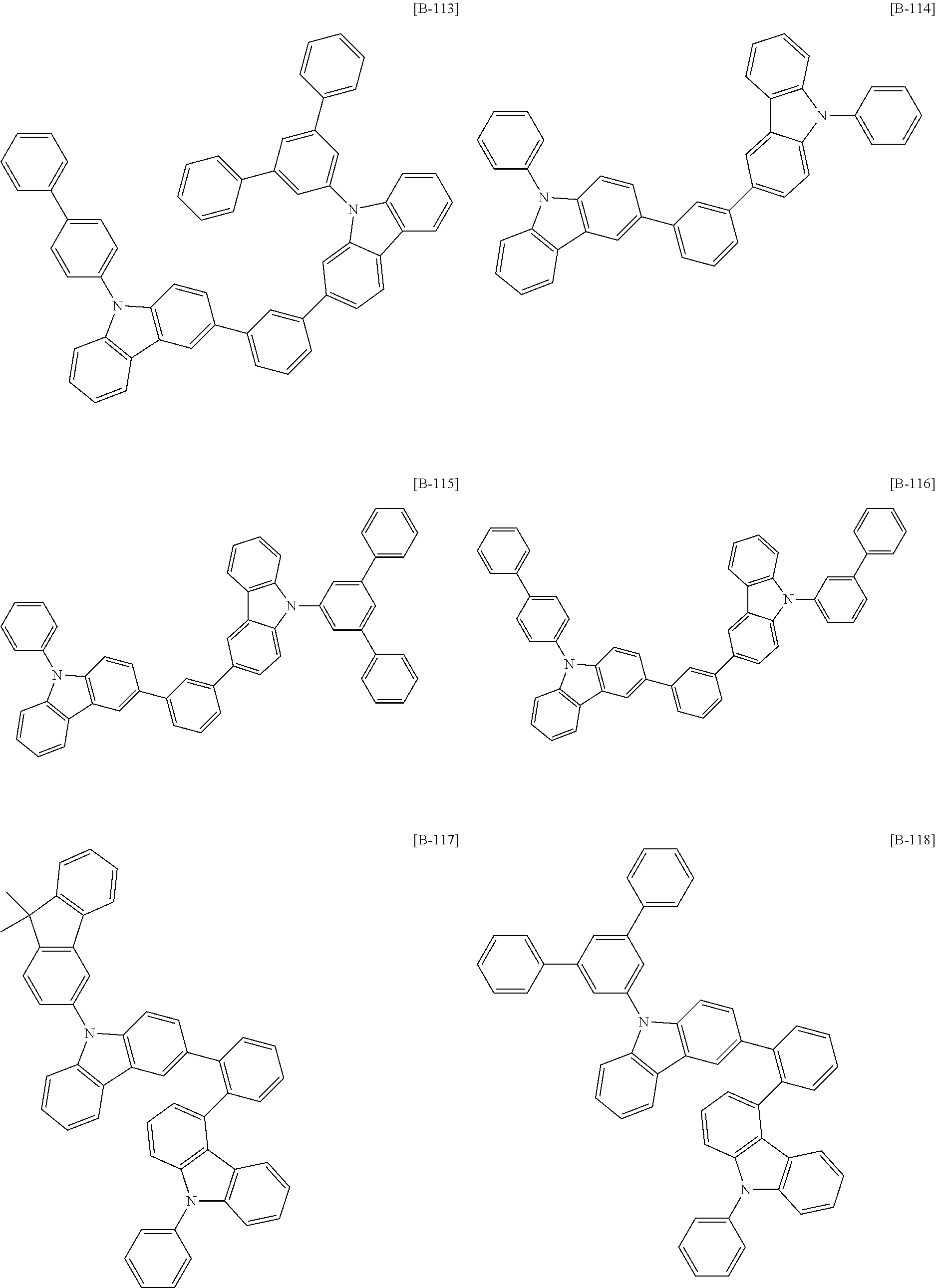

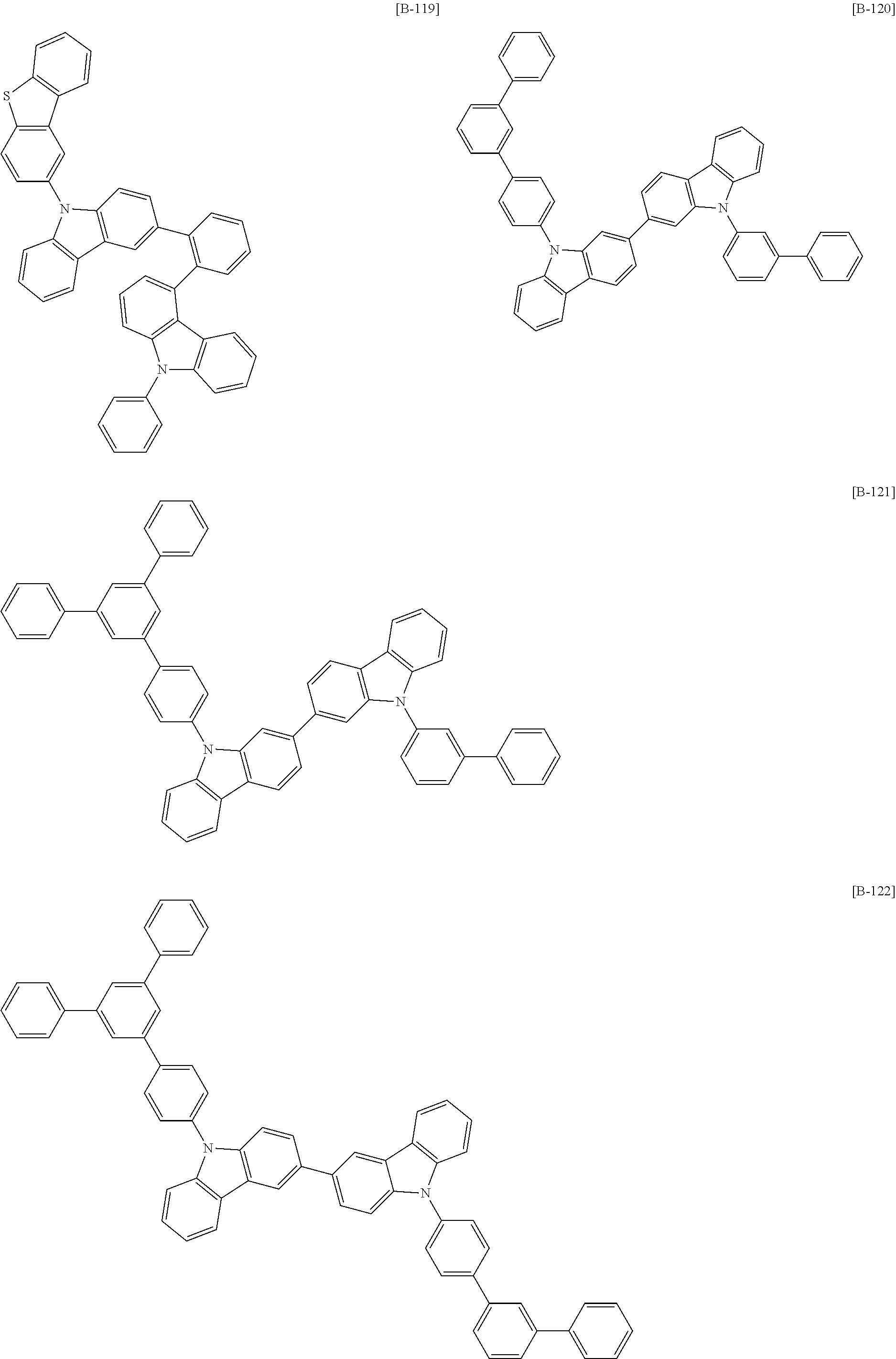 [SMILES: c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

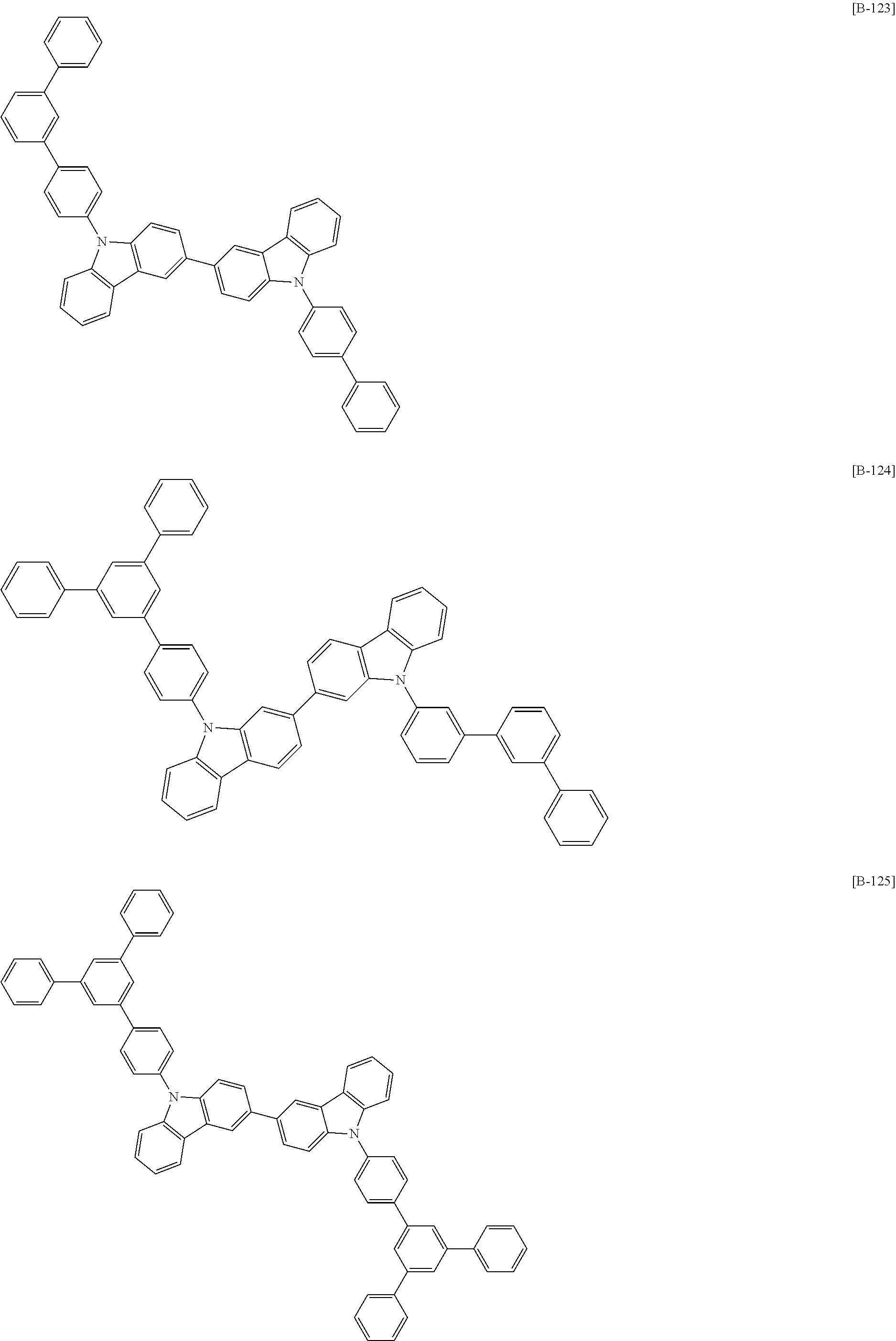 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1 ; c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

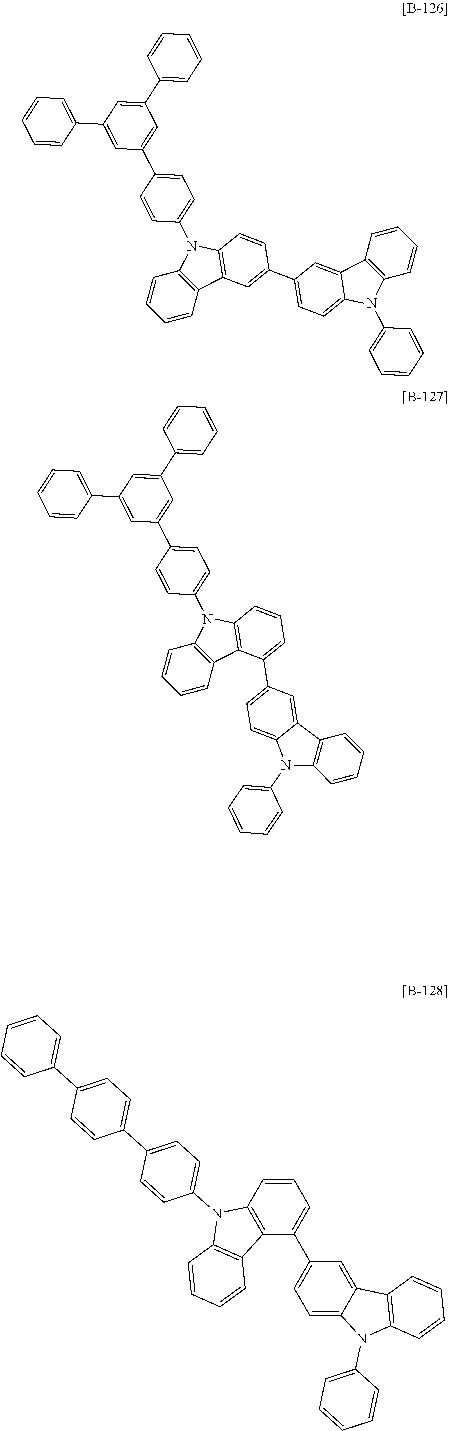 [SMILES: c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1]

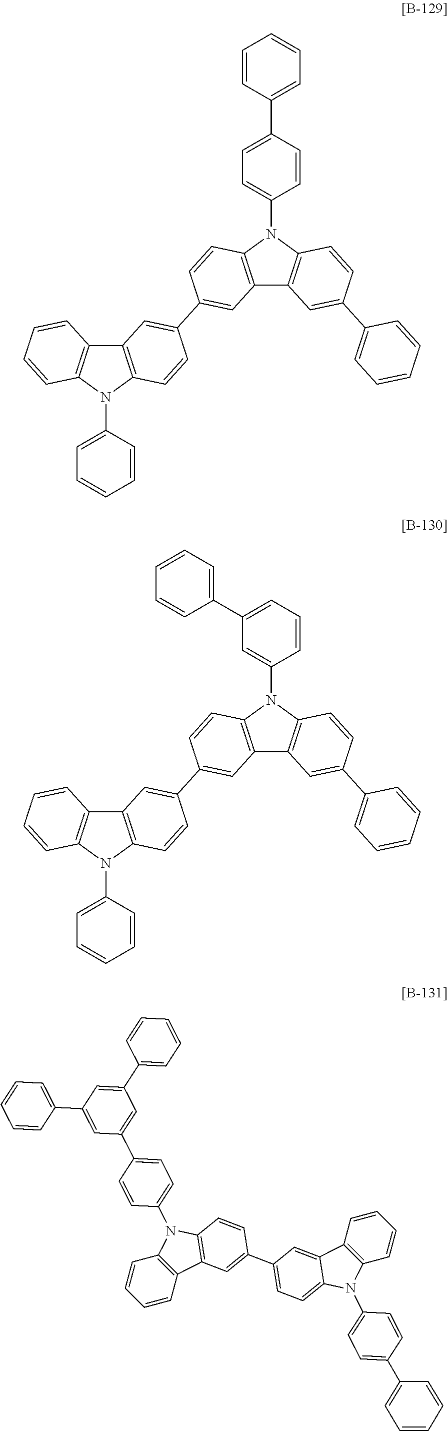 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1]

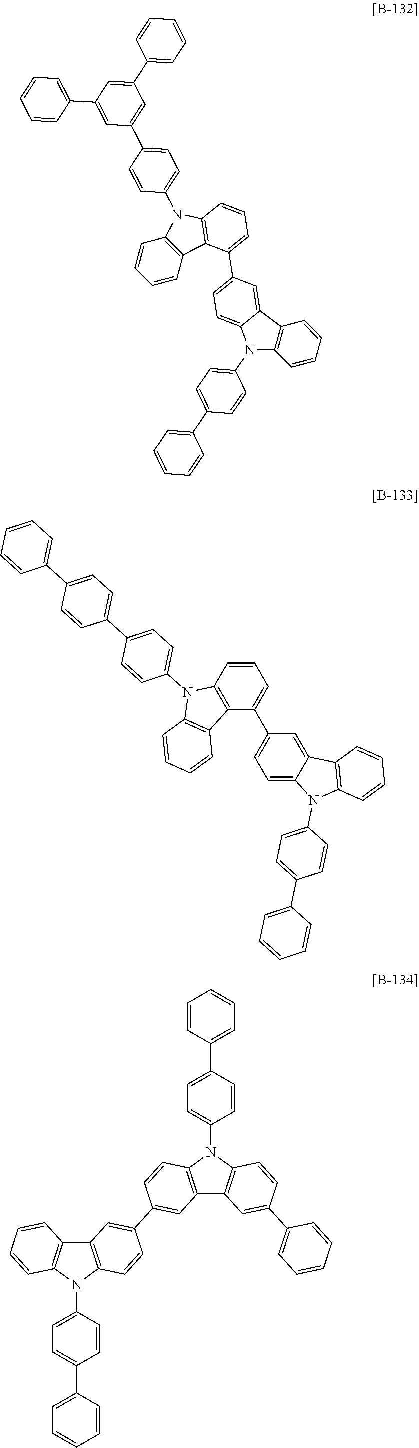 [SMILES: c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1 ; c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1]

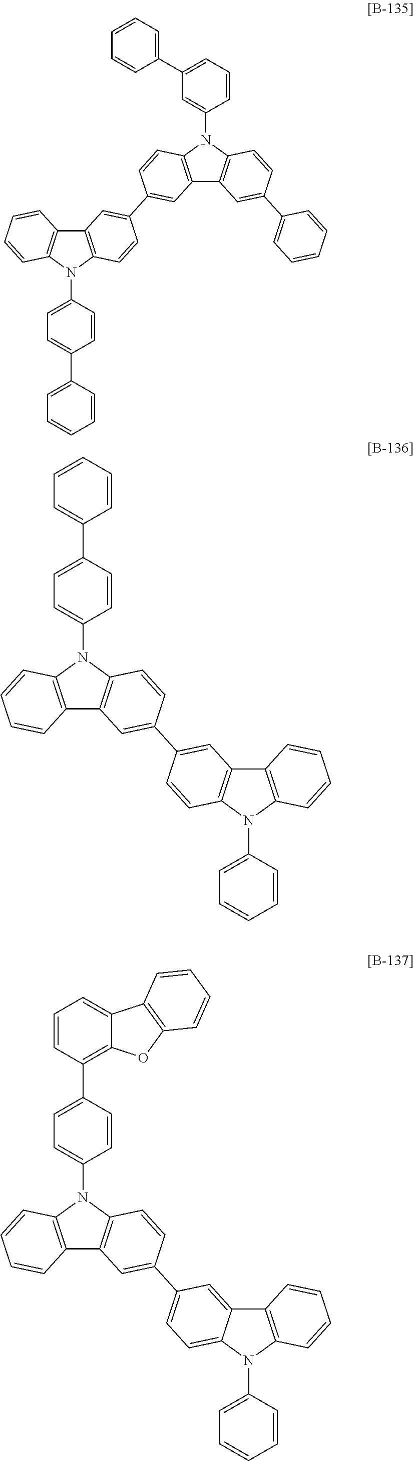

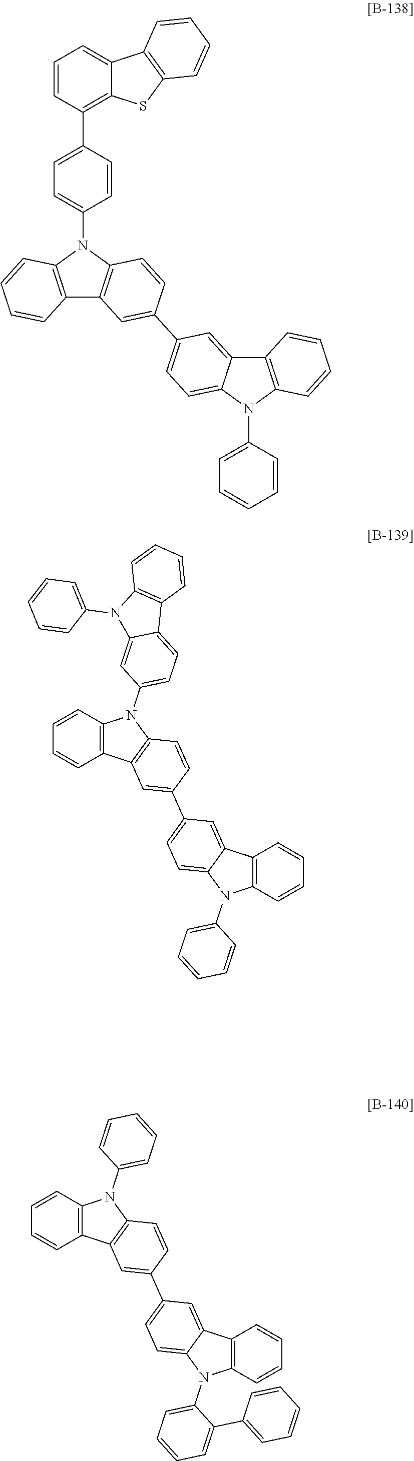 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccccc2-n2c3ccccc3c3cc(-c4ccc5c(c4)c4ccccc4n5-c4ccccc4)ccc32)cc1]

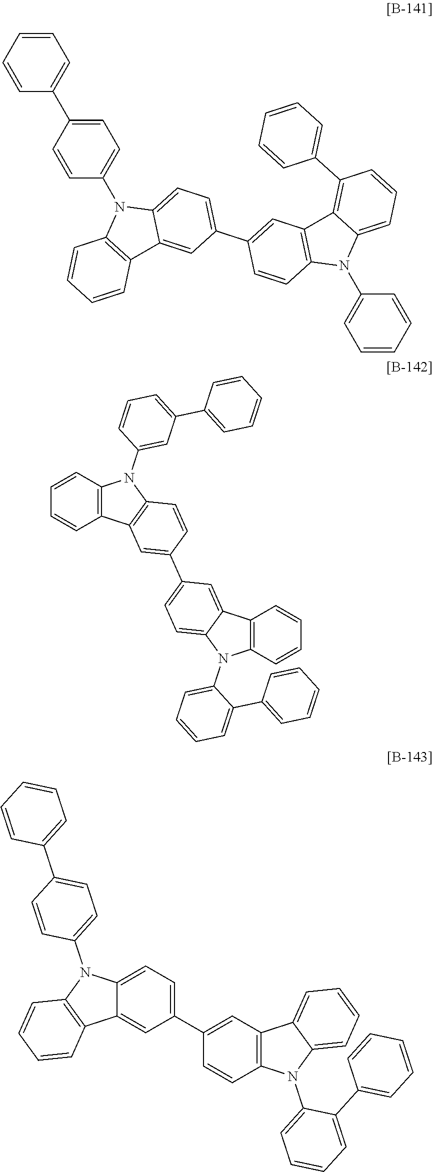 [SMILES: c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

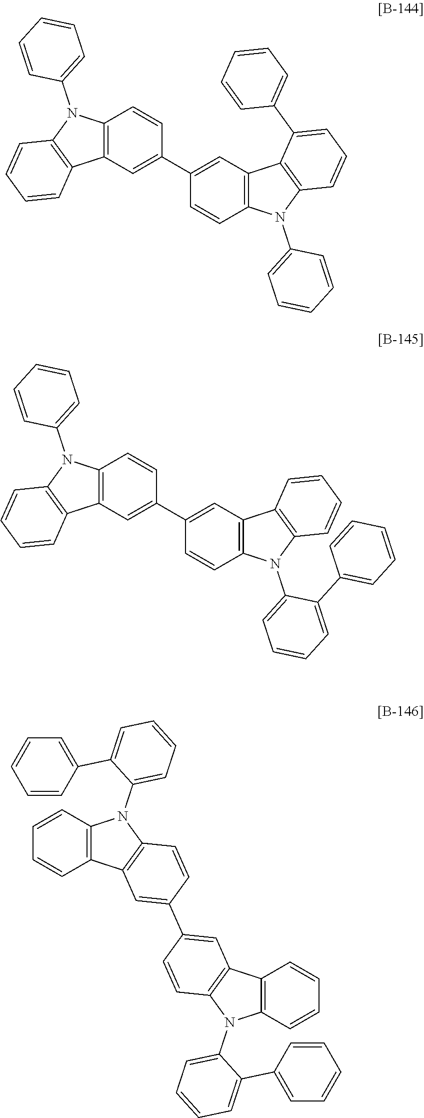 [SMILES: c1ccc(-c2cccc3c2c2cc(-c4ccc5c(c4)c4ccccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4-c4ccccc4)ccc32)cc1]

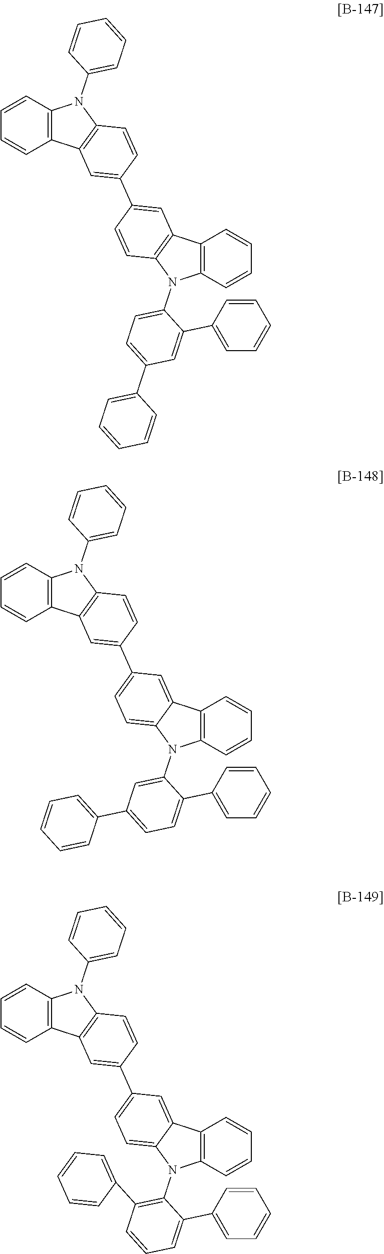

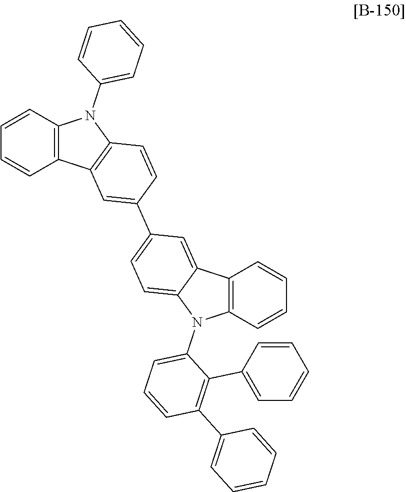 [SMILES: c1ccc(-c2cccc(-n3c4ccccc4c4cc(-c5ccc6c(c5)c5ccccc5n6-c5ccccc5)ccc43)c2-c2ccccc2)cc1]

In an implementation, examples of Compound B-1 to Compound B-150 listed in Group 2 in which at least one hydrogen is replaced with deuterium are given below.

In an implementation, deuterium may be substituted, e.g., as shown in the compound B-151 to compound B-195 exemplified below.

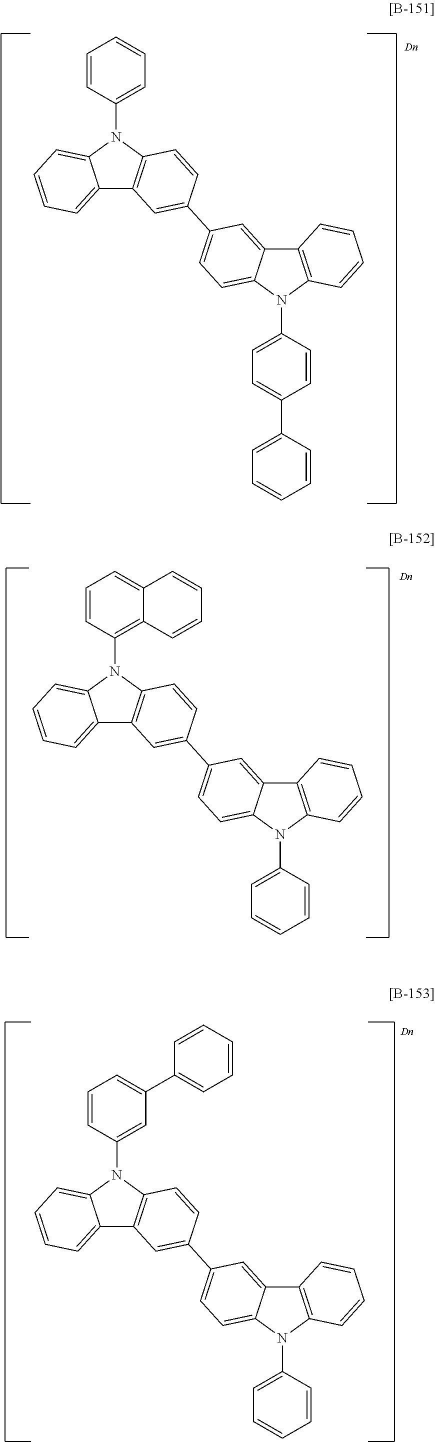

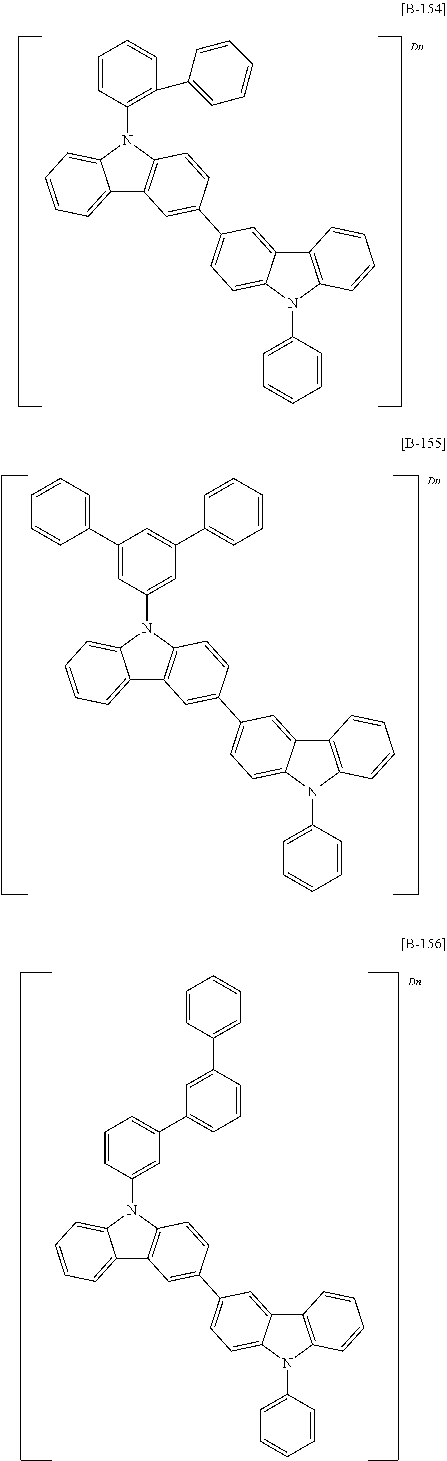

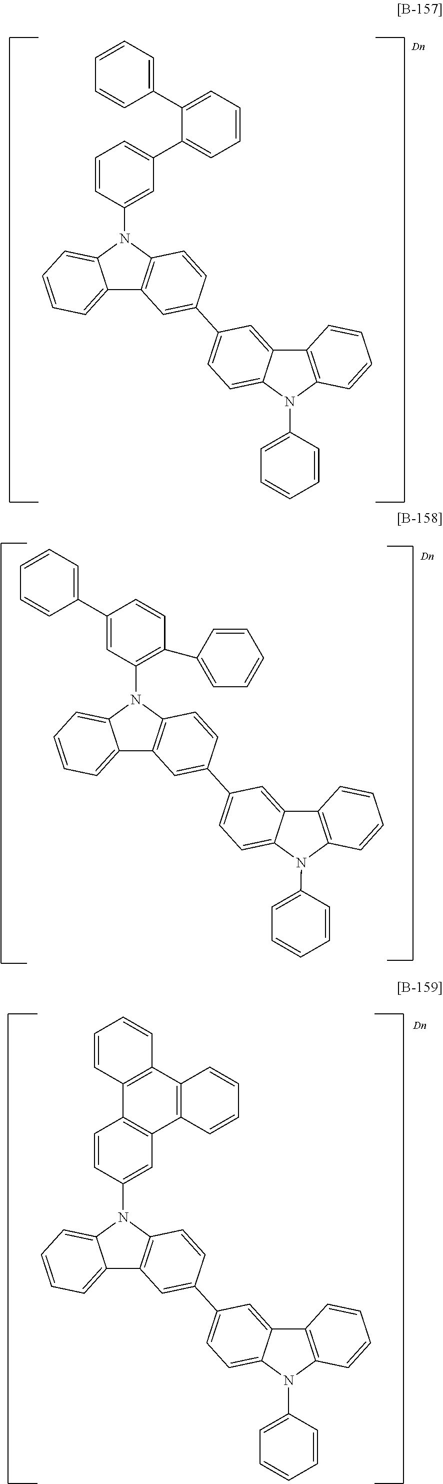

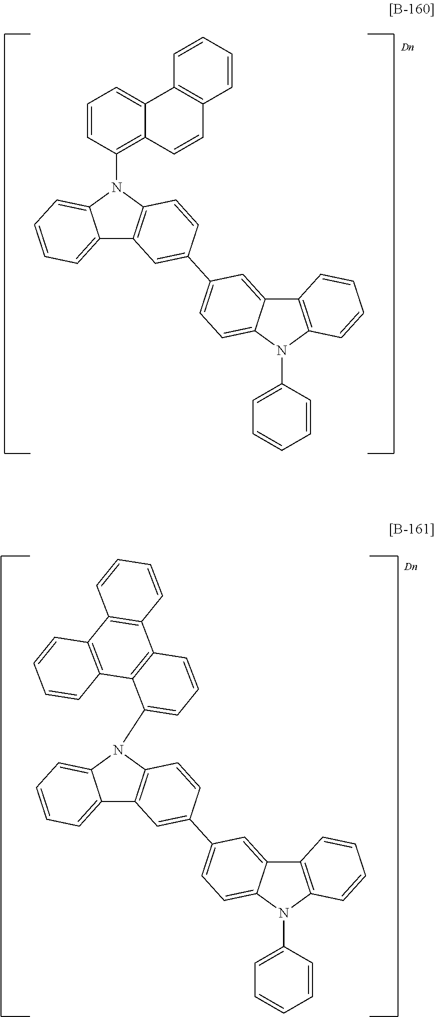

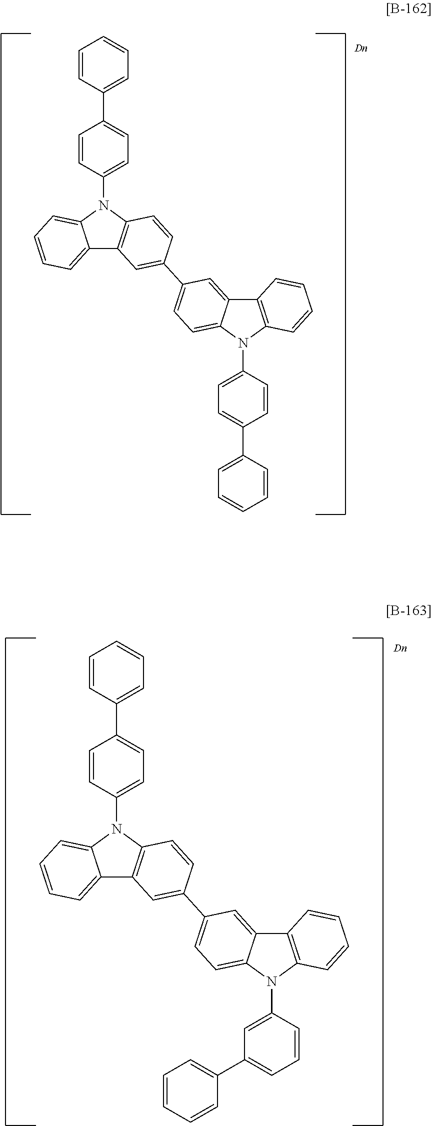

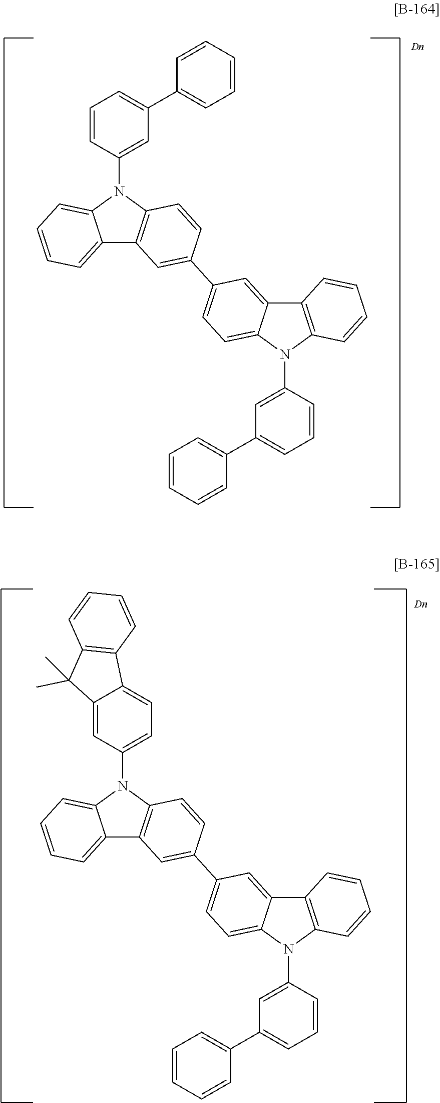

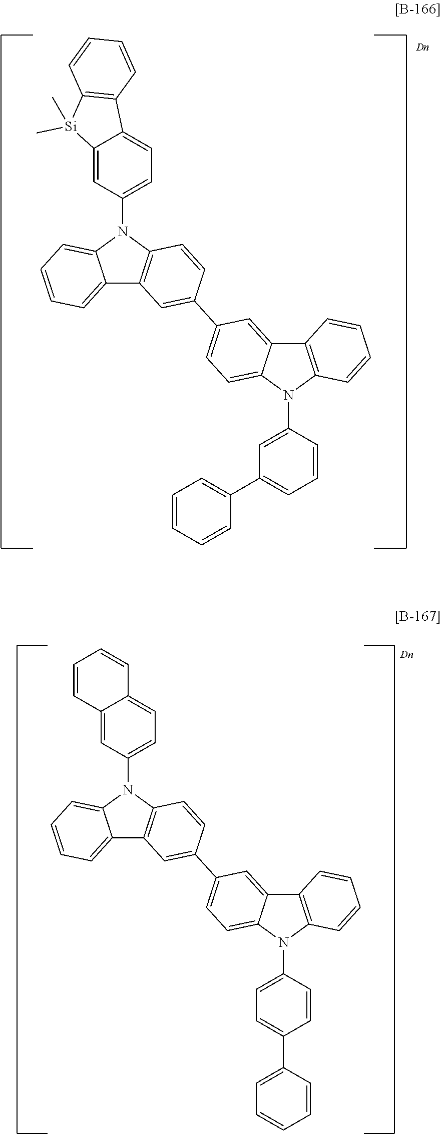

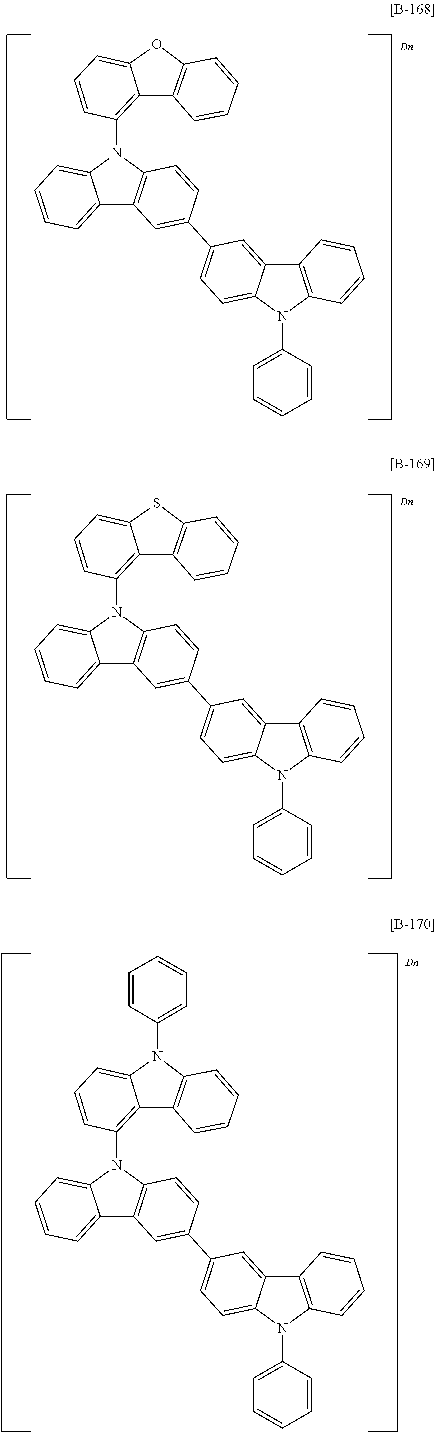

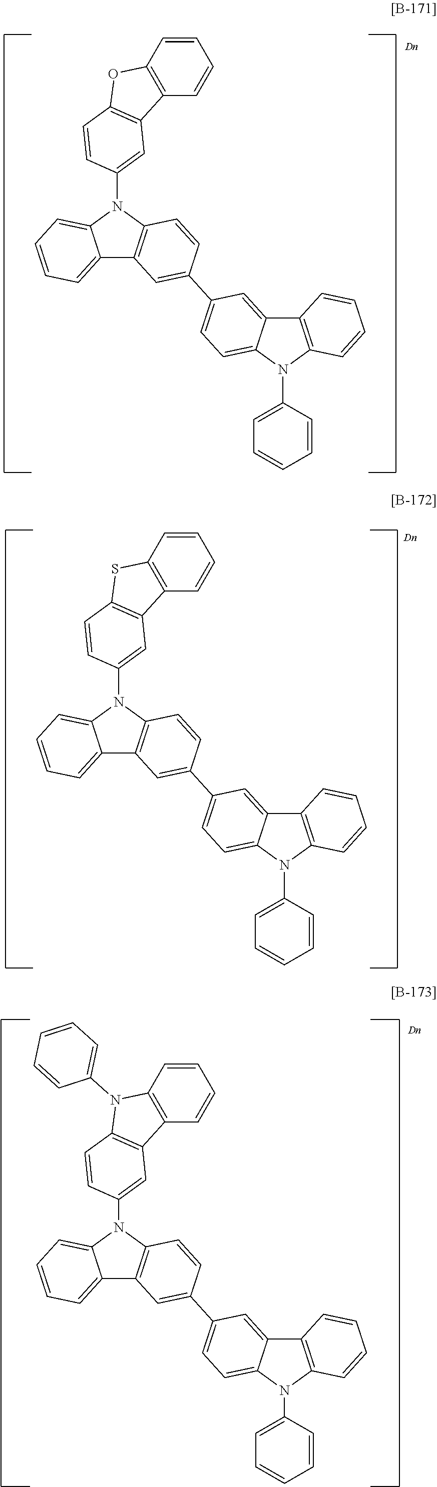

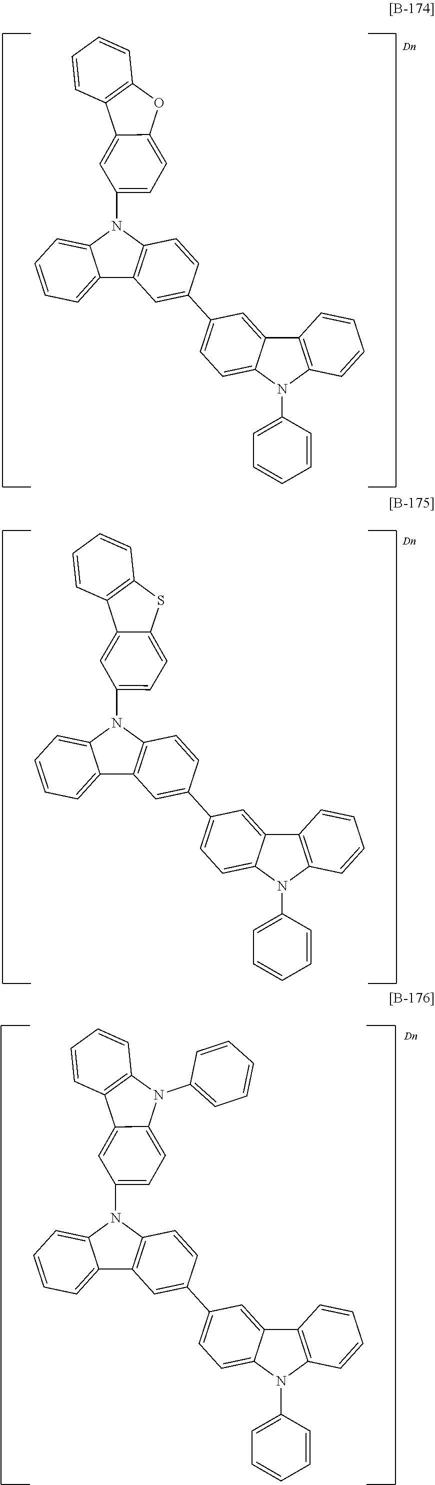

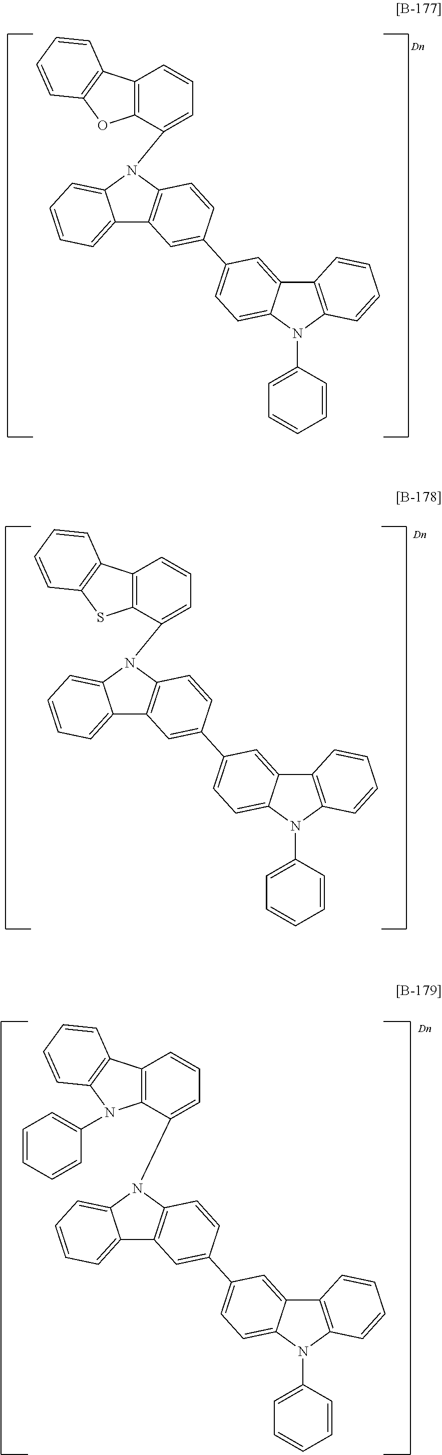

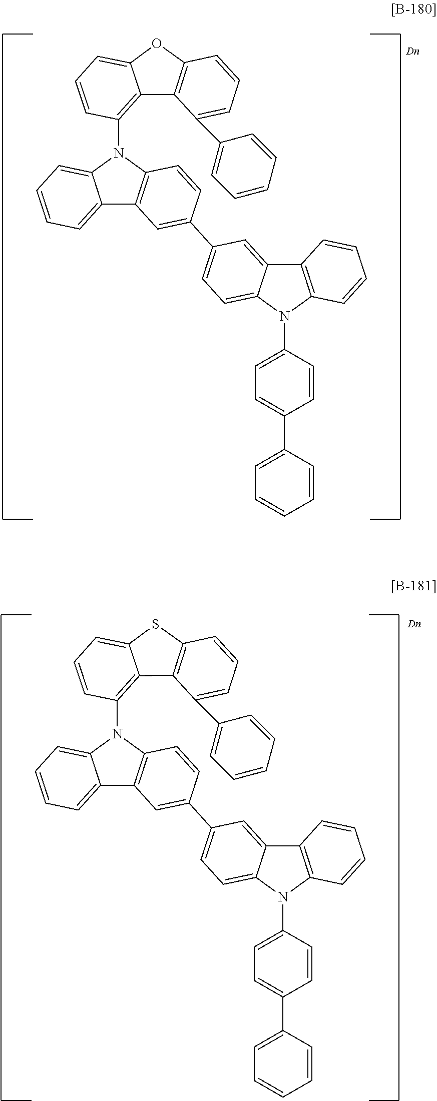

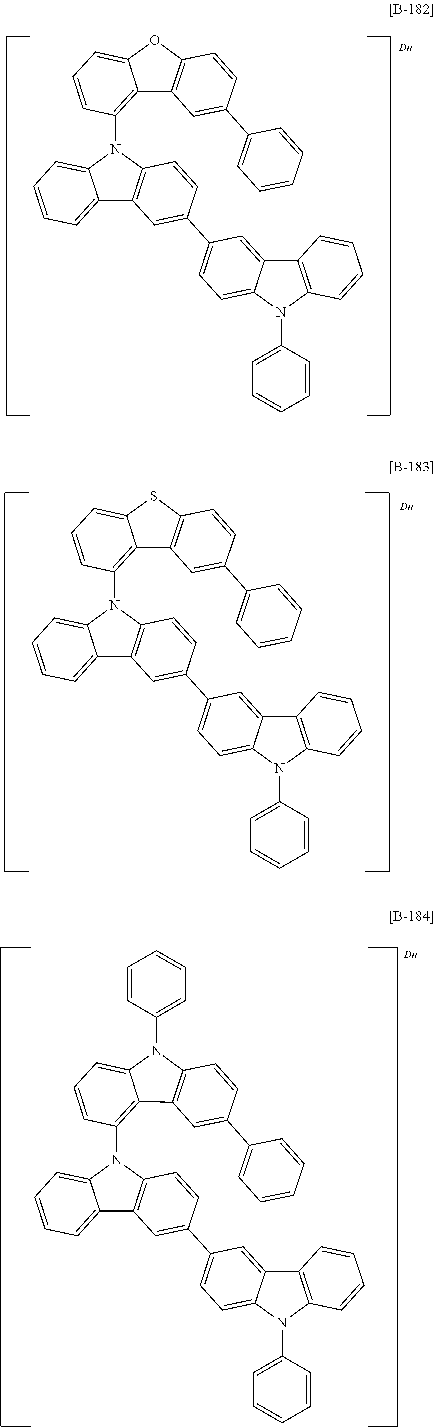

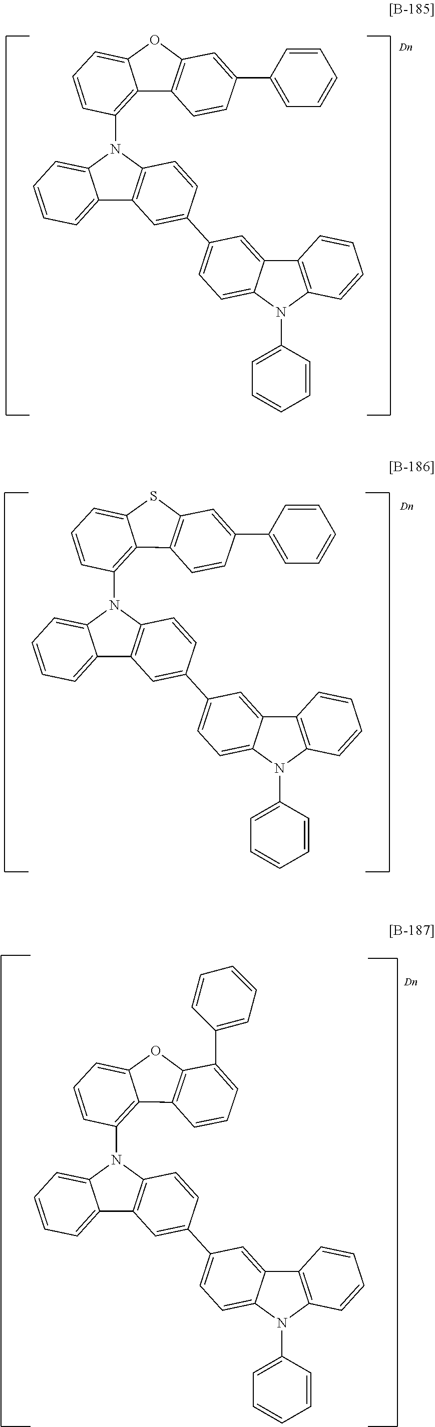

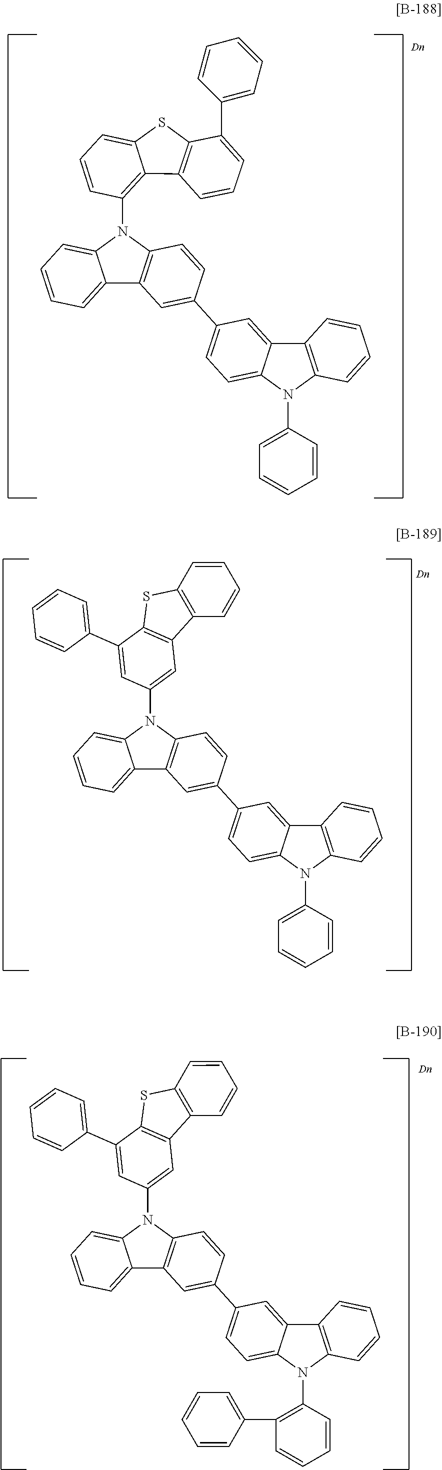

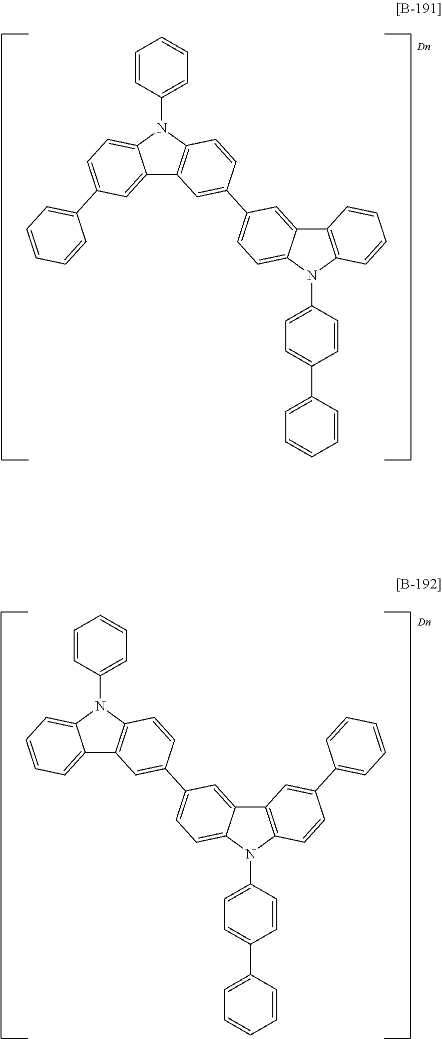

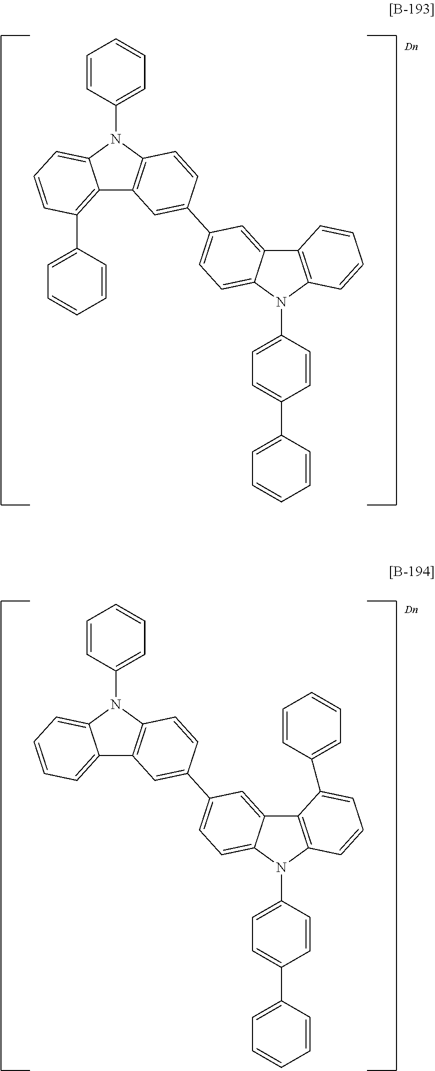

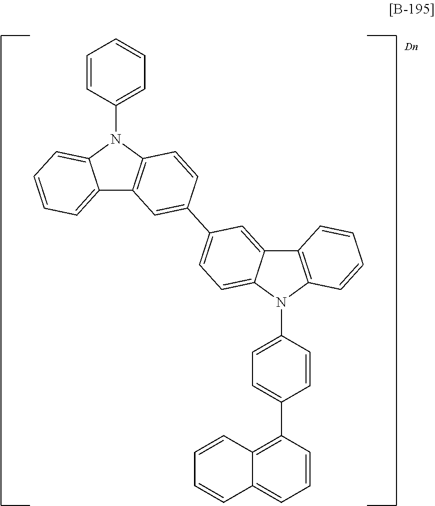

(Dn refers to the number of deuterium substitutions and indicates a structure substituted with one or more deuterium atoms)

The deuterium substitution position and deuterium substitution ratio may include all changeable ranges within the range of Compound B-1 to Compound B-195 (e.g., any hydrogen in any compound may be a protium or a deuterium).

The most specific structures for Compound B-151 to Compound B-195 are presented below as examples according to the position and substitution rate of deuterium substitution.

In an implementation, deuterium may be substituted, e.g., as shown in the compound B-196 to compound 234.

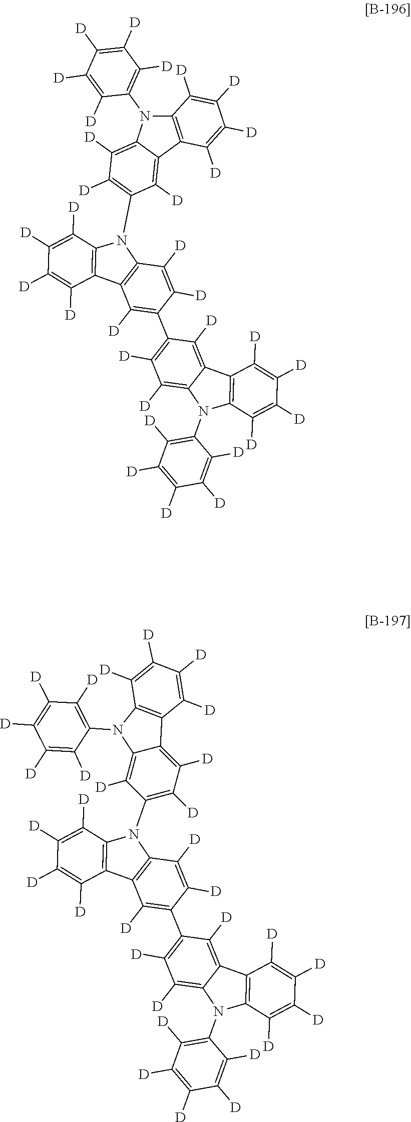

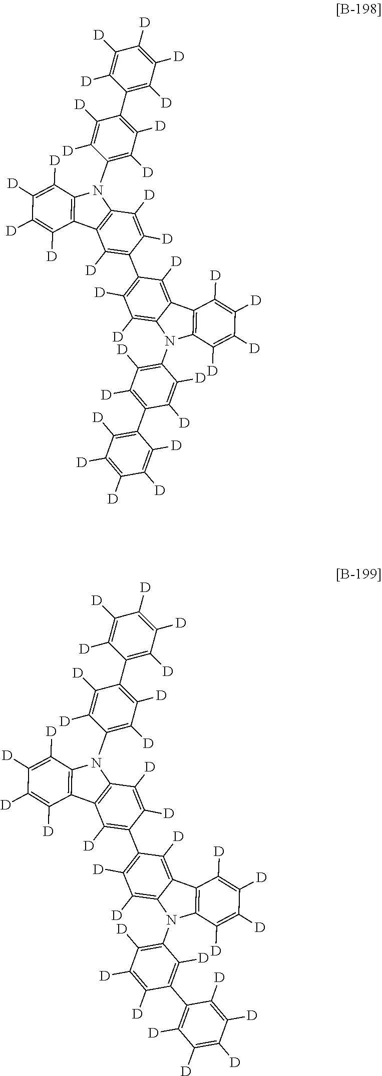

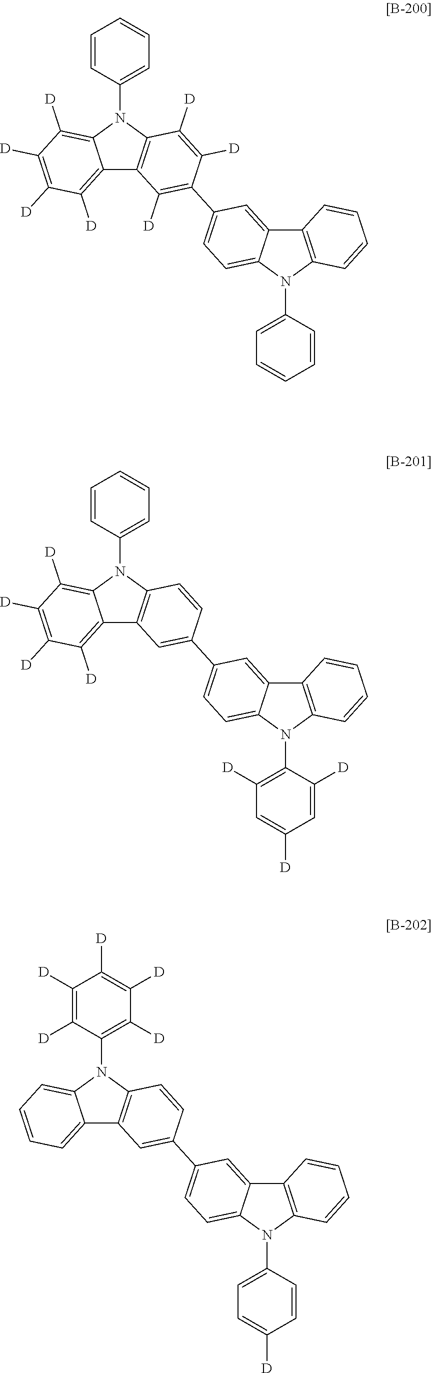 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccccc1 ; [2H]c1cc([2H])c(-n2c3ccccc3c3cc(-c4ccc5c(c4)c4c([2H])c([2H])c([2H])c([2H])c4n5-c4ccccc4)ccc32)c([2H])c1 ; [2H]c1ccc(-n2c3ccccc3c3cc(-c4ccc5c(c4)c4ccccc4n5-c4c([2H])c([2H])c([2H])c([2H])c4[2H])ccc32)cc1]

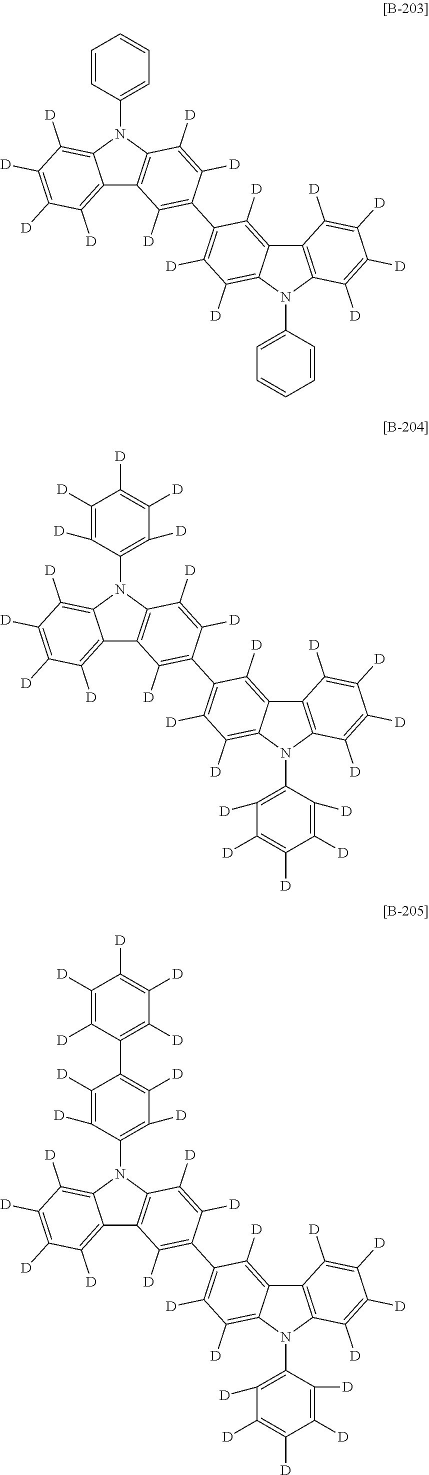

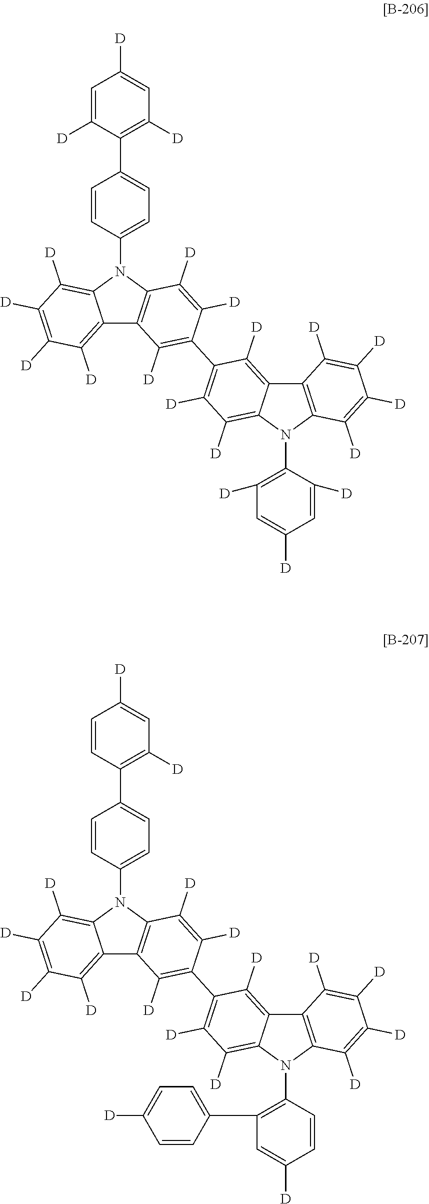 [SMILES: [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1 ; [2H]c1ccc(-c2cc([2H])ccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1]

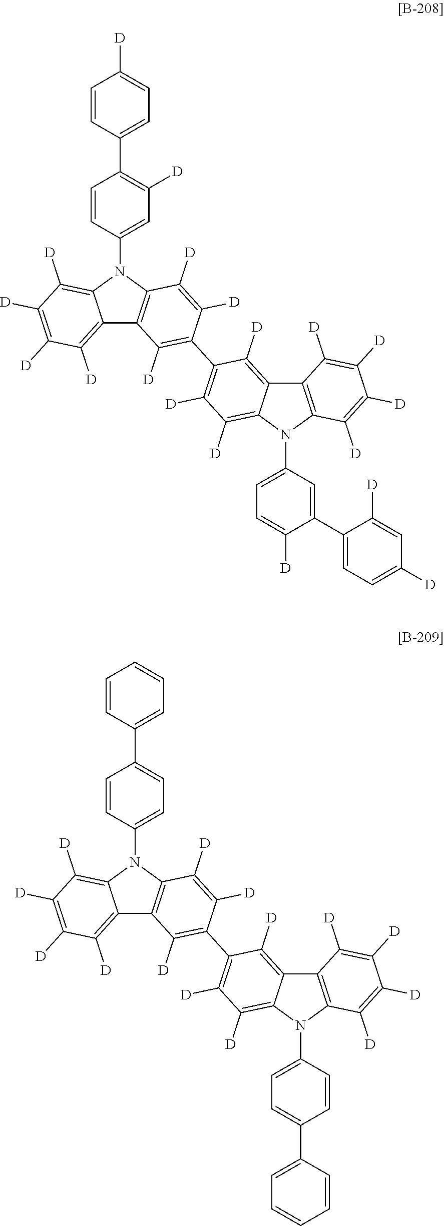 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1]

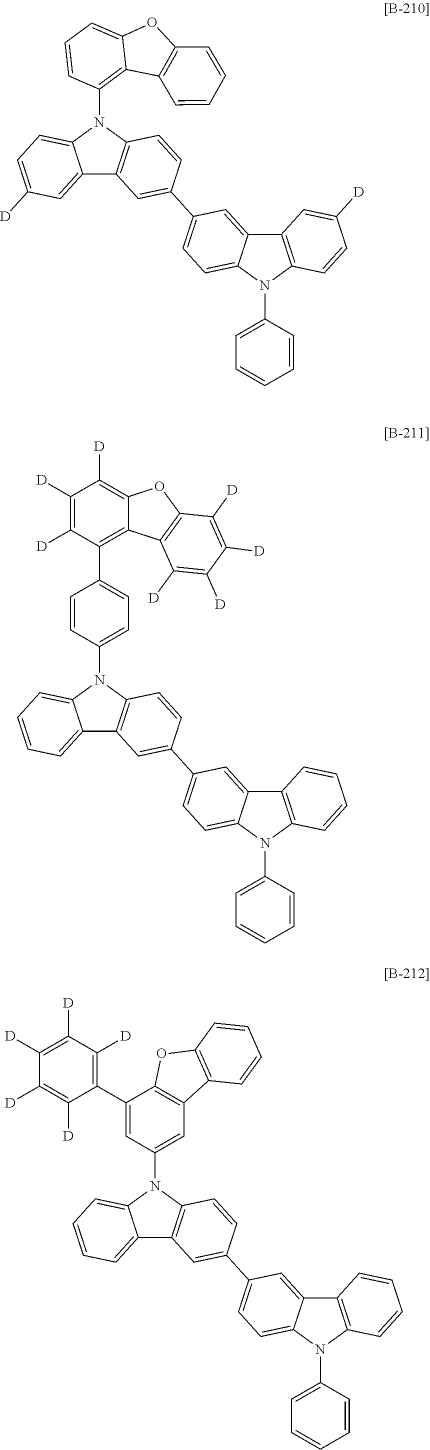 [SMILES: [2H]c1ccc2c(c1)c1cc(-c3ccc4c(c3)c3cc([2H])ccc3n4-c3cccc4oc5ccccc5c34)ccc1n2-c1ccccc1 ; [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H] ; [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H]]

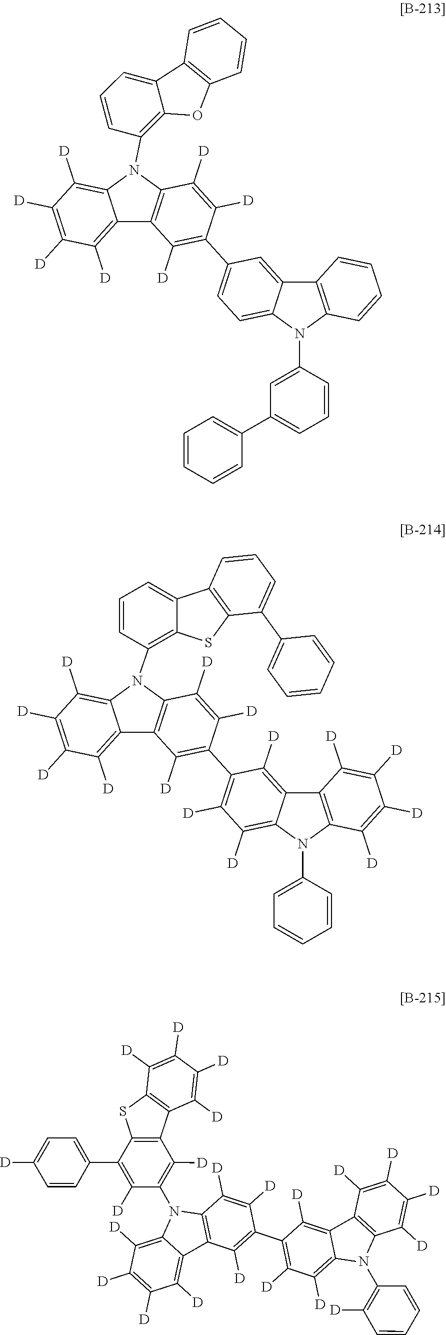

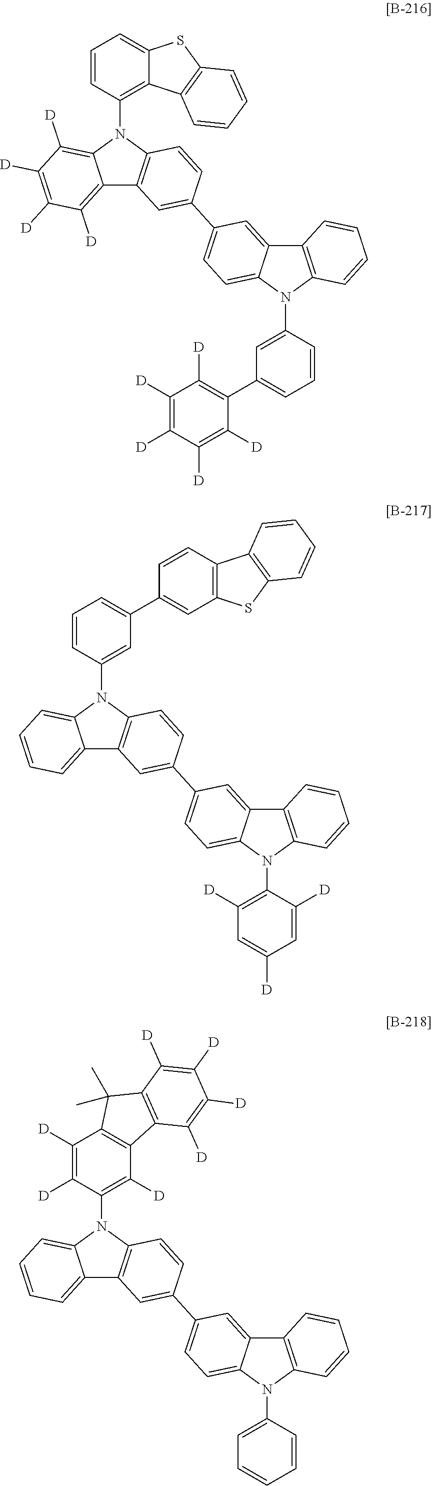 [SMILES: [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1 ; [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C]

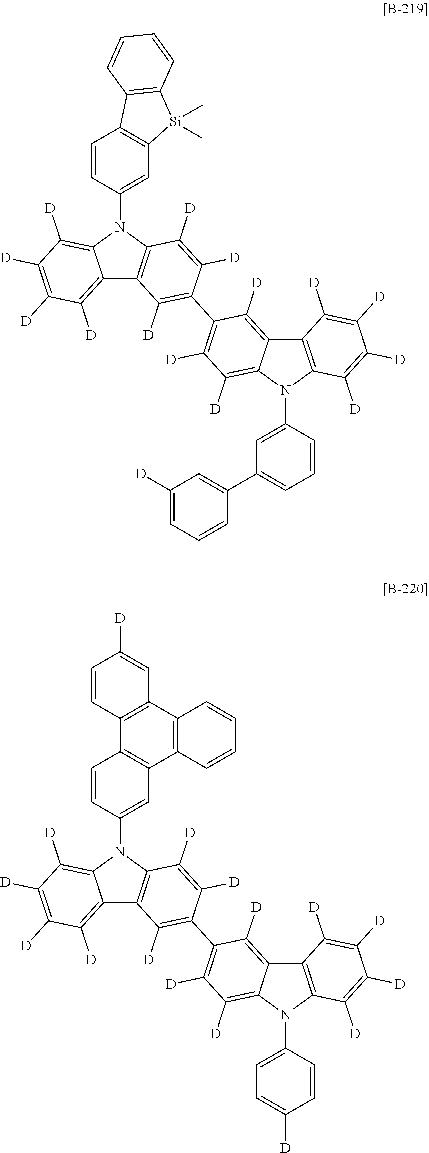 [SMILES: [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1 ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1]

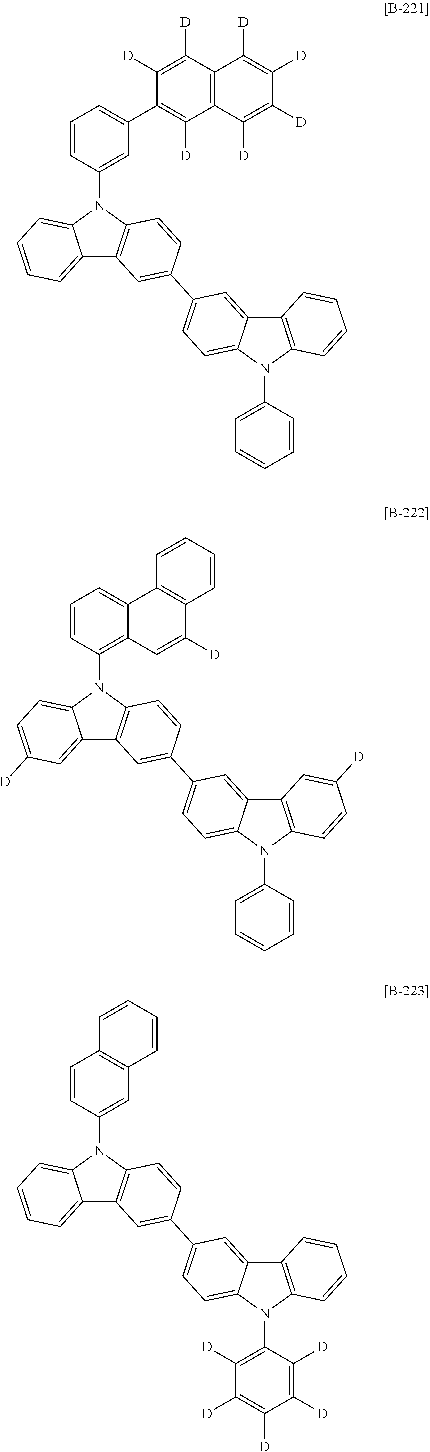 [SMILES: [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H] ; [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H]]

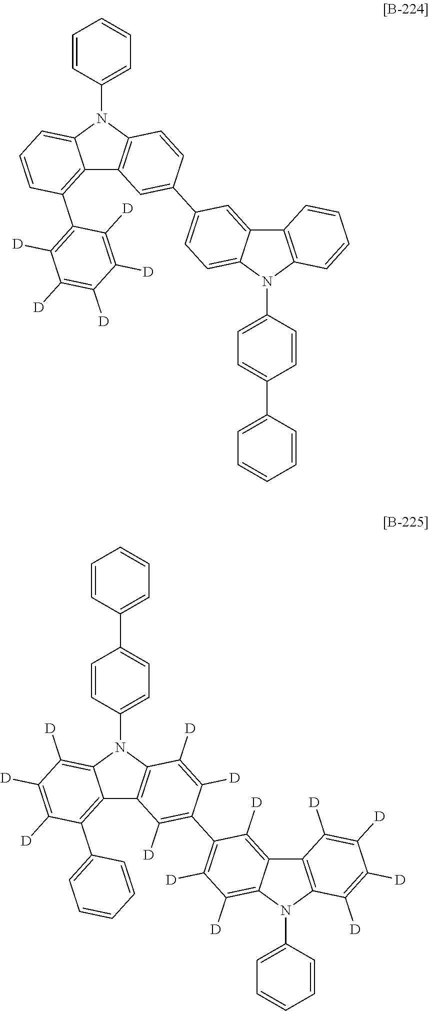 [SMILES: [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1]

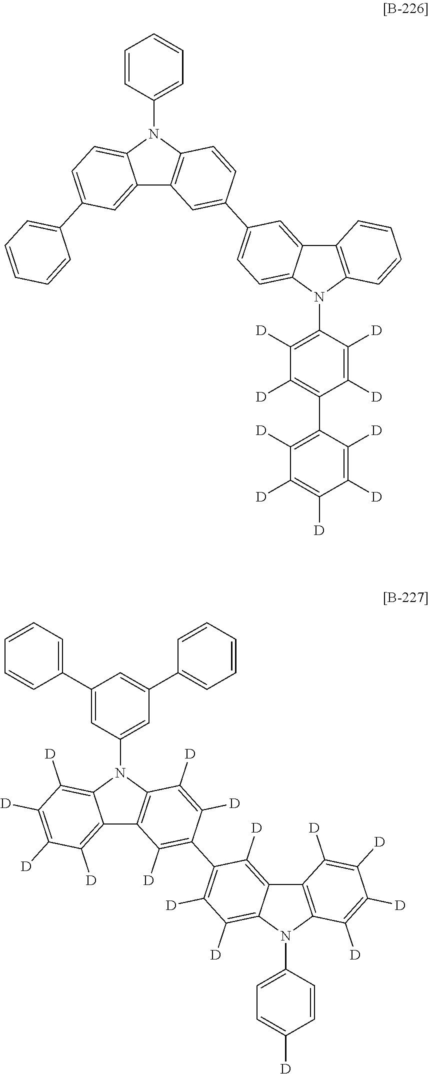 [SMILES: [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H] ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1]

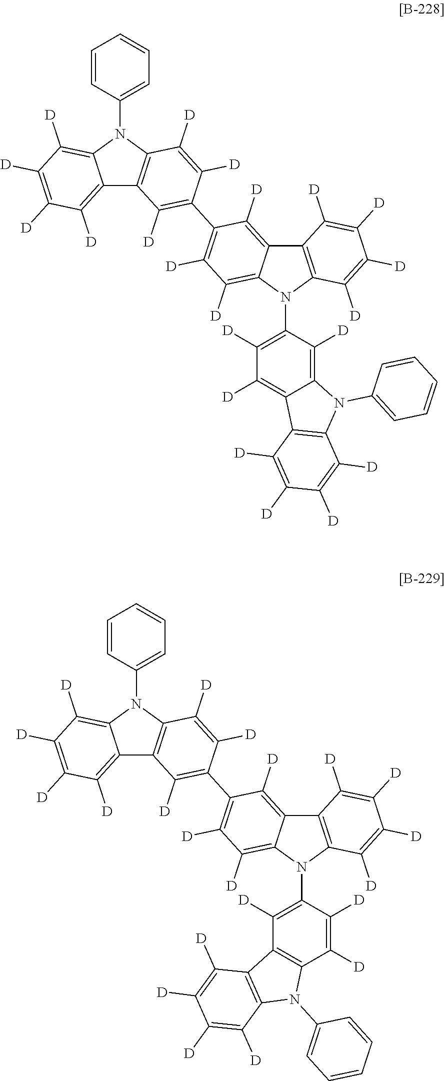 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1]

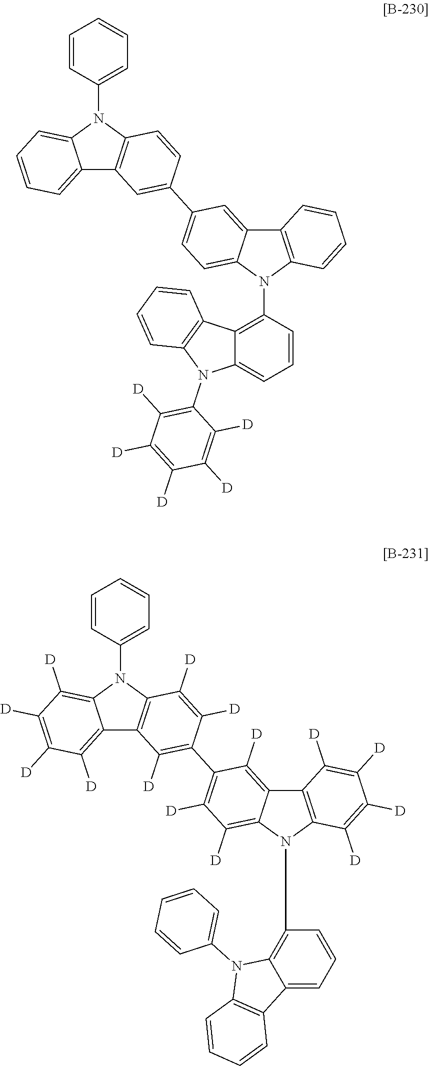 [SMILES: [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1]

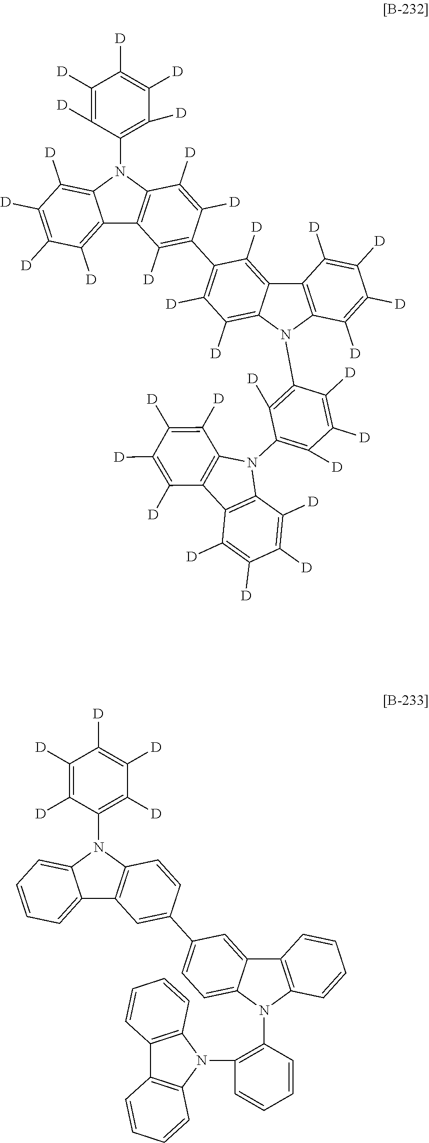 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H] ; [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H]]

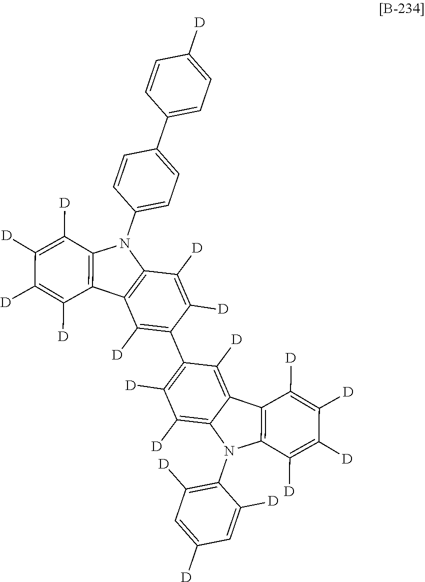 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1]

In an implementation, the second organic optoelectronic device compound may be, e.g., a compound of Group 3.

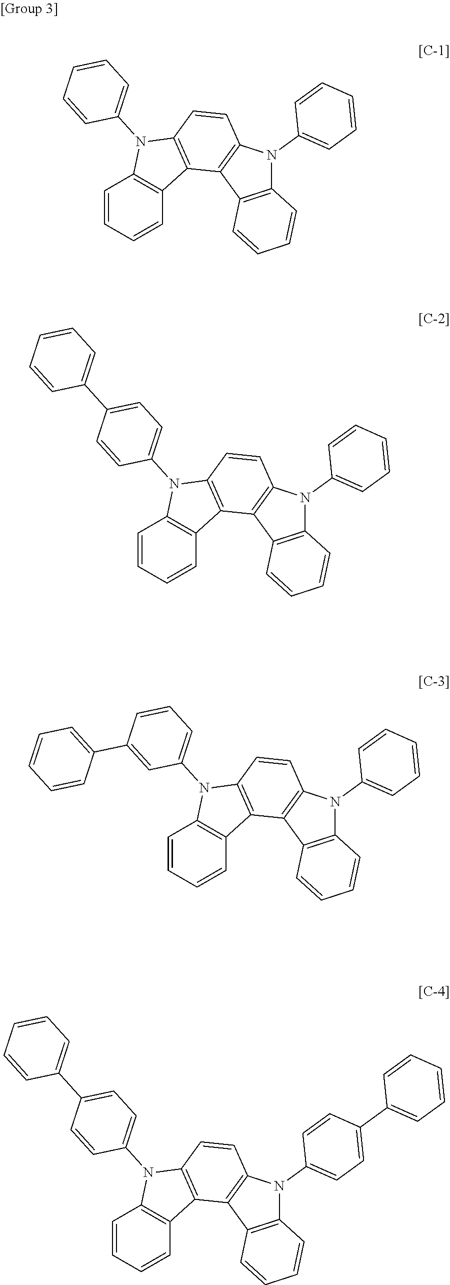 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

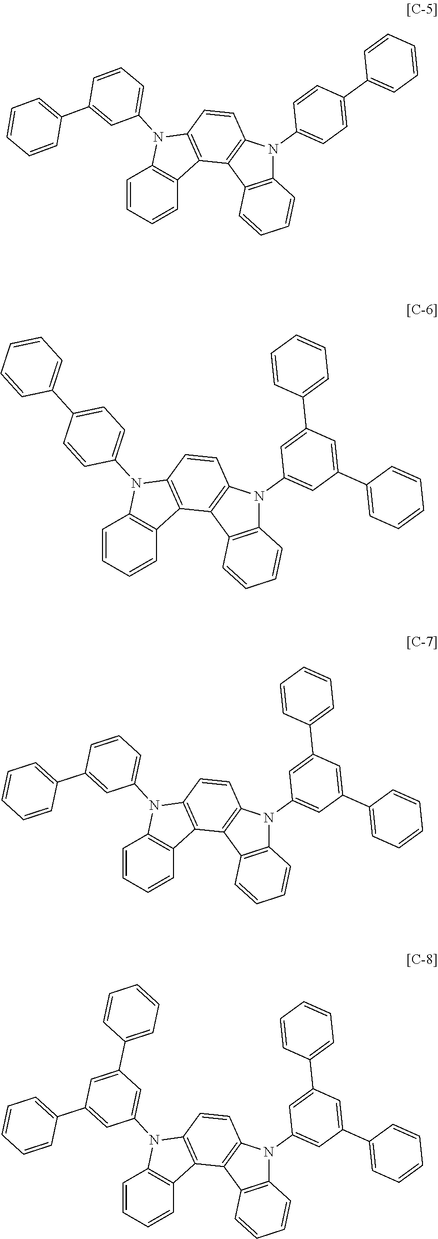 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1]

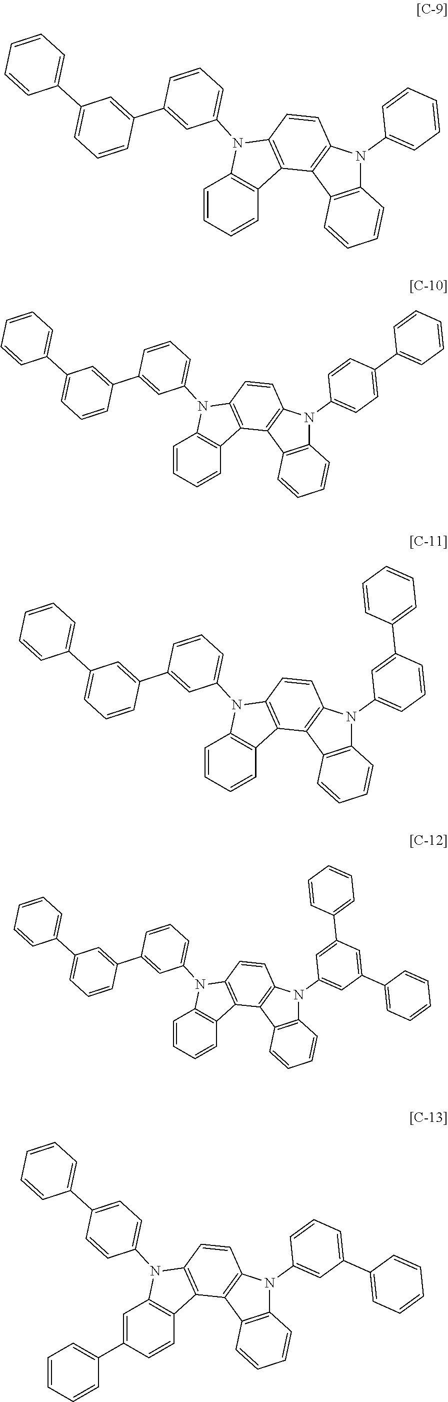 [SMILES: c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1 ; c1ccc(-c2ccc(-n3c4cc(-c5ccccc5)ccc4c4c5c6ccccc6n(-c6cccc(-c7ccccc7)c6)c5ccc43)cc2)cc1]

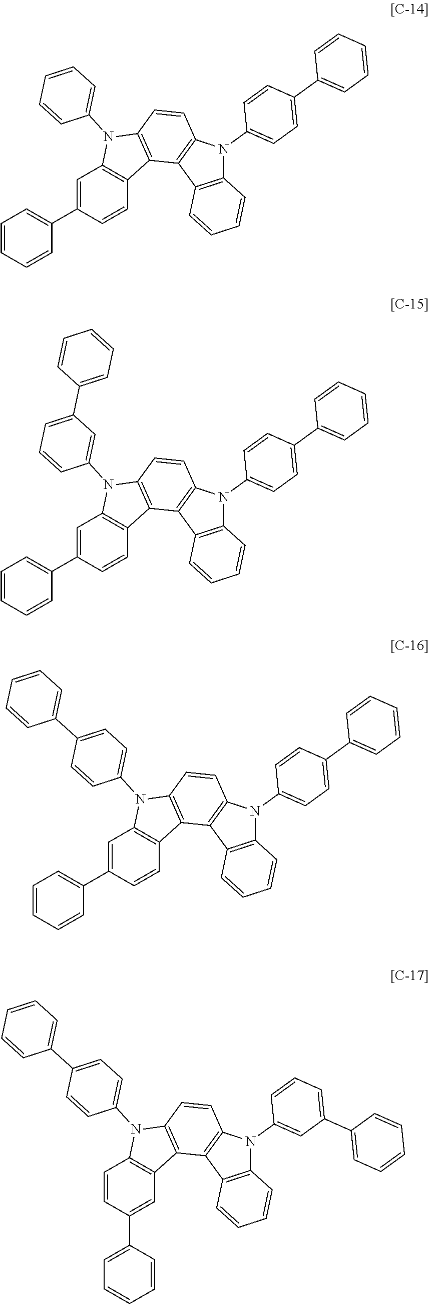 [SMILES: c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1 ; c1ccc(-c2ccc(-n3c4ccc(-c5ccccc5)cc4c4c5c6ccccc6n(-c6cccc(-c7ccccc7)c6)c5ccc43)cc2)cc1]

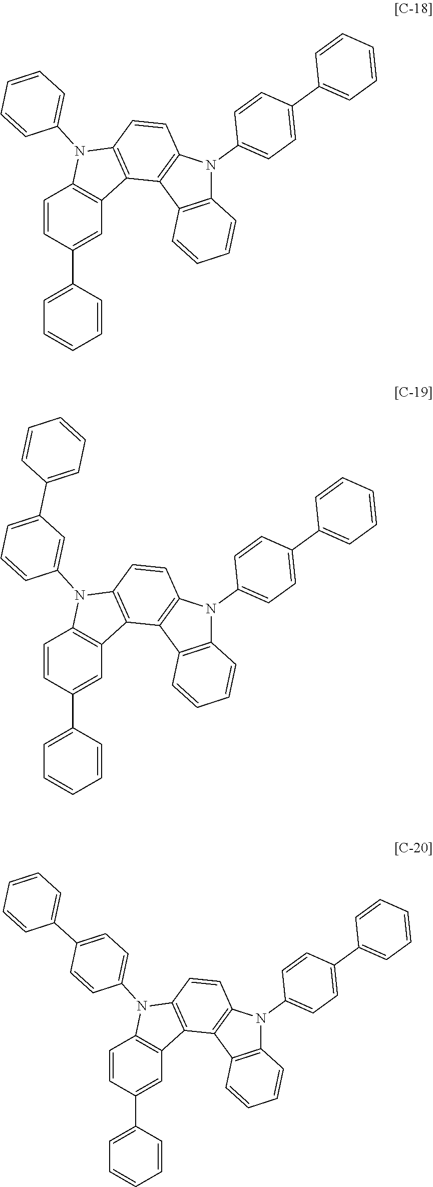 [SMILES: c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccc(-c7ccccc7)cc6)c5ccc43)cc2)cc1]

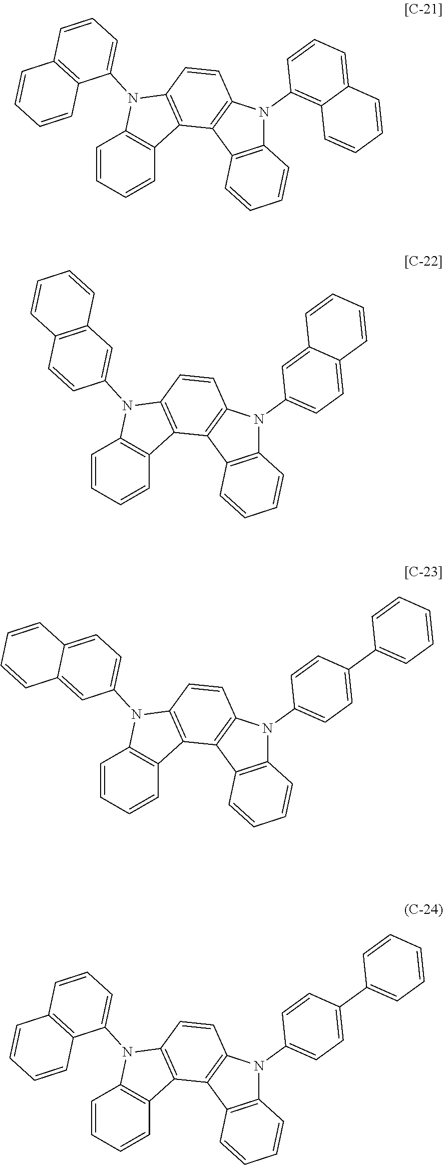 [SMILES: c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1 ; c1ccc(-c2ccc(-n3c4ccccc4c4c5c6ccccc6n(-c6cccc7ccccc67)c5ccc43)cc2)cc1]

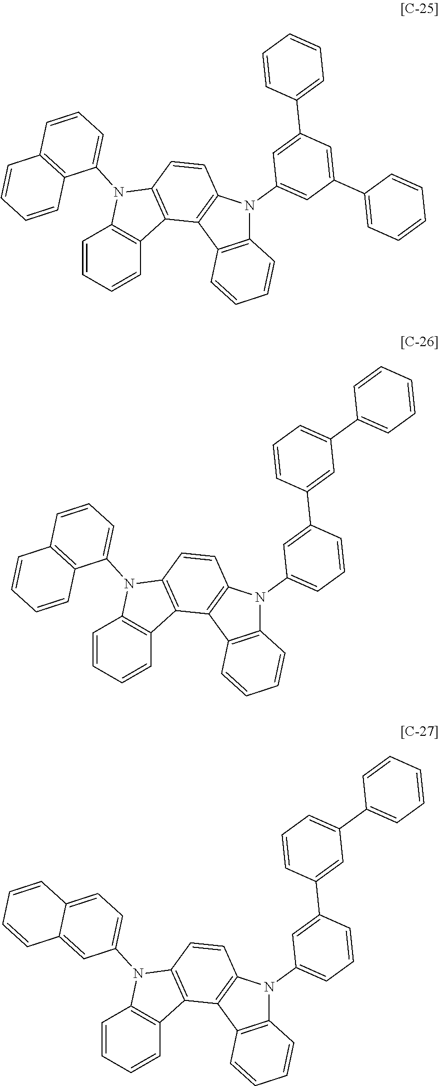 [SMILES: c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1]

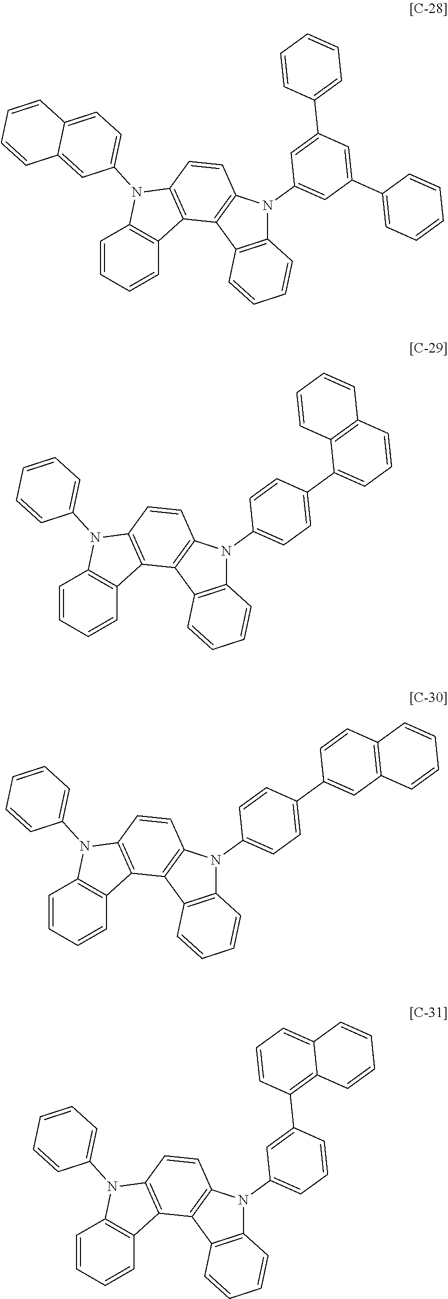 [SMILES: c1ccc(-c2cc(-c3ccccc3)cc(-n3c4ccccc4c4c5c6ccccc6n(-c6ccc7ccccc7c6)c5ccc43)c2)cc1 ; c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1]

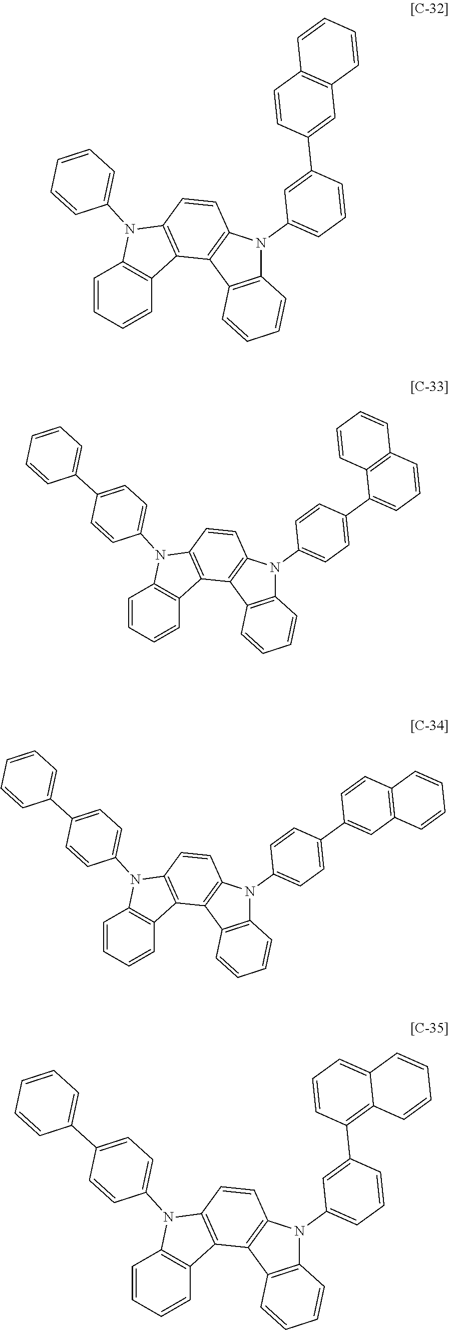 [SMILES: c1ccc(-n2c3ccccc3c3c4c5ccccc5n(-c5cccc(-c6ccc7ccccc7c6)c5)c4ccc32)cc1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1]

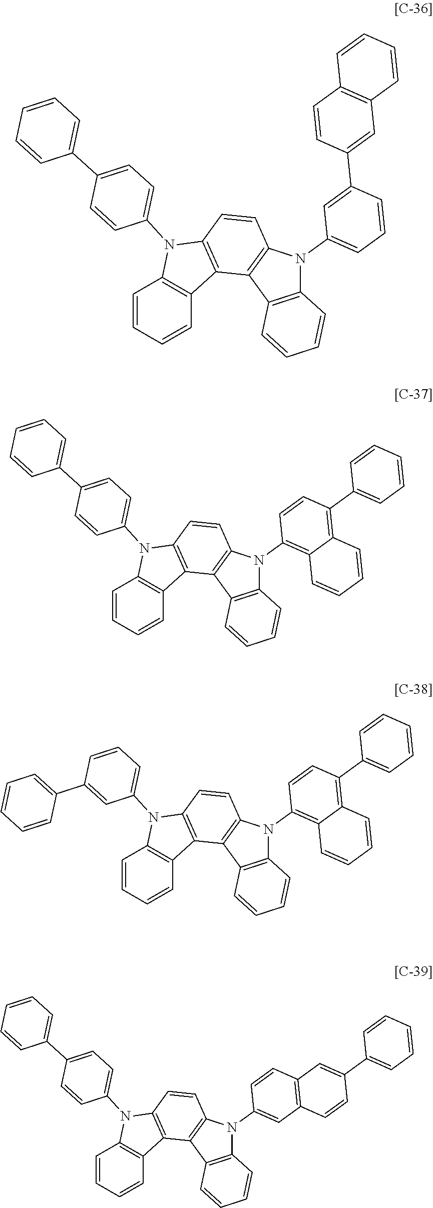 [SMILES: c1ccc(-c2ccc(-n3c4ccccc4c4c5c6ccccc6n(-c6cccc(-c7ccc8ccccc8c7)c6)c5ccc43)cc2)cc1 ; c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1]

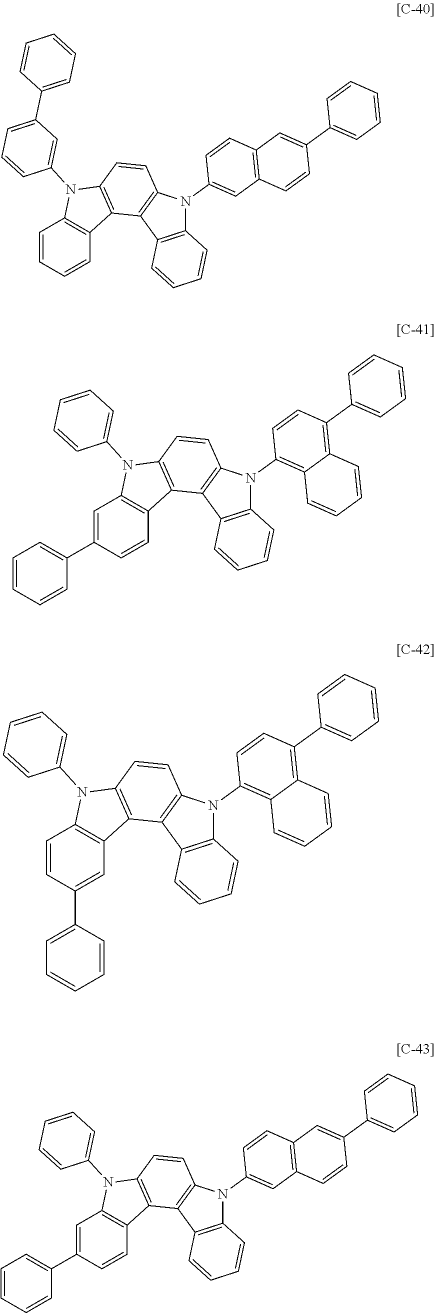 [SMILES: c1ccc(-c2cccc(-n3c4ccccc4c4c5c6ccccc6n(-c6ccc7cc(-c8ccccc8)ccc7c6)c5ccc43)c2)cc1 ; c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1]

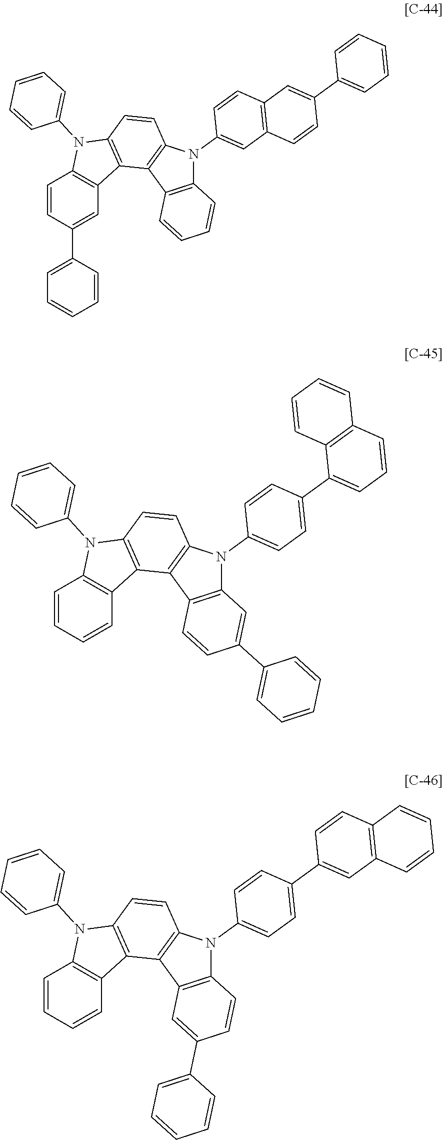 [SMILES: c1ccc(-c2ccc3cc(-n4c5ccccc5c5c6c7cc(-c8ccccc8)ccc7n(-c7ccccc7)c6ccc54)ccc3c2)cc1 ; c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1]

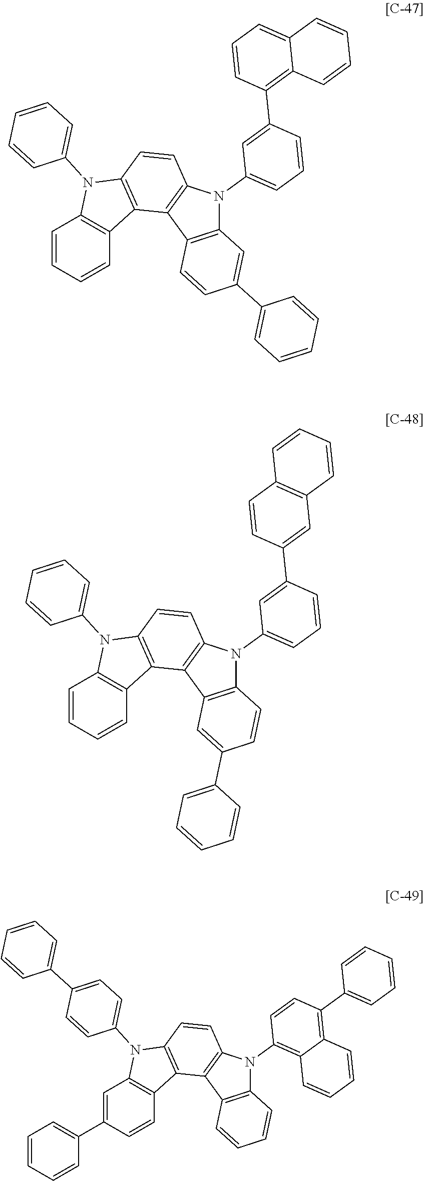 [SMILES: c1ccc(-c2ccc3c4c5c6ccccc6n(-c6ccccc6)c5ccc4n(-c4cccc(-c5cccc6ccccc56)c4)c3c2)cc1 ; c1ccc(-c2ccc3c(c2)c2c4c5ccccc5n(-c5ccccc5)c4ccc2n3-c2cccc(-c3ccc4ccccc4c3)c2)cc1 ; c1ccc(-c2ccc(-n3c4cc(-c5ccccc5)ccc4c4c5c6ccccc6n(-c6ccc(-c7ccccc7)c7ccccc67)c5ccc43)cc2)cc1]

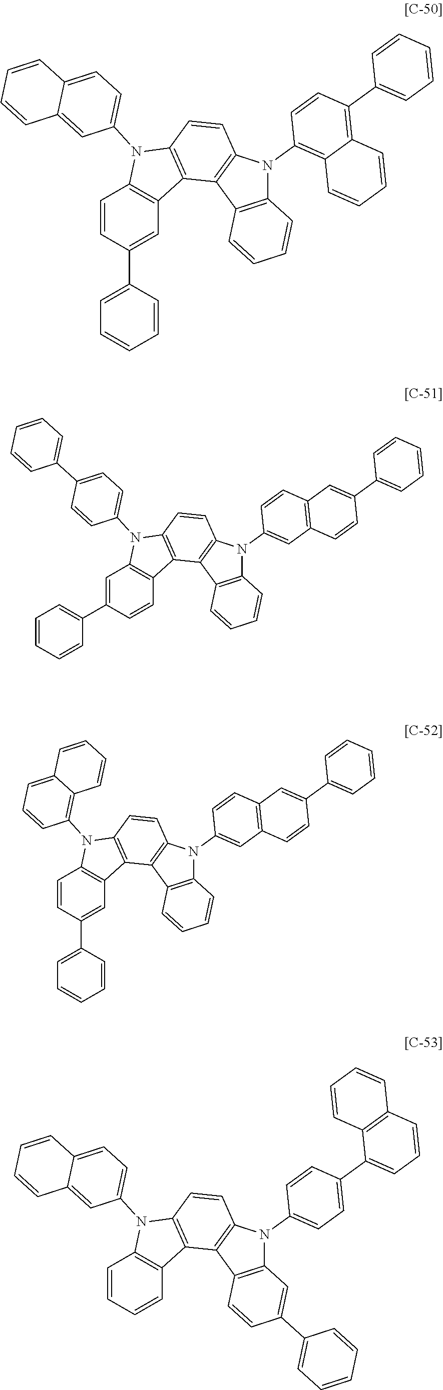 [SMILES: c1ccc(-c2ccc3c(c2)c2c4c5ccccc5n(-c5ccc(-c6ccccc6)c6ccccc56)c4ccc2n3-c2ccc3ccccc3c2)cc1 ; c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1 ; c1ccc(-c2ccc3c4c5c6ccccc6n(-c6ccc7ccccc7c6)c5ccc4n(-c4ccc(-c5cccc6ccccc56)cc4)c3c2)cc1]

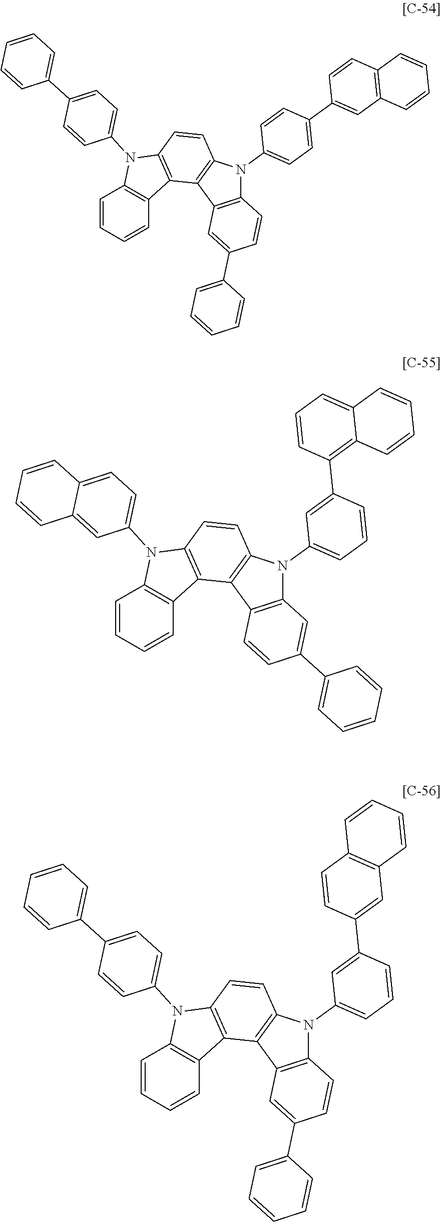 [SMILES: c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1 ; c1ccc(-c2ccc(-n3c4ccccc4c4c5c6cc(-c7ccccc7)ccc6n(-c6cccc(-c7ccc8ccccc8c7)c6)c5ccc43)cc2)cc1]

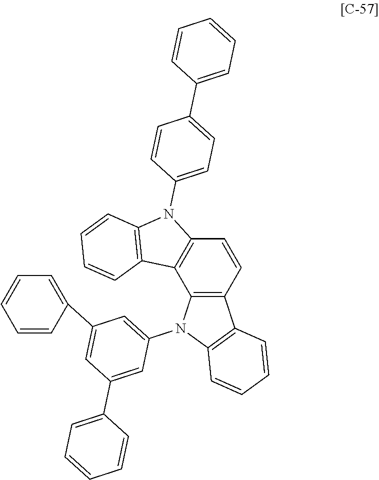 [SMILES: c1ccc(-c2ccc(-n3c4ccccc4c4c3ccc3c5ccccc5n(-c5cc(-c6ccccc6)cc(-c6ccccc6)c5)c34)cc2)cc1]

In an implementation, examples of Compound C-1 to Compound C-57 listed in Group 3 in which at least one hydrogen is replaced with deuterium are given below.

In an implementation, deuterium may be substituted, e.g., as shown in the compound C-58 to compound C-72 exemplified below.

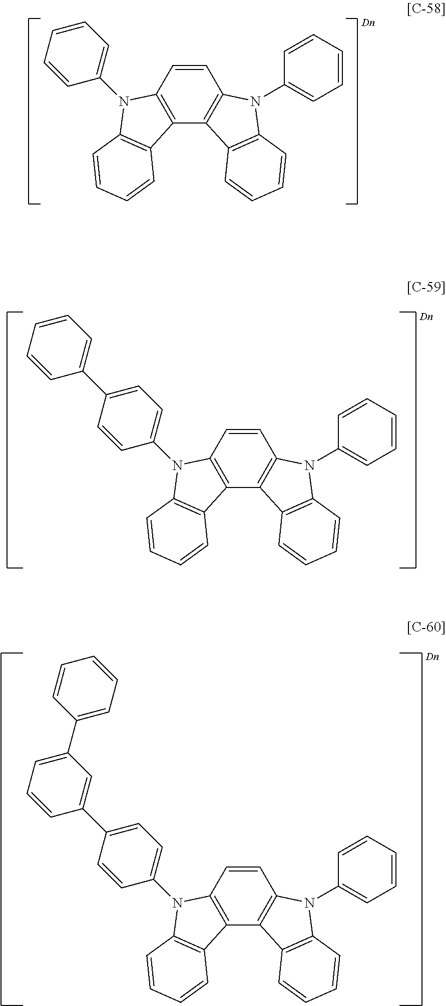

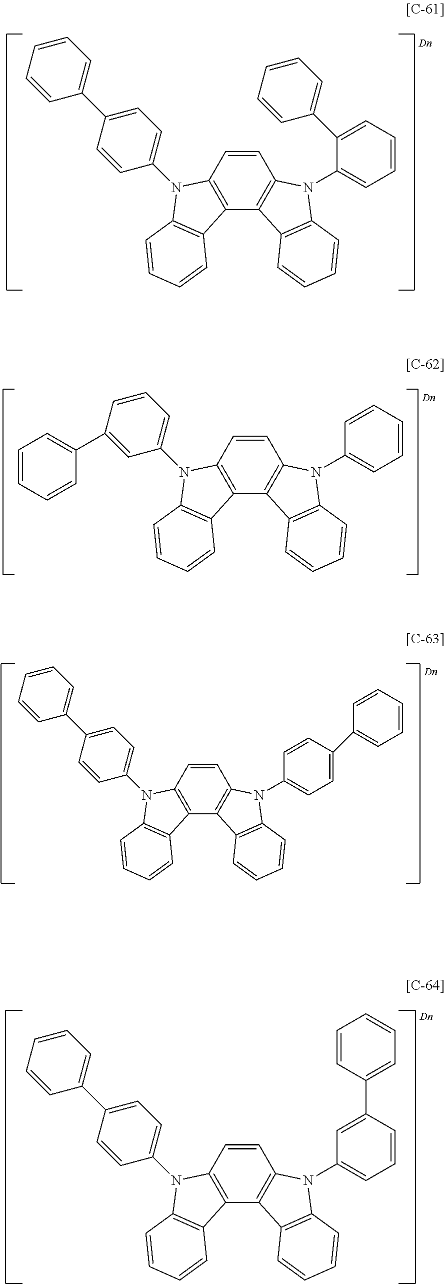

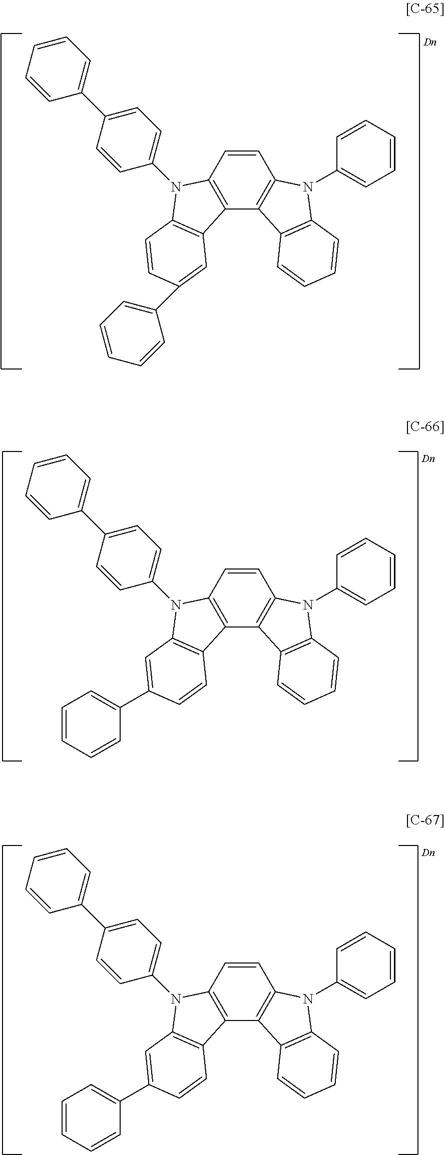

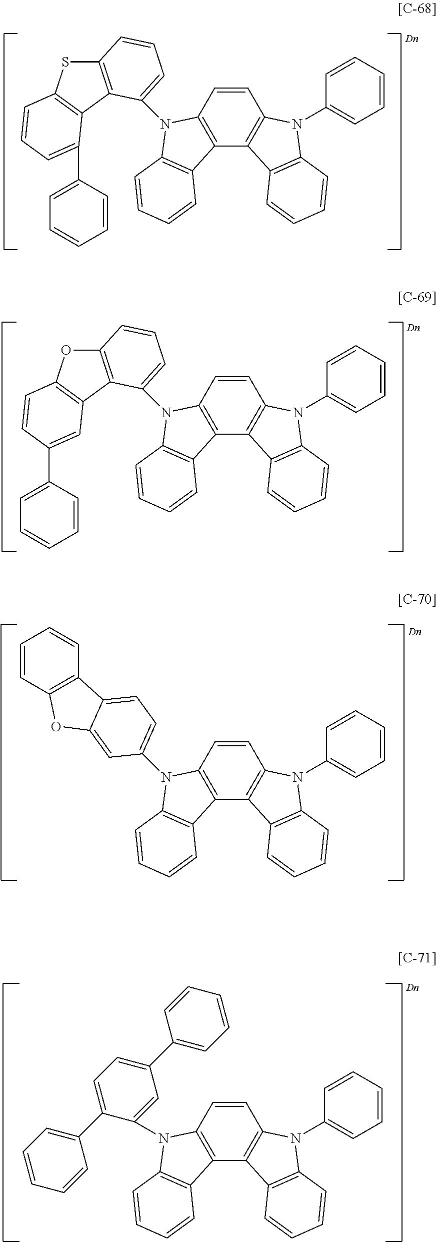

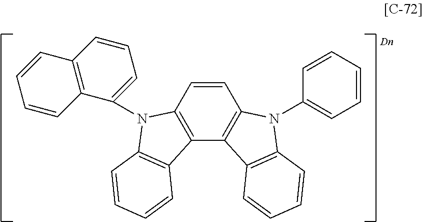

(Dn refers to the number of deuterium substitutions and indicates a structure substituted with one or more deuterium atoms)

The deuterium substitution position and deuterium substitution ratio may include all changeable ranges within the range of Compound C-1 to Compound C-72 (e.g., any hydrogen in any compound may be a protium or a deuterium).

The most specific structures for Compound C-58 to Compound C-72 are presented below as examples according to the position and substitution ratio of deuterium substitution.

In an implementation, deuterium may be substituted, as shown in the compound C-73 to compound C-102.

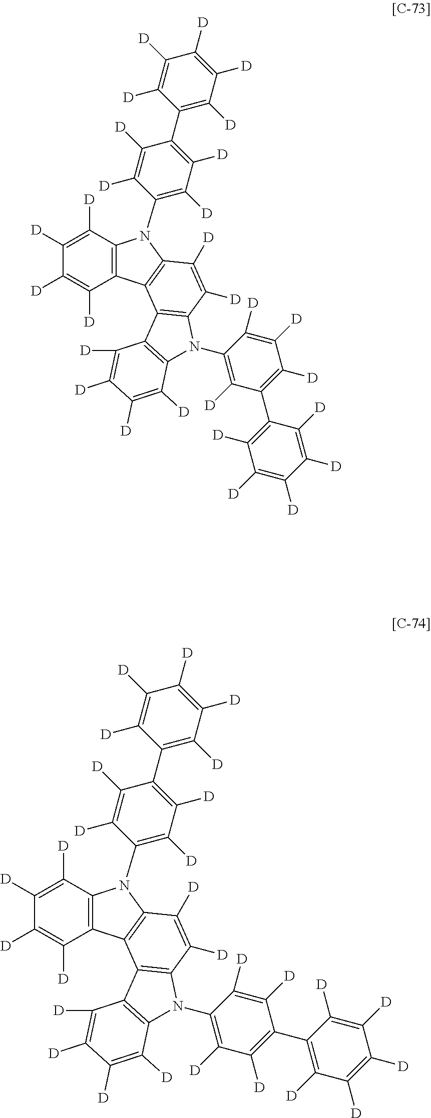 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

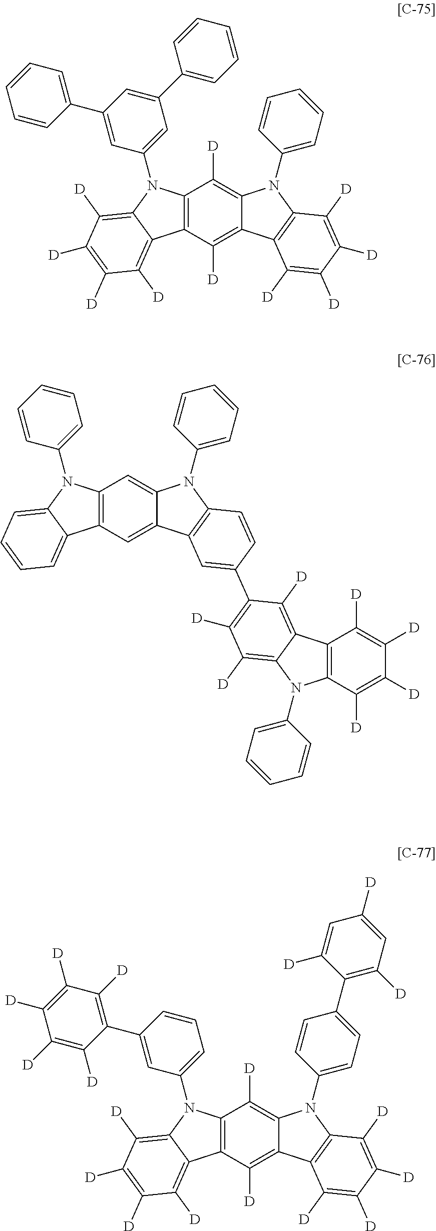 [SMILES: [2H]c1c([2H])c([2H])c2c(c1[2H])c1c([2H])c3c4c([2H])c([2H])c([2H])c([2H])c4n(-c4cc(-c5ccccc5)cc(-c5ccccc5)c4)c3c([2H])c1n2-c1ccccc1 ; [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1]

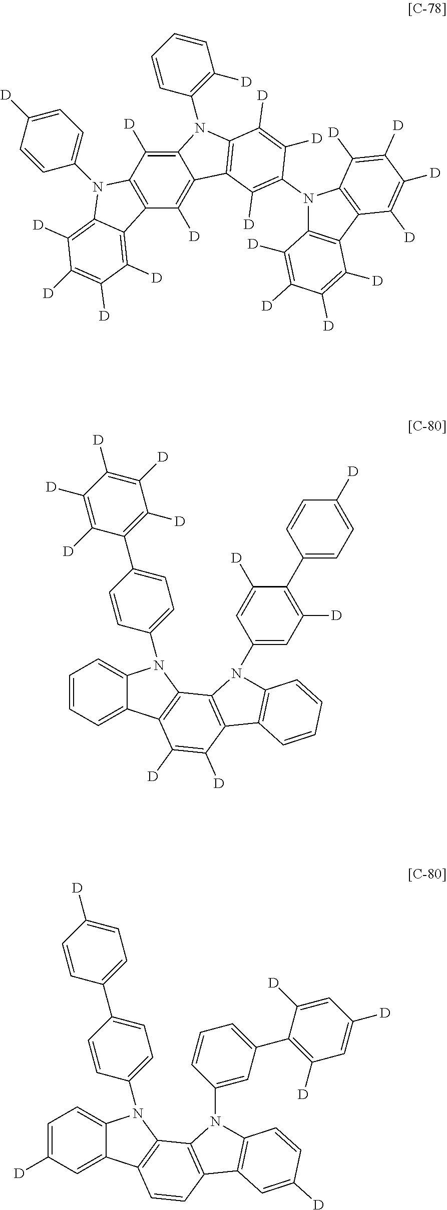 [SMILES: [2H]c1ccc(-n2c3c([2H])c([2H])c([2H])c([2H])c3c3c([2H])c4c5c([2H])c(-n6c7c([2H])c([2H])c([2H])c([2H])c7c7c([2H])c([2H])c([2H])c([2H])c76)c([2H])c([2H])c5n(-c5ccccc5[2H])c4c([2H])c32)cc1 ; [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1 ; [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1]

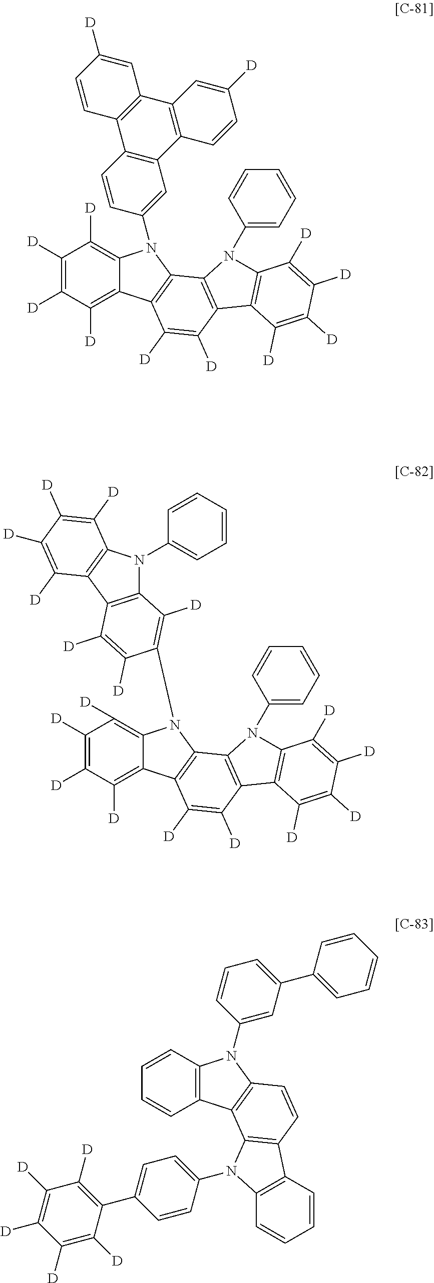 [SMILES: [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1 ; [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1 ; [2H]c1c([2H])c([2H])c(-c2ccc(-n3c4ccccc4c4ccc5c(c6ccccc6n5-c5cccc(-c6ccccc6)c5)c43)cc2)c([2H])c1[2H]]

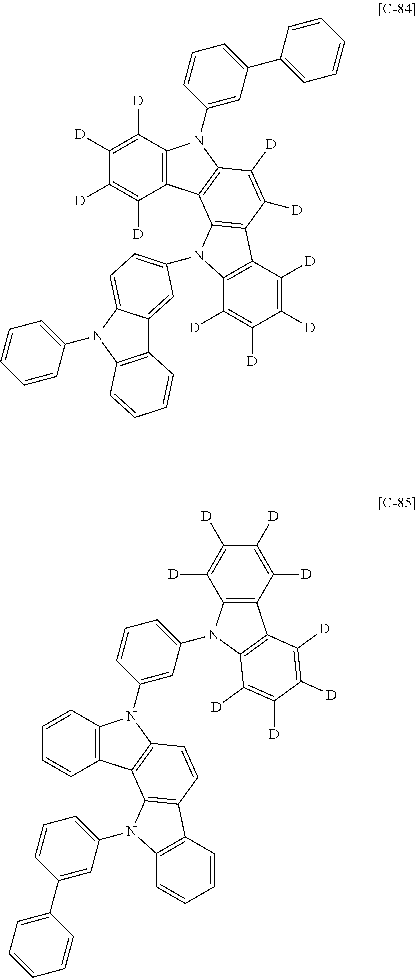 [SMILES: [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1 ; [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1]

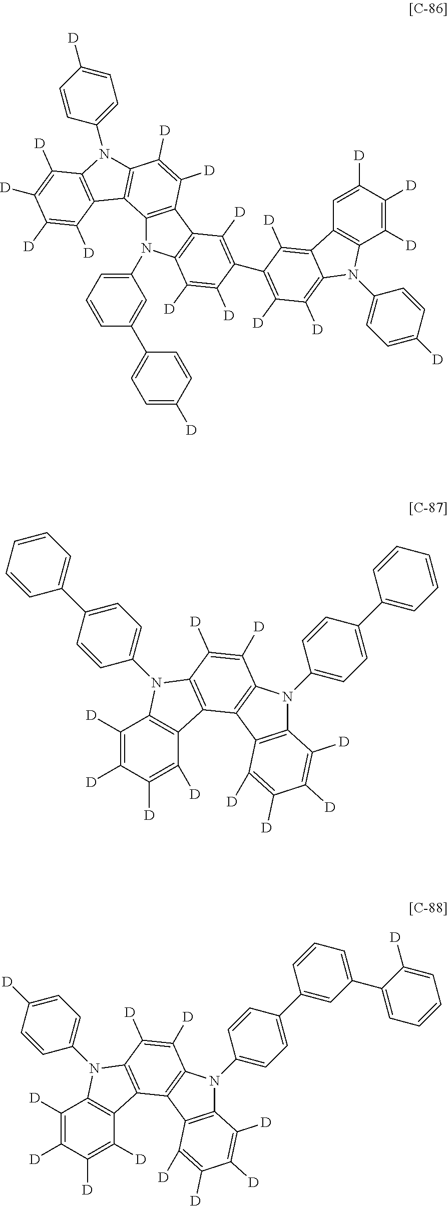 [SMILES: [2H]c1ccc(-c2cccc(-n3c4c([2H])c([2H])c(-c5c([2H])c([2H])c6c(c5[2H])c5cc([2H])c([2H])c([2H])c5n6-c5ccc([2H])cc5)c([2H])c4c4c([2H])c([2H])c5c(c6c([2H])c([2H])c([2H])c([2H])c6n5-c5ccc([2H])cc5)c43)c2)cc1 ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1 ; [2H]c1ccc(-n2c3c([2H])c([2H])c([2H])c([2H])c3c3c4c5c([2H])c([2H])c([2H])c([2H])c5n(-c5ccc(-c6cccc(-c7ccccc7[2H])c6)cc5)c4c([2H])c([2H])c32)cc1]

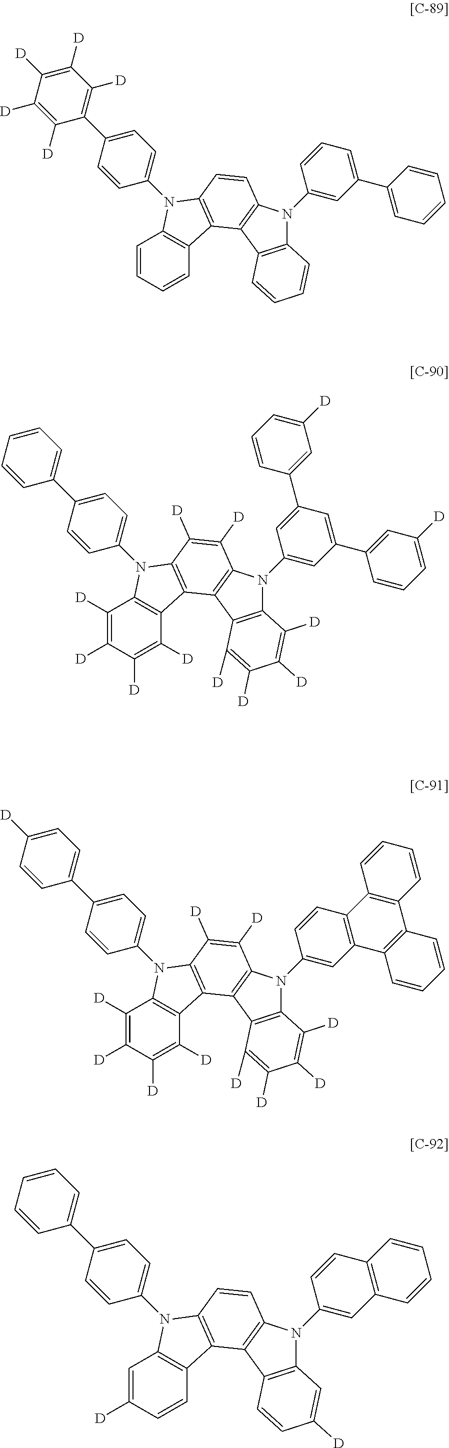 [SMILES: [2H]c1c([2H])c([2H])c(-c2ccc(-n3c4ccccc4c4c5c6ccccc6n(-c6cccc(-c7ccccc7)c6)c5ccc43)cc2)c([2H])c1[2H] ; [2H]c1cccc(-c2cc(-c3cccc([2H])c3)cc(-n3c4c([2H])c([2H])c([2H])c([2H])c4c4c5c6c([2H])c([2H])c([2H])c([2H])c6n(-c6ccc(-c7ccccc7)cc6)c5c([2H])c([2H])c43)c2)c1 ; [2H]c1ccc(-c2ccc(-n3c4c([2H])c([2H])c([2H])c([2H])c4c4c5c6c([2H])c([2H])c([2H])c([2H])c6n(-c6ccc7c8ccccc8c8ccccc8c7c6)c5c([2H])c([2H])c43)cc2)cc1 ; [2H]c1ccc2c3c4c5ccc([2H])cc5n(-c5ccc6ccccc6c5)c4ccc3n(-c3ccc(-c4ccccc4)cc3)c2c1]

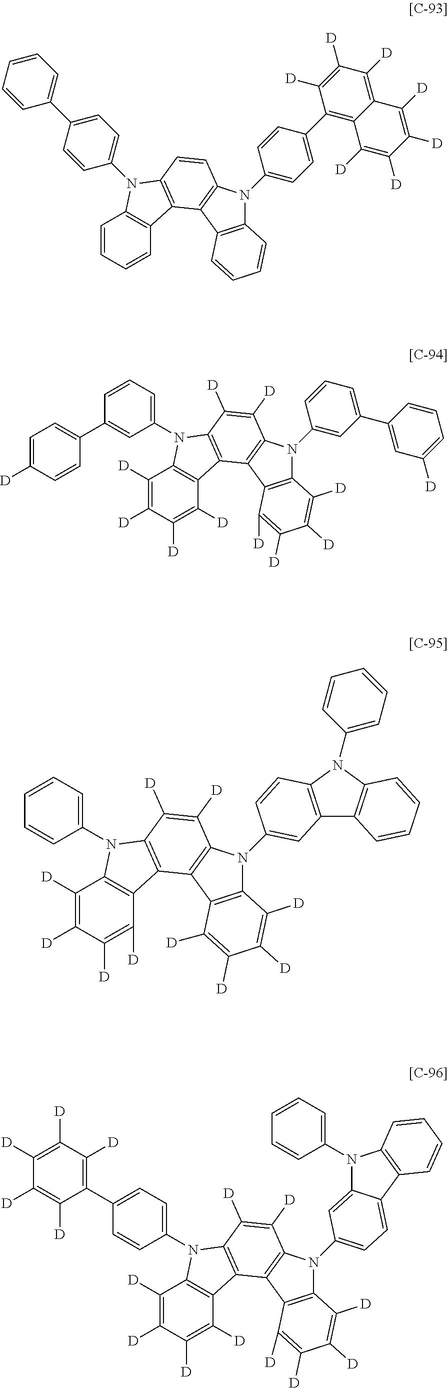

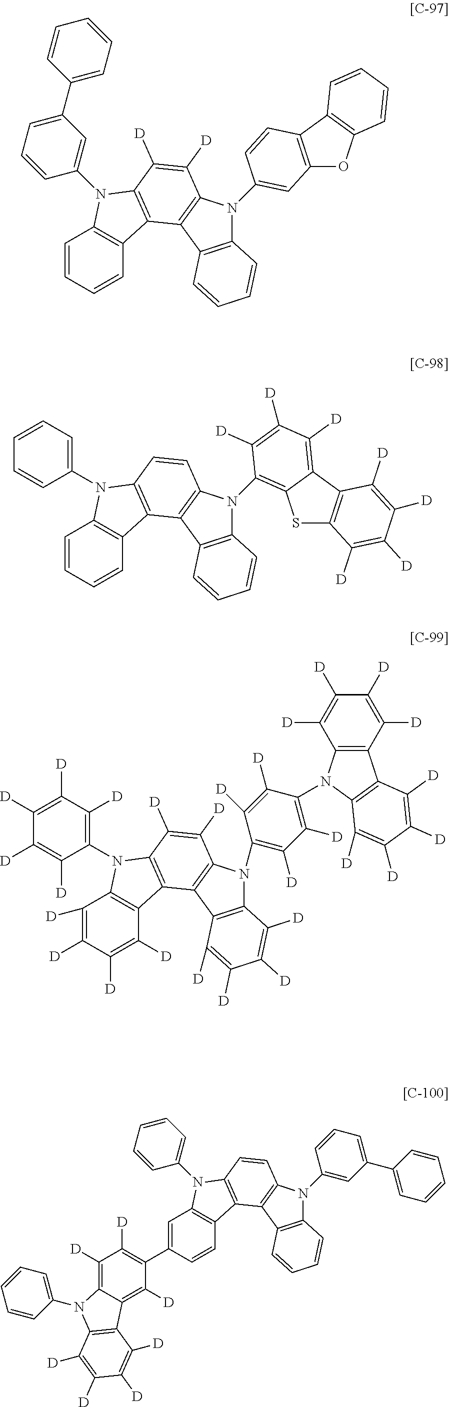

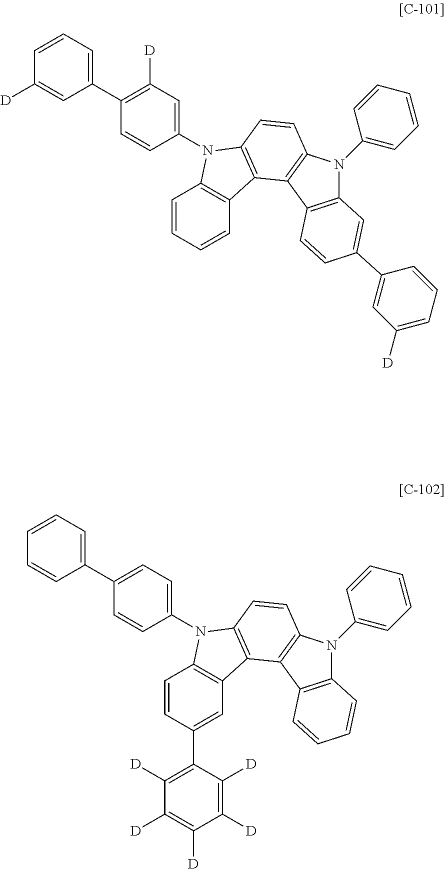 [SMILES: [2H]c1cccc(-c2ccc3c4c5c6ccccc6n(-c6ccc(-c7cccc([2H])c7)c([2H])c6)c5ccc4n(-c4ccccc4)c3c2)c1 ; [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

In an implementation, the second organic optoelectronic device compound may be, e.g., a compound of Group 4.

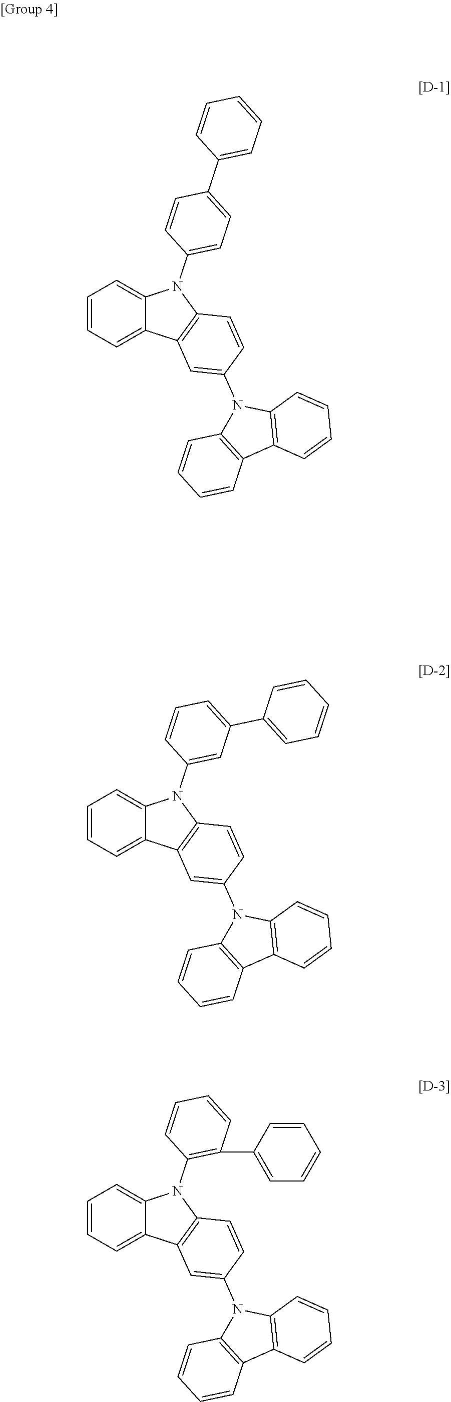 [SMILES: c1ccc(-c2ccc(-n3c4ccccc4c4cc(-n5c6ccccc6c6ccccc65)ccc43)cc2)cc1 ; c1ccc(-c2cccc(-n3c4ccccc4c4cc(-n5c6ccccc6c6ccccc65)ccc43)c2)cc1 ; c1ccc(-c2ccccc2-n2c3ccccc3c3cc(-n4c5ccccc5c5ccccc54)ccc32)cc1]

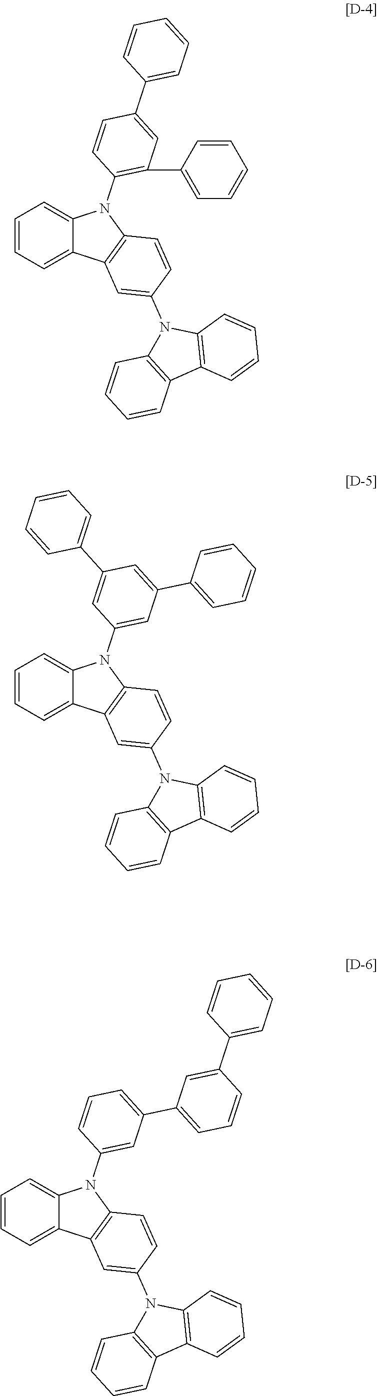 [SMILES: c1ccc(-c2ccc(-n3c4ccccc4c4cc(-n5c6ccccc6c6ccccc65)ccc43)c(-c3ccccc3)c2)cc1 ; c1ccc(-c2cc(-c3ccccc3)cc(-n3c4ccccc4c4cc(-n5c6ccccc6c6ccccc65)ccc43)c2)cc1 ; c1ccc(-c2cccc(-c3cccc(-n4c5ccccc5c5cc(-n6c7ccccc7c7ccccc76)ccc54)c3)c2)cc1]

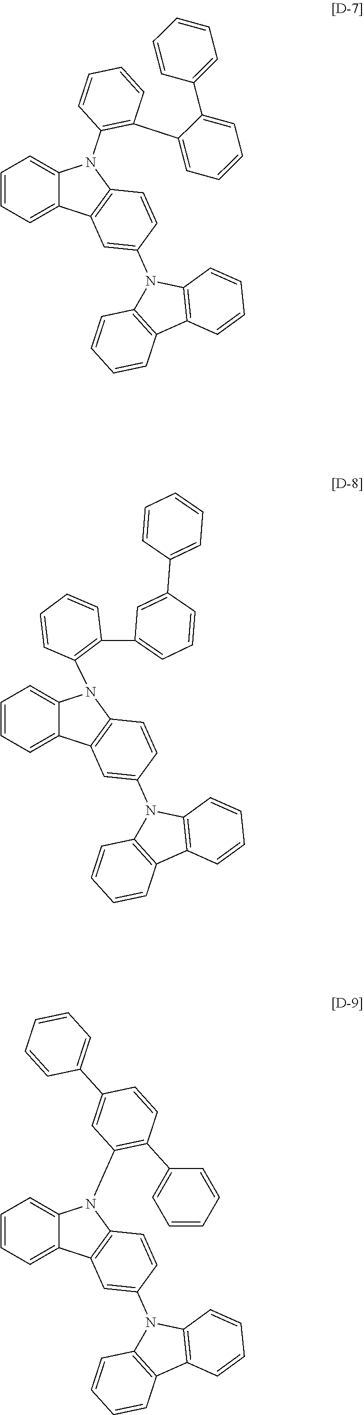 [SMILES: c1ccc(-c2ccccc2-c2ccccc2-n2c3ccccc3c3cc(-n4c5ccccc5c5ccccc54)ccc32)cc1 ; c1ccc(-c2cccc(-c3ccccc3-n3c4ccccc4c4cc(-n5c6ccccc6c6ccccc65)ccc43)c2)cc1 ; c1ccc(-c2ccc(-c3ccccc3)c(-n3c4ccccc4c4cc(-n5c6ccccc6c6ccccc65)ccc43)c2)cc1]

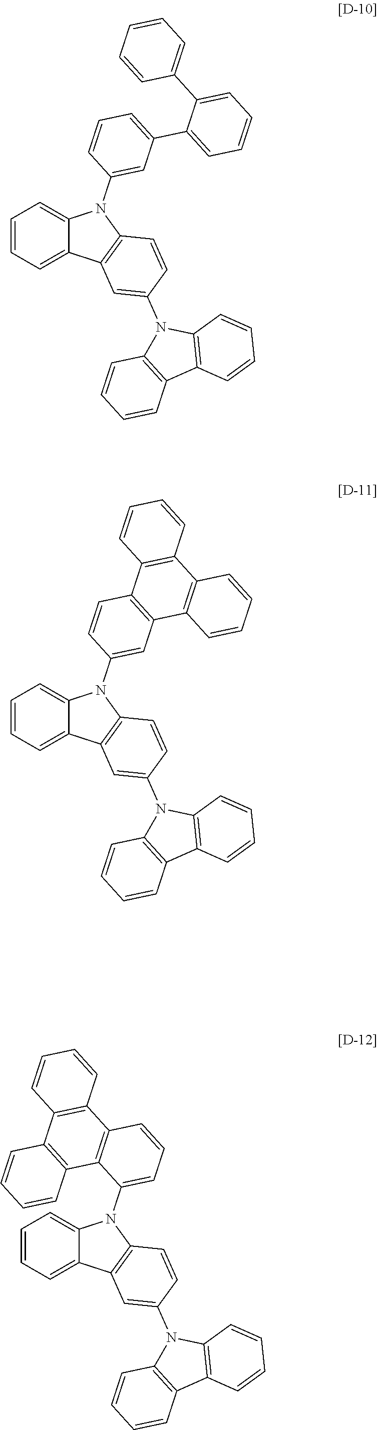 [SMILES: c1ccc(-c2ccccc2-c2cccc(-n3c4ccccc4c4cc(-n5c6ccccc6c6ccccc65)ccc43)c2)cc1 ; c1ccc2c(c1)c1ccccc1c1cc(-n3c4ccccc4c4cc(-n5c6ccccc6c6ccccc65)ccc43)ccc21 ; c1ccc2c(c1)c1ccccc1c1c(-n3c4ccccc4c4cc(-n5c6ccccc6c6ccccc65)ccc43)cccc21]

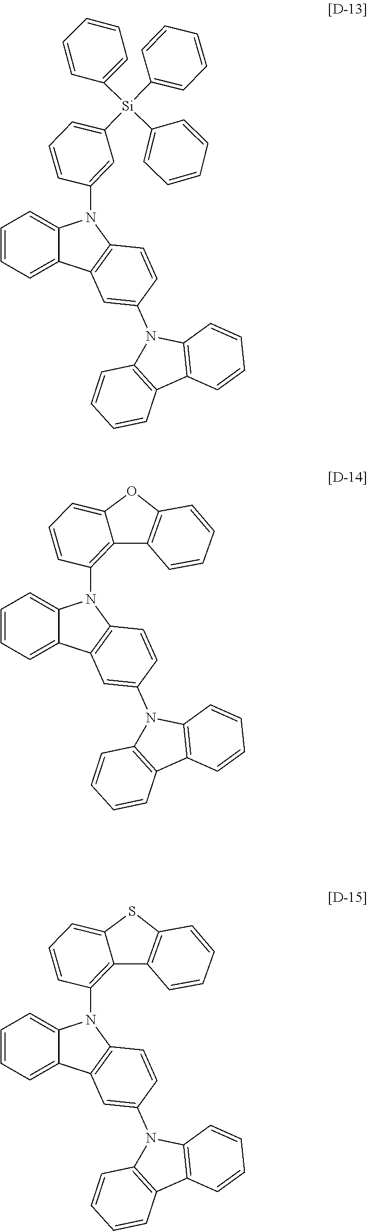 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc2c(c1)oc1cccc(-n3c4ccccc4c4cc(-n5c6ccccc6c6ccccc65)ccc43)c12 ; c1ccc2c(c1)sc1cccc(-n3c4ccccc4c4cc(-n5c6ccccc6c6ccccc65)ccc43)c12]

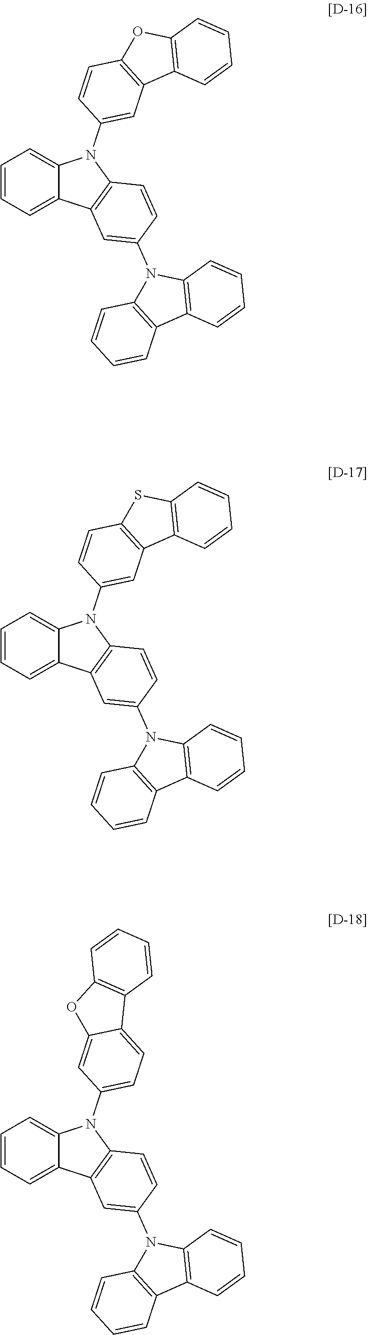 [SMILES: c1ccc2c(c1)oc1ccc(-n3c4ccccc4c4cc(-n5c6ccccc6c6ccccc65)ccc43)cc12 ; c1ccc2c(c1)sc1ccc(-n3c4ccccc4c4cc(-n5c6ccccc6c6ccccc65)ccc43)cc12 ; c1ccc2c(c1)oc1cc(-n3c4ccccc4c4cc(-n5c6ccccc6c6ccccc65)ccc43)ccc12]

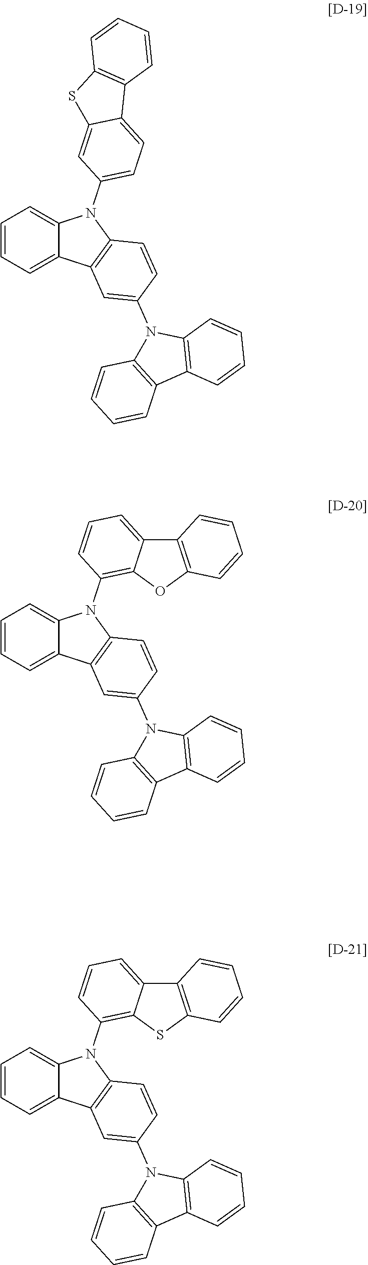 [SMILES: c1ccc2c(c1)sc1cc(-n3c4ccccc4c4cc(-n5c6ccccc6c6ccccc65)ccc43)ccc12 ; c1ccc2c(c1)oc1c(-n3c4ccccc4c4cc(-n5c6ccccc6c6ccccc65)ccc43)cccc12 ; c1ccc2c(c1)sc1c(-n3c4ccccc4c4cc(-n5c6ccccc6c6ccccc65)ccc43)cccc12]

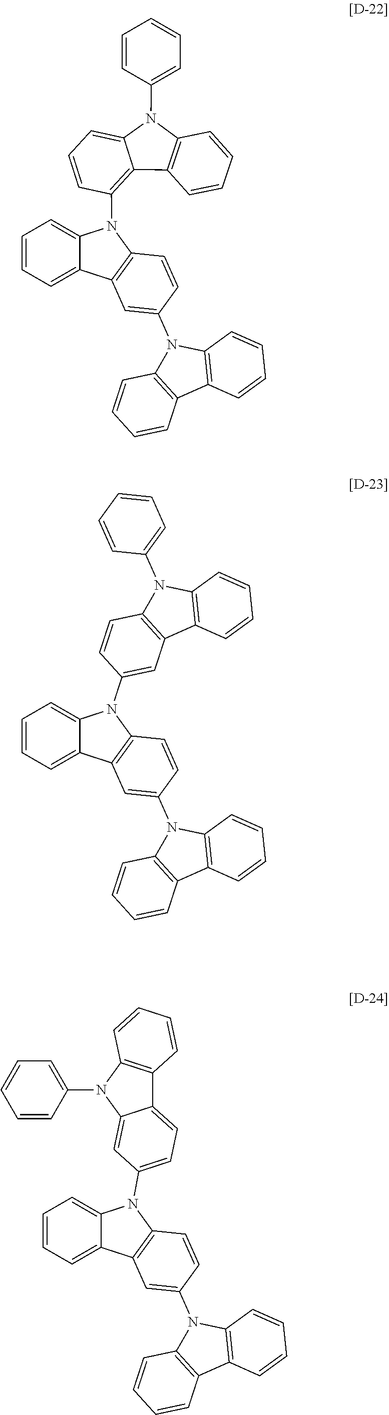 [SMILES: c1ccc(-n2c3ccccc3c3c(-n4c5ccccc5c5cc(-n6c7ccccc7c7ccccc76)ccc54)cccc32)cc1 ; c1ccc(-n2c3ccccc3c3cc(-n4c5ccccc5c5cc(-n6c7ccccc7c7ccccc76)ccc54)ccc32)cc1 ; c1ccc(-n2c3ccccc3c3ccc(-n4c5ccccc5c5cc(-n6c7ccccc7c7ccccc76)ccc54)cc32)cc1]

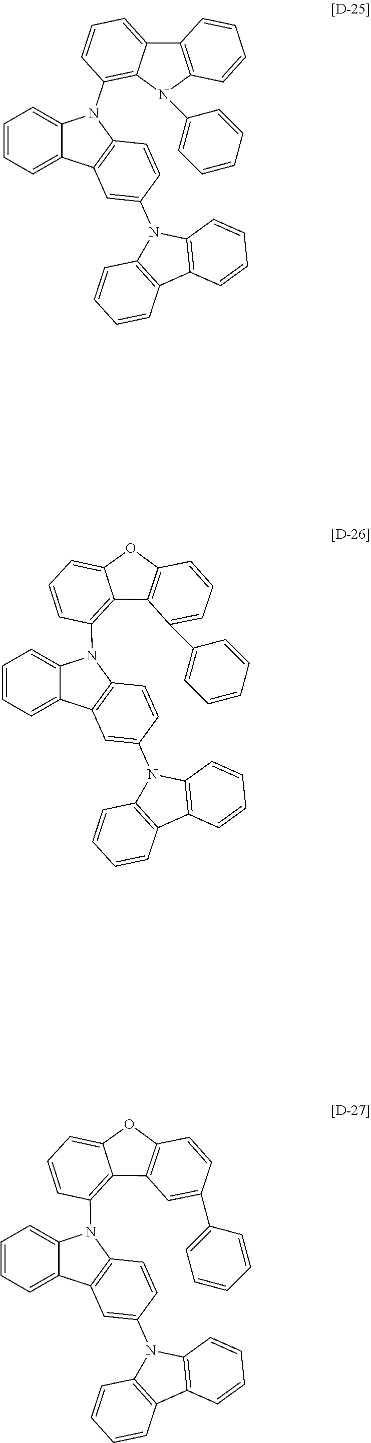 [SMILES: c1ccc(-n2c3ccccc3c3cccc(-n4c5ccccc5c5cc(-n6c7ccccc7c7ccccc76)ccc54)c32)cc1 ; c1ccc(-c2cccc3oc4cccc(-n5c6ccccc6c6cc(-n7c8ccccc8c8ccccc87)ccc65)c4c23)cc1 ; c1ccc(-c2ccc3oc4cccc(-n5c6ccccc6c6cc(-n7c8ccccc8c8ccccc87)ccc65)c4c3c2)cc1]

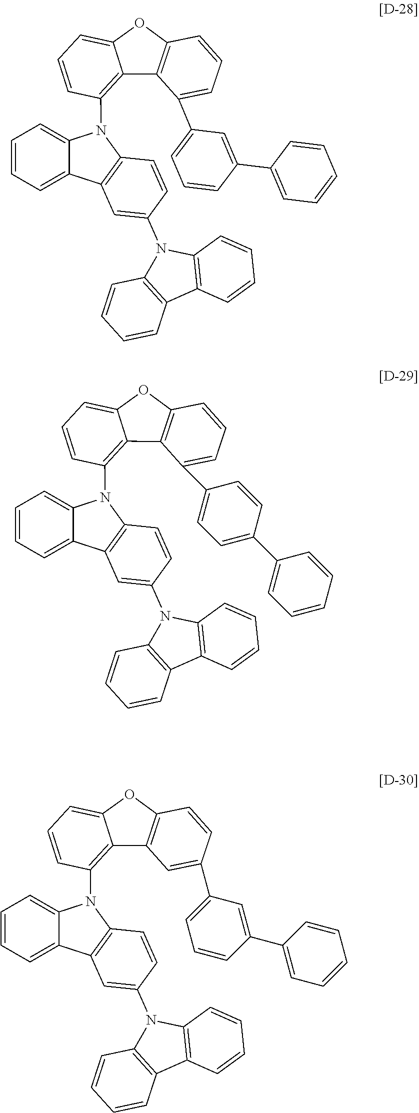 [SMILES: c1ccc(-c2cccc(-c3cccc4oc5cccc(-n6c7ccccc7c7cc(-n8c9ccccc9c9ccccc98)ccc76)c5c34)c2)cc1 ; c1ccc(-c2ccc(-c3cccc4oc5cccc(-n6c7ccccc7c7cc(-n8c9ccccc9c9ccccc98)ccc76)c5c34)cc2)cc1 ; c1ccc(-c2cccc(-c3ccc4oc5cccc(-n6c7ccccc7c7cc(-n8c9ccccc9c9ccccc98)ccc76)c5c4c3)c2)cc1]

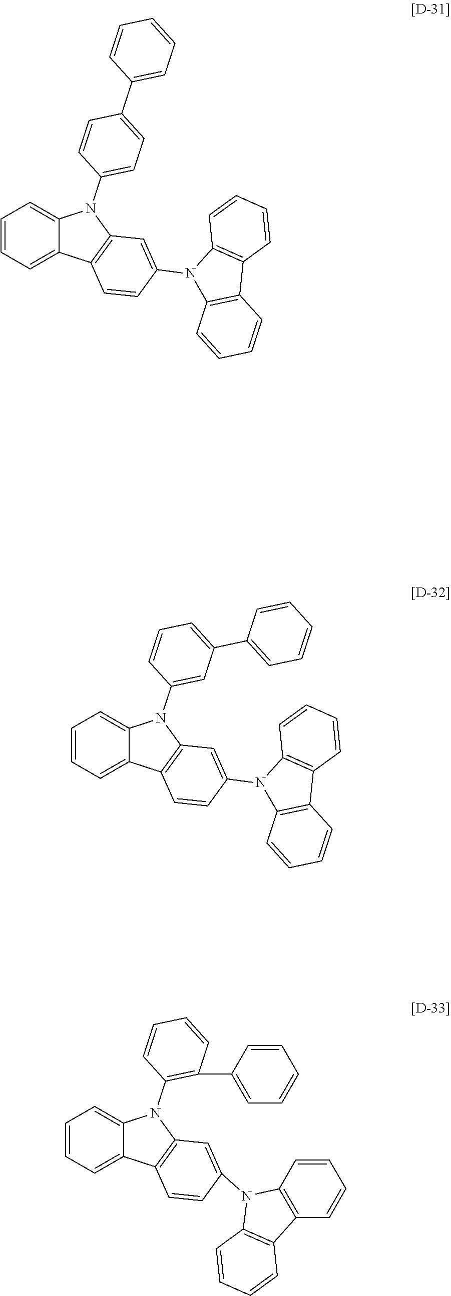 [SMILES: c1ccc(-c2ccc(-n3c4ccccc4c4ccc(-n5c6ccccc6c6ccccc65)cc43)cc2)cc1 ; c1ccc(-c2cccc(-n3c4ccccc4c4ccc(-n5c6ccccc6c6ccccc65)cc43)c2)cc1 ; c1ccc(-c2ccccc2-n2c3ccccc3c3ccc(-n4c5ccccc5c5ccccc54)cc32)cc1]

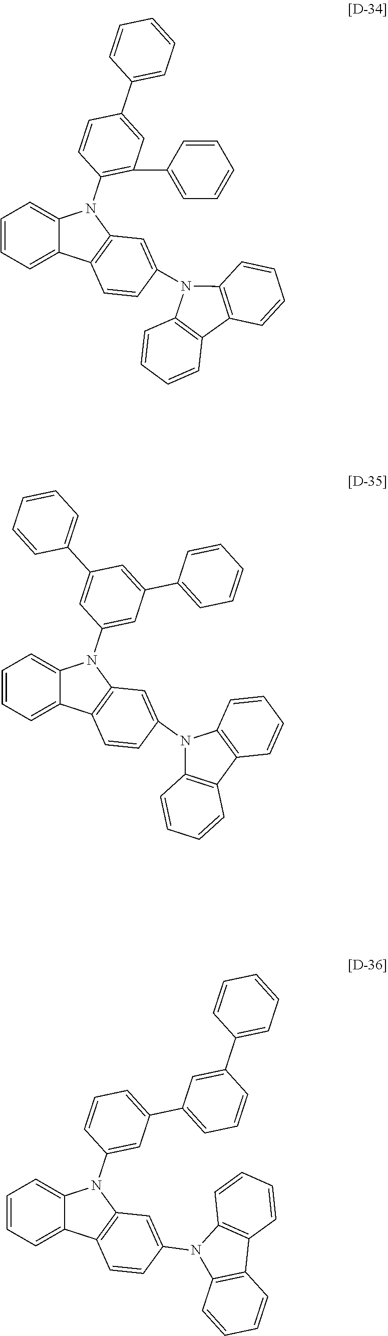 [SMILES: c1ccc(-c2ccc(-n3c4ccccc4c4ccc(-n5c6ccccc6c6ccccc65)cc43)c(-c3ccccc3)c2)cc1 ; c1ccc(-c2cc(-c3ccccc3)cc(-n3c4ccccc4c4ccc(-n5c6ccccc6c6ccccc65)cc43)c2)cc1 ; c1ccc(-c2cccc(-c3cccc(-n4c5ccccc5c5ccc(-n6c7ccccc7c7ccccc76)cc54)c3)c2)cc1]

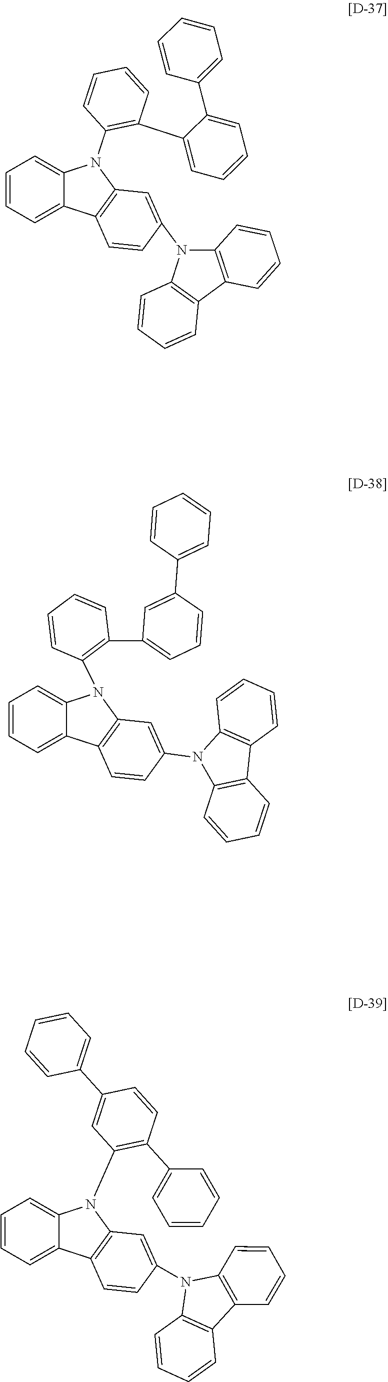 [SMILES: c1ccc(-c2ccccc2-c2ccccc2-n2c3ccccc3c3ccc(-n4c5ccccc5c5ccccc54)cc32)cc1 ; c1ccc(-c2cccc(-c3ccccc3-n3c4ccccc4c4ccc(-n5c6ccccc6c6ccccc65)cc43)c2)cc1 ; c1ccc(-c2ccc(-c3ccccc3)c(-n3c4ccccc4c4ccc(-n5c6ccccc6c6ccccc65)cc43)c2)cc1]

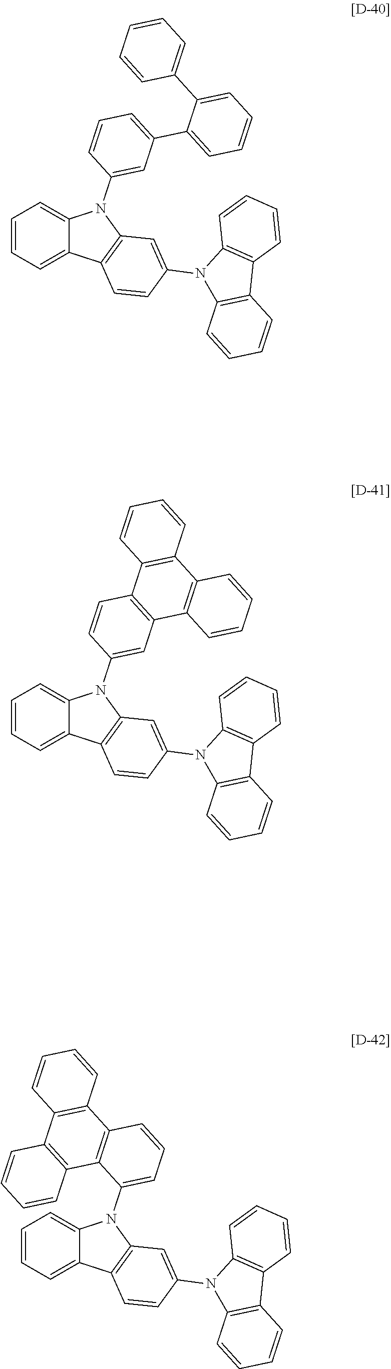 [SMILES: c1ccc(-c2ccccc2-c2cccc(-n3c4ccccc4c4ccc(-n5c6ccccc6c6ccccc65)cc43)c2)cc1 ; c1ccc2c(c1)c1ccccc1c1cc(-n3c4ccccc4c4ccc(-n5c6ccccc6c6ccccc65)cc43)ccc21 ; c1ccc2c(c1)c1ccccc1c1c(-n3c4ccccc4c4ccc(-n5c6ccccc6c6ccccc65)cc43)cccc21]

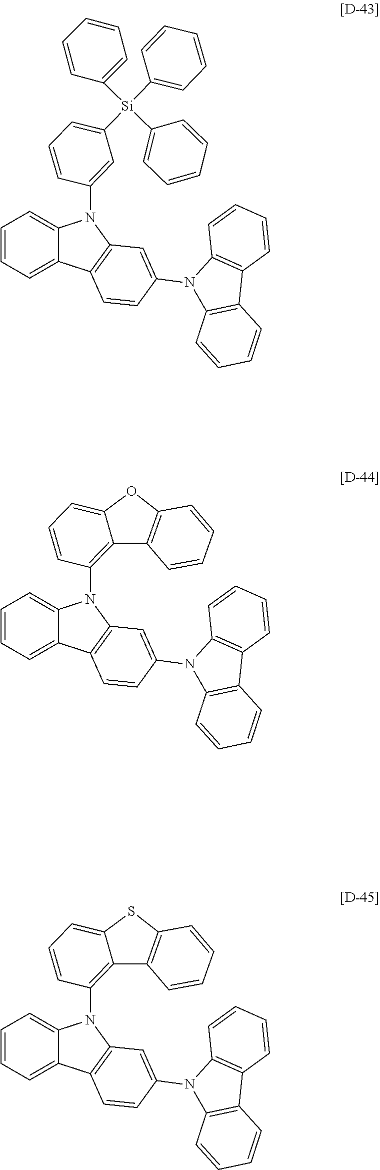 [SMILES: c1ccc([Si](c2ccccc2)(c2ccccc2)c2cccc(-n3c4ccccc4c4ccc(-n5c6ccccc6c6ccccc65)cc43)c2)cc1 ; c1ccc2c(c1)oc1cccc(-n3c4ccccc4c4ccc(-n5c6ccccc6c6ccccc65)cc43)c12 ; c1ccc2c(c1)sc1cccc(-n3c4ccccc4c4ccc(-n5c6ccccc6c6ccccc65)cc43)c12]

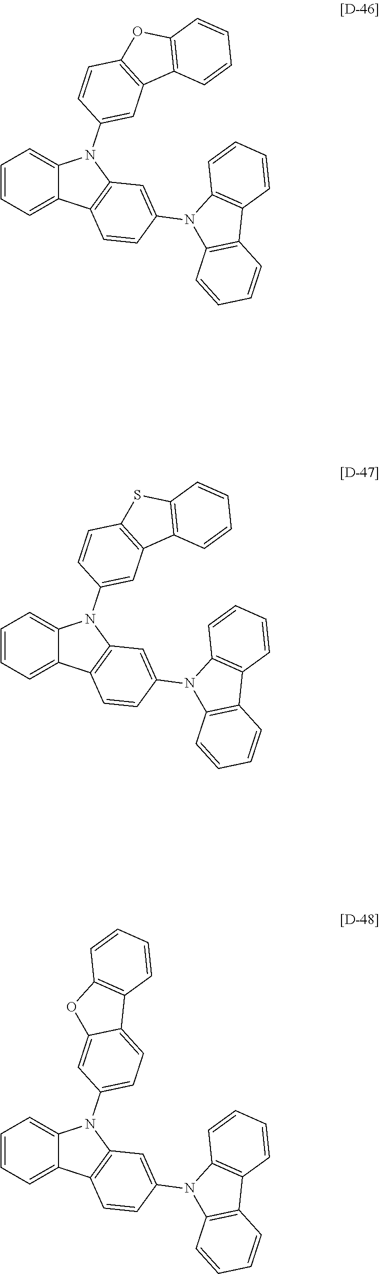 [SMILES: c1ccc2c(c1)oc1ccc(-n3c4ccccc4c4ccc(-n5c6ccccc6c6ccccc65)cc43)cc12 ; c1ccc2c(c1)sc1ccc(-n3c4ccccc4c4ccc(-n5c6ccccc6c6ccccc65)cc43)cc12 ; c1ccc2c(c1)oc1cc(-n3c4ccccc4c4ccc(-n5c6ccccc6c6ccccc65)cc43)ccc12]

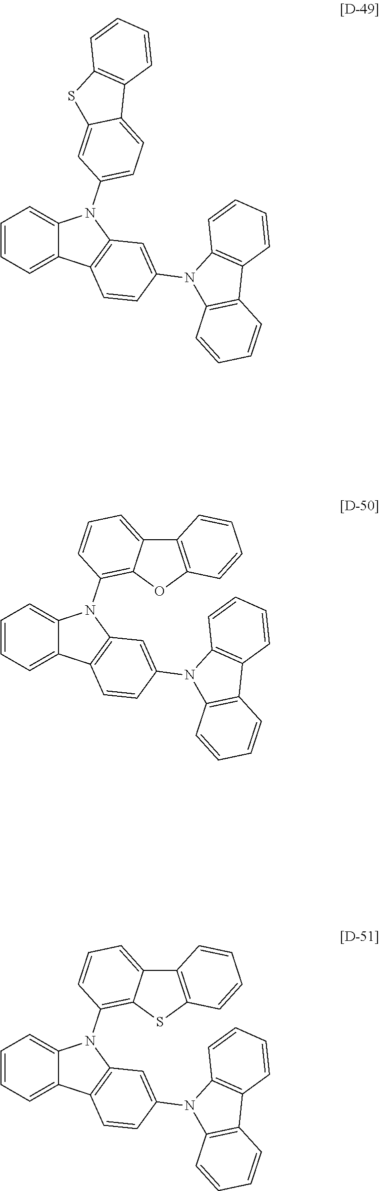 [SMILES: c1ccc2c(c1)sc1cc(-n3c4ccccc4c4ccc(-n5c6ccccc6c6ccccc65)cc43)ccc12 ; c1ccc2c(c1)oc1c(-n3c4ccccc4c4ccc(-n5c6ccccc6c6ccccc65)cc43)cccc12 ; c1ccc2c(c1)sc1c(-n3c4ccccc4c4ccc(-n5c6ccccc6c6ccccc65)cc43)cccc12]

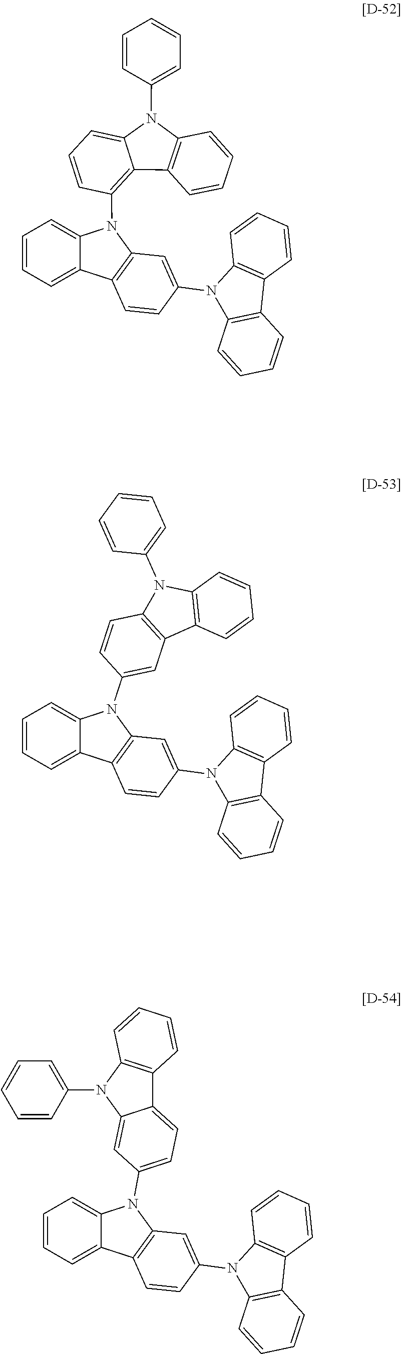 [SMILES: c1ccc(-n2c3ccccc3c3c(-n4c5ccccc5c5ccc(-n6c7ccccc7c7ccccc76)cc54)cccc32)cc1 ; c1ccc(-n2c3ccccc3c3cc(-n4c5ccccc5c5ccc(-n6c7ccccc7c7ccccc76)cc54)ccc32)cc1 ; c1ccc(-n2c3ccccc3c3ccc(-n4c5ccccc5c5ccc(-n6c7ccccc7c7ccccc76)cc54)cc32)cc1]

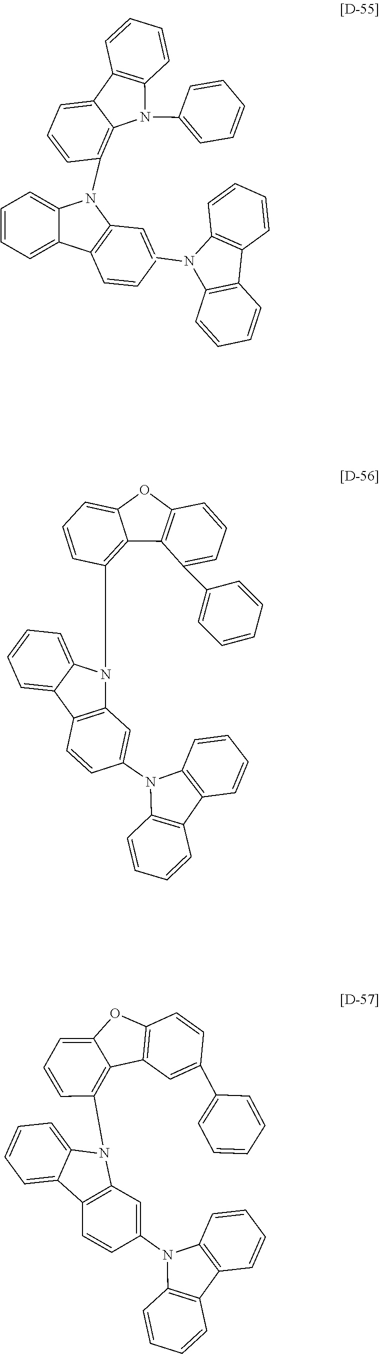

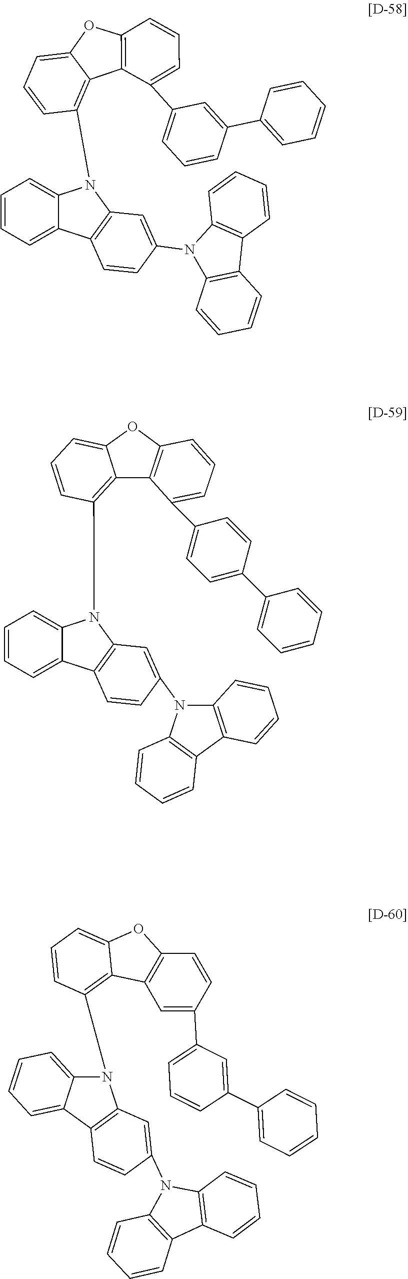 [SMILES: c1ccc(-c2cccc(-c3cccc4oc5cccc(-n6c7ccccc7c7ccc(-n8c9ccccc9c9ccccc98)cc76)c5c34)c2)cc1 ; c1ccc(-c2ccc(-c3cccc4oc5cccc(-n6c7ccccc7c7ccc(-n8c9ccccc9c9ccccc98)cc76)c5c34)cc2)cc1 ; c1ccc(-c2cccc(-c3ccc4oc5cccc(-n6c7ccccc7c7ccc(-n8c9ccccc9c9ccccc98)cc76)c5c4c3)c2)cc1]

In an implementation, examples of Compound D-1 to Compound D-60 listed in Group 4 in which at least one hydrogen is replaced with deuterium are given below.

In an implementation, deuterium may be substituted, e.g., as shown in the compound D-61 to compound D-120 exemplified below.

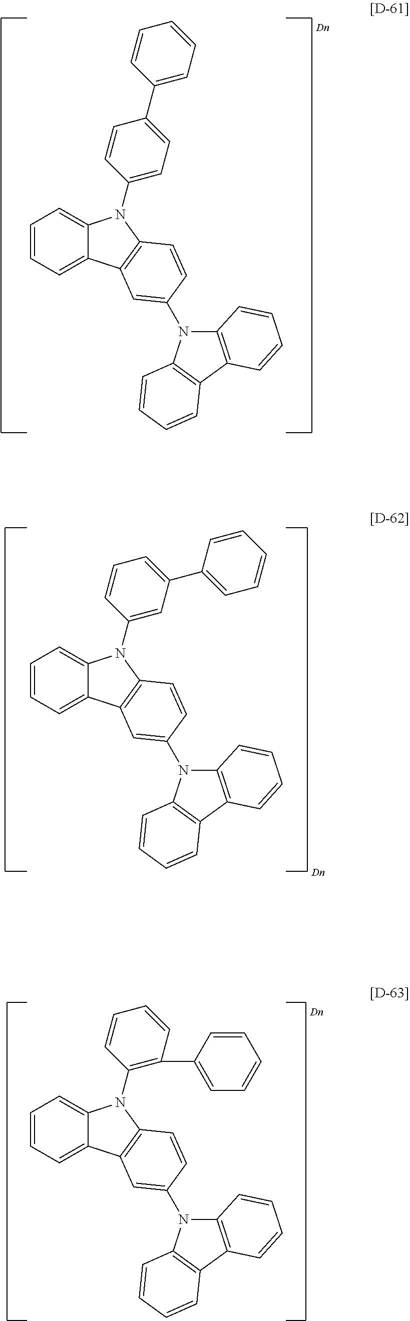

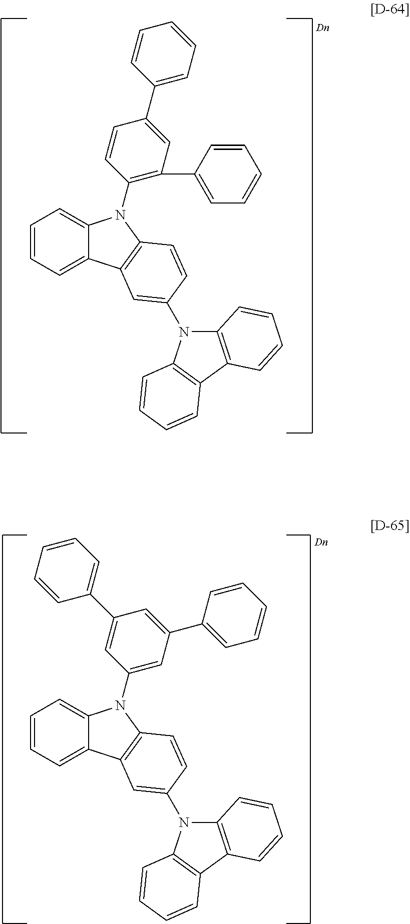

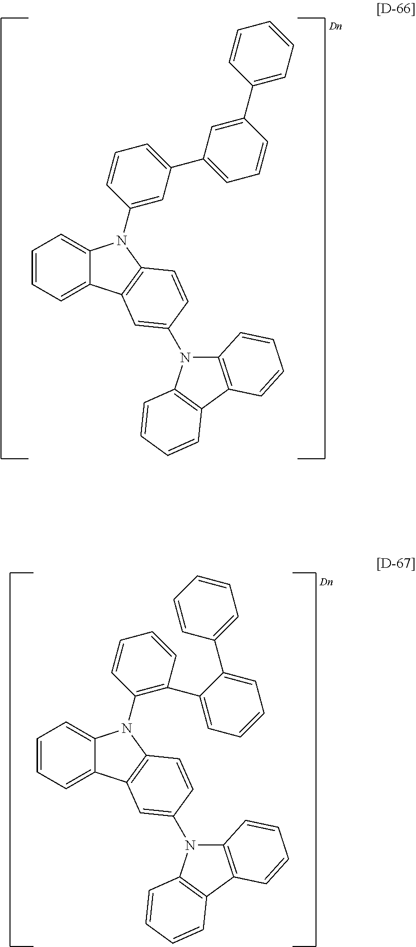

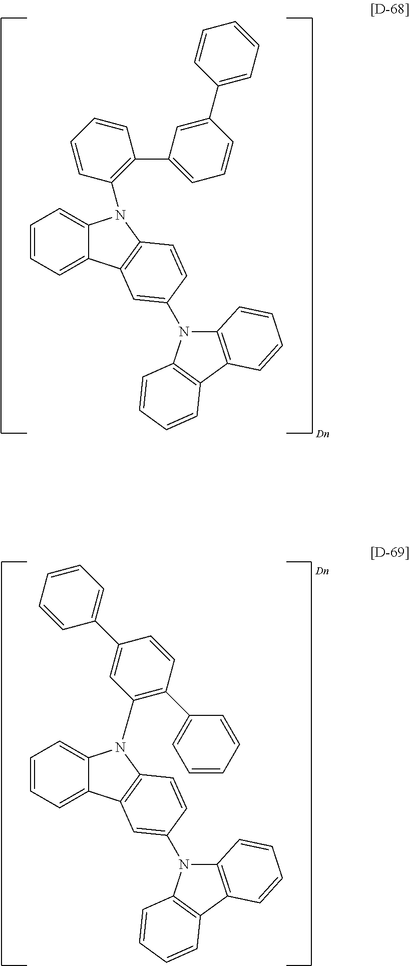

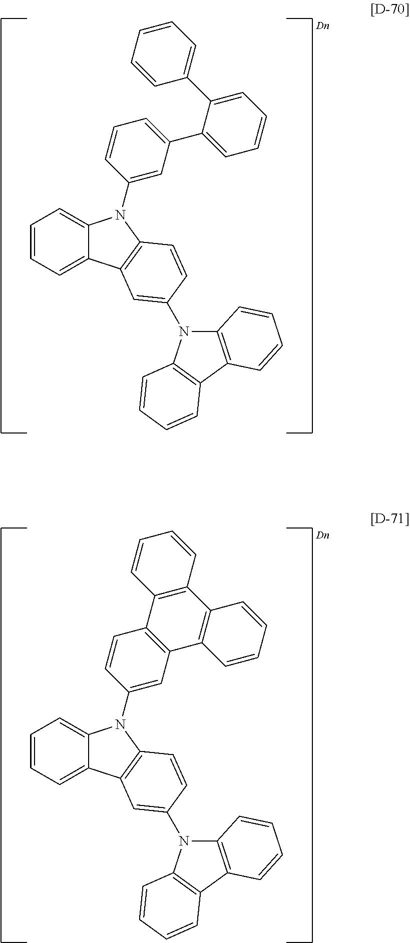

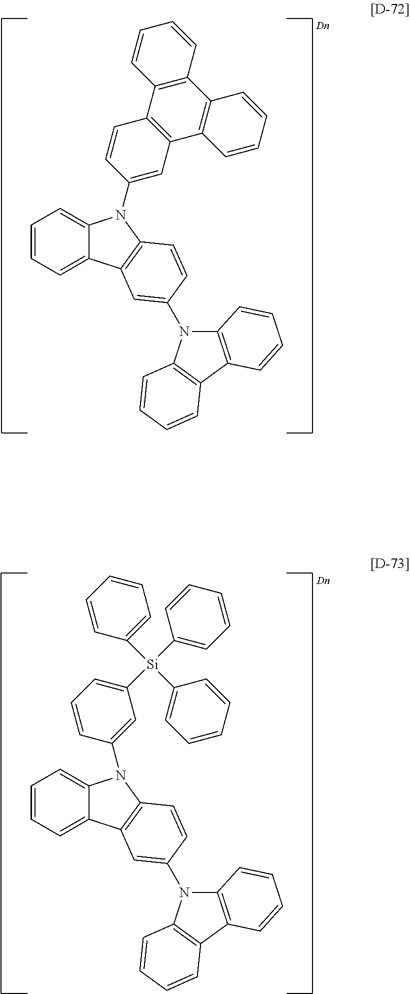

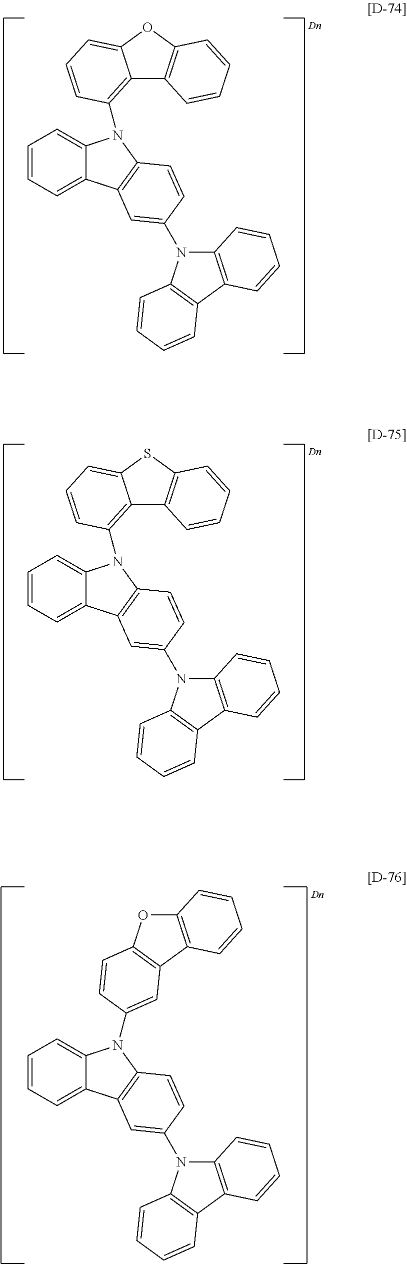

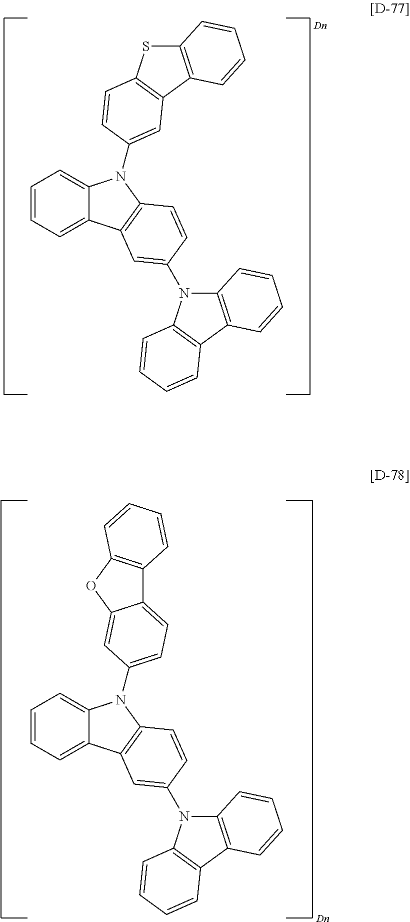

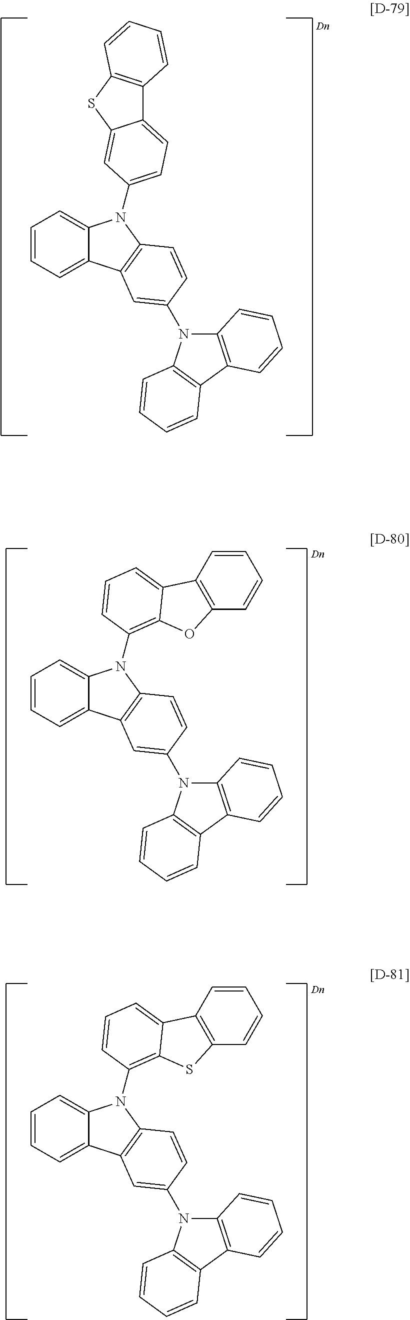

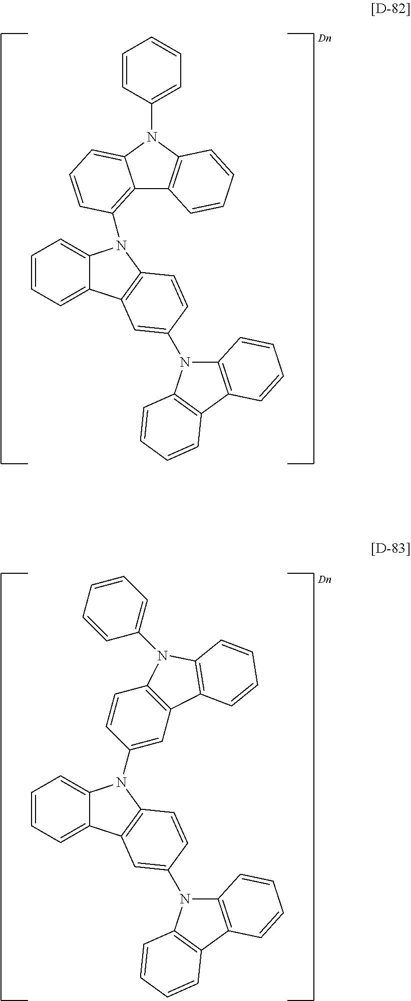

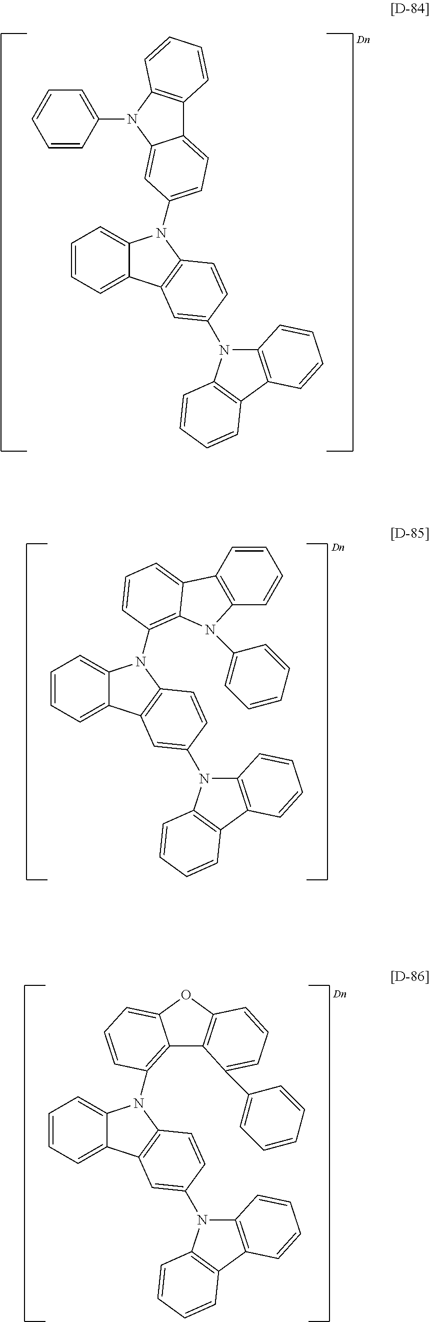

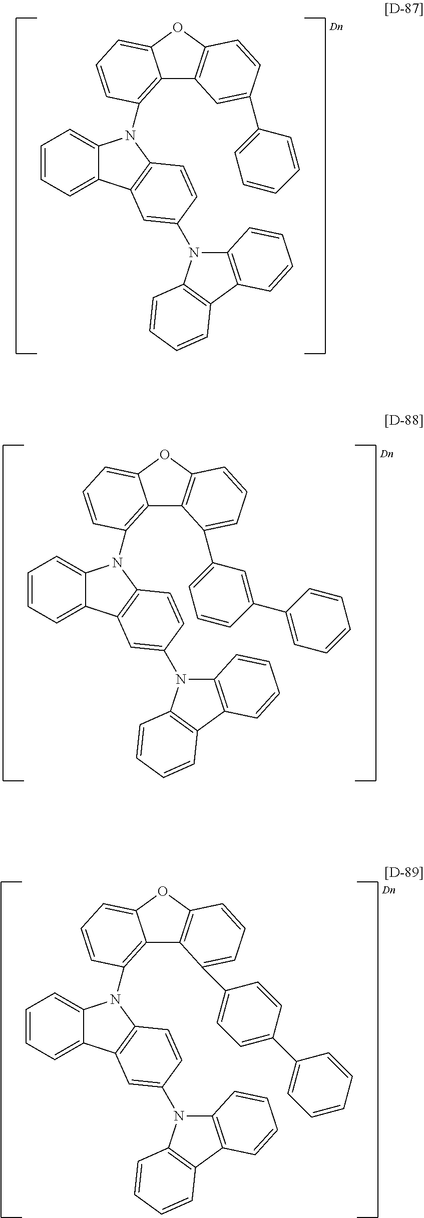

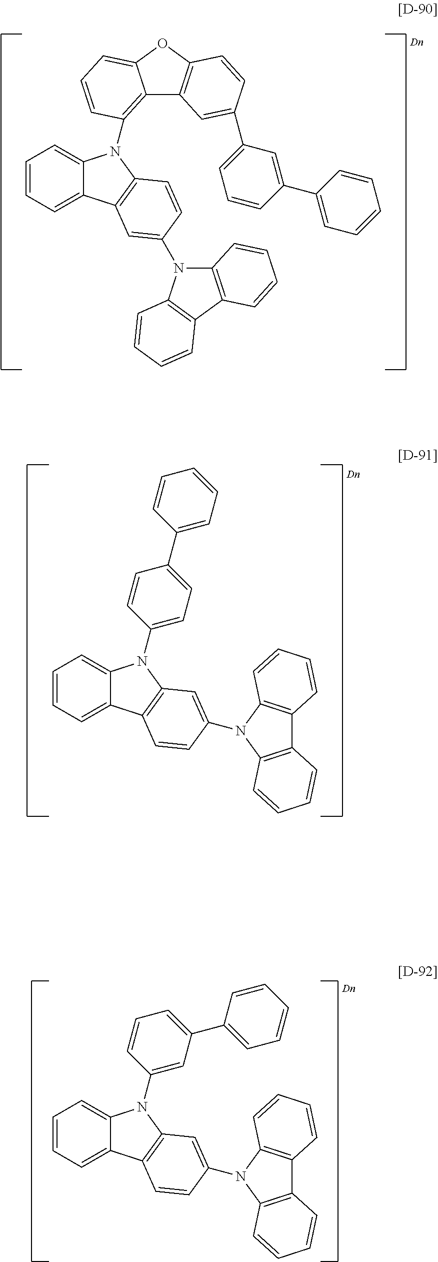

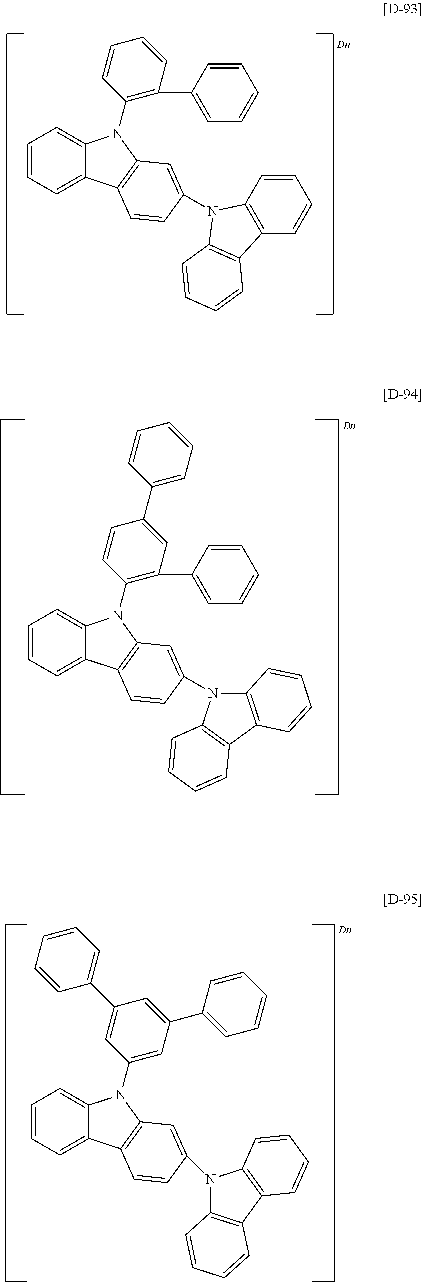

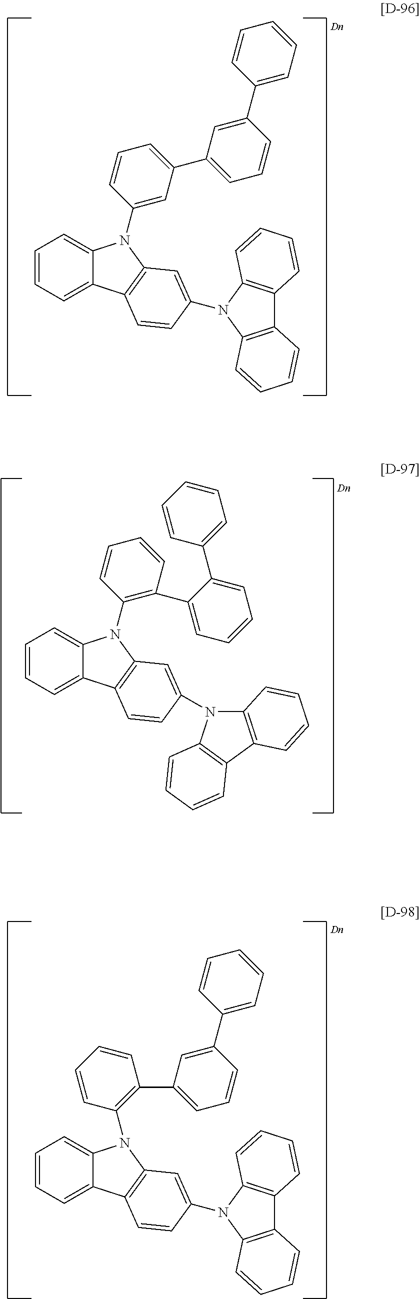

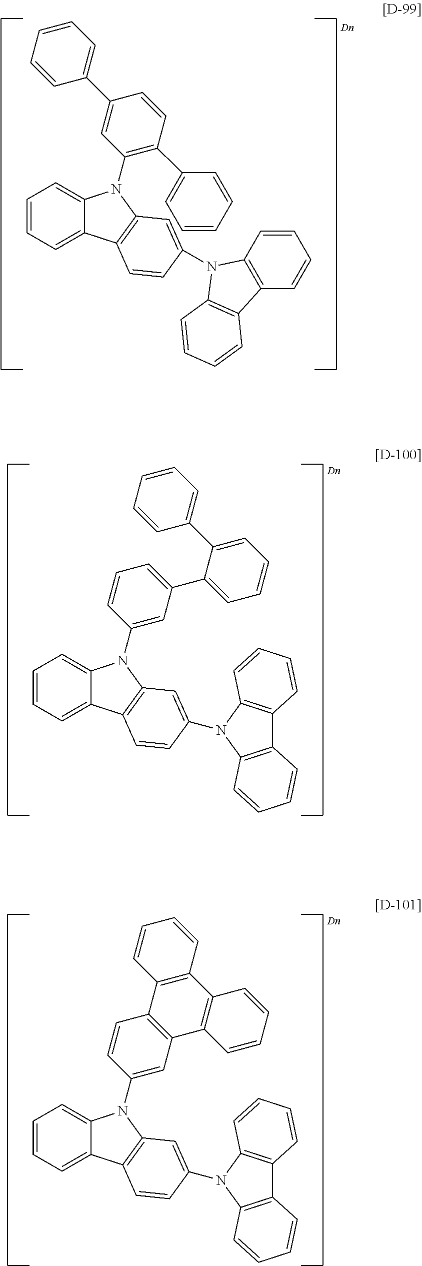

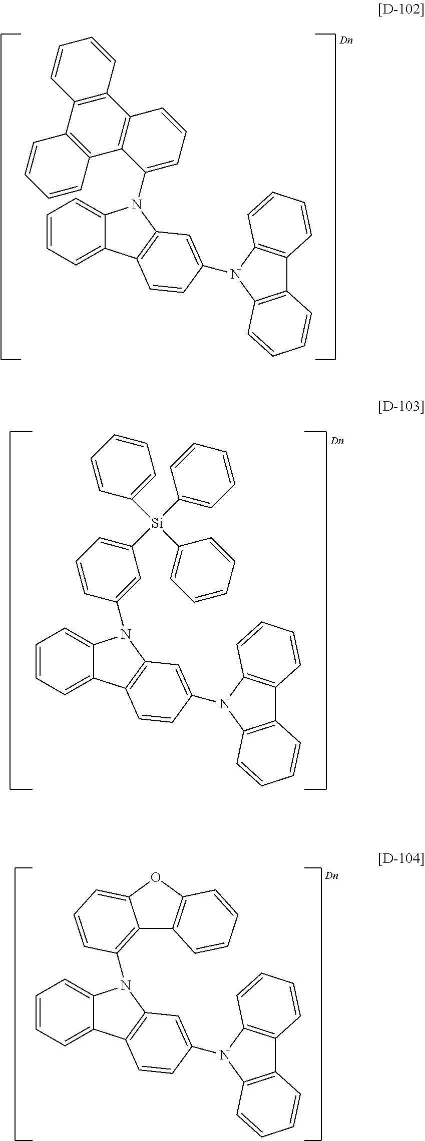

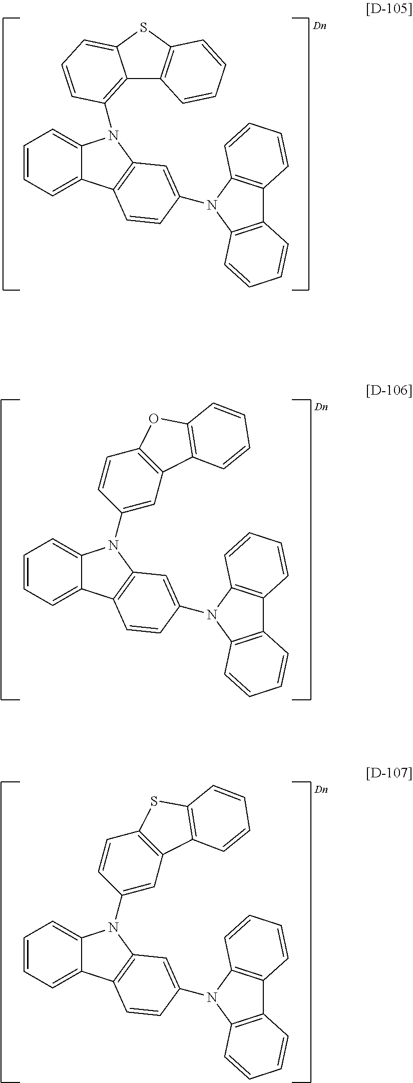

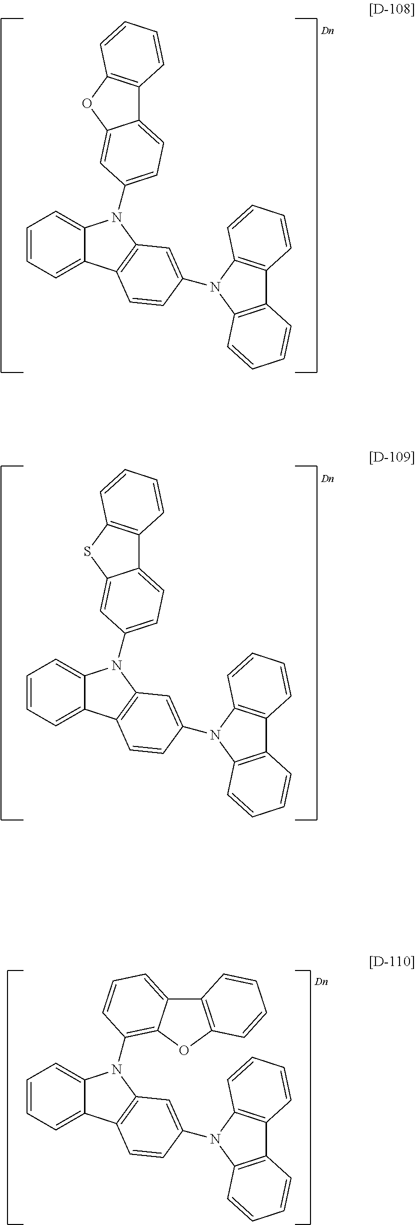

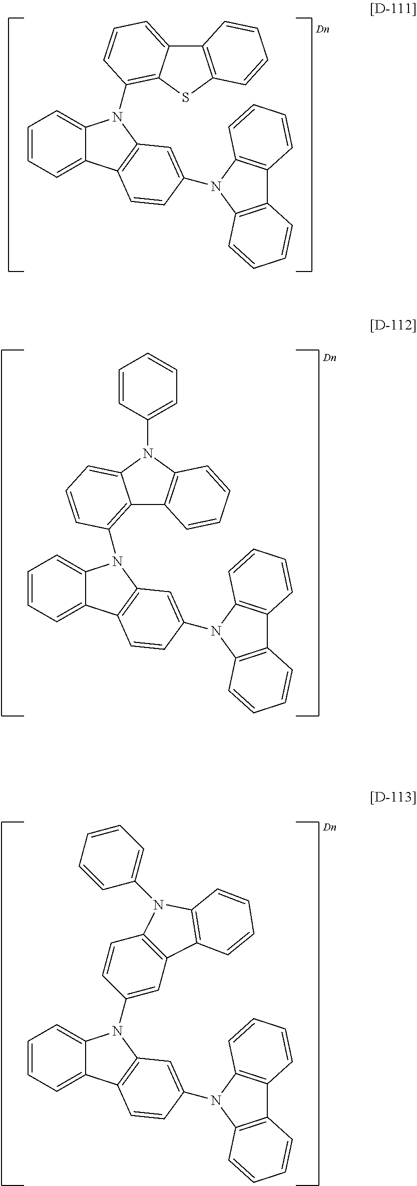

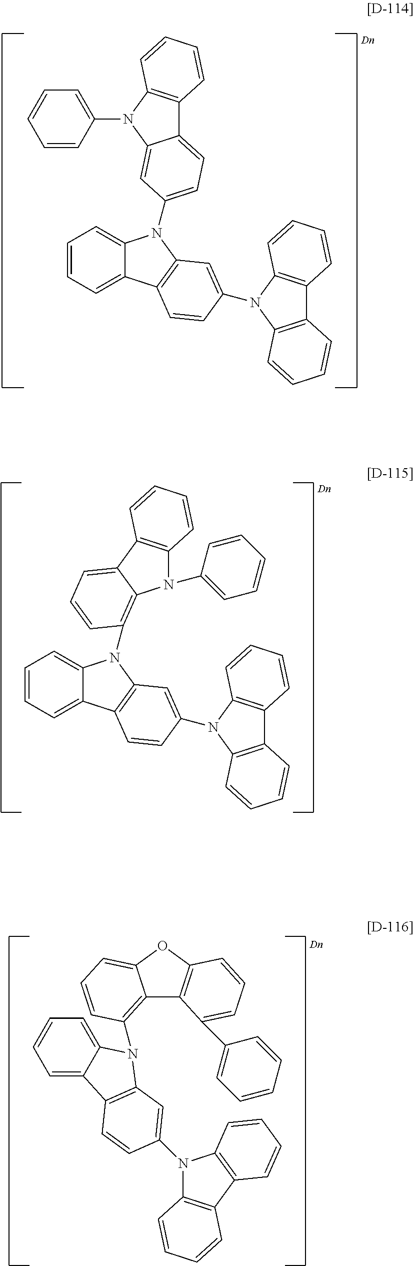

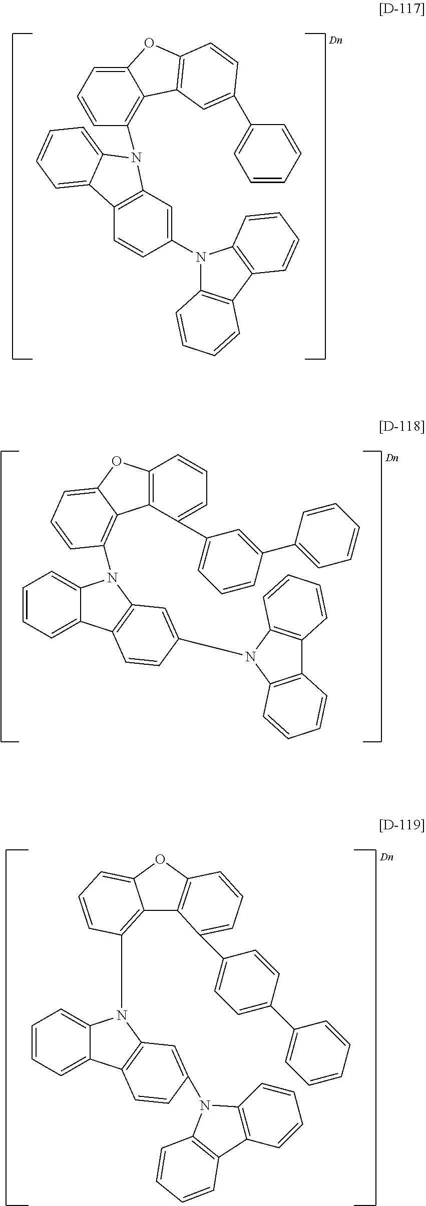

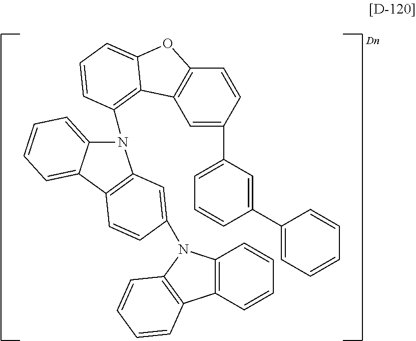

(Dn refers to the number of deuterium substitutions and indicates a structure substituted with one or more deuterium atoms)

The deuterium substitution position and deuterium substitution ratio may include all changeable ranges within the range of Compound D-1 to Compound D-120 (e.g., any hydrogen in any compound may be a protium or a deuterium).

In an implementation, the second compound may be represented by one of Chemical Formula 2-8, Chemical Formula 3C and Chemical Formula 5-3.

The first compound and the second compound may be included (e.g., mixed), e.g., in a weight ratio of about 1:99 to about 99:1. By being included in the above range, efficiency and life-span may be improved by implementing bipolar characteristics by adjusting the appropriate weight ratio using the electron transport capability of the first compound and the hole transport capability of the second compound. Within the above range, they may be included in a weight ratio of, e.g., about 10:90 to about 90:10, about 20:80 to about 80:20, about 20:80 to about 70:30, about 20:80 to about 60:40, and about 30:70 to about 60:40. In an implementation, they may be included in a weight ratio of about 40:60, about 50:50, or about 60:40.

Hereinafter, an organic optoelectronic device including the aforementioned compound for an organic optoelectronic device or composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to the drawing.

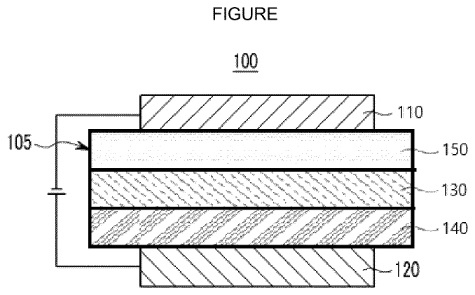

The FIG. 1s a cross-sectional view showing an organic light emitting diode according to some embodiments.

Referring to the FIGURE, an organic light emitting diode 100 according to some embodiments may include an anode 120 and a cathode 110 facing each other and an organic layer 105 between the anode 120 and cathode 110.

The anode 120 may be made of a conductor having a large work function to help hole injection, and may be, e.g., a metal, a metal oxide or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal or an oxide such as ZnO and Al or SnO₂ or Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; a multi-layer structure material such as LiF/Al, LiO₂/Al, LiF/Ca, or BaF₂/Ca.

The organic layer 105 may include the aforementioned compound for an organic optoelectronic device or composition for an organic optoelectronic device.

The organic layer 105 may include a light emitting layer 130, and the light emitting layer 130 may include a host and a dopant, the host may include the aforementioned compound for an organic optoelectronic device or composition for an organic optoelectronic device, and the dopant may be, e.g., a phosphorescent dopant, for example a red, green or blue phosphorescent dopant, for example a red or green phosphorescent dopant.

The dopant may be a material mixed with the compound or composition for an organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may include a phosphorescent dopant and examples of the phosphorescent dopant may include an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L⁹MX³ [Chemical Formula Z]

In Chemical Formula Z, M may be a metal, and L⁹ and X³ may be the same or different, and may each independently be ligands forming a complex compound with M.

The M may be, e.g., Ir, Pt, Os, Ti, Zr, Hf, Eu, Tb, Tm, Fe, Co, Ni, Ru, Rh, Pd, or a combination thereof, and L⁹ and X³ may be, e.g., a bidentate ligand.

Examples of the ligands represented by L⁹ and X³ may include ligands of Group A.

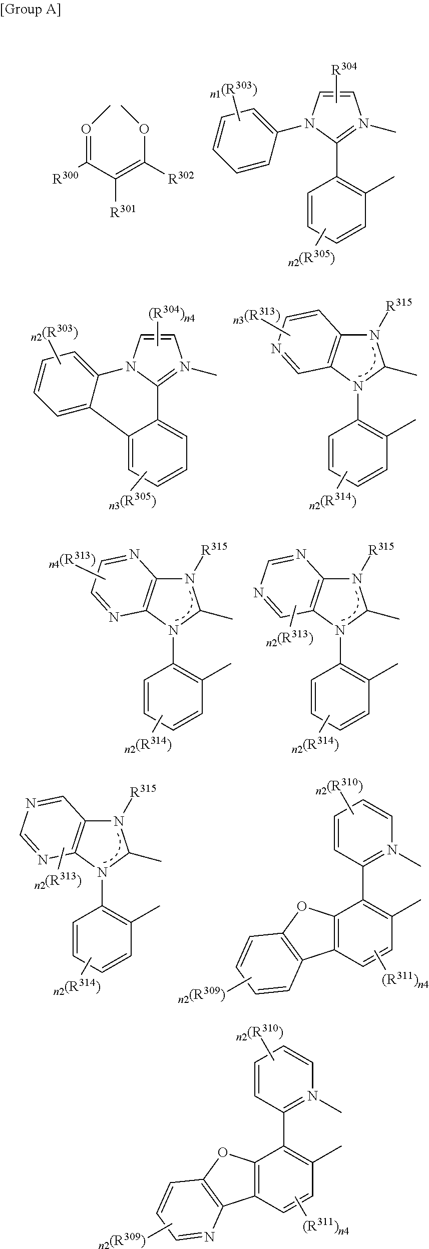

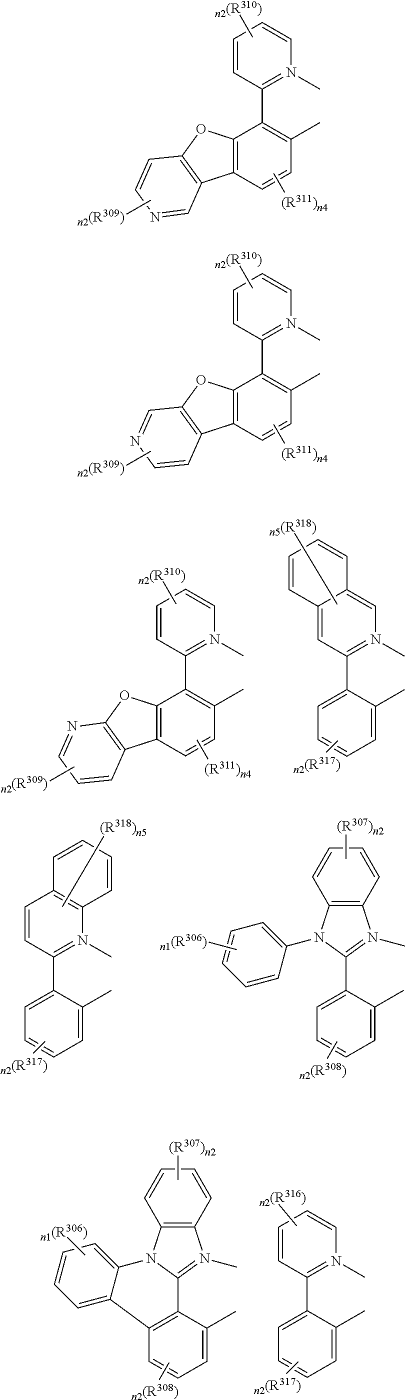

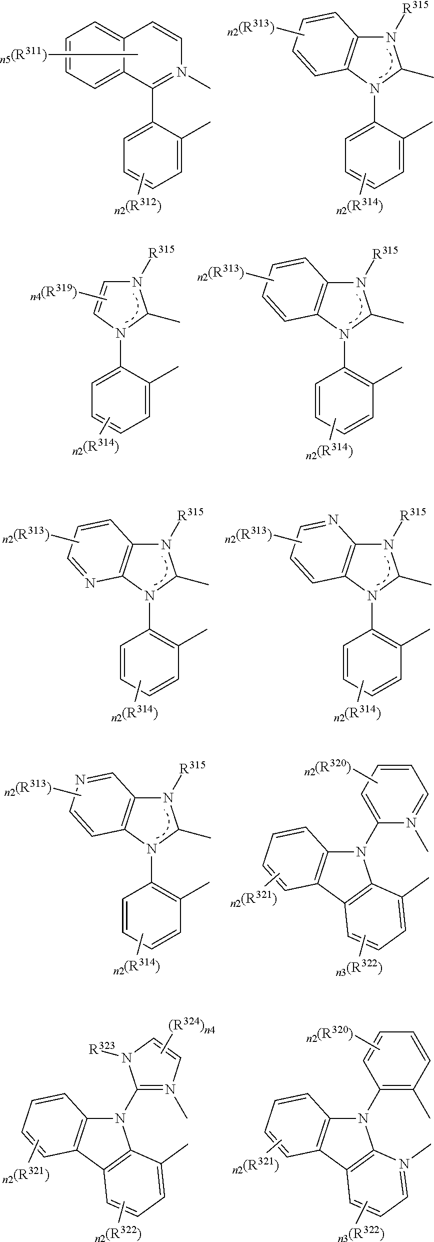

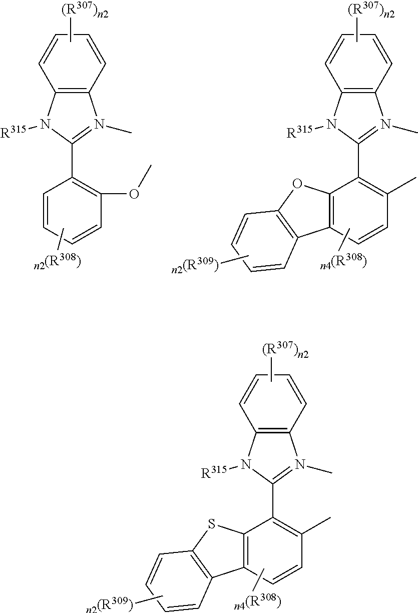

In Group A, R³⁰⁰ to R³⁰² may each independently be hydrogen, deuterium, a C1 to C30 alkyl group that may be substituted or unsubstituted with a halogen, a C6 to C30 aryl group that may be substituted or unsubstituted with a C1 to C30 alkyl, or a halogen.

R³⁰³ to R³²⁴ may each independently be hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

n1 may be, e.g., an integer of 1 to 5.

n2 may be, e.g., an integer of 1 to 4.

n3 may be, e.g., an integer of 1 to 3.

n4 may be, e.g., an integer of 1 or 2.

n5 may be, e.g., an integer of 1 to 6.

The dopant according to some embodiments may be an iridium complex, and may be represented, e.g., by Chemical Formula 6-1 or Chemical Formula 6-2.

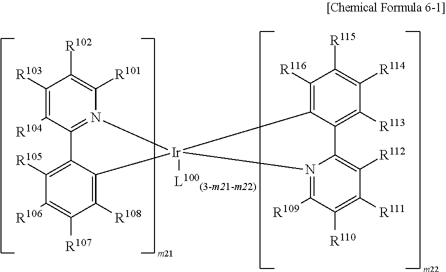

In Chemical Formula 6-1, R¹⁰¹ to R¹¹⁶ may each independently be hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be a substituted or unsubstituted C1 to C6 alkyl group.

At least one of R¹⁰¹ to R¹¹⁶ may be a functional group represented by Chemical Formula V-1.

L¹⁰⁰ may be a bidentate ligand of a monovalent anion, and may be a ligand that coordinates to iridium through a lone pair of carbons or heteroatoms.

m21 and m22 may each independently be an integer of 0 to 3, and m21+m22 may be an integer of 1 to 3,

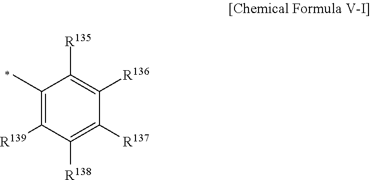

In Chemical Formula V-1, R¹³⁵ to R¹³⁹ may each independently be hydrogen, deuterium, a substituted or unsubstituted C1 to C10 alkyl group, a substituted or unsubstituted C6 to C20 aryl group, or —SiR¹³²R¹³³R¹³⁴, and * means a portion linked to a carbon atom.

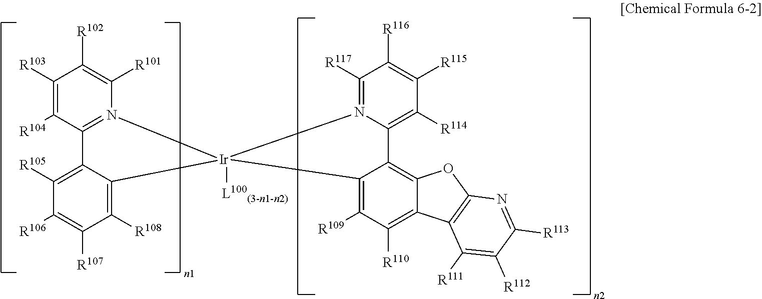

In Chemical Formula 6-2, R¹⁰¹ to R¹¹⁷ may each independently be hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be a substituted or unsubstituted C1 to C6 alkyl group.

L¹⁰⁰ may be a bidentate ligand of a monovalent anion, and may be a ligand that coordinates to iridium through a lone pair of carbons or heteroatoms.

n1 and n2 may each independently be an integer of 0 to 3, and n1+n2 may be an integer of 1 to 3.

The dopant according to some embodiments may be a platinum complex, and may be represented, e.g., by Chemical Formula Z-1.

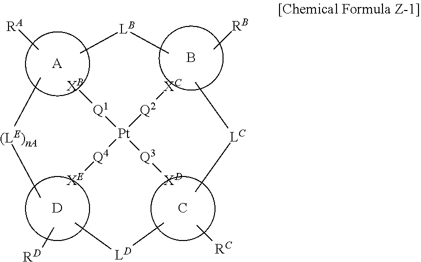

In Chemical Formula Z-1, rings A, B, C, and D may each independently be a 5-membered or 6-membered carbocyclic or heterocyclic ring.

R^{A}, R^{B}, R^{C}, and R^{D} may each independently be mono-, di-, tri-, or tetra-substitution, or unsubstitution.

L^{B}, L^{C}, and L^{D} may each independently be a direct bond, BR, NR, PR, O, S, Se, C═O, S═O, SO₂, CRR′, SiRR′, GeRR′, or a combination thereof.

In an implementation, nA may be 1, L^{E} may be a direct bond, BR, NR, PR, O, S, Se, C═O, S═O, SO₂, CRR′, SiRR′, GeRR′, or a combination thereof. In an implementation, nA may be 0 and L^{E} may not exist.

R^{A}, R^{B}, R^{C}, R^{D}, R, and R′ may each independently be hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ may optionally linked to each other to provide a ring; X^{B}, X^{C}, X^{D}, and X^{E} may each independently be carbon or nitrogen; and Q¹, Q², Q³, and Q⁴ may each independently be oxygen or a direct bond.

The platinum complex may be represented, e.g., by Chemical Formula 7-1 or Chemical Formula 7-2.

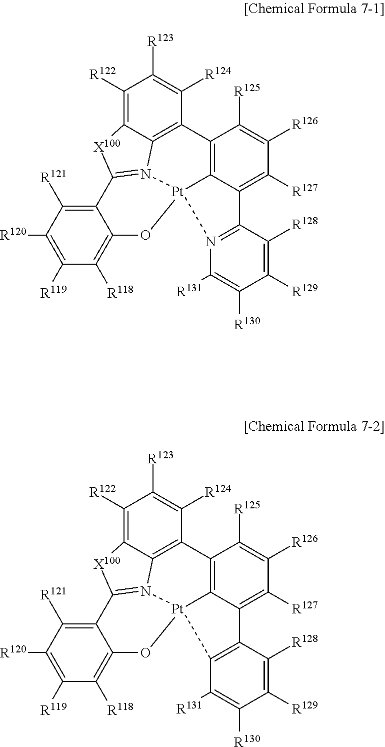

In Chemical Formula 7-1 and Chemical Formula 7-2, X¹⁰⁰ may be selected from O, S, or NR¹³².

R¹¹⁸ to R¹³² may each independently be hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

In an implementation, at least one of R¹¹⁸ to R¹³² may be —SiR¹³³R¹³⁴R¹³⁵ or a tert-butyl group.

R¹³³ to R¹³⁵ may each independently be a substituted or unsubstituted C1 to C6 alkyl group.

The organic layer may further include a charge transport region in addition to the light emitting layer.

The charge transport region may be, e.g., the hole transport region 140.

The hole transport region 140 may help further increase hole injection or hole mobility between the anode 120 and the light emitting layer 130 and block electrons.

In an implementation, the hole transport region 140 may include a hole transport layer between the anode 120 and the light emitting layer 130, and a hole transport auxiliary layer between the light emitting layer 130 and the hole transport layer, and a compound of Group B may be included in at least one of the hole transport layer and the hole transport auxiliary layer.

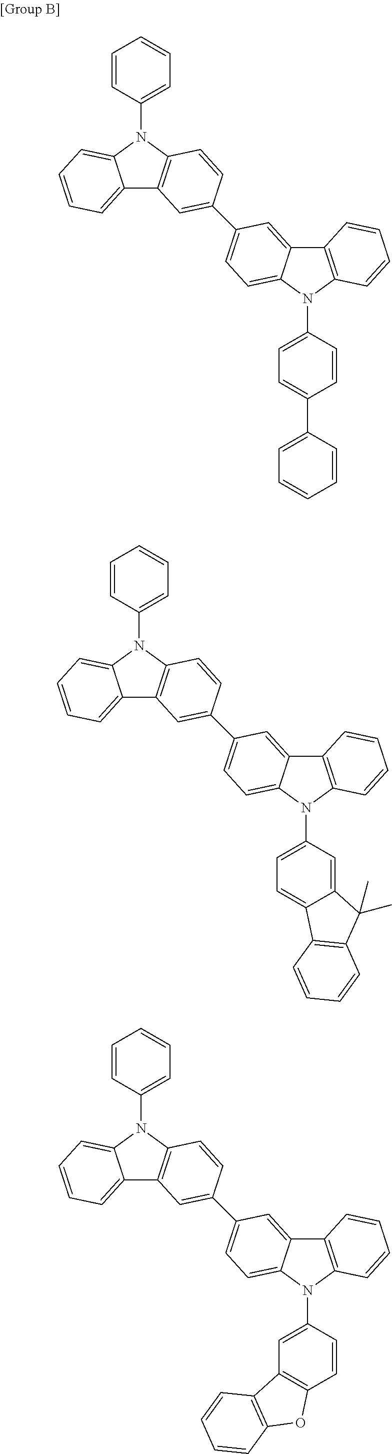 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

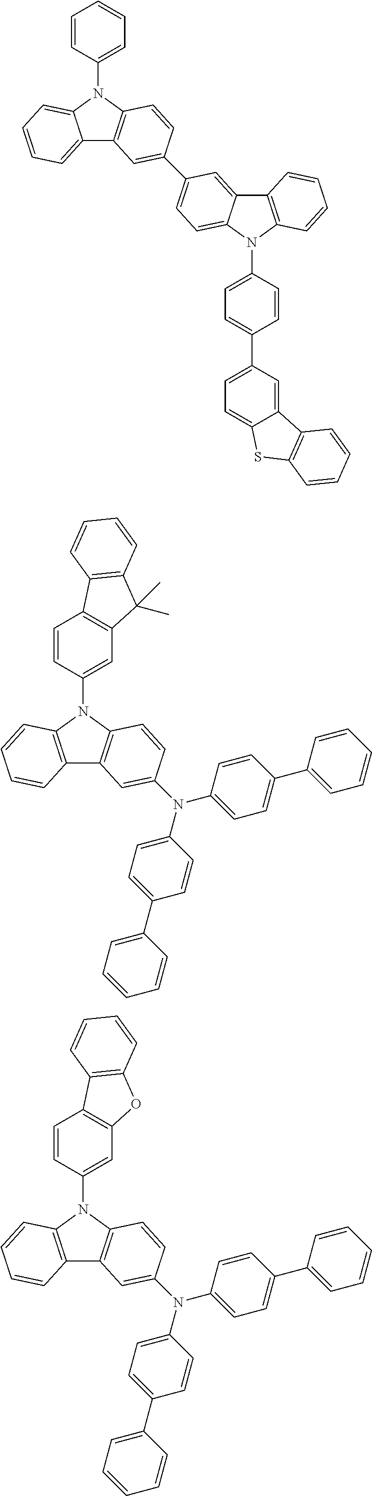

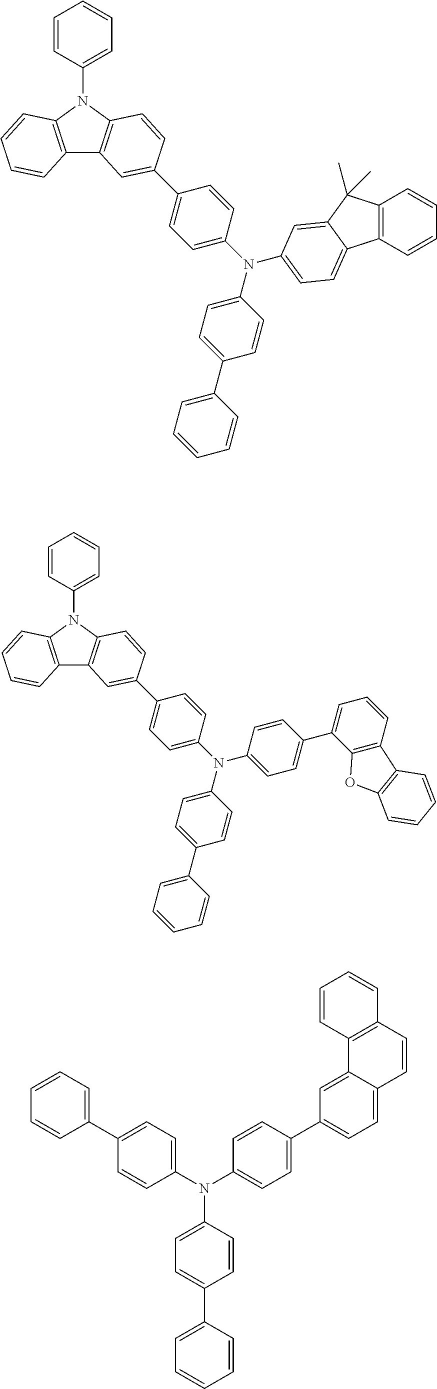 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

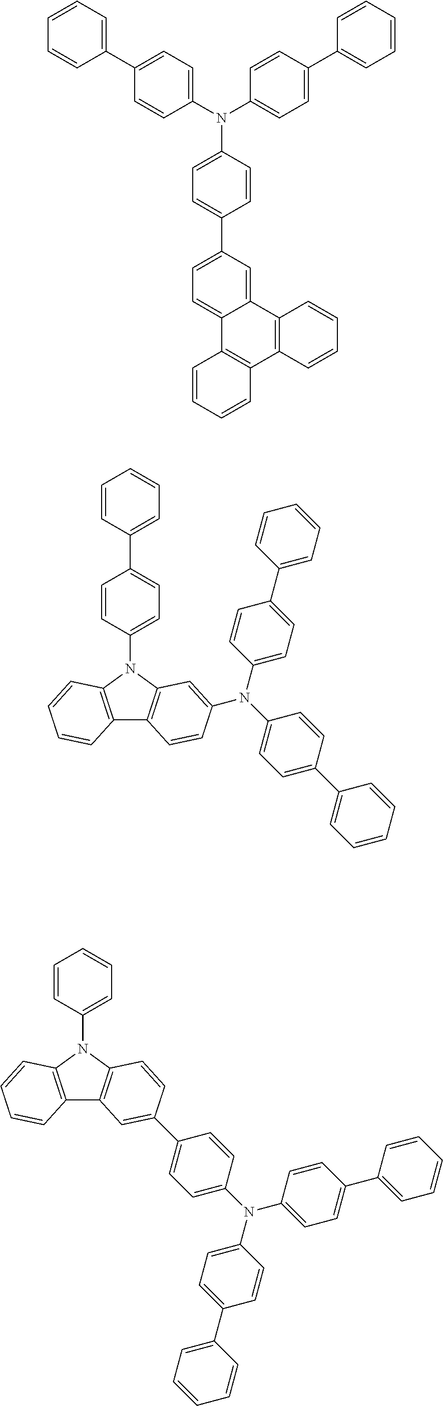 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

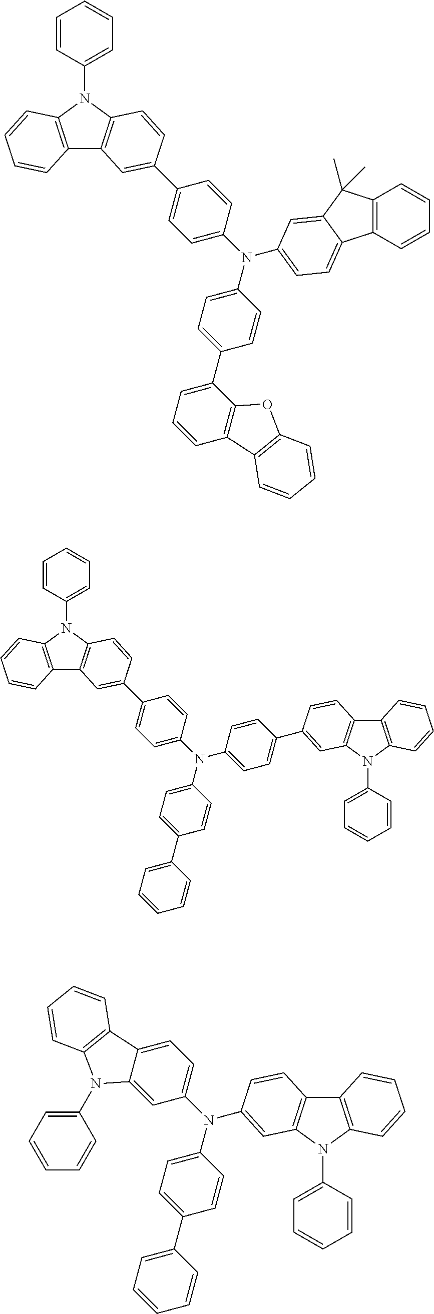 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1]

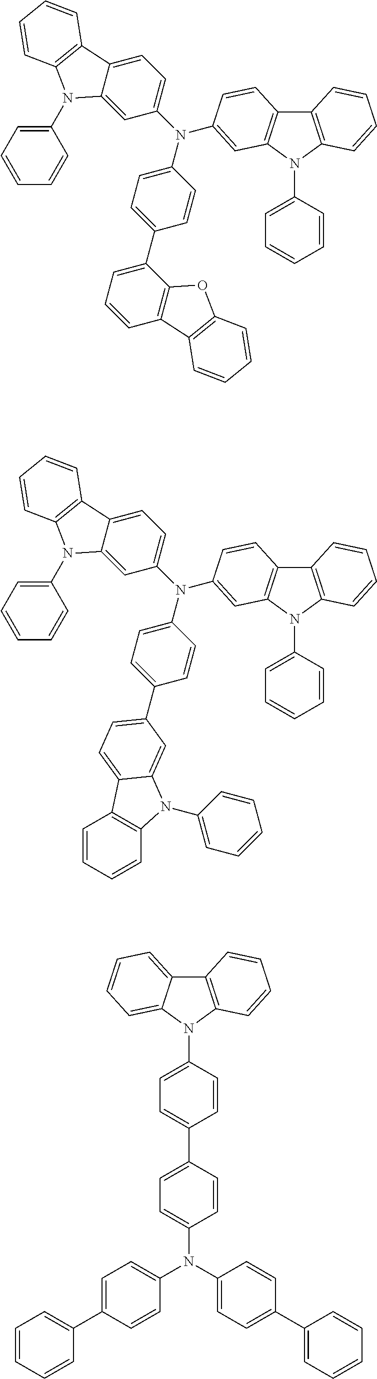 [SMILES: c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1]

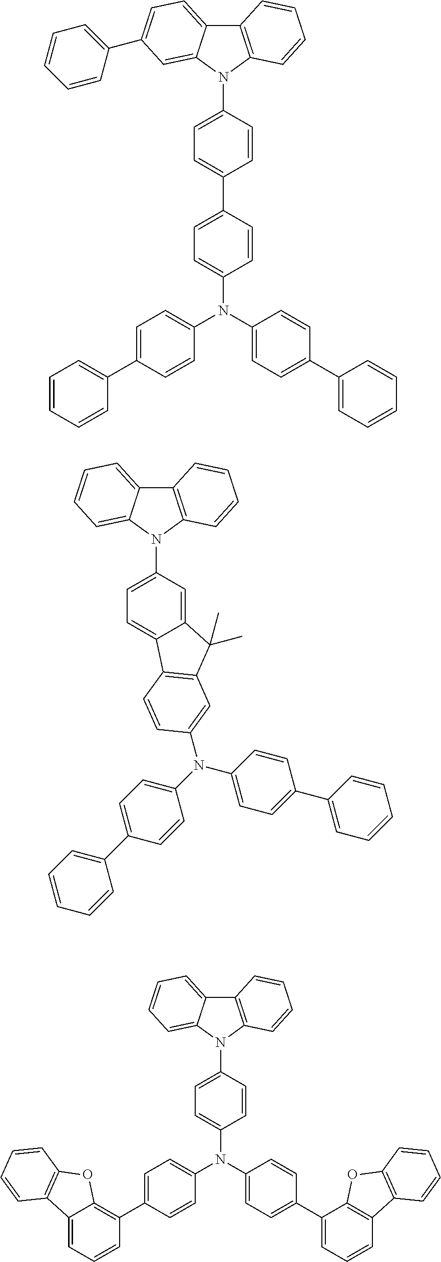

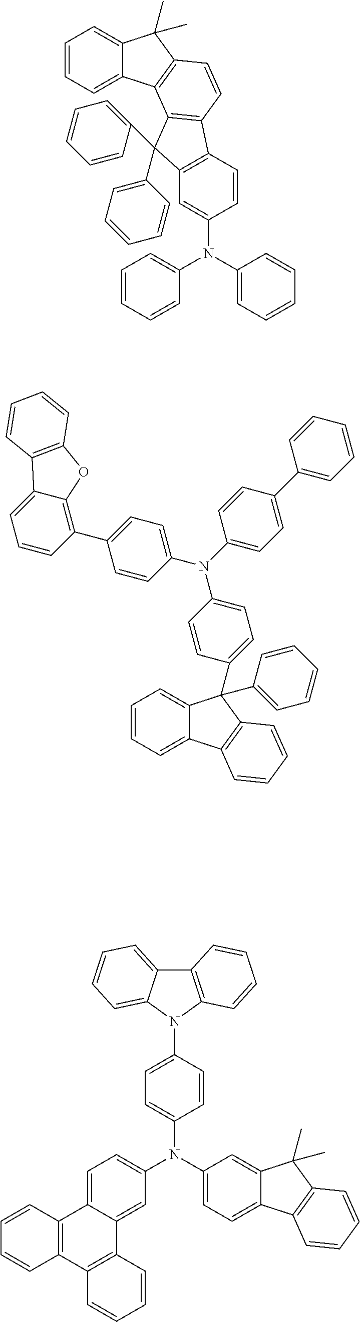 [SMILES: CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; CC1(C)c2ccccc2-c2ccc(N(c3ccc(-n4c5ccccc5c5ccccc54)cc3)c3ccc4c5ccccc5c5ccccc5c4c3)cc21]

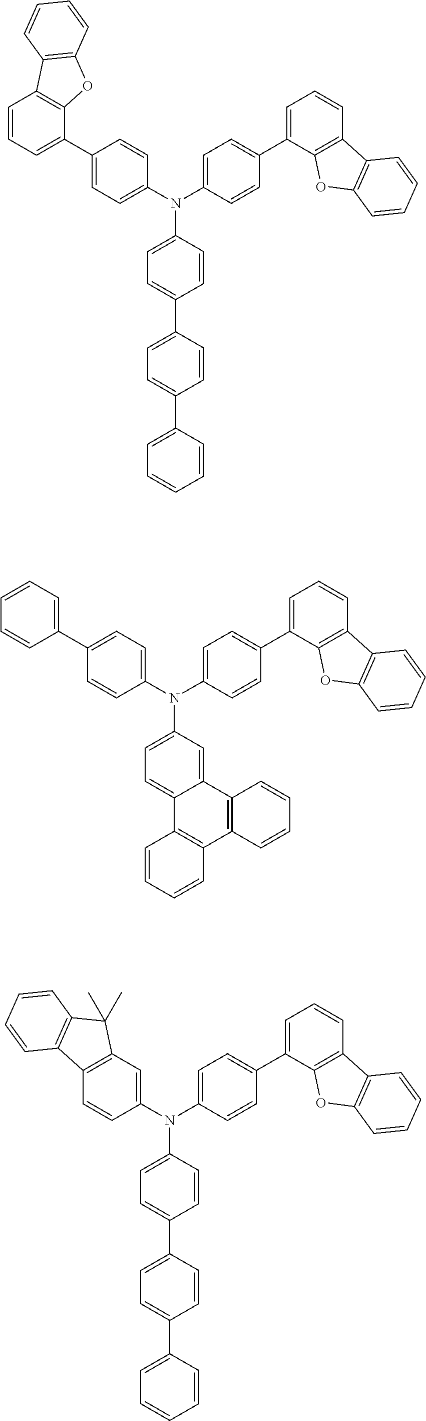 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21]

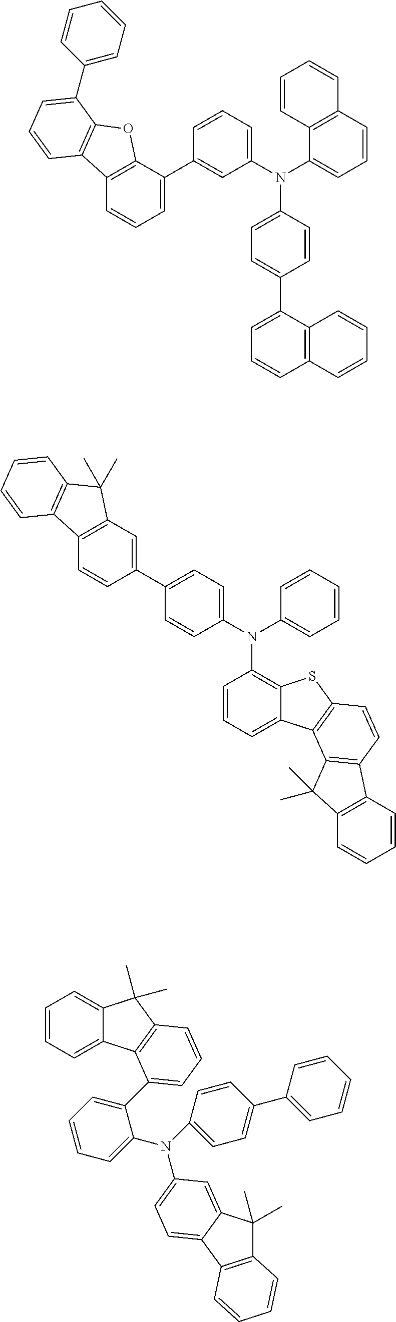 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

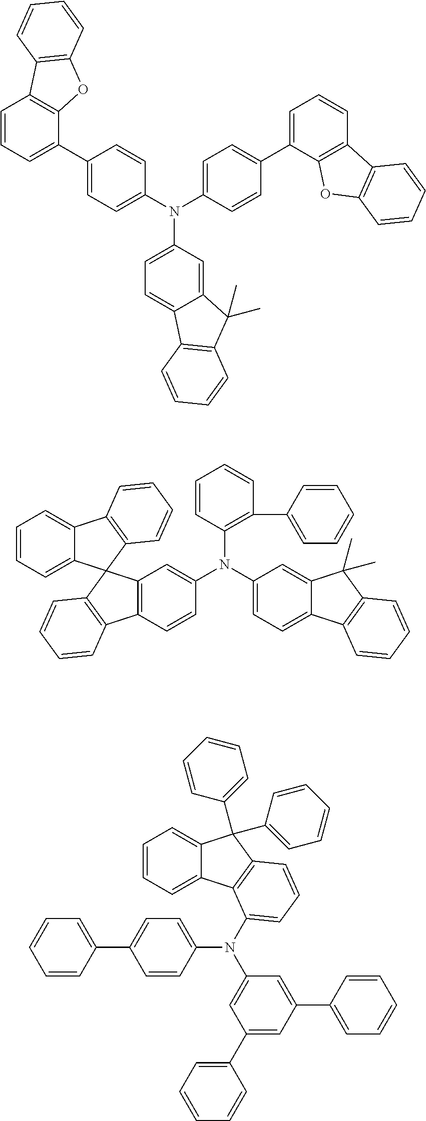 [SMILES: CC1(C)c2ccccc2-c2ccc(N(c3ccc(-c4cccc5c4oc4ccccc45)cc3)c3ccc(-c4cccc5c4oc4ccccc45)cc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3cc(-c4ccccc4)cc(-c4ccccc4)c3)c3cccc4c3-c3ccccc3C4(c3ccccc3)c3ccccc3)cc2)cc1]

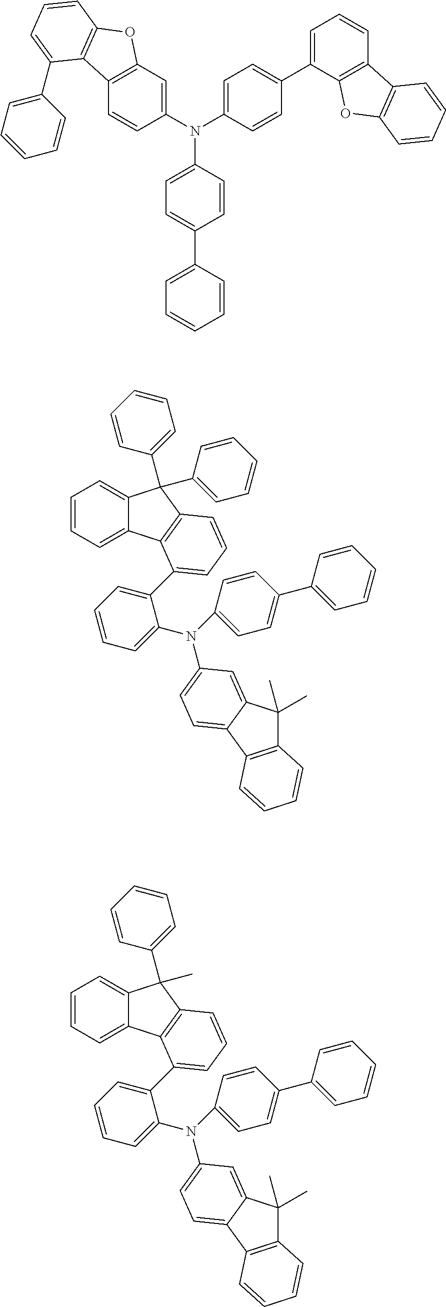 [SMILES: c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

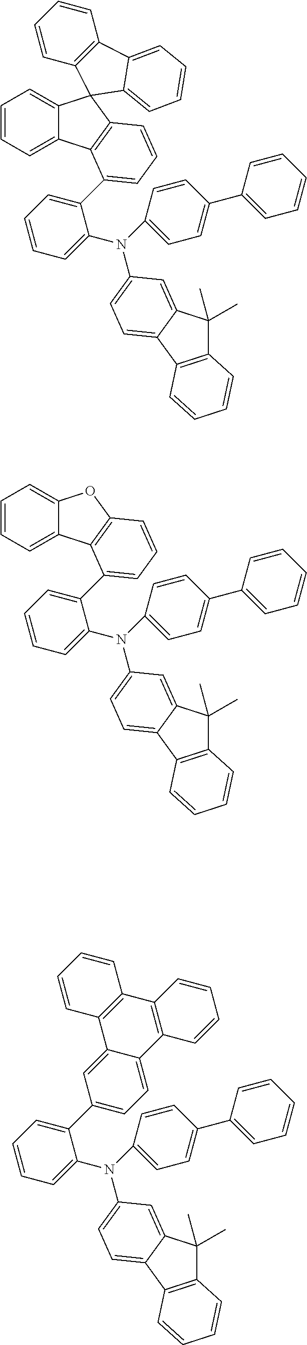 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21 ; CC1(C)c2ccccc2-c2ccc(N(c3ccc(-c4ccccc4)cc3)c3ccccc3-c3ccc4c5ccccc5c5ccccc5c4c3)cc21]

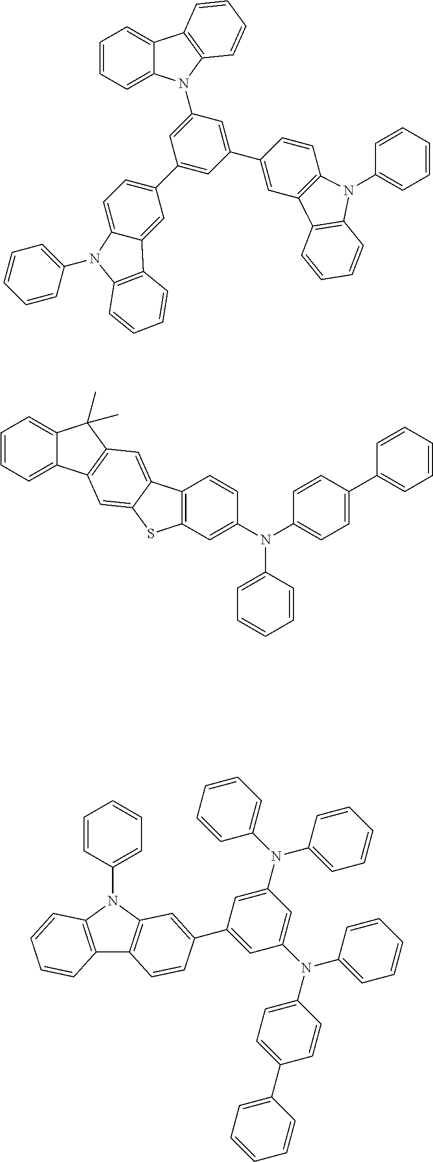 [SMILES: c1ccc(-n2c3ccccc3c3cc(-c4cc(-c5ccc6c(c5)c5ccccc5n6-c5ccccc5)cc(-n5c6ccccc6c6ccccc65)c4)ccc32)cc1 ; CC1(C)c2ccccc2-c2cc3sc4cc(N(c5ccccc5)c5ccc(-c6ccccc6)cc5)ccc4c3cc21 ; c1ccc(-c2ccc(N(c3ccccc3)c3cc(-c4ccc5c6ccccc6n(-c6ccccc6)c5c4)cc(N(c4ccccc4)c4ccccc4)c3)cc2)cc1]

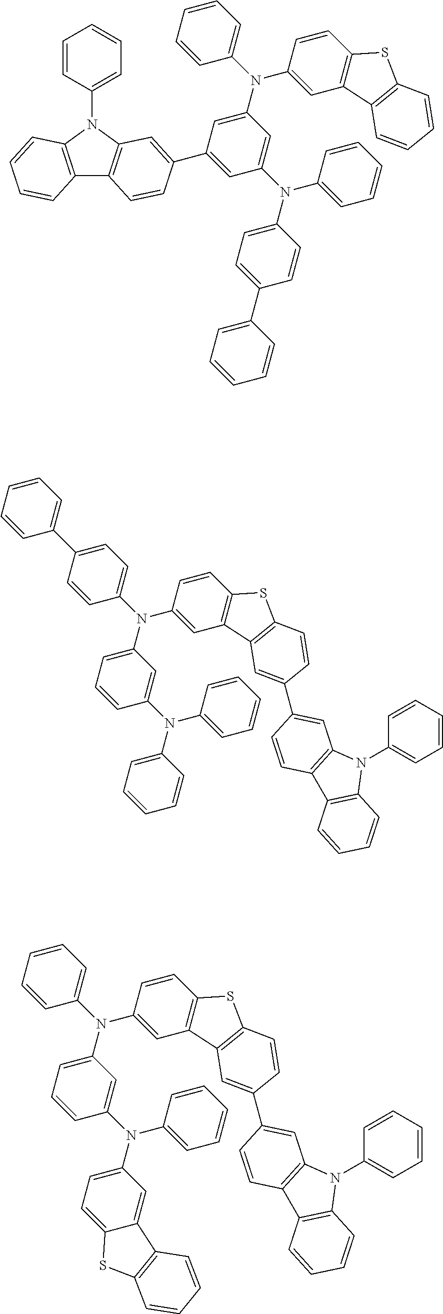 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1]

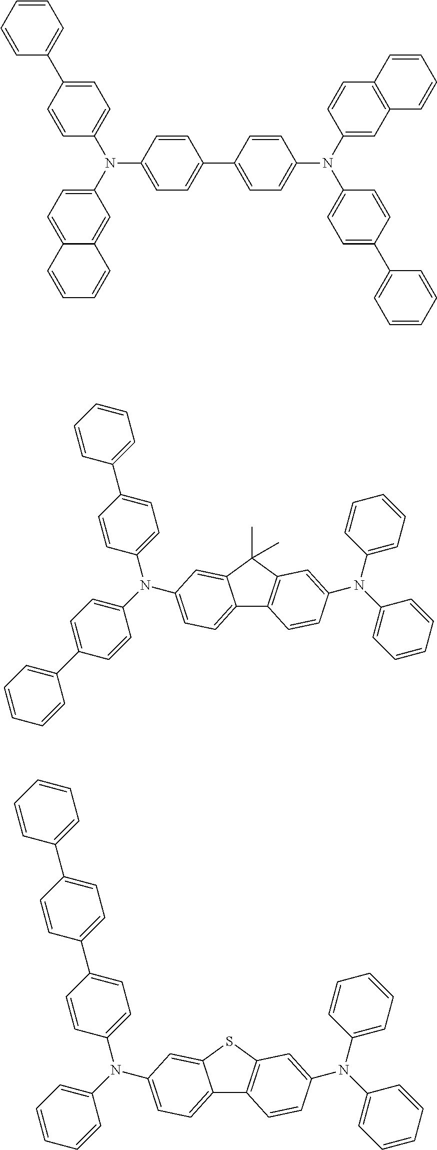 [SMILES: c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21 ; c1ccc(-c2ccc(-c3ccc(N(c4ccccc4)c4ccc5c(c4)sc4cc(N(c6ccccc6)c6ccccc6)ccc45)cc3)cc2)cc1]

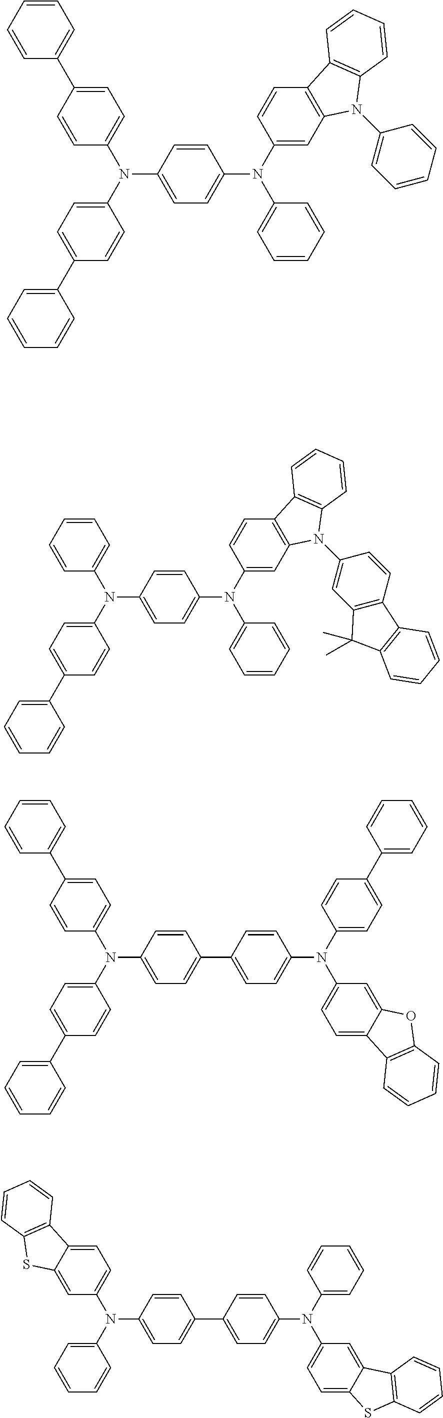 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21 ; c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1]

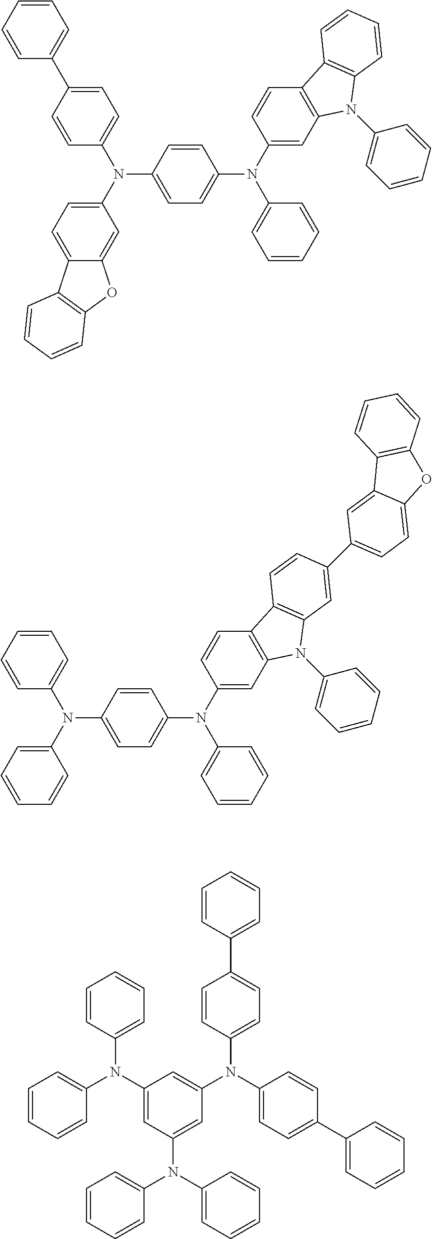 [SMILES: c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1]

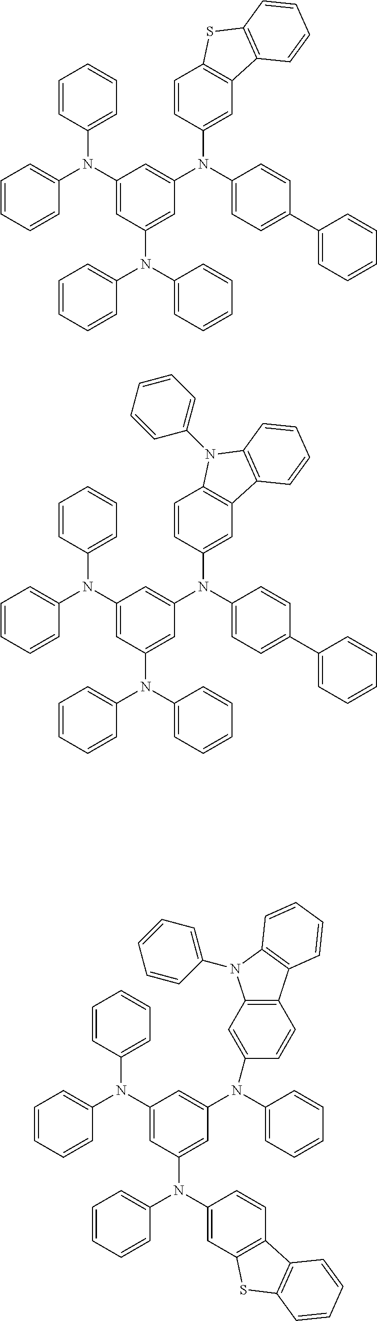 [SMILES: c1ccc(-c2ccc(N(c3cc(N(c4ccccc4)c4ccccc4)cc(N(c4ccccc4)c4ccccc4)c3)c3ccc4sc5ccccc5c4c3)cc2)cc1 ; c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1]

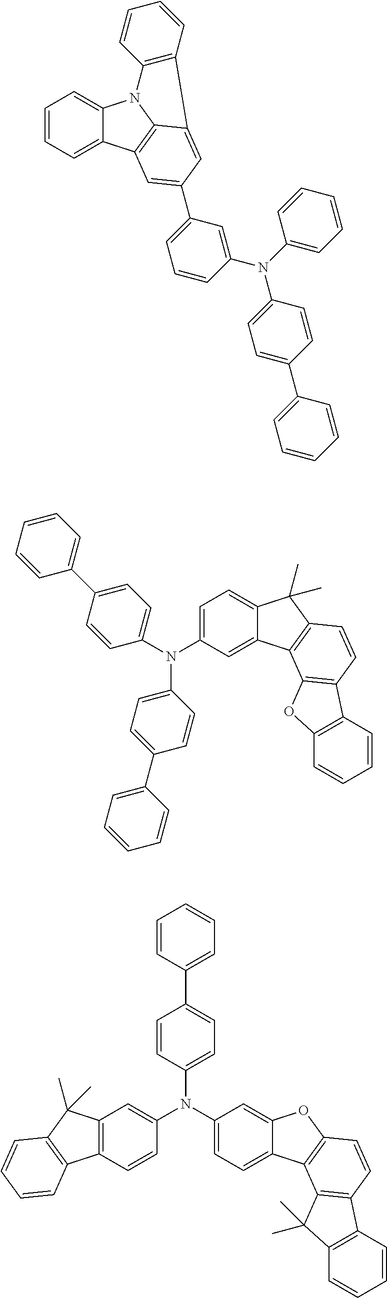 [SMILES: c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21]

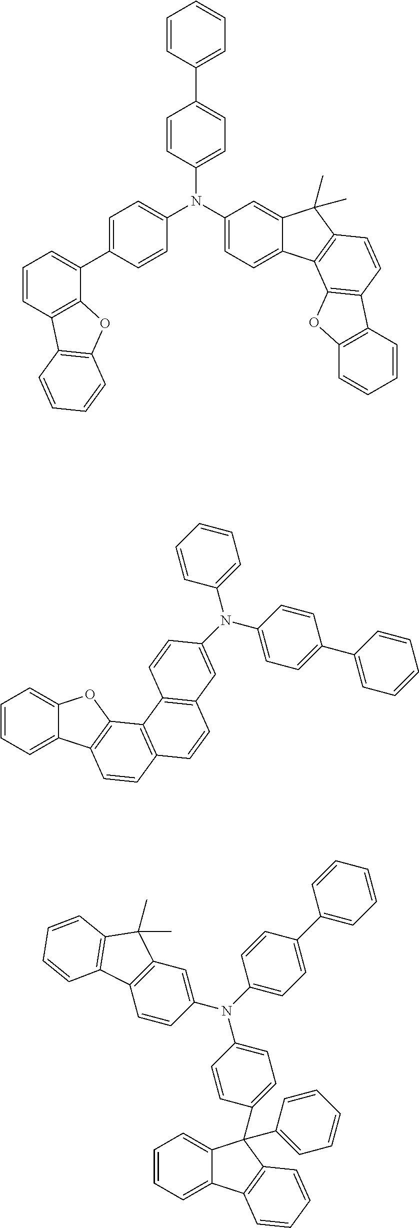 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

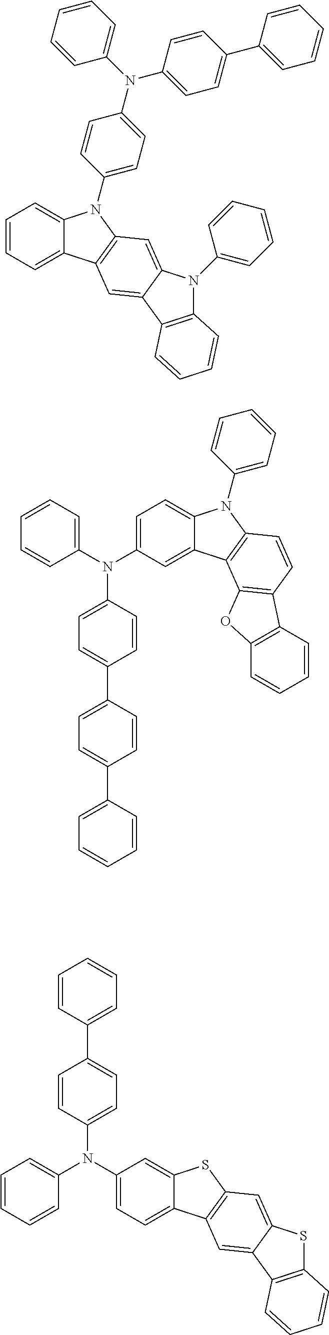 [SMILES: c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1]

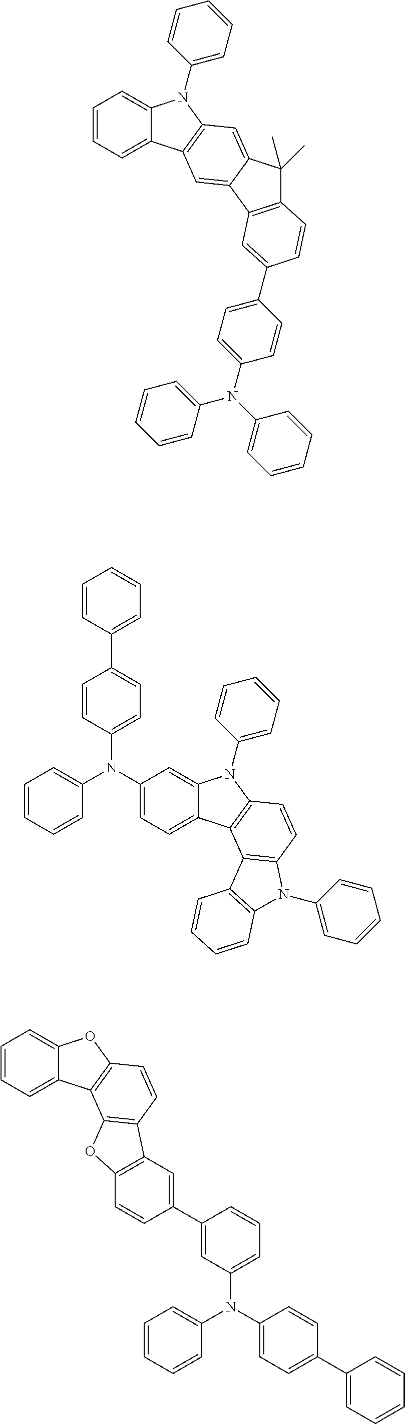 [SMILES: CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1]

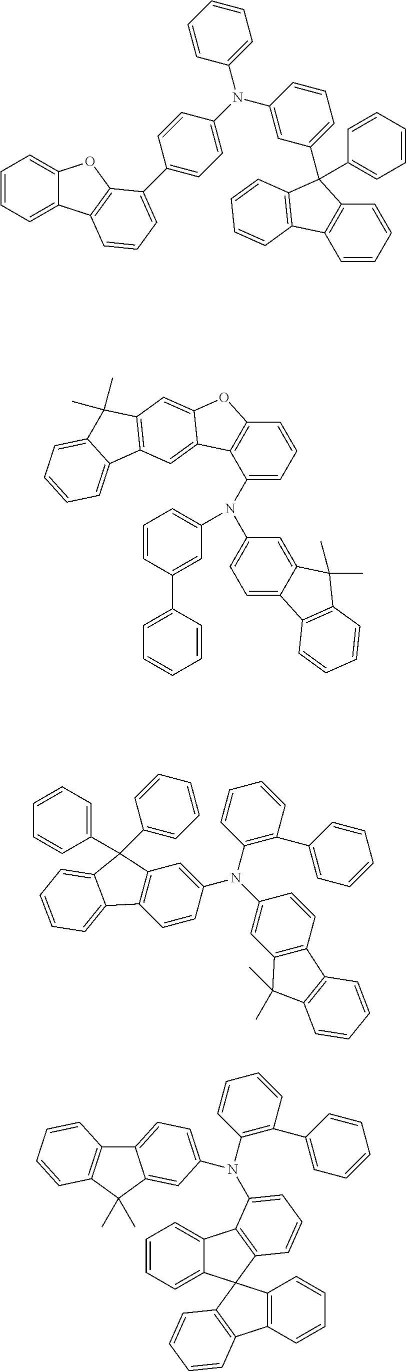 [SMILES: c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21]

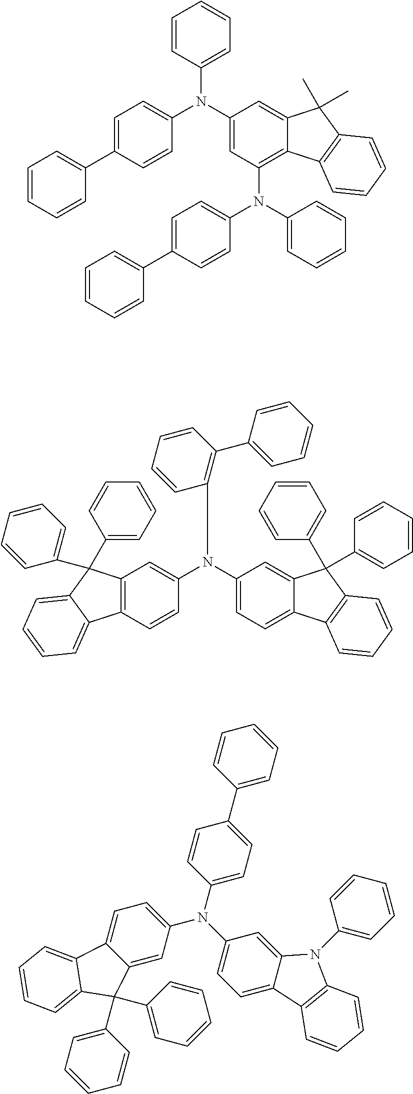 [SMILES: CC1(C)c2ccccc2-c2c(N(c3ccccc3)c3ccc(-c4ccccc4)cc3)cc(N(c3ccccc3)c3ccc(-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1]

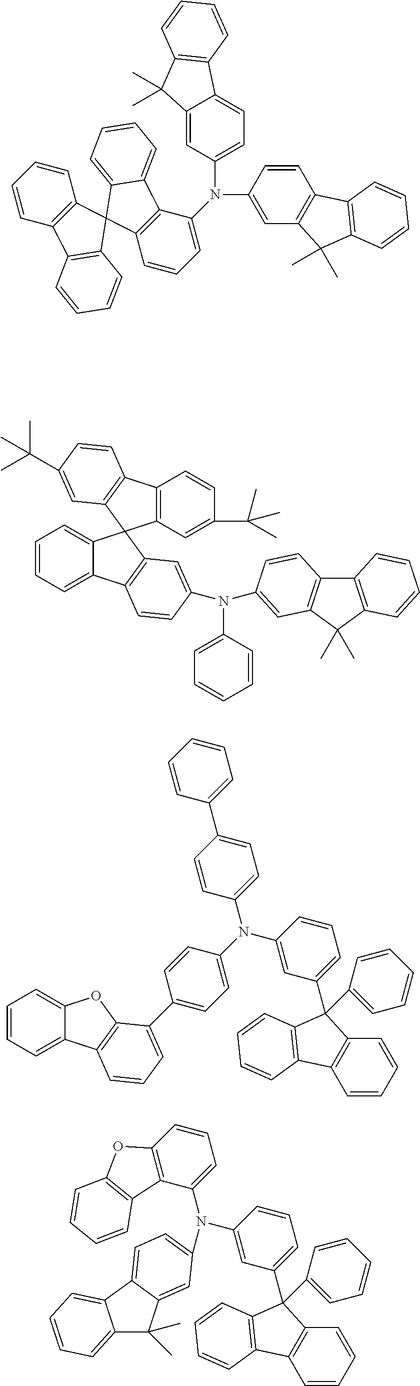

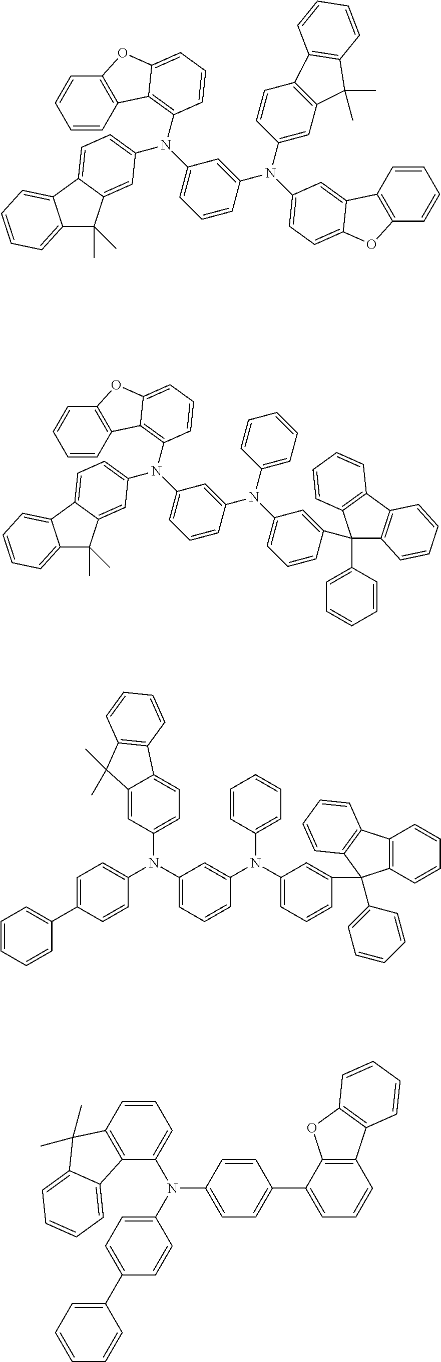 [SMILES: CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21 ; CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21]

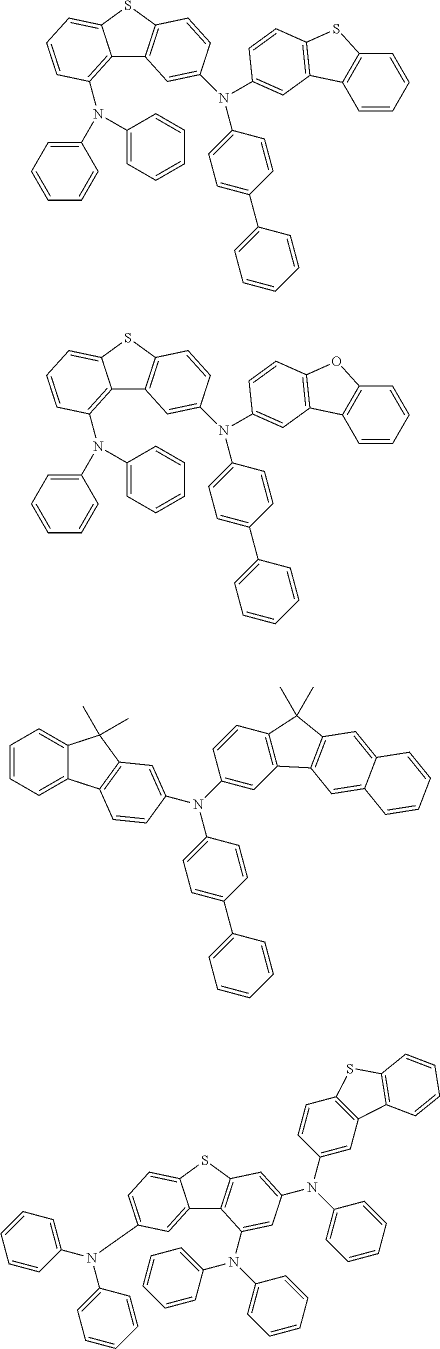 [SMILES: c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1 ; CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1]

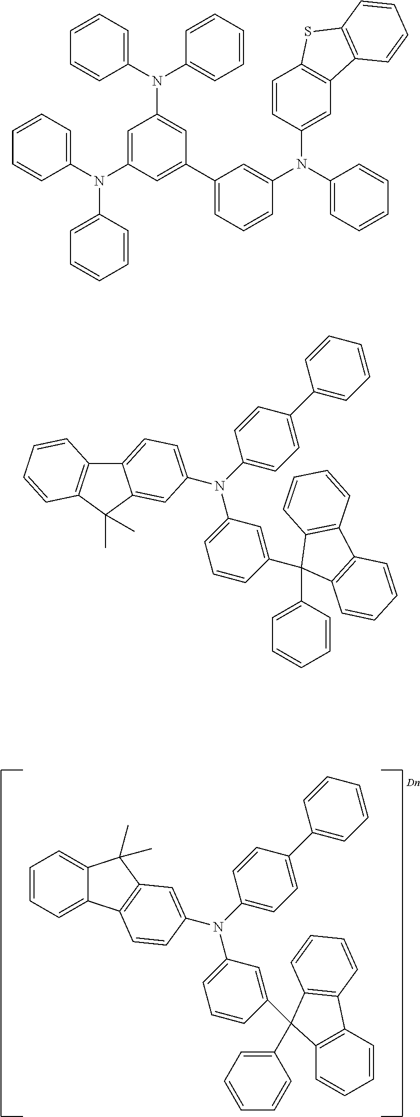

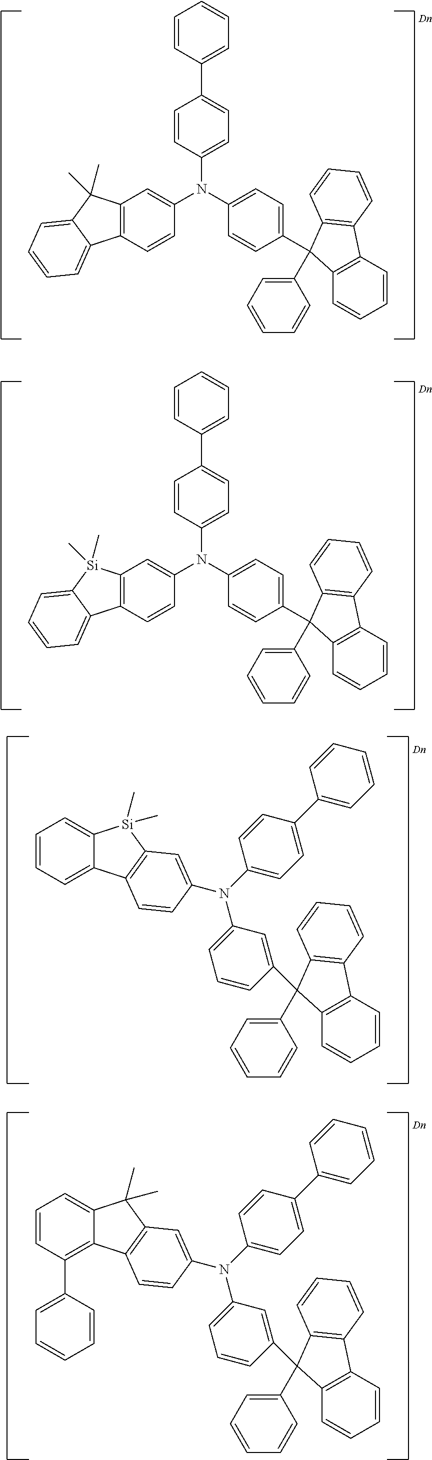

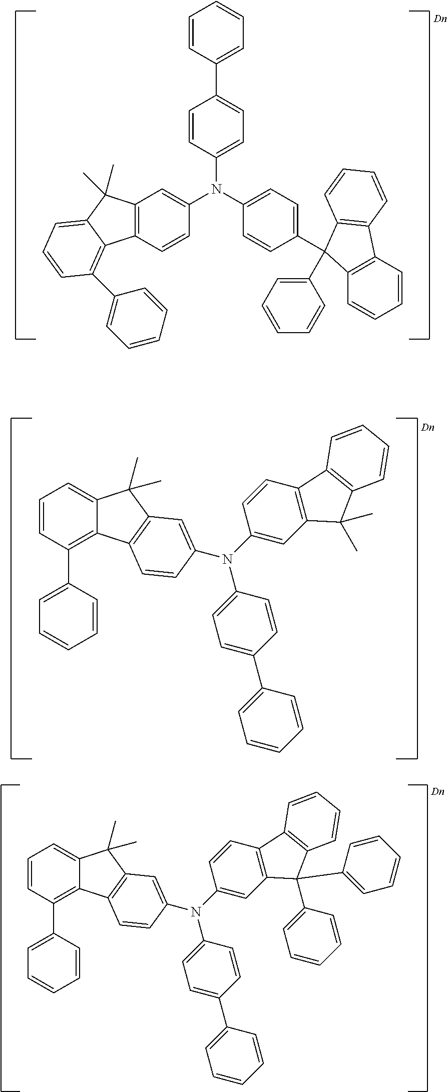

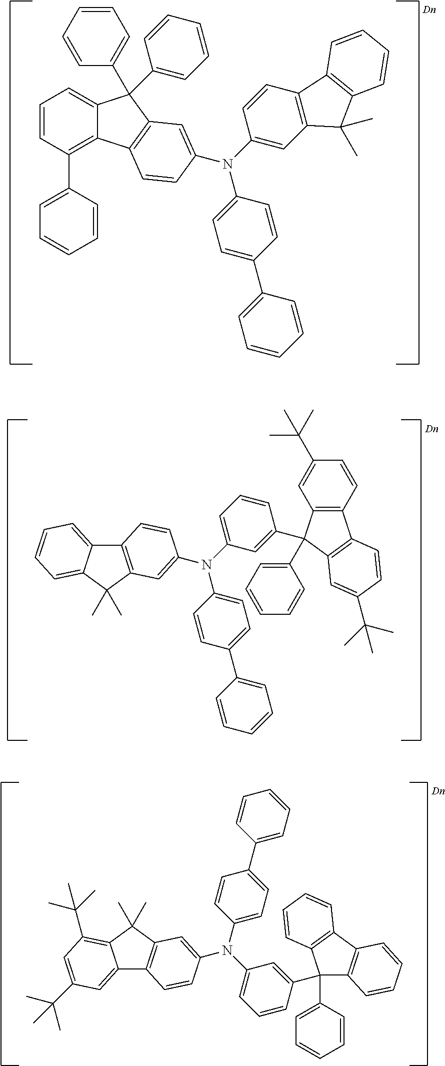

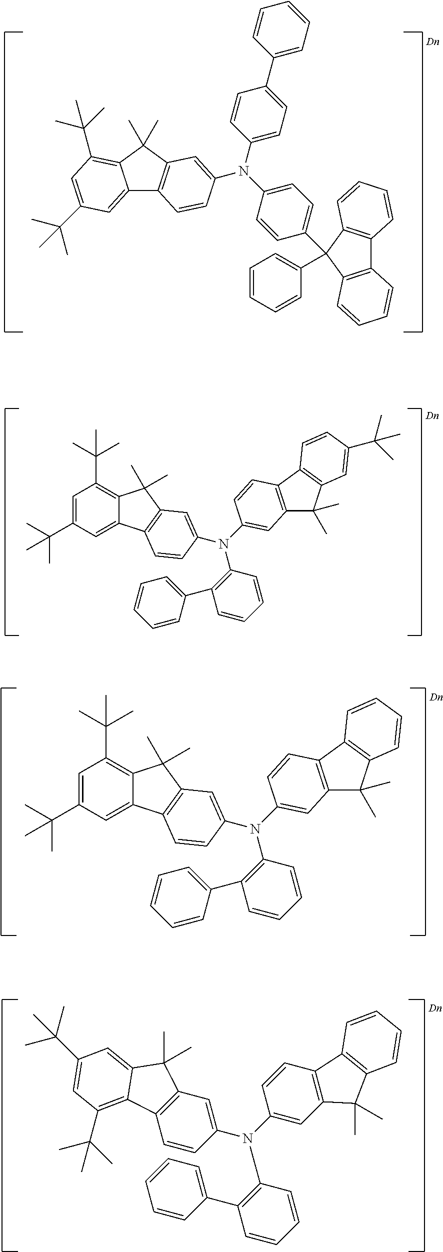

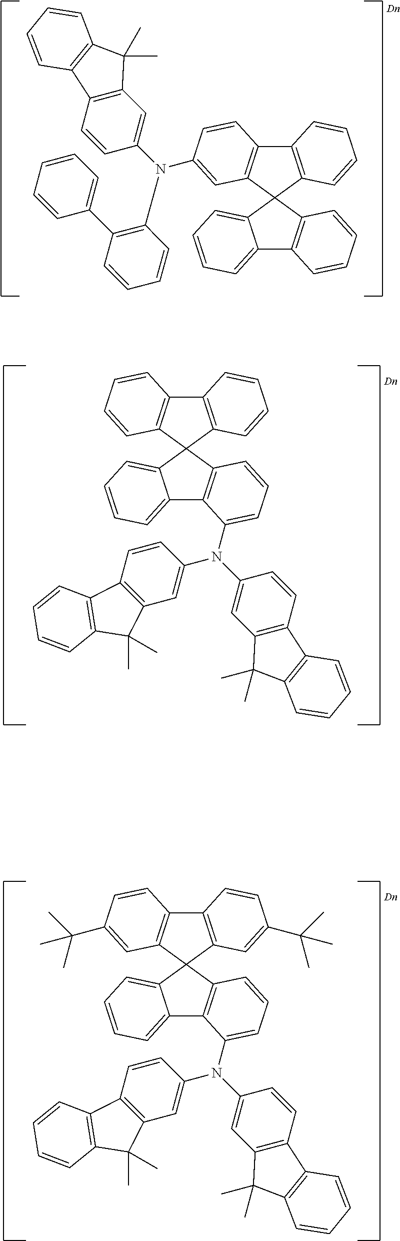

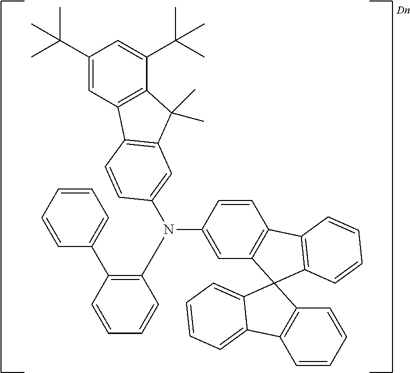

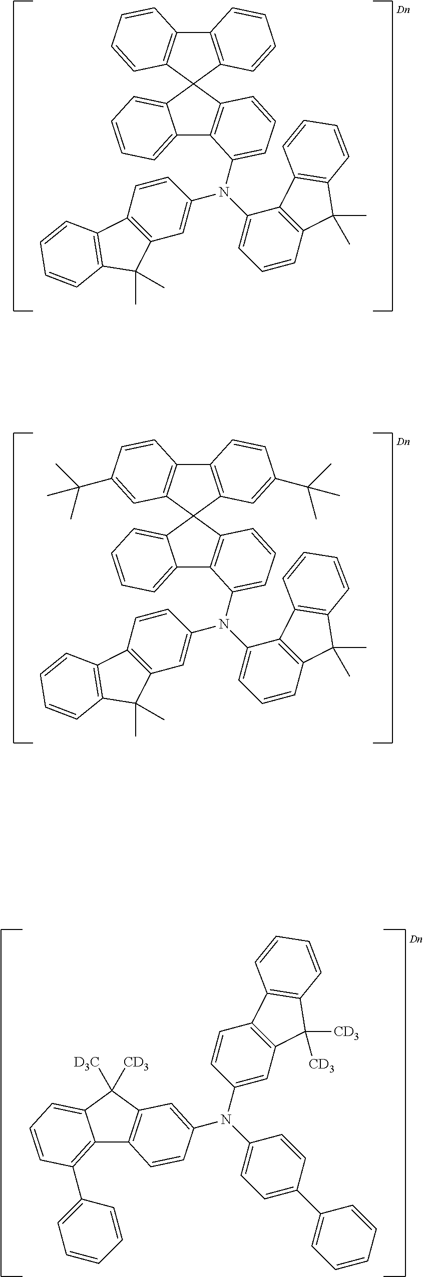

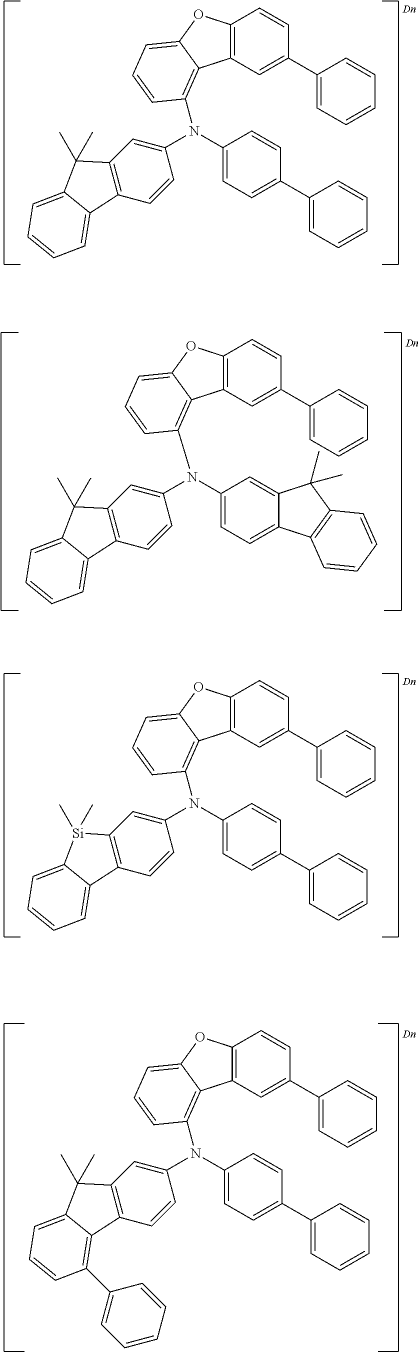

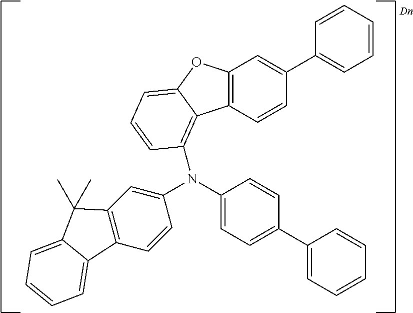

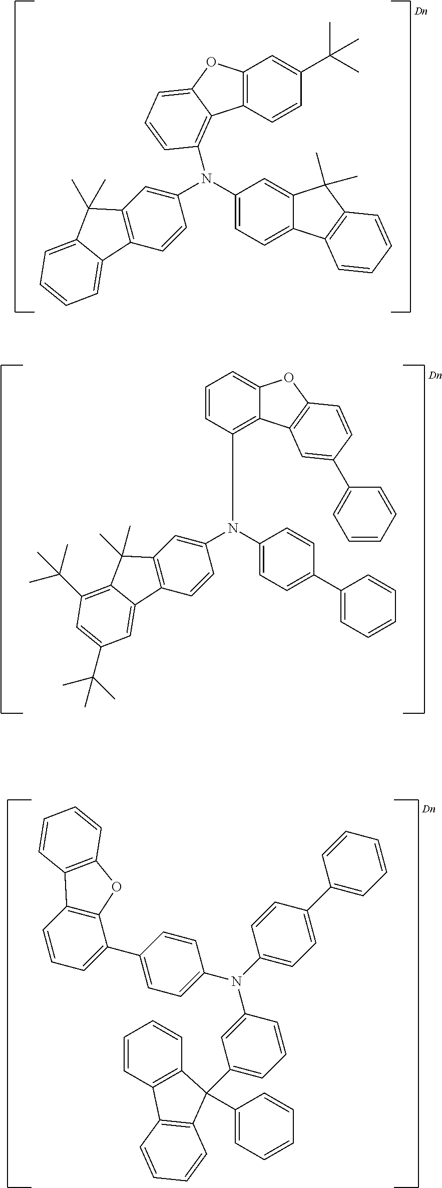

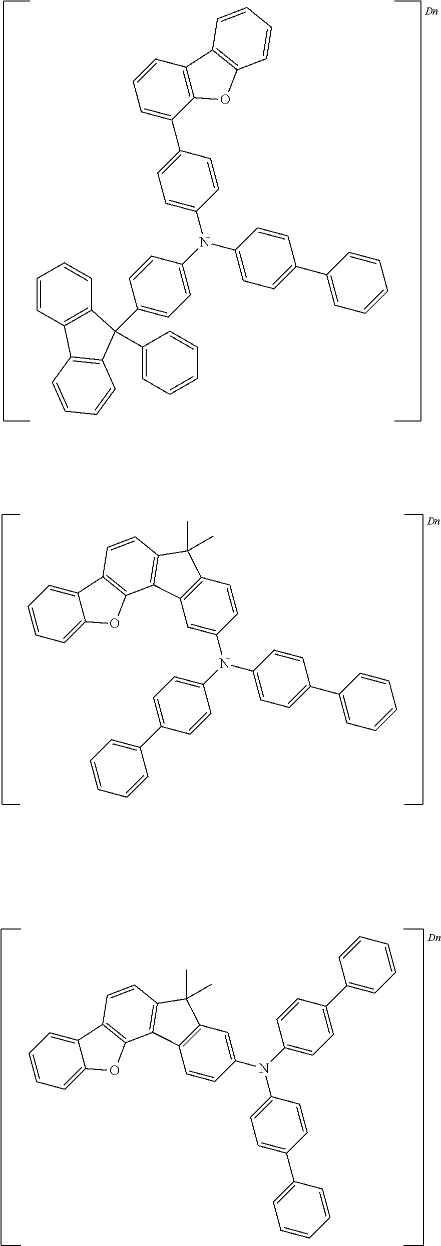

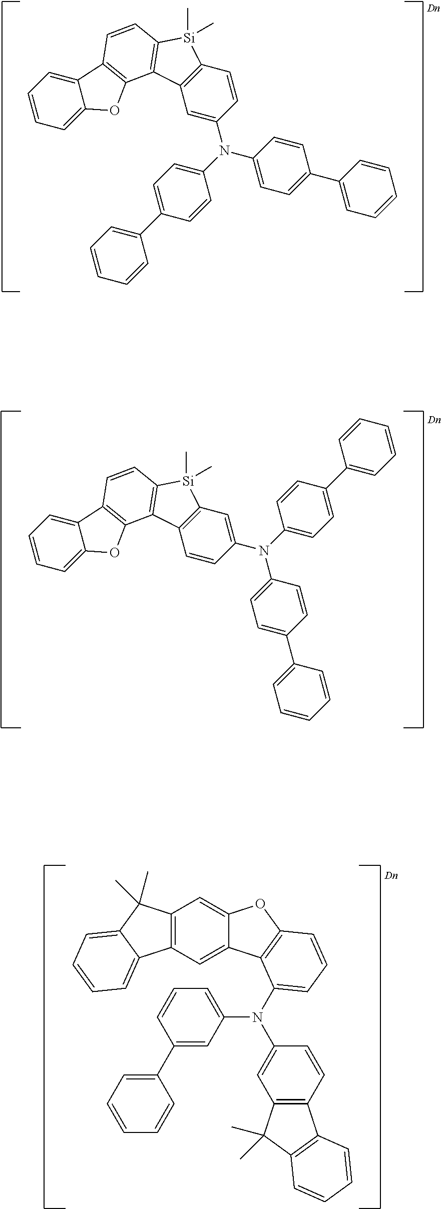

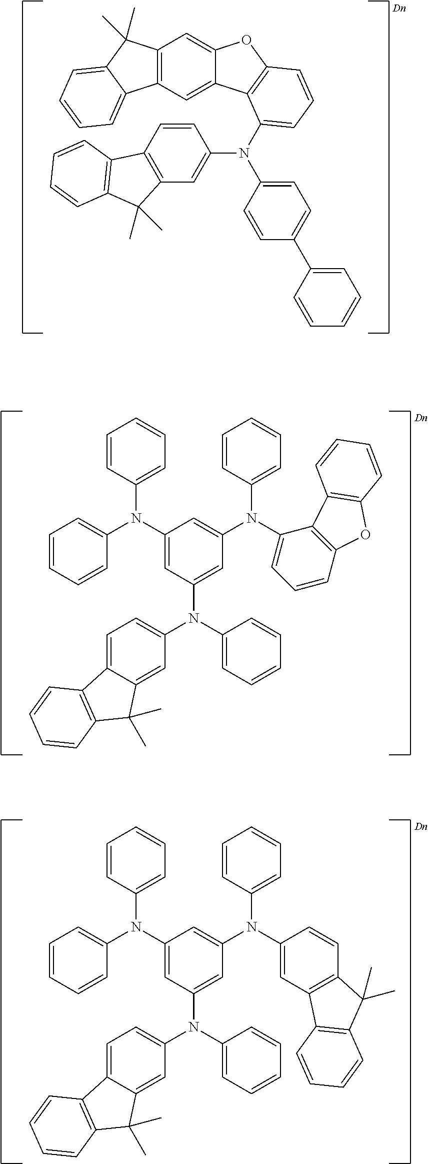

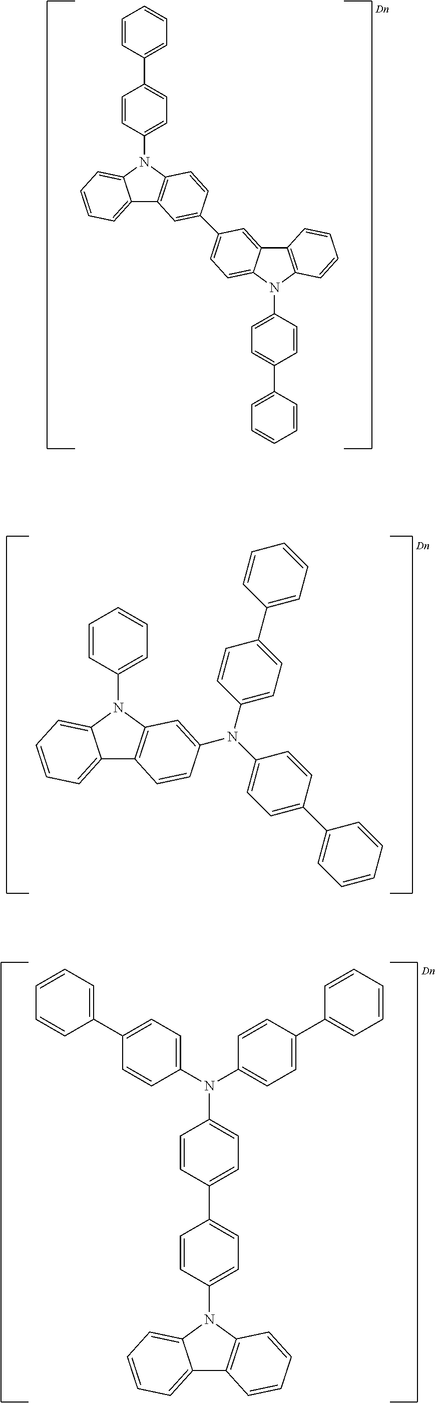

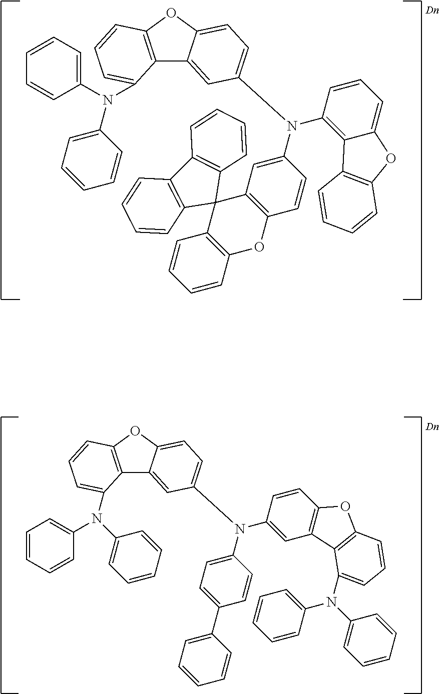

(Dn refers to the number of deuterium substitutions and indicates a structure substituted with one or more deuterium atoms)

In the hole transport region 140, in addition to the compounds described above, other suitable compounds having a similar structure may also be used.

In an implementation, the charge transport region may be, e.g., the electron transport region 150.

The electron transport region 150 may help further increase electron injection or electron mobility and block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

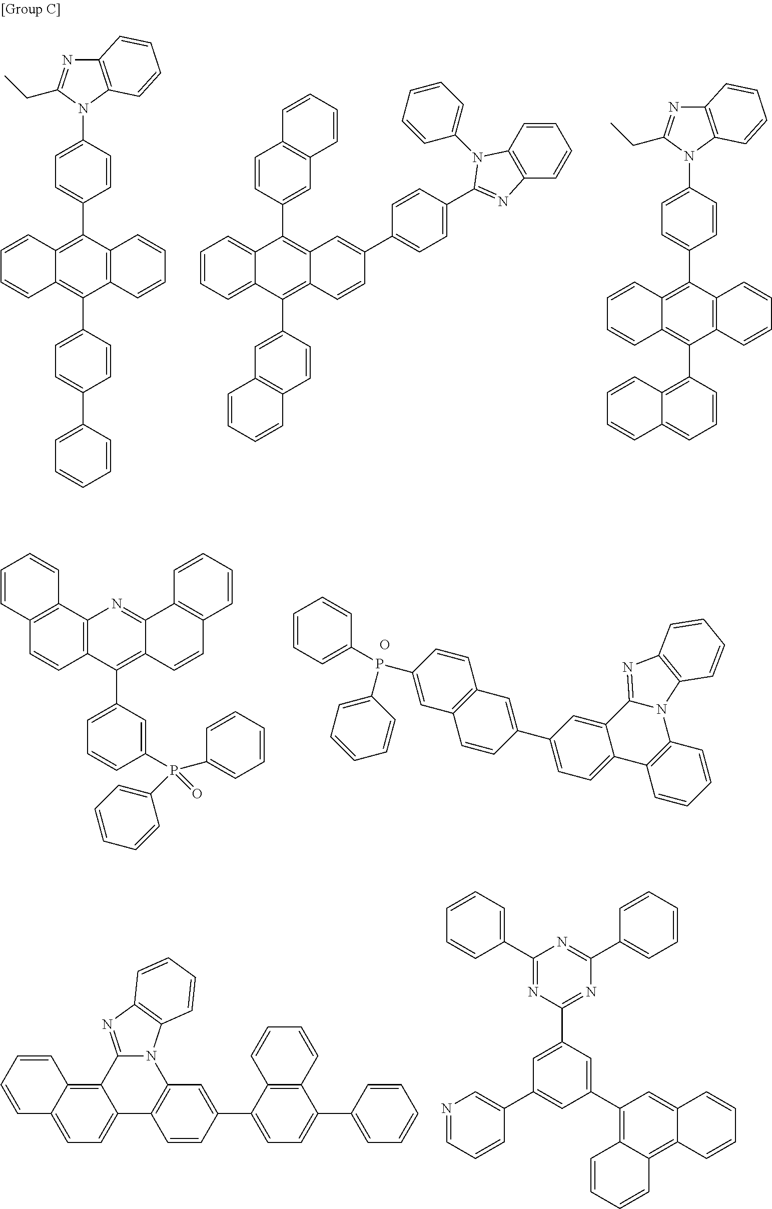

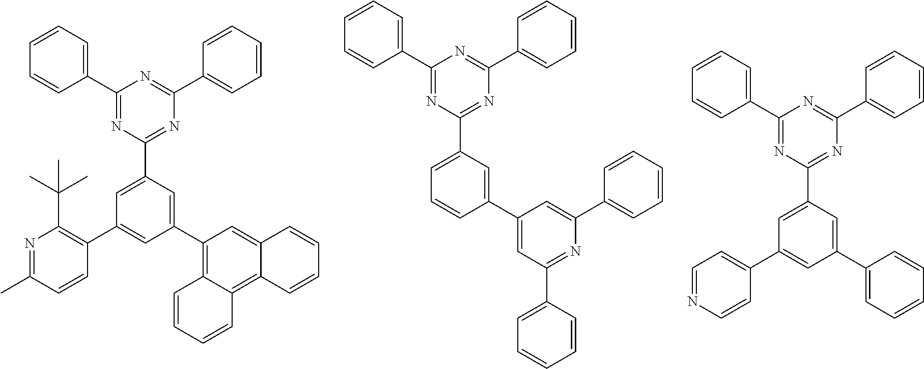 [SMILES: Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1]

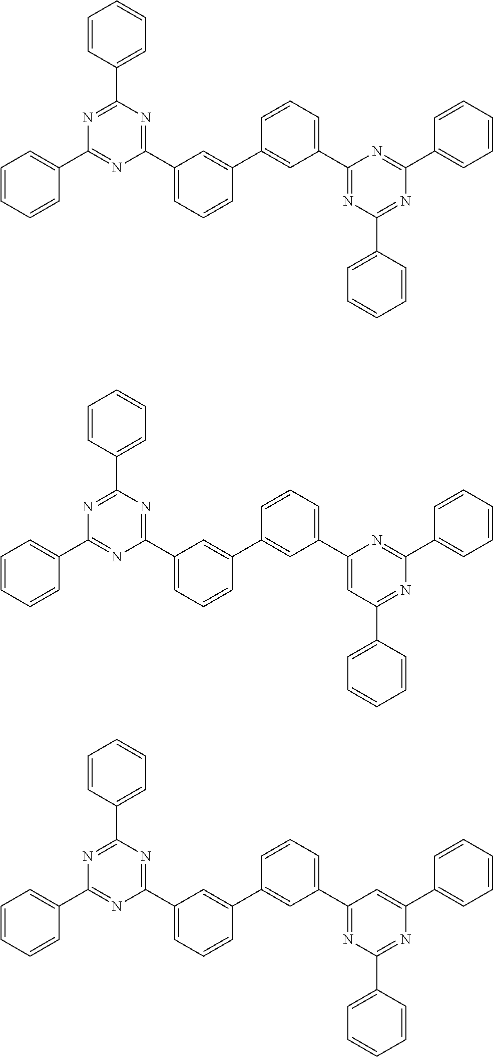 [SMILES: c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

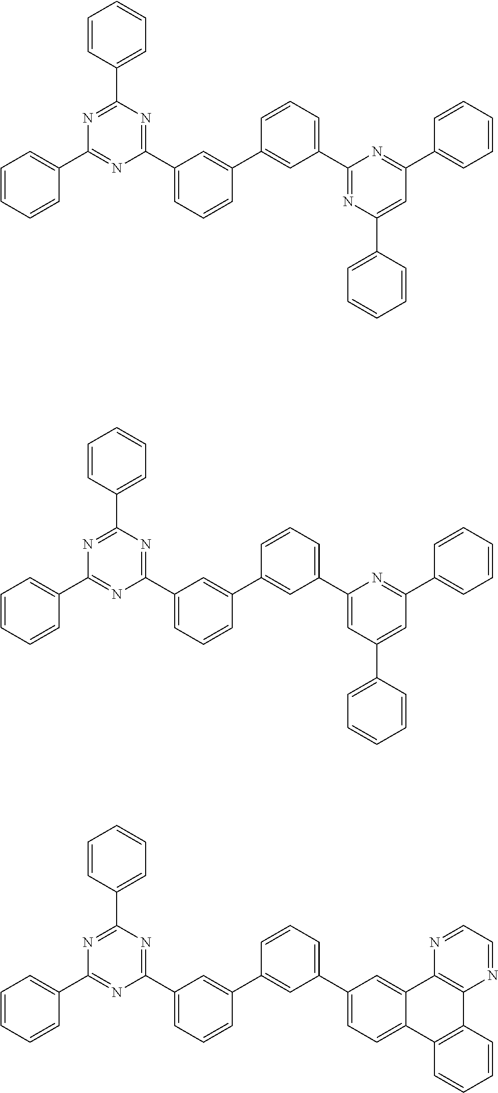 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1]

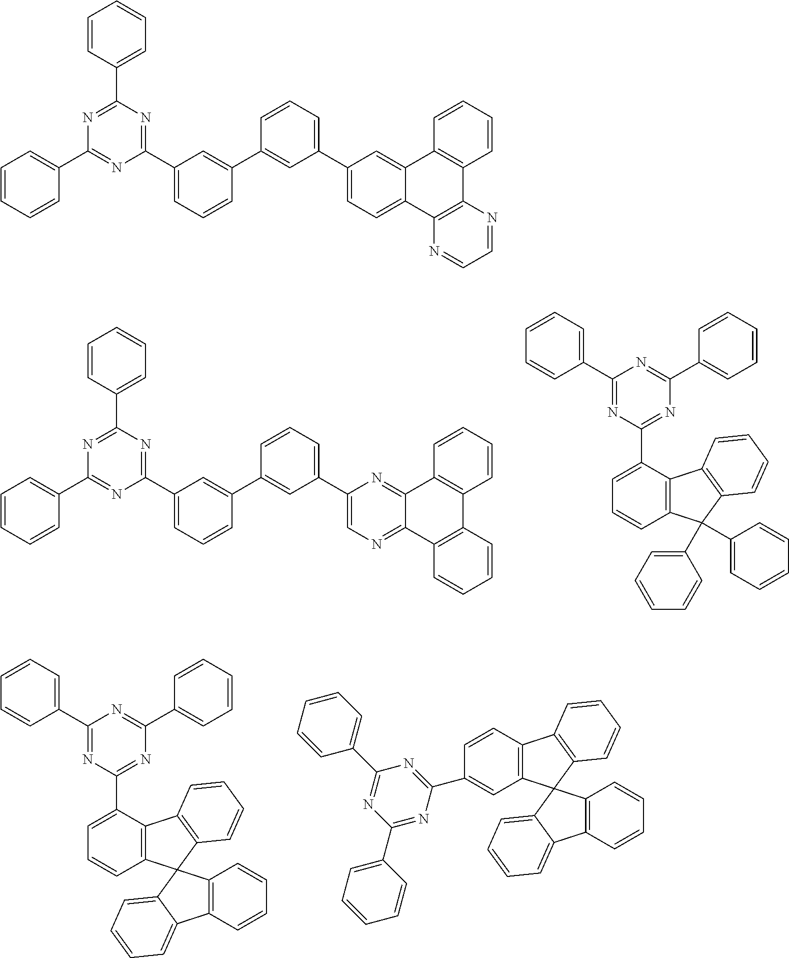 [SMILES: c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1]

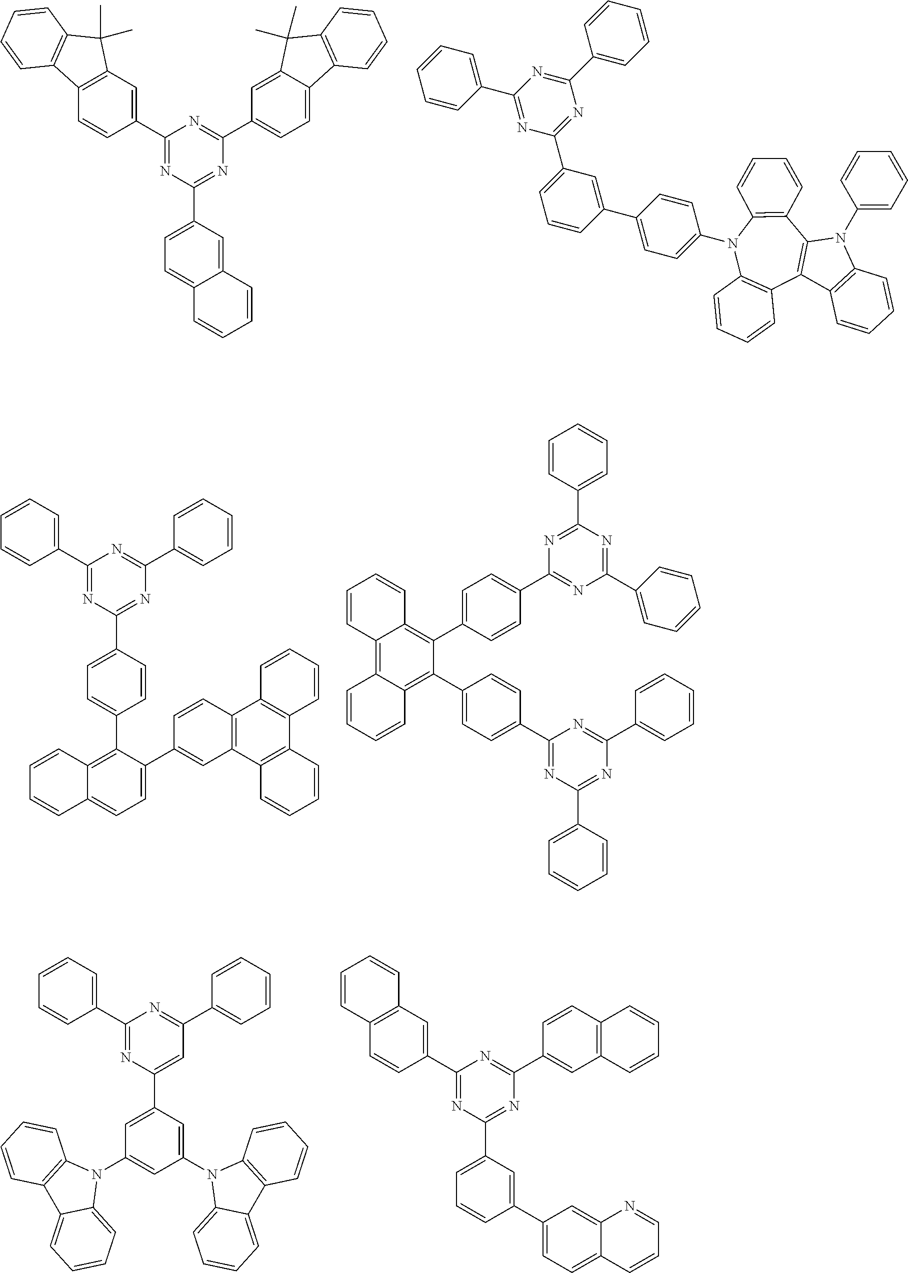

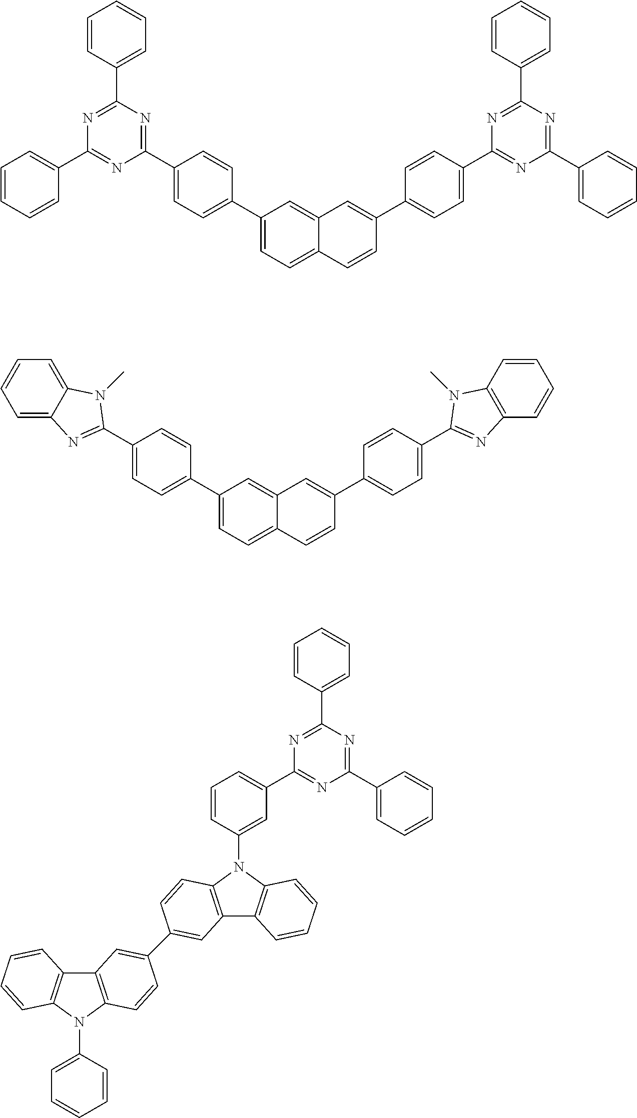 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

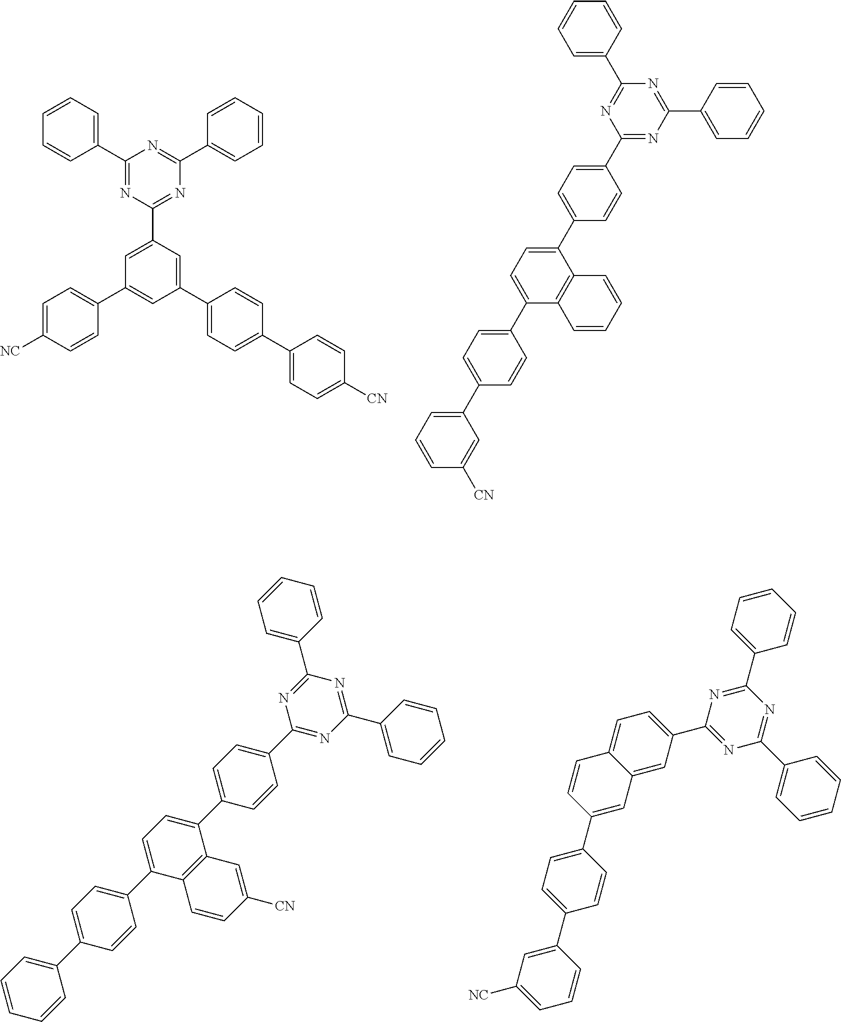 [SMILES: N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1]

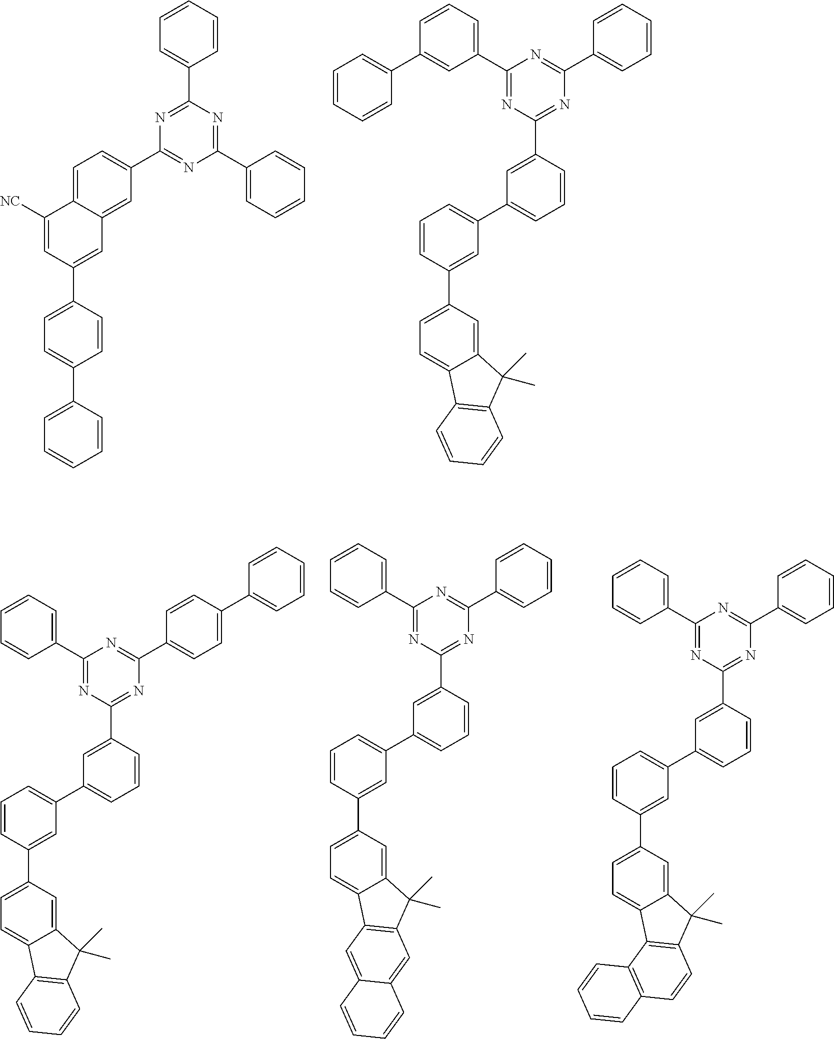

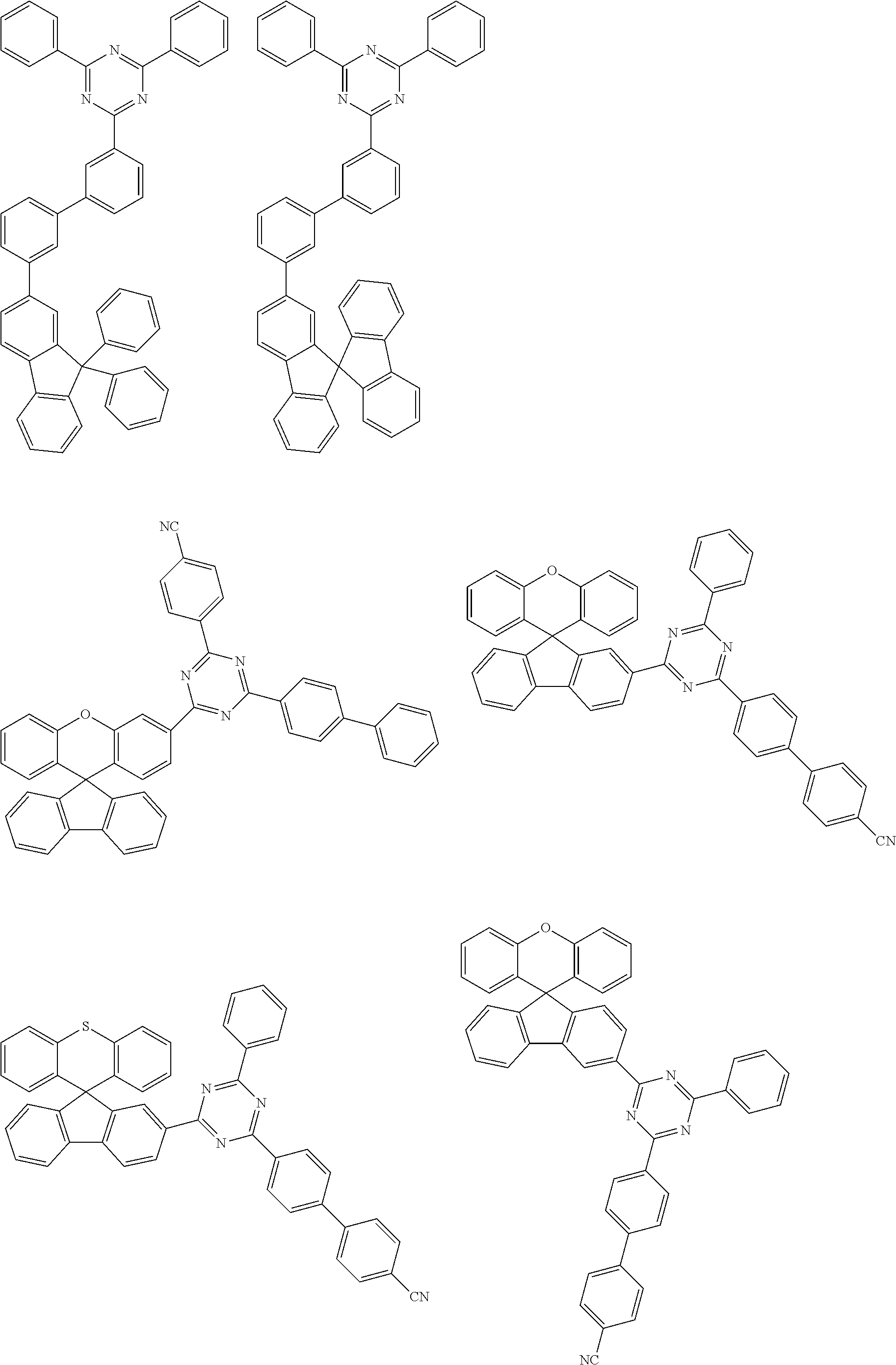

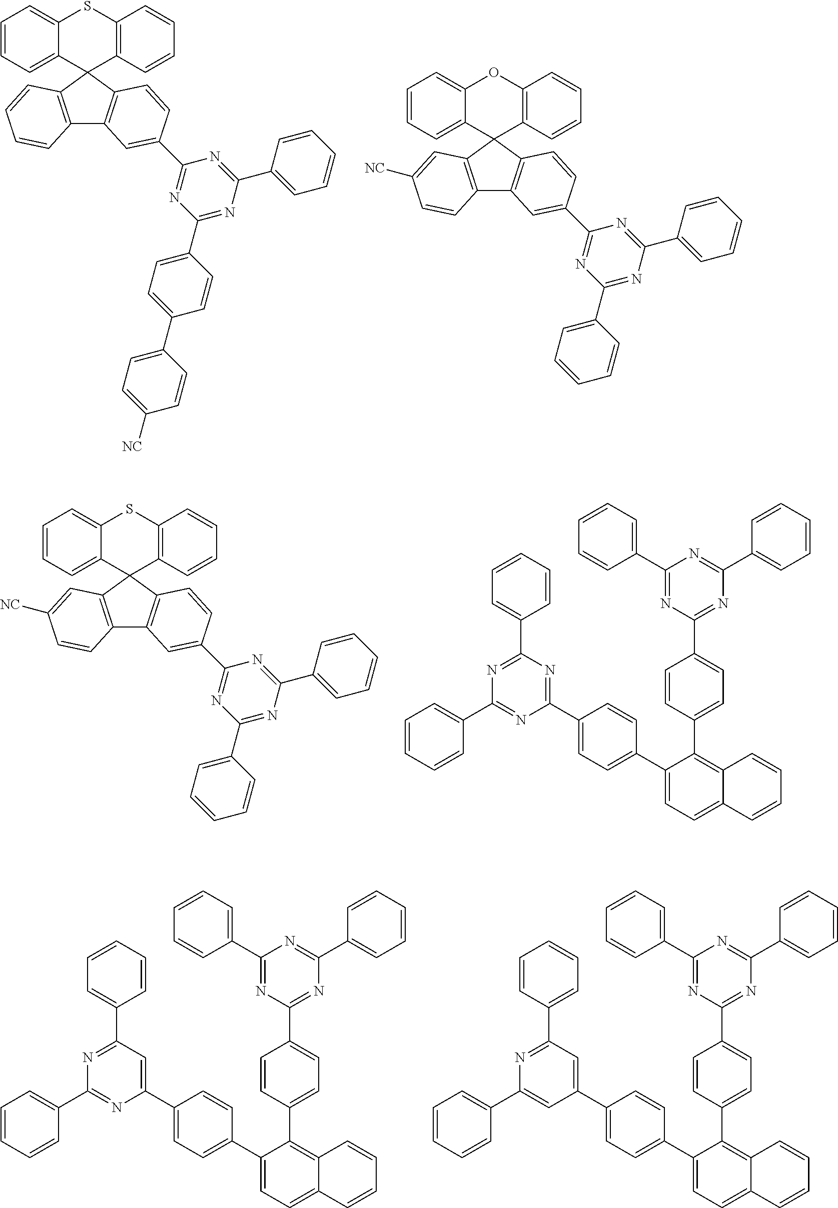

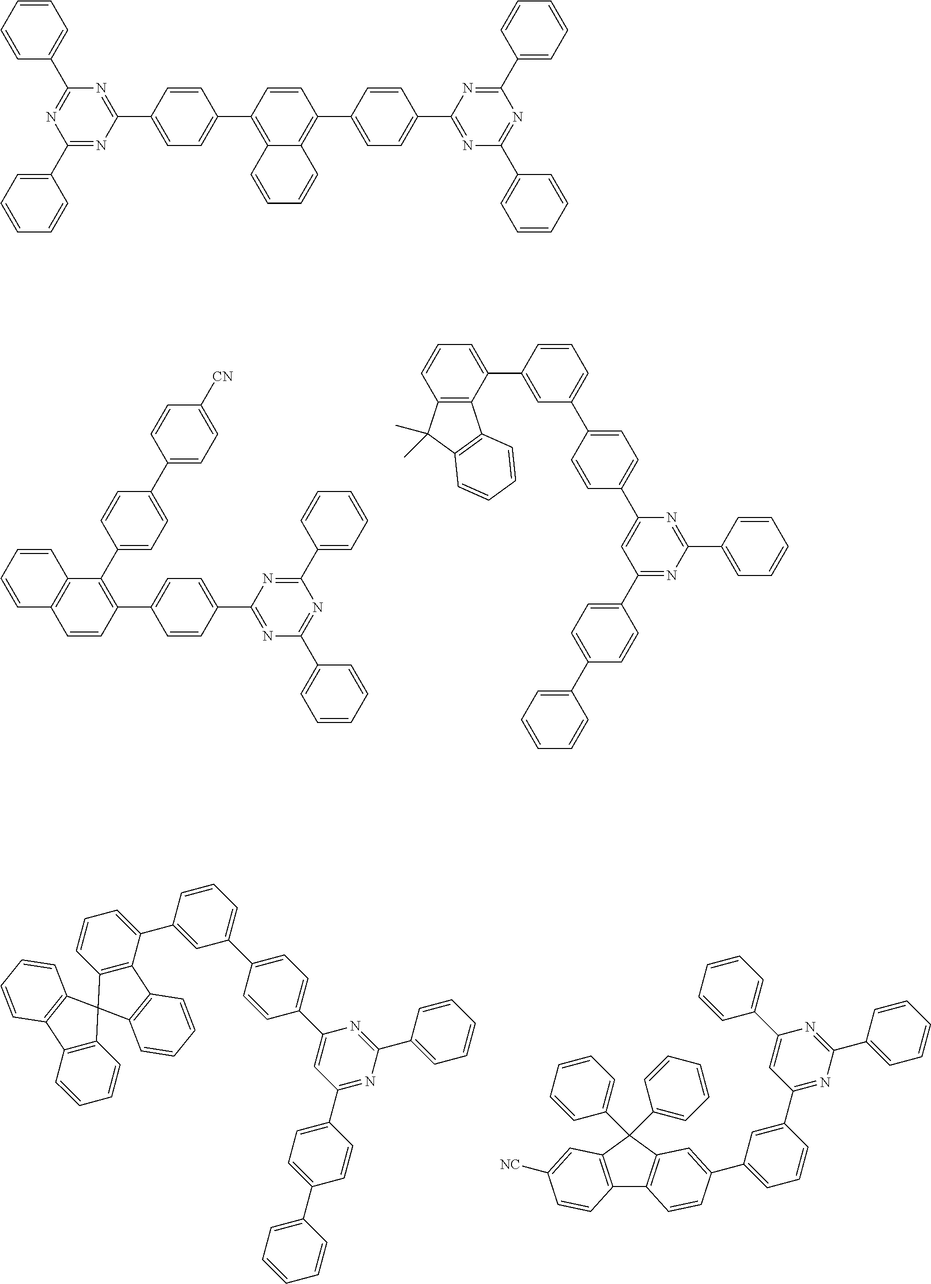 [SMILES: c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1 ; N#Cc1ccc(-c2ccc(-c3c(-c4ccc(-c5nc(-c6ccccc6)nc(-c6ccccc6)n5)cc4)ccc4ccccc34)cc2)cc1 ; CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2]

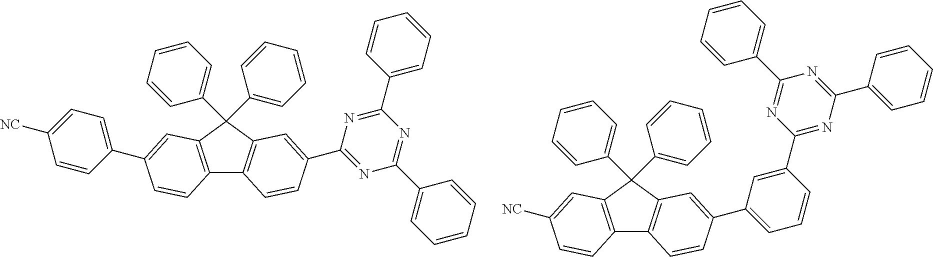

Some embodiments may provide an organic light emitting diode including the light emitting layer as the organic layer.

Some embodiments may provide an organic light emitting diode including a light emitting layer and a hole transport region as the organic layer.

Some embodiments may provide an organic light emitting diode including a light emitting layer and an electron transport region as the organic layer.

An organic light emitting diode according to some embodiments includes a hole transport region 140 and an electron transport region 150 in addition to the light emitting layer 130 as the organic layer 105, as shown in the FIGURE.

In an implementation, an organic light emitting diode may further include an electron injection layer, a hole injection layer, or the like, in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by known methods.

### Synthesis of Compound for Organic Optoelectronic Device

### Synthesis Example 1: Synthesis of Intermediate I-1

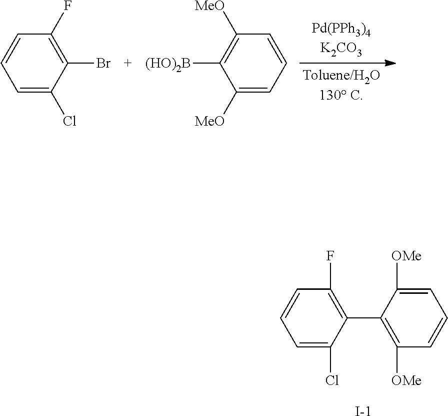 [SMILES: Fc1cccc(Cl)c1Br ; COc1cccc(OC)c1B(O)O ; COc1cccc(OC)c1-c1c(F)cccc1Cl ; [I-]]

2-Bromo-1-chloro-3-fluorobenzene (1000 g, 4,775 mmol) purchased from Henan Tianfu Chemical (www.tianfuchem.net) was dissolved in 10 L of Toluene in a nitrogen environment, and then 2,6-dimethoxyphenylboronic acid (1043 g, 5,730 mmol) and tetrakis(triphenylphosphine) palladium (110 g, 95.5 mmol) purchased from Bide pharma (https://jlchem) were added and stirred. Then, potassium carbonate (1,650 g, 11,938 mmol) saturated in water was added and heated under reflux at 130° C. for 3 days. When a reaction was completed, after adding water thereto, extraction with dichloromethane (DCM) was performed, moisture was removed with magnesium sulfate anhydrous, filtered, and concentrated under reduced pressure. The residue thus obtained was separated and purified by flash column chromatography to obtain Intermediate I-1 (535 g, 42%).

HRMS (70 eV, EI+): m/z calcd for C14H12ClFO2: 266.0510, found: 266.

Elemental Analysis: C, 63%; H, 5%

### Synthesis Example 2: Synthesis of Intermediate I-2

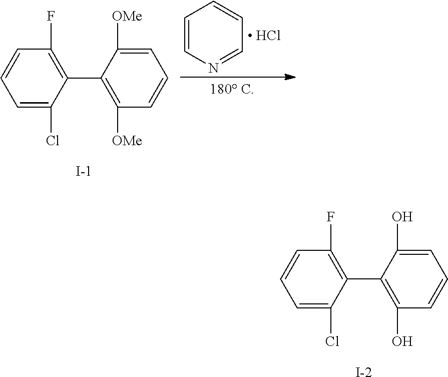

Intermediate I-1 (500 g, 1,875 mmol) and pyridine hydrochloride (1,483 g, 18,748 mmol) were added in a nitrogen environment and heated under reflux at 180° C. for 12 hours. After completion of the reaction, water was added to the reaction solution, the mixture was extracted with ethylacetate (EA), treated with magnesium sulfate anhydrous to remove moisture, filtered, and concentrated under reduced pressure. The residue thus obtained was separated and purified by flash column chromatography to obtain Intermediate I-2 (361 g, 81%).

HRMS (70 eV, EI+): m/z calcd for C12H8ClFO2: 238.0197, found: 238.

Elemental Analysis: C, 60%; H, 3%

### Synthesis Example 3: Synthesis of Intermediate I-3

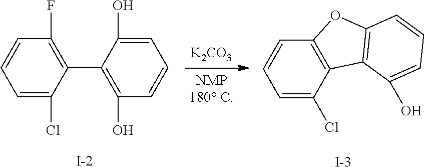

Intermediate I-2 (350 g, 1,467 mmol) was dissolved in 0.3 L of N-methyl-2-pyrrolidone (NMP) in a nitrogen environment, then potassium carbonate (406 g, 2,934 mmol) was added thereto and heated under reflux for 3 hours. After completion of the reaction, the solvent was removed by distillation, water was added to the reaction solution, the mixture was extracted with dichloromethane (DCM), treated with magnesium sulfate anhydrous to remove moisture, filtered, and concentrated under reduced pressure. The residue thus obtained was separated and purified by flash column chromatography to obtain Intermediate I-3 (103 g, 32%).

HRMS (70 eV, EI+): m/z calcd for C12H7ClO2: 218.0135, found: 218.

Elemental Analysis: C, 66%; H, 3%

### Synthesis Example 4: Synthesis of Intermediate I-4

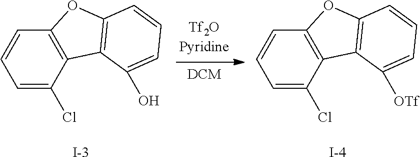

Intermediate I-3 (100 g, 457 mmol) was dissolved in 1.0 L of dichloromethane (DCM) in a nitrogen environment, and then the temperature was lowered to 0° C. Pyridine (43.4 g, 549 mmol) was added here and stirred for 30 minutes, then tifluoromethanesulfonic anhydride (155 g, 549 mmol) was slowly added thereto and stirred. After 3 hours, the temperature of the reaction solution was lowered to 0° C., water was slowly added for 30 minutes, the mixture was extracted with dichloromethane (DCM), treated with magnesium sulfate anhydrous to remove moisture, filtered, and concentrated under reduced pressure. The residue thus obtained was separated and purified by flash column chromatography to obtain Intermediate I-4 (157 g, 98%).

HRMS (70 eV, EI+): m/z calcd for C13H6ClF3O4S: 349.9627, found: 350.

Elemental Analysis: C, 45%; H, 2%

### Synthesis Example 5: Synthesis of Intermediate I-5

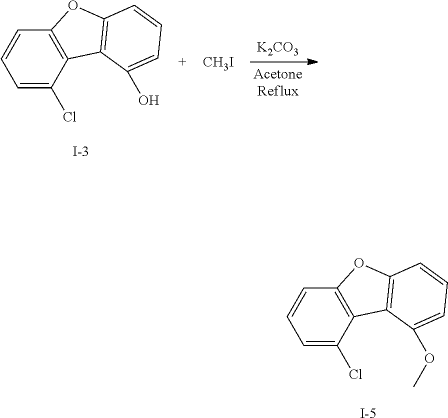

Intermediate I-3 (100 g, 457 mmol) was dissolved in 1.0 L of acetone in a nitrogen environment, then iodomethane (71.4, 503 mmol) and potassium carbonate (69.5 g, 503 mmol) were added and heated under reflux for 6 hours. After a reaction was completed, after adding water thereto, the mixture was extracted with dichloromethane (DCM), treated with magnesium sulfate anhydrous to remove moisture, filtered, and concentrated under reduced pressure. The residue thus obtained was separated and purified by flash column chromatography to obtain Intermediate I-5 (101 g, 95%).

HRMS (70 eV, EI+): m/z calcd for C13H9ClO2: 232.0291, found: 232.

Elemental Analysis: C, 67%; H, 4%

### Synthesis Example 6: Synthesis of Intermediate I-6

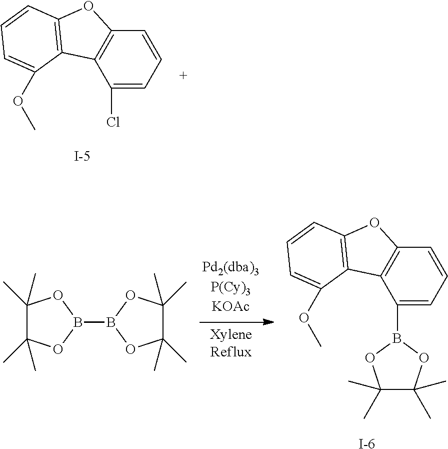

After dissolving Intermediate I-5 (100 g, 430 mmol) in 1.0 L of xylene in a nitrogen environment, bis(pinacolato)diboron (131 g, 516 mmol), tris(dibenzylideneacetone) dipalladium (0) (3.94 g, 4.3 mmol), tricyclohexylphosphine (4.82 g, 17.2 mmol), and potassium acetate (127 g, 1,290 mmol) were added thereto and heated under reflux for 8 hours. After completion of the reaction, water was added to the reaction solution, the mixture was extracted with dichloromethane (DCM), treated with magnesium sulfate anhydrous to remove moisture, filtered, and concentrated under reduced pressure. The residue thus obtained was separated and purified by flash column chromatography to obtain Intermediate I-6 (121 g, 87%).

HRMS (70 eV, EI+): m/z calcd for C19H21BO4: 324.1533, found: 324.

Elemental Analysis: C, 70%; H, 7%

### Synthesis Example 7: Synthesis of Intermediate I-7

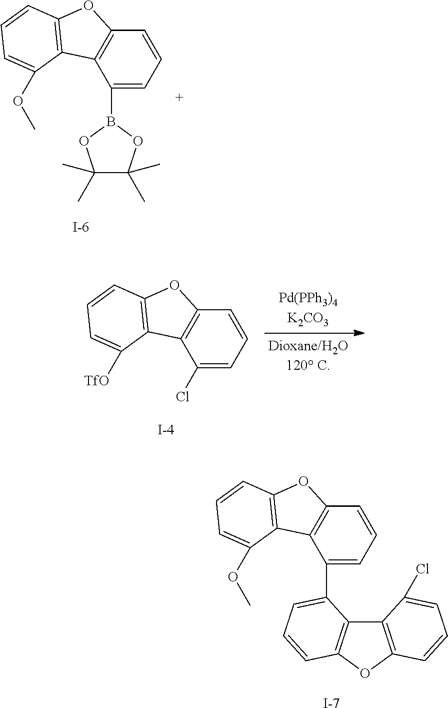

After dissolving intermediate I-6 (120 g, 370 mmol) in 1.2 L of dioxane in a nitrogen environment, Intermediate I-4 (130 g, 370 mmol) and tetrakis(triphenylphosphine) palladium (8.55 g, 7.4 mmol) were added thereto and stirred. Then, potassium carbonate (128 g, 925 mmol) saturated in water was added and heated under reflux at 100° C. for 8 hours. After a reaction was completed, after adding water thereto, the mixture was extracted with dichloromethane (DCM), treated with magnesium sulfate anhydrous to remove moisture, and concentrated under reduced pressure. The residue thus obtained was separated and purified by flash column chromatography to obtain Intermediate I-7 (145 g, 98%).

HRMS (70 eV, EI+): m/z calcd for C25H15ClO3: 398.0710, found: 398.

Elemental Analysis: C, 75%; H, 4%

### Synthesis Example 8: Synthesis of Intermediate I-8

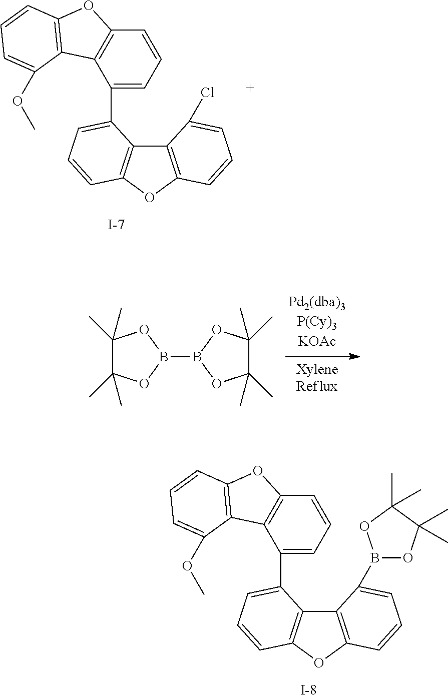

Intermediate I-8 (86.1 g, 50%) was obtained using Intermediate I-7 (140 g, 351 mmol) in the same manner as in Synthesis Example 6.

HRMS (70 eV, EI+): m/z calcd for C31H27BO5: 490.1952, found: 490.

Elemental Analysis: C, 76%; H, 6%

### Synthesis Example 9: Synthesis of Intermediate I-9

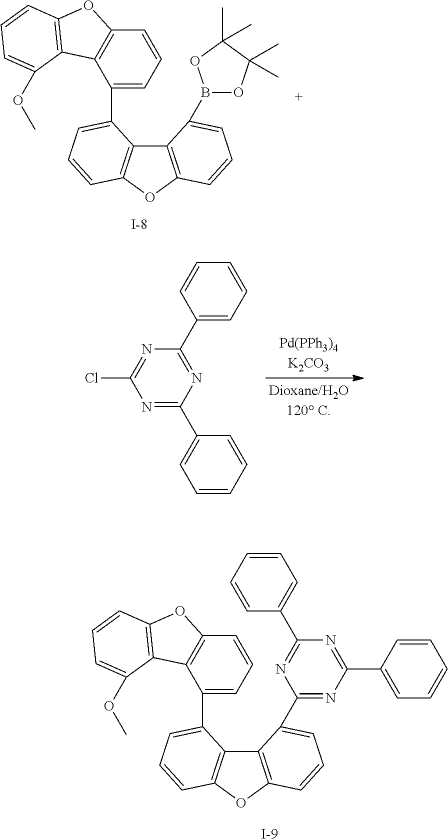

Intermediate I-9 (89.7 g, 87%) was obtained in the same manner as Synthesis Example 7 by using Intermediate I-8 (85 g, 173 mmol) and 2-chloro-4,6-diphenyl-1,3,5-triazine (55.7 g, 208 mmol) purchased from Tokyo Chemical Industries (http://www.tcichemicals.com/).

HRMS (70 eV, EI+): m/z calcd for C40H25N3O3: 595.1896, found: 595.

Elemental Analysis: C, 81%; H, 4%

### Synthesis Example 10: Synthesis of Intermediate I-10

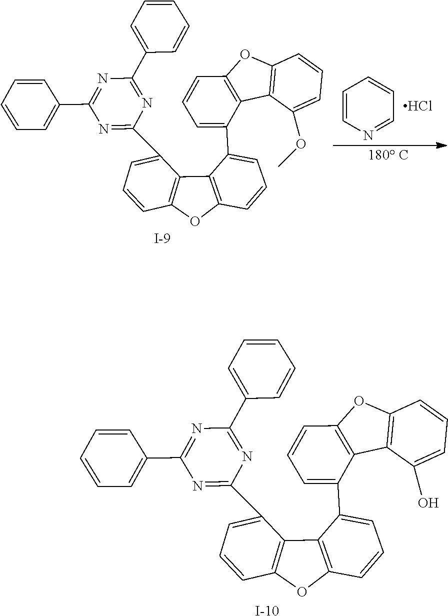

Intermediate I-10 (85.2 g, 99%) was obtained using Intermediate I-9 (88 g, 148 mmol) in the same manner as in Synthesis Example 2.

HRMS (70 eV, EI+): m/z calcd for C39H23N3O3: 581.1739, found: 581.

Elemental Analysis: C, 81%; H, 4%

### Synthesis Example 11: Synthesis of Intermediate I-11

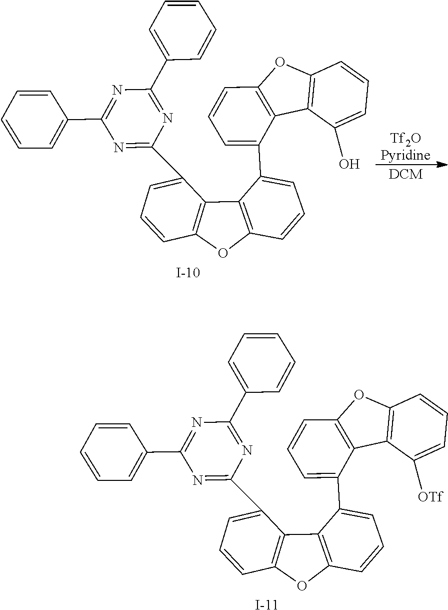

Intermediate I-11 (93.8 g, 91%) was obtained using Intermediate I-10 (84 g, 144 mmol) in the same manner as in Synthesis Example 4.

HRMS (70 eV, EI+): m/z calcd for C40H22F3N3O5S: 713.1232, found: 713.

Elemental Analysis: C, 67%; H, 3%

### Synthesis Example 12: Synthesis of Intermediate I-12

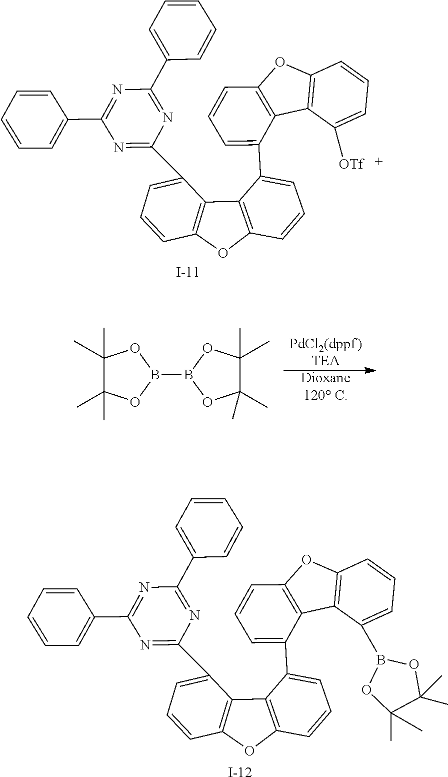

After dissolving Intermediate I-11 (92 g, 129 mmol) in 1.0 L of dioxane in a nitrogen environment, bis(pinacolato)diboron (49.1 g, 193 mmol) and 1,1-bis(diphenylphosphino) ferrocenedichloropalladium (3.16 g, 3.87 mmol) were added thereto, and triethylamine (39.2 g, 387 mmol) was added and heated under reflux for 15 hours. After completion of the reaction, water was added to the reaction solution, the mixture was extracted with dichloromethane (DCM), treated with magnesium sulfate anhydrous to remove moisture, filtered, and concentrated under reduced pressure. The residue thus obtained was separated and purified by flash column chromatography to obtain Intermediate I-12 (43.7 g, 49%).

HRMS (70 eV, EI+): m/z calcd for C45H34BN3O4: 691.2642, found: 691.

Elemental Analysis: C, 78%; H, 5%

### Synthesis Example 13: Synthesis of Compound 1

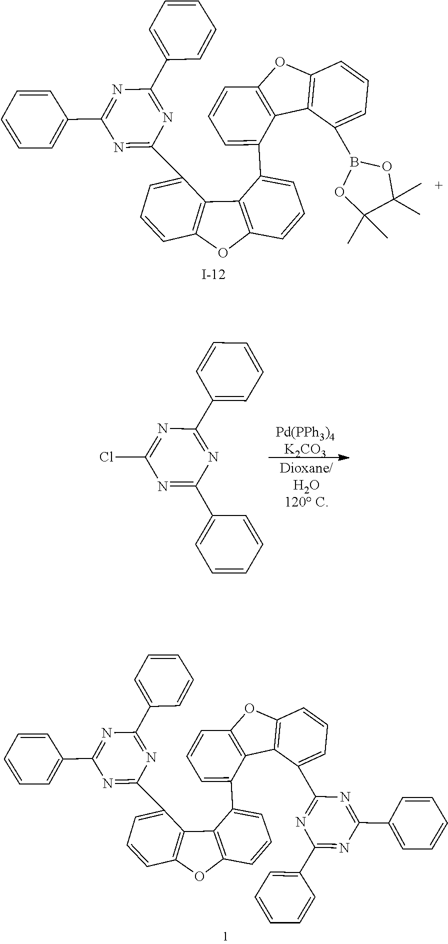

Compound 1 (18.4 g, 80%) was obtained in the same manner as Synthesis Example 7 by using Intermediate I-12 (20 g, 28.9 mmol) and 2-chloro-4,6-diphenyl-1,3,5-triazine (9.29 g, 34.7 mmol) purchased from Tokyo Chemical Industries.

HRMS (70 eV, EI+): m/z calcd for C54H32N6O2: 796.2587, found: 796.

Elemental Analysis: C, 81%; H, 4%

### Synthesis Example 14: Synthesis of Compound 2

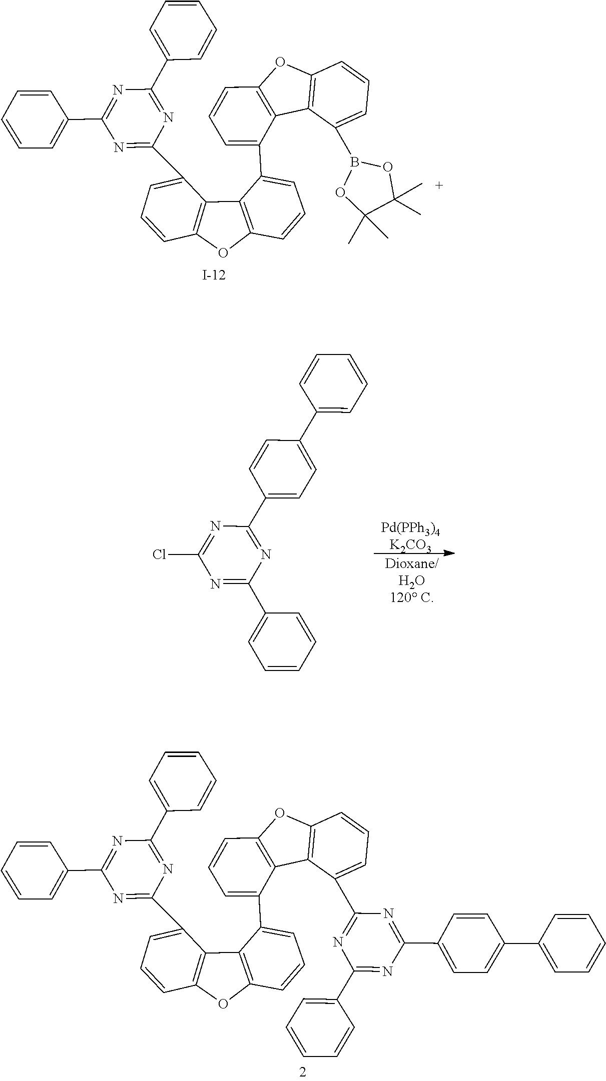

Compound 2 (20.9 g, 83%) was obtained in the same manner as Synthesis Example 7 by using Intermediate I-12 (20 g, 28.9 mmol) and 2-([1,1′-biphenyl]-4-yl)-4-chloro-6-phenyl-1,3,5-triazine (11.9 g, 34.7 mmol) purchased from Tokyo Chemical Industries.

HRMS (70 eV, EI+): m/z calcd for C60H36N6O2: 872.2900, found: 872.

Elemental Analysis: C, 83%; H, 4%

### Synthesis Example 15: Synthesis of Intermediate I-13

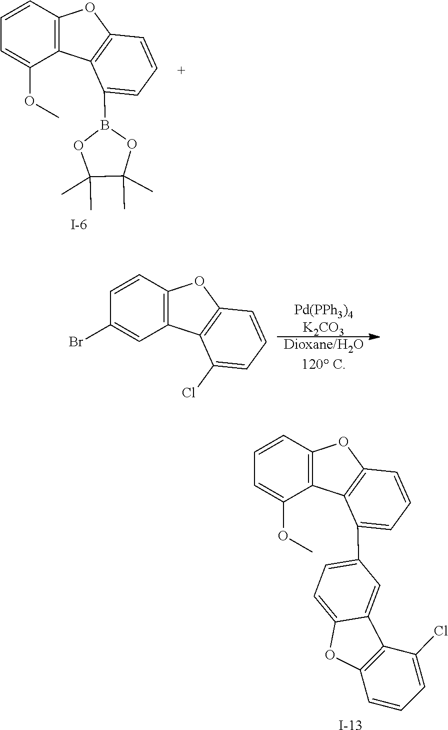

Intermediate I-13 (117 g, 95%) was obtained in the same manner as Synthesis Example 7 by using Intermediate I-6 (100 g, 308 mmol) and 8-bromo-1-chlorodibenzofuran (86.8 g, 308 mmol) purchased from P&H tech (http://www.phtech.co.kr/).

HRMS (70 eV, EI+): m/z calcd for C25H15ClO3: 398.0710, found: 398.

Elemental Analysis: C, 75%; H, 4%

### Synthesis Example 16: Synthesis of Intermediate I-14

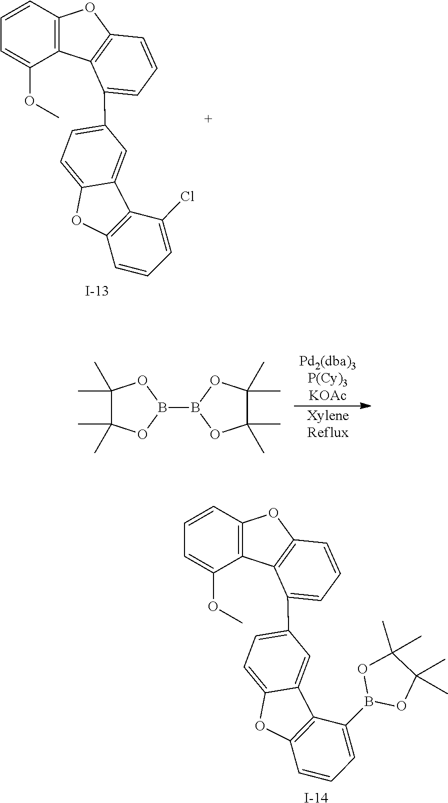

Intermediate I-14 (101 g, 75%) was obtained using Intermediate I-13 (110 g, 276 mmol) in the same manner as in Synthesis Example 6.

HRMS (70 eV, EI+): m/z calcd for C31H27BO5: 490.1952, found: 490.

Elemental Analysis: C, 76%; H, 6%

### Synthesis Example 17: Synthesis of Intermediate I-15

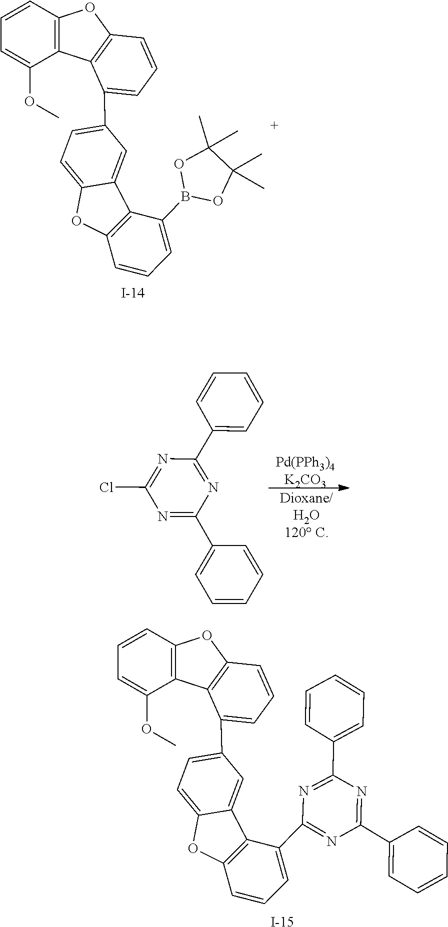

Intermediate I-15 (109 g, 90%) was obtained in the same manner as Synthesis Example 7 by using Intermediate I-14 (100 g, 204 mmol) and 2-chloro-4,6-diphenyl-1,3,5-triazine (54.6 g, 204 mmol) purchased from Tokyo Chemical Industries.

HRMS (70 eV, EI+): m/z calcd for C40H25N3O3: 595.1896, found: 595.

Elemental Analysis: C, 81%; H, 4%

### Synthesis Example 18: Synthesis of Intermediate I-16

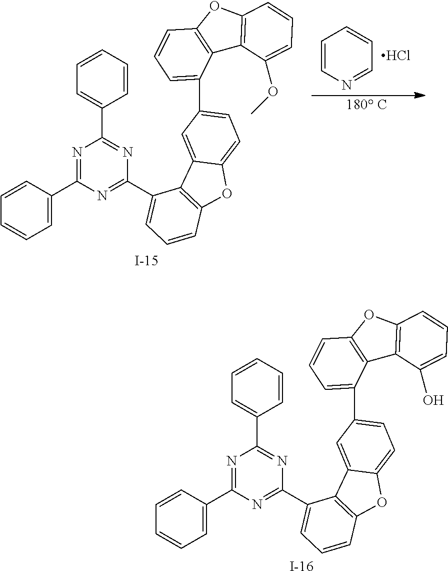

Intermediate I-16 (98.3 g, 96%) was obtained using Intermediate I-15 (105 g, 176 mmol) in the same manner as Synthesis Example 2.

HRMS (70 eV, EI+): m/z calcd for C39H23N3O3: 581.1739, found: 581.

Elemental Analysis: C, 81%; H, 4%

### Synthesis Example 19: Synthesis of Intermediate I-17

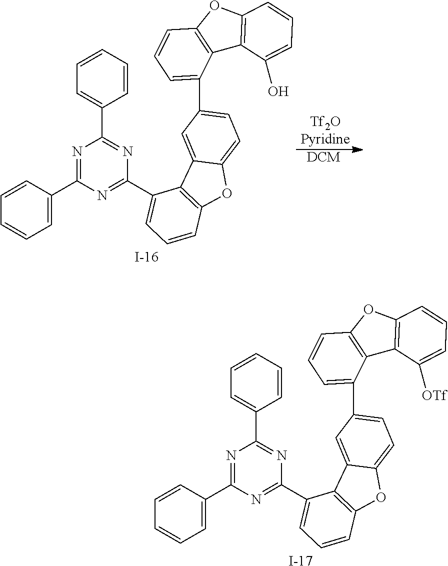

Intermediate I-17 (111 g, 95%) was obtained using Intermediate I-16 (95 g, 163 mmol) in the same manner as in Synthesis Example 4.

HRMS (70 eV, EI+): m/z calcd for C40H22F3N3O5S: 713.1232, found: 713.

Elemental Analysis: C, 67%; H, 3%

### Synthesis Example 20: Synthesis of Intermediate I-18

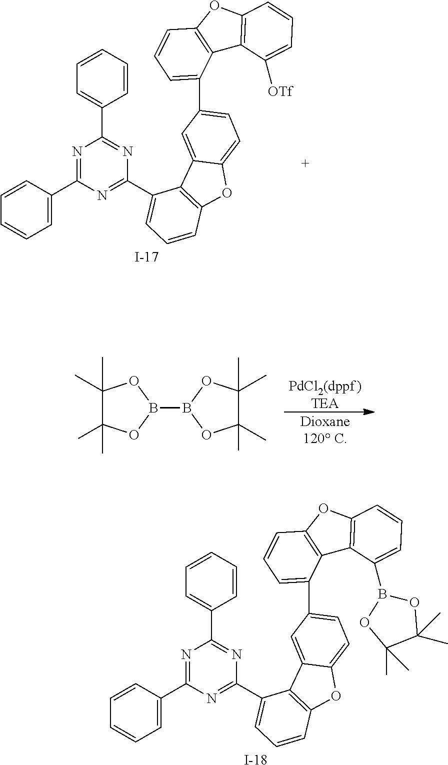

Intermediate I-18 (54.3 g, 51%) was obtained using Intermediate I-17 (110 g, 154 mmol) in the same manner as in Synthesis Example 12.

HRMS (70 eV, EI+): m/z calcd for C45H34BN3O4: 691.2642, found: 691.

Elemental Analysis: C, 78%; H, 5%

### Synthesis Example 21: Synthesis of Compound 9

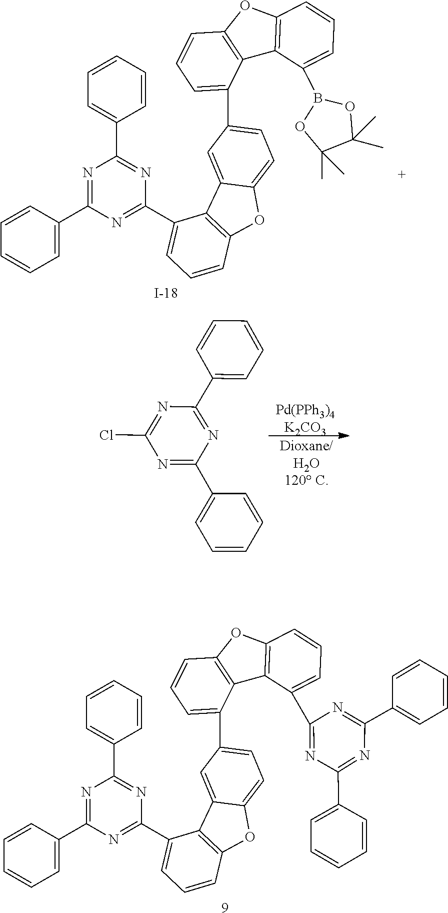

Compound 9 (17.5 g, 76%) was obtained in the same manner as Synthesis Example 7 by using Intermediate I-18 (20 g, 28.9 mmol) and 2-chloro-4,6-diphenyl-1,3,5-triazine (9.28 g, 34.7 mmol) purchased from Tokyo Chemical Industries.

HRMS (70 eV, EI+): m/z calcd for C54H32N6O2: 796.2587, found: 796.

Elemental Analysis: C, 81%; H, 4%

### Synthesis Example 22: Synthesis of Intermediate I-19

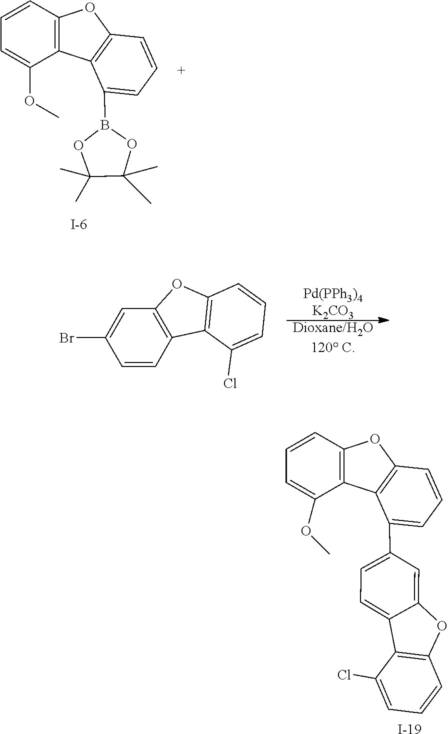

Intermediate I-19 (112 g, 91%) was obtained in the same manner as Synthesis Example 7 by using Intermediate I-6 (100 g, 308 mmol) and 7-bromo-1-chlorodibenzofuran (86.8 g, 308 mmol) purchased from P&H tech.

HRMS (70 eV, EI+): m/z calcd for C25H15ClO3: 398.0710, found: 398.

Elemental Analysis: C, 75%; H, 4%

### Synthesis Example 23: Synthesis of Intermediate I-20

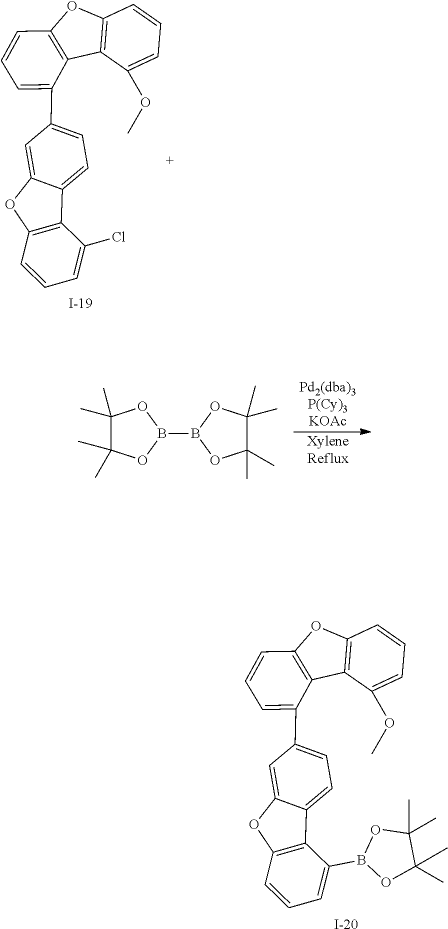

Intermediate I-20 (89.3 g, 66%) was obtained using Intermediate I-19 (110 g, 276 mmol) in the same manner as in Synthesis Example 6.

HRMS (70 eV, EI+): m/z calcd for C31H27BO5: 490.1952, found: 490.

Elemental Analysis: C, 76%; H, 6%

### Synthesis Example 24: Synthesis of Intermediate I-21

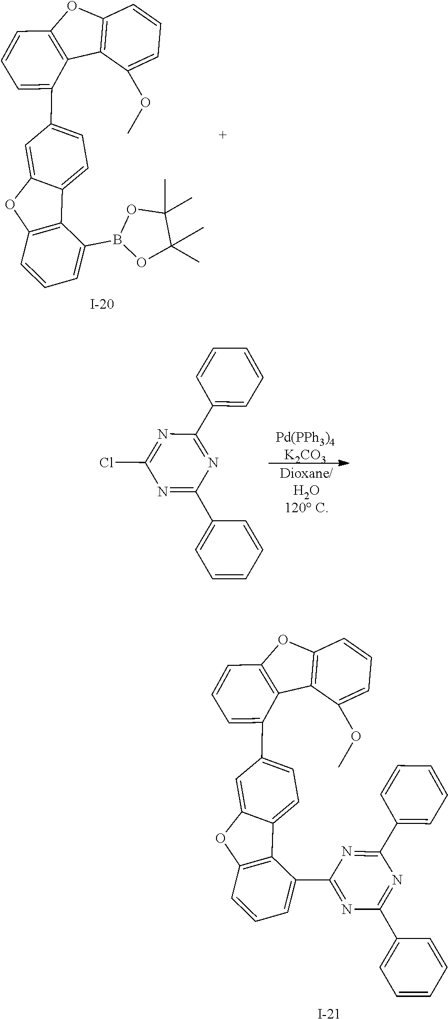

Intermediate I-21 (94.8 g, 92%) was obtained in the same manner as Synthesis Example 7 by using Intermediate I-20 (85 g, 173 mmol) and 2-chloro-4,6-diphenyl-1,3,5-triazine (51.0 g, 191 mmol) purchased from Tokyo Chemical Industries.

HRMS (70 eV, EI+): m/z calcd for C40H25N3O3: 595.1896, found: 595.

Elemental Analysis: C, 81%; H, 4%

### Synthesis Example 25: Synthesis of Intermediate I-22

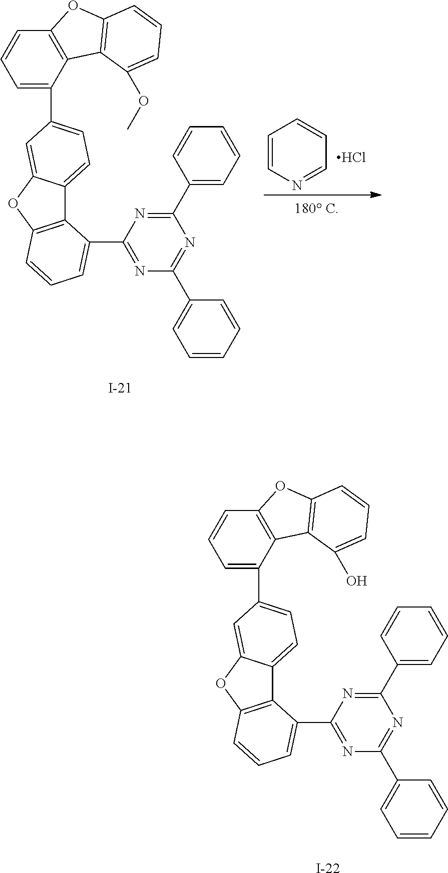

Intermediate I-22 (80.8 g, 90%) was obtained using Intermediate I-21 (92 g, 154 mmol) in the same manner as in Synthesis Example 2.

HRMS (70 eV, EI+): m/z calcd for C39H23N3O3: 581.1739, found: 581.

Elemental Analysis: C, 81%; H, 4%

### Synthesis Example 26: Synthesis of Intermediate I-23

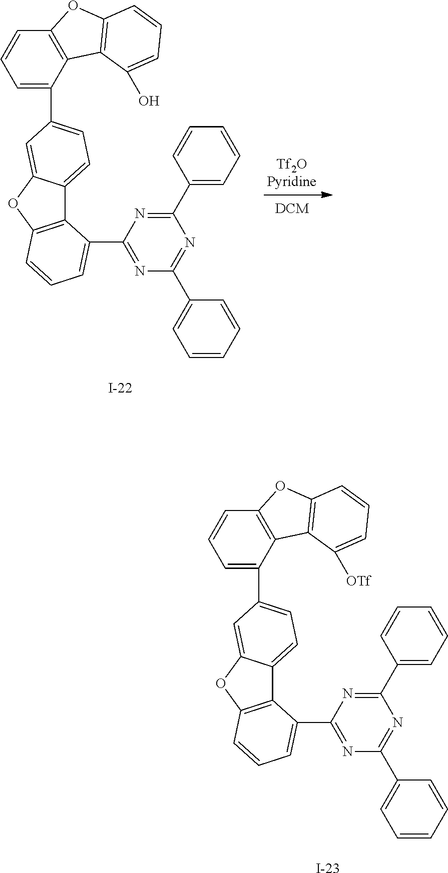

Intermediate I-23 (86.4 g, 89%) was obtained using Intermediate I-22 (79 g, 136 mmol) in the same manner as in Synthesis Example 4.

HRMS (70 eV, EI+): m/z calcd for C40H22F3N3O5S: 713.1232, found: 713.

Elemental Analysis: C, 67%; H, 3%

### Synthesis Example 27: Synthesis of Intermediate I-24

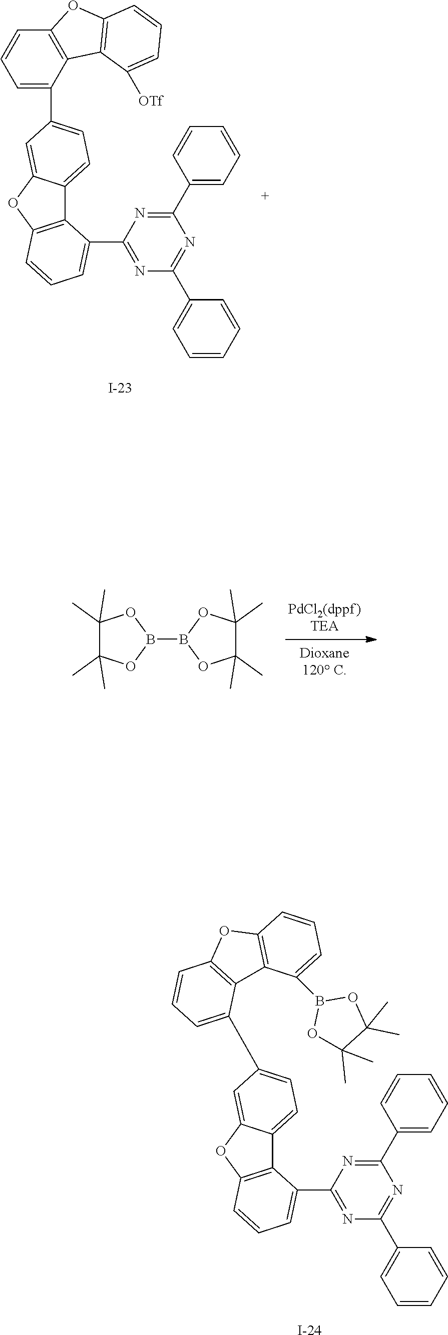

Intermediate I-24 (26.4 g, 32%) was obtained using Intermediate I-23 (85 g, 119 mmol) in the same manner as in Synthesis Example 12.

HRMS (70 eV, EI+): m/z calcd for C45H34BN3O4: 691.2642, found: 691.

Elemental Analysis: C, 78%; H, 5%

### Synthesis Example 28: Synthesis of Compound 10

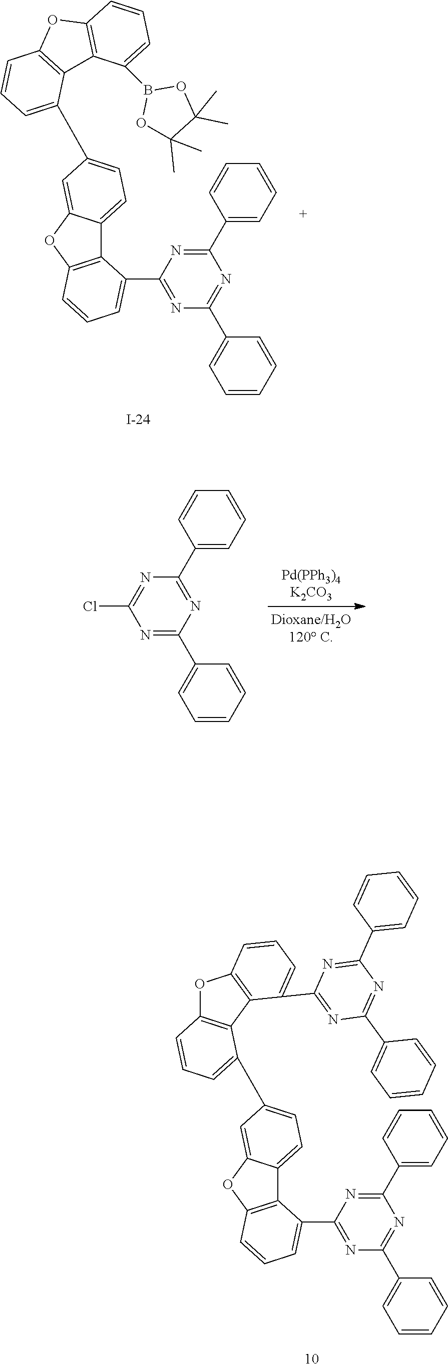

Compound 10 (11.5 g, 50%) was obtained in the same manner as Synthesis Example 7 by using Intermediate I-24 (20 g, 28.9 mmol) and 2-chloro-4,6-diphenyl-1,3,5-triazine (9.28 g, 34.7 mmol) purchased from Tokyo Chemical Industries.

HRMS (70 eV, EI+): m/z calcd for C54H32N6O2: 796.2587, found: 796.

Elemental Analysis: C, 81%; H, 4%

### Synthesis Example 29: Synthesis of Intermediate I-25

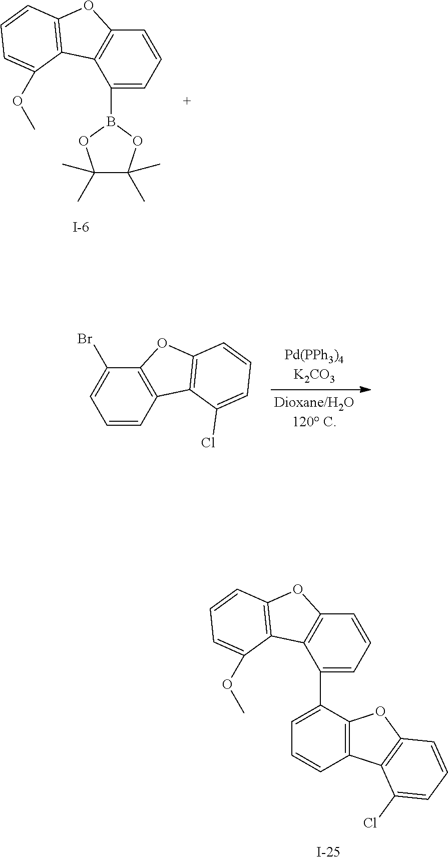

Intermediate I-25 (117 g, 95%) was obtained in the same manner as Synthesis Example 7 by using Intermediate I-6 (100 g, 308 mmol) and 6-bromo-1-chlorodibenzofuran (86.8 g, 308 mmol) purchased from P&H tech.

HRMS (70 eV, EI+): m/z calcd for C25H15ClO3: 398.0710, found: 398.

Elemental Analysis: C, 75%; H, 4%

### Synthesis Example 30: Synthesis of Intermediate I-26

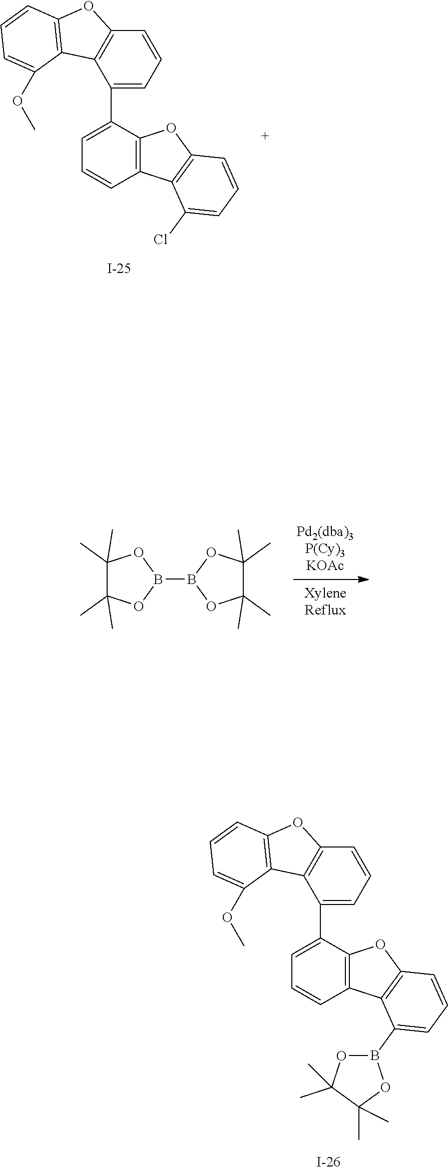

Intermediate I-26 (113 g, 80%) was obtained using Intermediate I-25 (115 g, 288 mmol) in the same manner as in Synthesis Example 6.

HRMS (70 eV, EI+): m/z calcd for C31H27BO5: 490.1952, found: 490.

Elemental Analysis: C, 76%; H, 6%

### Synthesis Example 31: Synthesis of Intermediate I-27

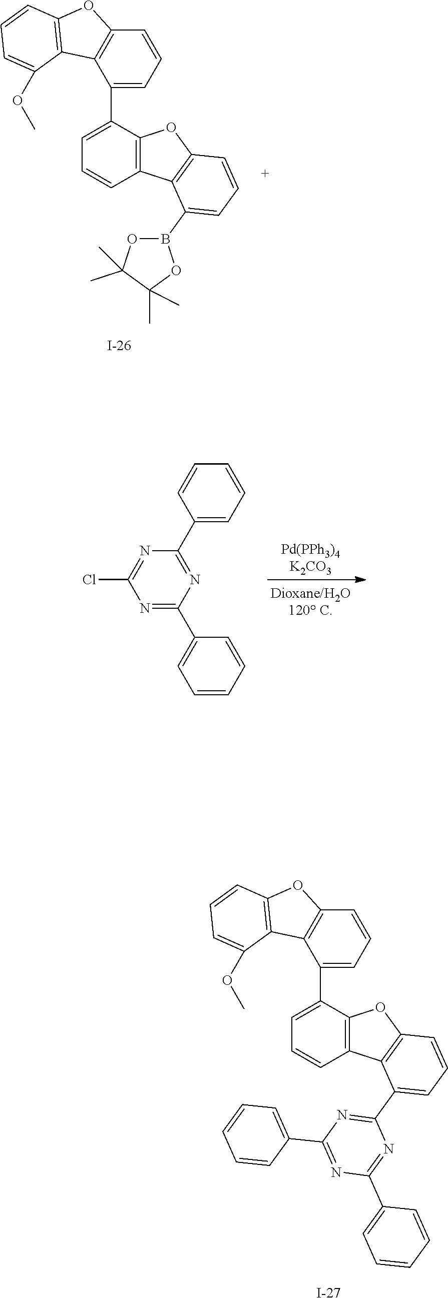

Intermediate I-27 (123 g, 92%) was obtained in the same manner as Synthesis Example 7 by using Intermediate I-26 (110 g, 224 mmol) and 2-chloro-4,6-diphenyl-1,3,5-triazine (66.1 g, 247 mmol) purchased from Tokyo Chemical Industries.

HRMS (70 eV, EI+): m/z calcd for C40H25N3O3: 595.1896, found: 595.

Elemental Analysis: C, 81%; H, 4%

### Synthesis Example 32: Synthesis of Intermediate I-28

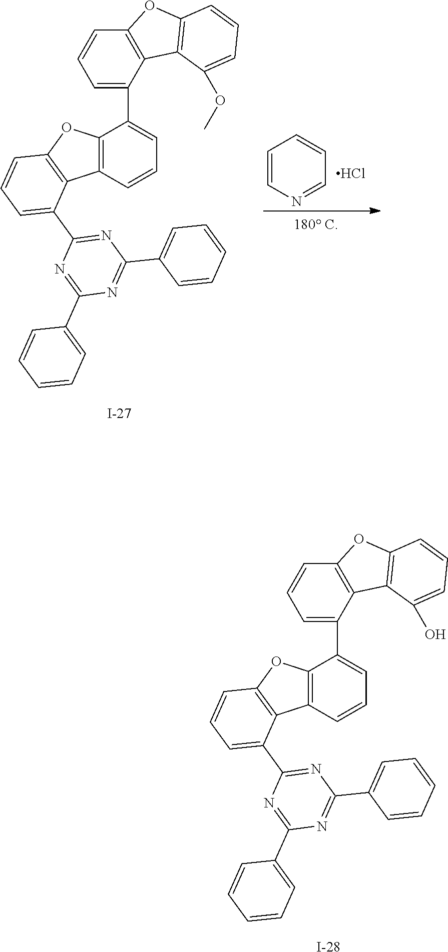

Intermediate I-28 (115 g, 98%) was obtained using Intermediate I-27 (120 g, 201 mmol) in the same manner as in Synthesis Example 2.

HRMS (70 eV, EI+): m/z calcd for C39H23N3O3: 581.1739, found: 581.

Elemental Analysis: C, 81%; H, 4%

### Synthesis Example 33: Synthesis of Intermediate I-29

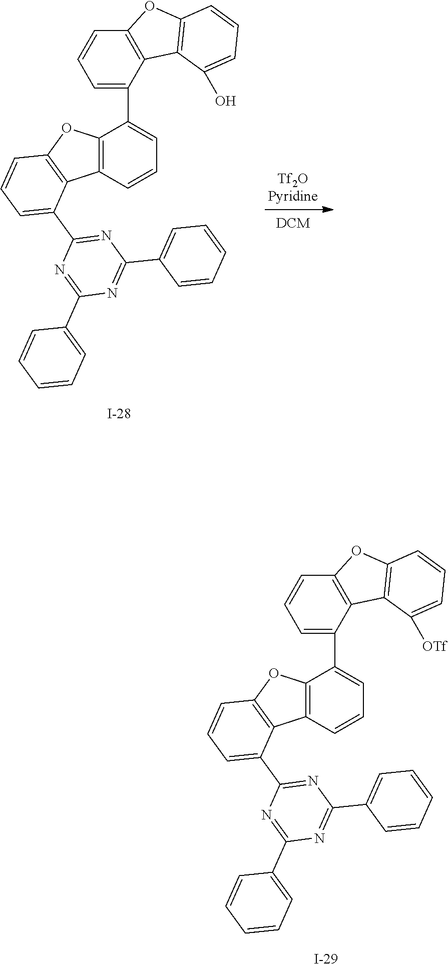

Intermediate I-29 (126 g, 91%) was obtained using Intermediate I-28 (113 g, 194 mmol) in the same manner as in Synthesis Example 4.

HRMS (70 eV, EI+): m/z calcd for C40H22F3N3O5S: 713.1232, found: 713.

Elemental Analysis: C, 67%; H, 3%

### Synthesis Example 34: Synthesis of Intermediate I-30

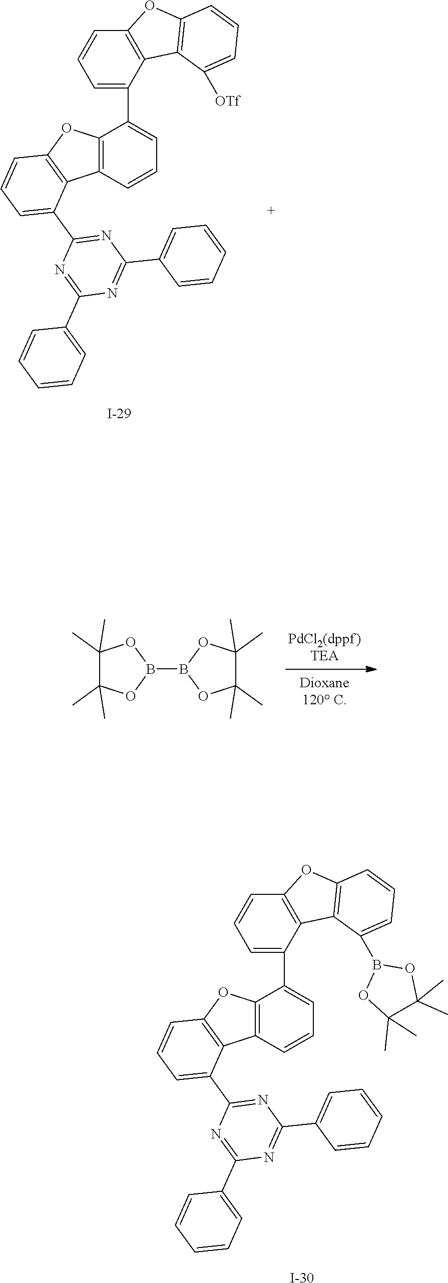

Intermediate I-30 (48.2 g, 40%) was obtained using Intermediate I-29 (125 g, 174 mmol) in the same manner as in Synthesis Example 12.

HRMS (70 eV, EI+): m/z calcd for C45H34BN3O4: 691.2642, found: 691.

Elemental Analysis: C, 78%; H, 5%

### Synthesis Example 35: Synthesis of Compound 11

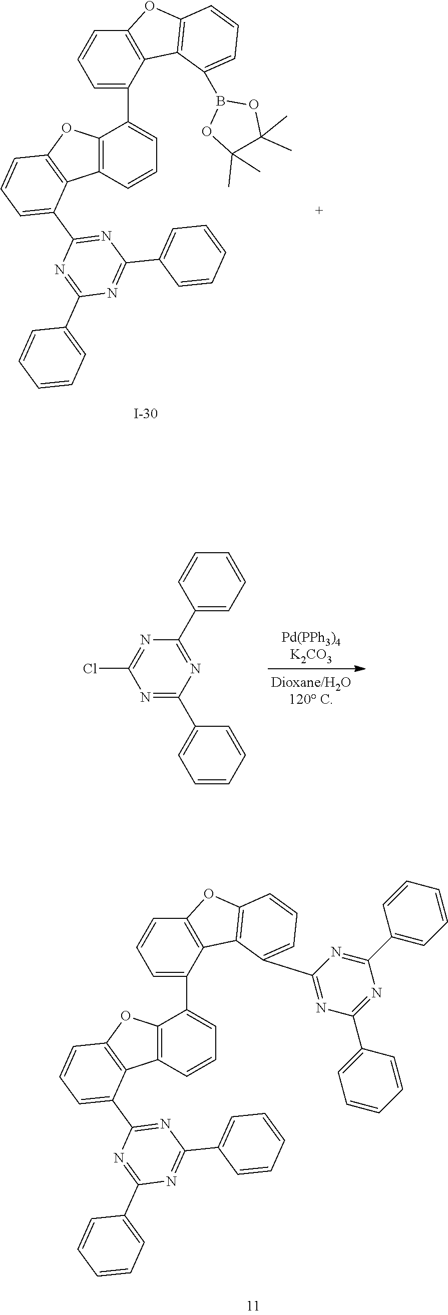

Compound 11 (20.0 g, 87%) was obtained in the same manner as Synthesis Example 7 by using Intermediate I-30 (20 g, 28.9 mmol) and 2-chloro-4,6-diphenyl-1,3,5-triazine (9.28 g, 34.7 mmol) purchased from Tokyo Chemical Industries.

HRMS (70 eV, EI+): m/z calcd for C54H32N6O2: 796.2587, found: 796.

Elemental Analysis: C, 81%; H, 4%

### Synthesis Example 36: Synthesis of Intermediate I-31

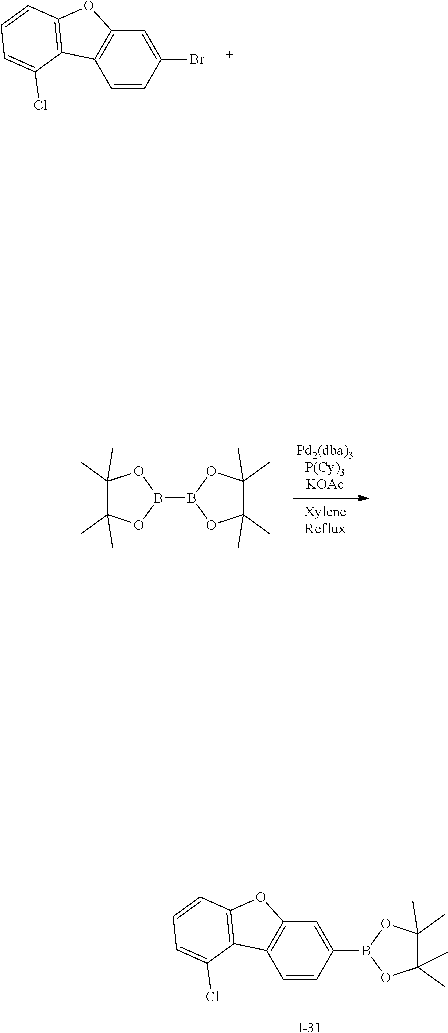

Intermediate I-31 (91.0 g, 78%) was obtained using 7-bromo-1-chlorodibenzofuran (100 g, 355 mmol) in the same manner as in Synthesis Example 6.

HRMS (70 eV, EI+): m/z calcd for C18H18BClO3: 328.1038, found: 328.

Elemental Analysis: C, 66%; H, 6%

### Synthesis Example 37: Synthesis of Intermediate I-32

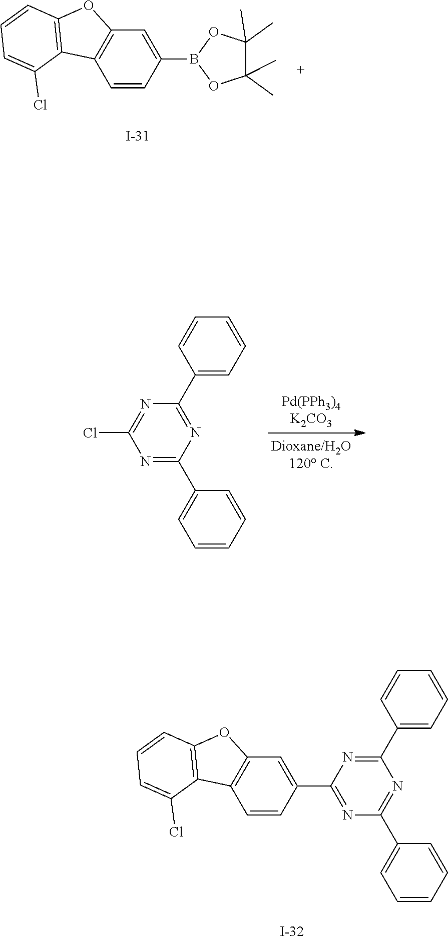

Intermediate I-32 (117 g, 98%) was obtained in the same manner as Synthesis Example 7 by using Intermediate I-31 (90 g, 274 mmol) and 2-chloro-4,6-diphenyl-1,3,5-triazine (80.7 g, 301 mmol) purchased from Tokyo Chemical Industries.

HRMS (70 eV, EI+): m/z calcd for C27H16ClN3O: 433.0982, found: 433.

Elemental Analysis: C, 74%; H, 4%

### Synthesis Example 38: Synthesis of Intermediate I-33

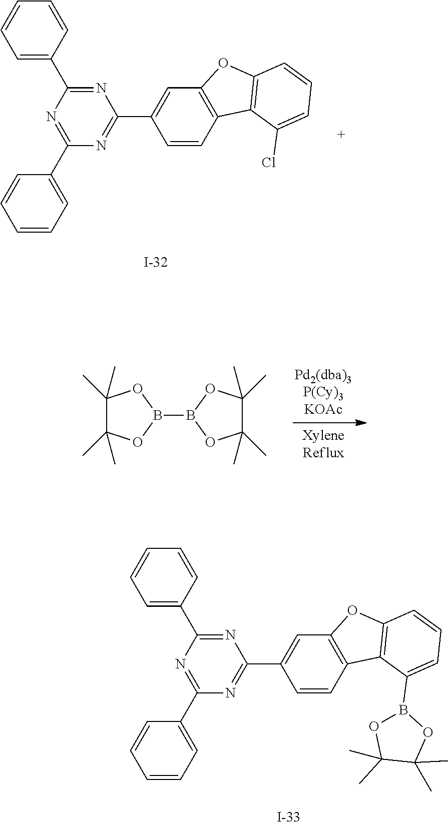

Intermediate I-33 (90.5 g, 65%) was obtained using Intermediate I-32 (115 g, 265 mmol) in the same manner as in Synthesis Example 6.

HRMS (70 eV, EI+): m/z calcd for C33H28BN3O3: 525.2224, found: 525.

Elemental Analysis: C, 75%; H, 5%

### Synthesis Example 39: Synthesis of Intermediate I-34

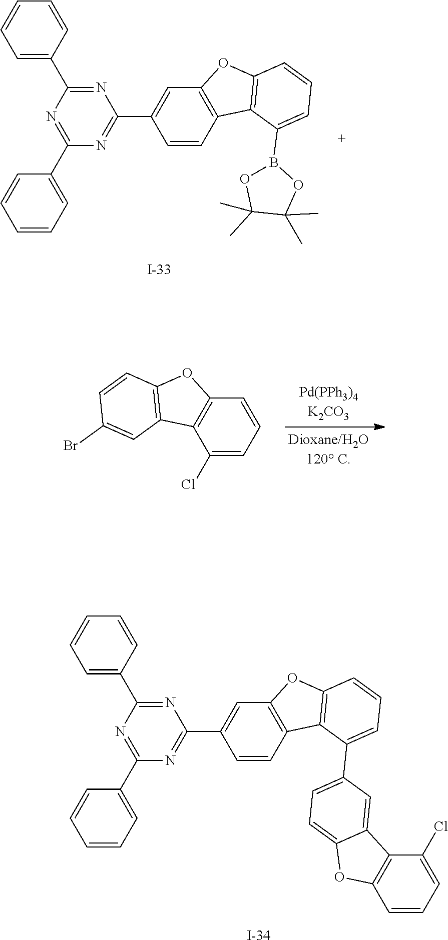

Intermediate I-34 (92.3 g, 91%) was obtained using Intermediate I-33 (89 g, 169 mmol) and 8-bromo-1-chlorodibenzofuran (47.7 g, 169 mmol) in the same manner as in Synthesis Example 7.

HRMS (70 eV, EI+): m/z calcd for C39H22ClN3O2: 599.1401, found: 599.

Elemental Analysis: C, 78%; H, 4%

### Synthesis Example 40: Synthesis of Intermediate I-35

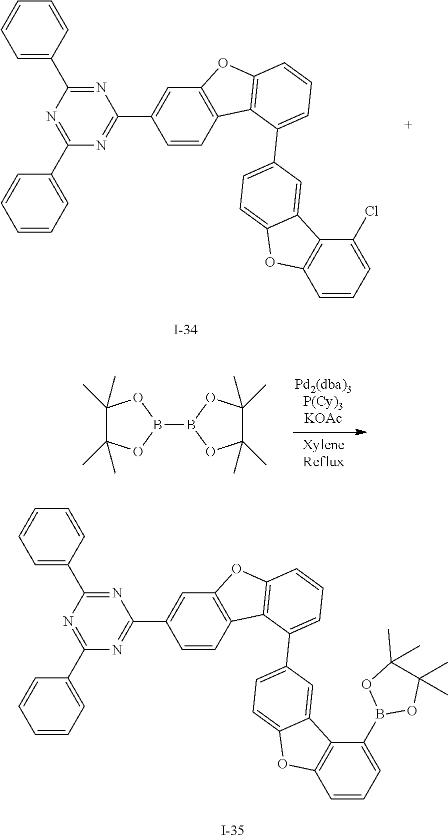

Intermediate I-35 (57.7 g, 55%) was obtained using Intermediate I-34 (91 g, 152 mmol) in the same manner as in Synthesis Example 6.

HRMS (70 eV, EI+): m/z calcd for C45H34BN3O4: 691.2642, found: 691.

Elemental Analysis: C, 78%; H, 5%

### Synthesis Example 41: Synthesis of Compound 17

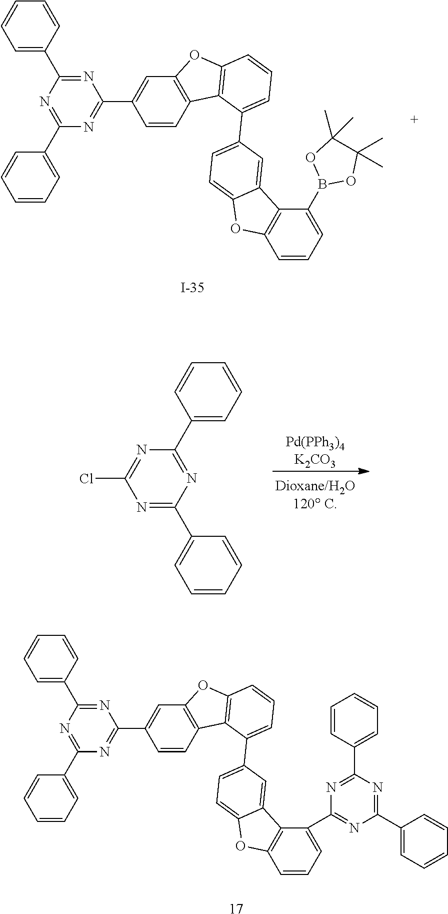 [SMILES: CC1(C)OB(c2cccc3oc4ccc(-c5cccc6oc7cc(-c8nc(-c9ccccc9)nc(-c9ccccc9)n8)ccc7c56)cc4c23)OC1(C)C ; "I-35" ; Clc1nc(-c2ccccc2)nc(-c2ccccc2)n1 ; c1ccc(-c2nc(-c3ccccc3)nc(-c3ccc4c(c3)oc3cccc(-c5ccc6oc7cccc(-c8nc(-c9ccccc9)nc(-c9ccccc9)n8)c7c6c5)c34)n2)cc1 ; "17"]

Compound 17 (18.4 g, 80%) was obtained in the same manner as Synthesis Example 7 by using Intermediate I-35 (20 g, 28.9 mmol) and 2-chloro-4,6-diphenyl-1,3,5-triazine (9.28 g, 34.7 mmol) purchased from Tokyo Chemical Industries.

HRMS (70 eV, EI+): m/z calcd for C54H32N6O2: 796.2587, found: 796.

Elemental Analysis: C, 81%; H, 4%

### Synthesis Example 42: Synthesis of Intermediate I-36

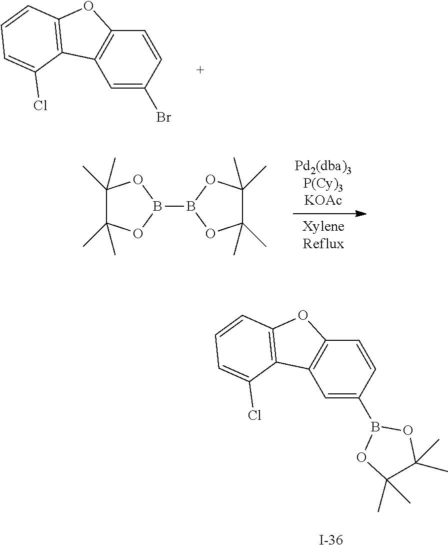

Intermediate I-36 (103 g, 88%) was obtained using 8-bromo-1-chlorodibenzofuran (100 g, 355 mmol) in the same manner as in Synthesis Example 6.

HRMS (70 eV, EI+): m/z calcd for C18H18BClO3: 328.1038, found: 328.

Elemental Analysis: C, 66%; H, 6%

### Synthesis Example 43: Synthesis of Intermediate I-37

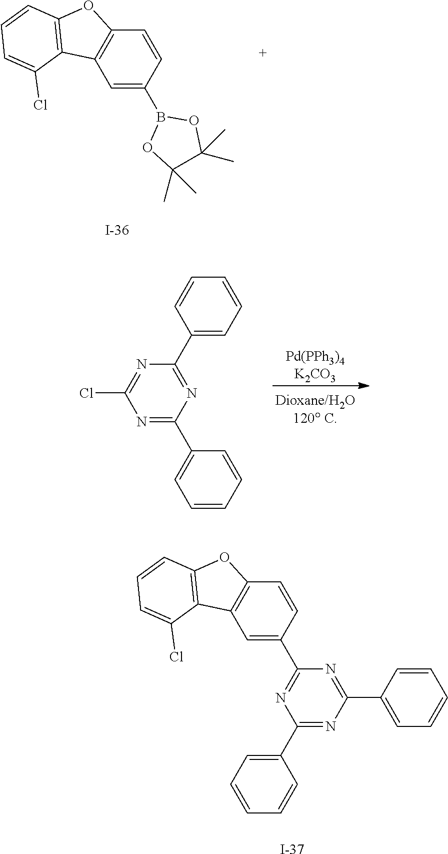

Intermediate I-37 (125 g, 95%) was obtained in the same manner as Synthesis Example 7 by using Intermediate I-36 (100 g, 304 mmol) and 2-chloro-4,6-diphenyl-1,3,5-triazine (89.6 g, 335 mmol) purchased from Tokyo Chemical Industries.

HRMS (70 eV, EI+): m/z calcd for C27H16ClN3O: 433.0982, found: 433.

Elemental Analysis: C, 74%; H, 4%

### Synthesis Example 44: Synthesis of Intermediate I-38

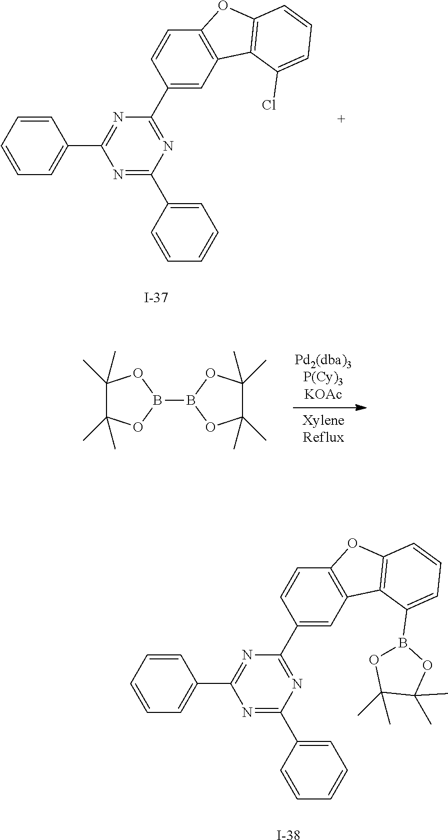

Intermediate I-38 (76.7 g, 72%) was obtained using Intermediate I-37 (88 g, 203 mmol) in the same manner as in Synthesis Example 6.

HRMS (70 eV, EI+): m/z calcd for C33H28BN3O3: 525.2224, found: 525.

Elemental Analysis: C, 75%; H, 5%

### Synthesis Example 45: Synthesis of Intermediate I-39

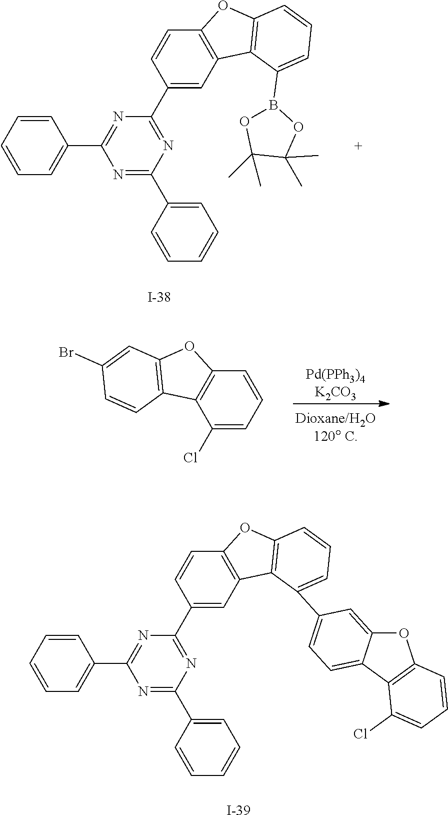

Intermediate I-39 (65.2 g, 76%) was obtained using Intermediate I-38 (75 g, 143 mmol) and 7-bromo-1-chlorodibenzofuran (40.2 g, 143 mmol) in the same manner as in Synthesis Example 7.

HRMS (70 eV, EI+): m/z calcd for C39H22ClN3O2: 599.1401, found: 599.

Elemental Analysis: C, 78%; H, 4%

### Synthesis Example 46: Synthesis of Intermediate I-40

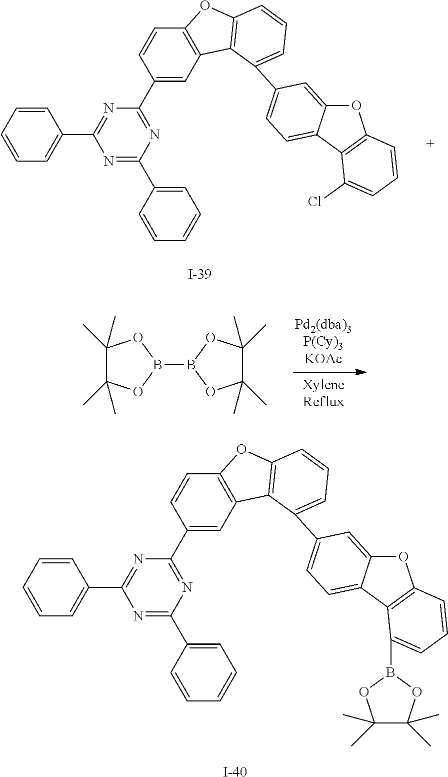

Intermediate I-40 (43.6 g, 60%) was obtained using Intermediate I-39 (63 g, 105 mmol) in the same manner as in Synthesis Example 6.

HRMS (70 eV, EI+): m/z calcd for C45H34BN3O4: 691.2642, found: 691.

Elemental Analysis: C, 78%; H, 5%

### Synthesis Example 41: Synthesis of Compound 18

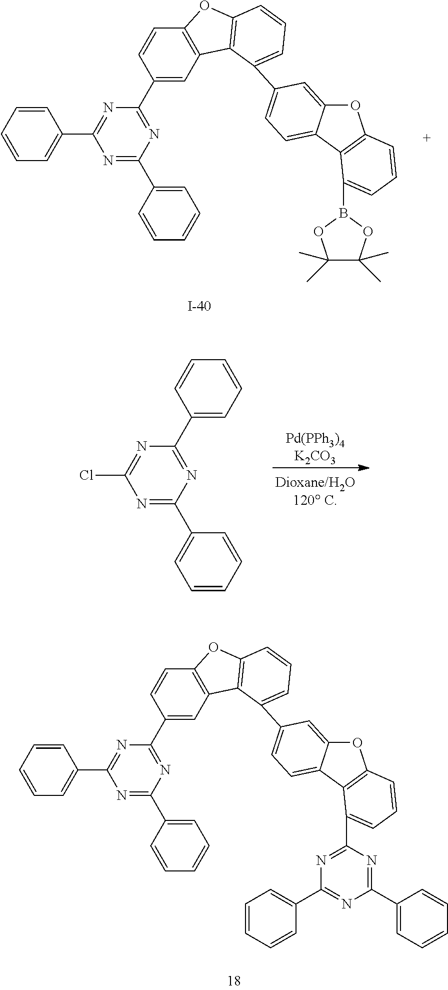

Compound 18 (17.7 g, 77%) was obtained in the same manner as Synthesis Example 7 by using Intermediate I-40 (20 g, 28.9 mmol) and 2-chloro-4,6-diphenyl-1,3,5-triazine (9.28 g, 34.7 mmol) purchased from Tokyo Chemical Industries.

HRMS (70 eV, EI+): m/z calcd for C54H32N6O2: 796.2587, found: 796.

Elemental Analysis: C, 81%; H, 4%

### Synthesis Example 42: Synthesis of Intermediate I-41

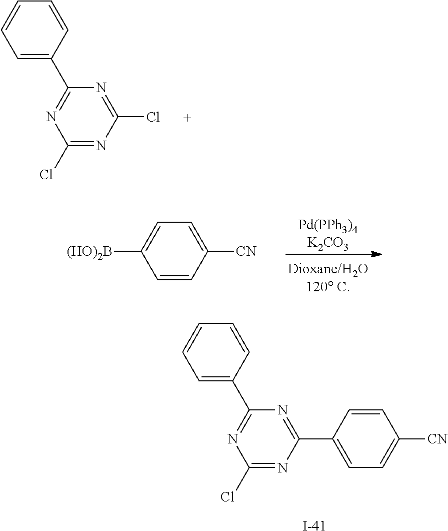

Intermediate I-41 (33.6 g, 52%) was obtained using 2,4-dichloro-6-phenyl-1,3,5-triazine (100 g, 442 mmol) and (4-cyanophenyl) boronic acid (32.5 g, 221 mmol) in the same manner as in Synthesis Example 7.

HRMS (70 eV, EI+): m/z calcd for C16H9ClN4: 292.0516, found: 292.

Elemental Analysis: C, 66%; H, 3%

### Synthesis Example 43: Synthesis of Compound 61

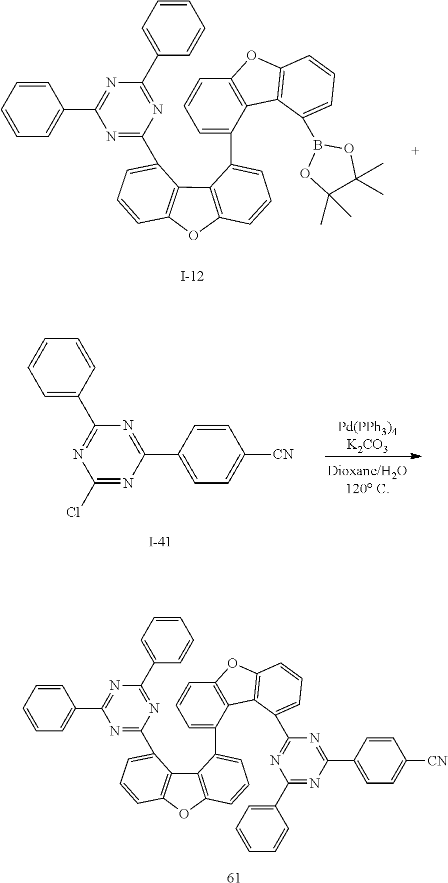

Compound 61 (15.7 g, 66%) was obtained using Intermediate I-12 (20 g, 28.9 mmol) and Intermediate I-41 (10.2 g, 34.7 mmol) in the same manner as in Synthesis Example 7.

HRMS (70 eV, EI+): m/z calcd for C55H31N7O2: 821.2539, found: 821.

Elemental Analysis: C, 80%; H, 4%

### Synthesis Example 44: Synthesis of Intermediate I-42

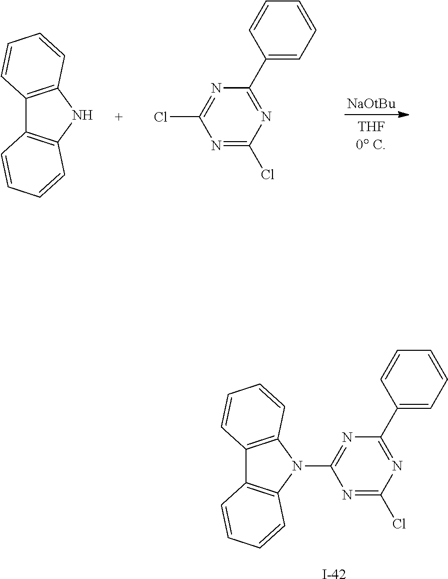

After dissolving 9H-carbazole (100 g, 598 mmol) and 2,4-dichloro-6-phenyl-1,3,5-triazine (203 g, 897 mmol) purchased from Tokyo Chemical Industries in a nitrogen environment in 1 L of tetrahydrofuran (THF), sodium tert-butoxide (63.2 g, 658 mmol) was slowly added at 0° C. and stirred. After 12 hours, water was added to the reaction solution and the mixture was filtered. The residue thus obtained was separated and purified by flash column chromatography to obtain Intermediate I-42 (194 g, 91%).

HRMS (70 eV, EI+): m/z calcd for C21H13ClN4: 356.0829, found: 356.

Elemental Analysis: C, 71%; H, 4%

### Synthesis Example 45: Synthesis of Compound 101

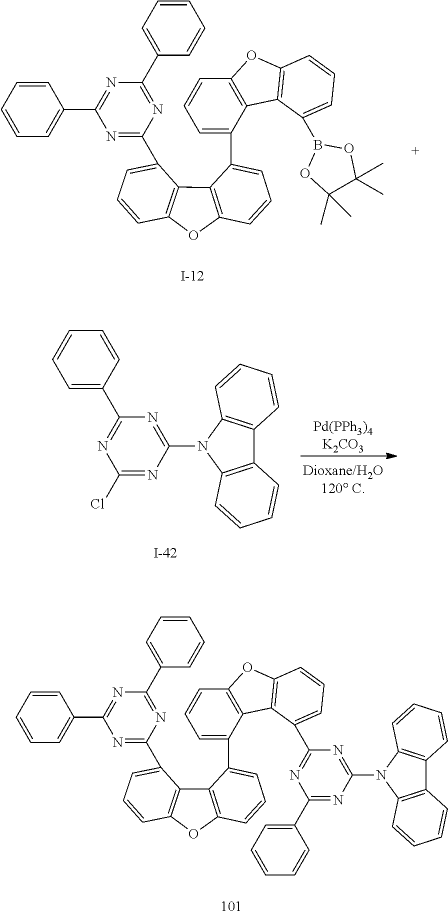

Compound 101 (12.8 g, 50%) was obtained using Intermediate I-12 (20 g, 28.9 mmol) and Intermediate I-41 (12.4 g, 34.7 mmol) in the same manner as in Synthesis Example 7.

HRMS (70 eV, EI+): m/z calcd for C60H35N7O2: 885.2852, found: 885.

Elemental Analysis: C, 81%; H, 4%

### Synthesis Example 46: Synthesis of Compound R-1

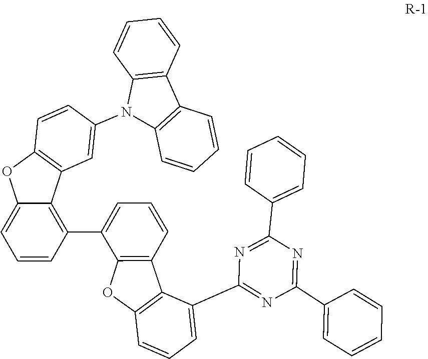 [SMILES: c1ccc(-c2nc(-c3ccccc3)nc(-c3cccc4oc5c(-c6cccc7oc8ccc(-n9c%10ccccc%10c%10ccccc%109)cc8c67)cccc5c34)n2)cc1]

Compound R-1 was synthesized with reference to Korean Patent KR2022-0013909.

HRMS (70 eV, EI+): m/z calcd for C51H30N4O2: 730.2369, found: 730.

Elemental Analysis: C, 84%; H, 4%

### Synthesis Example 47: Synthesis of Compound R-2

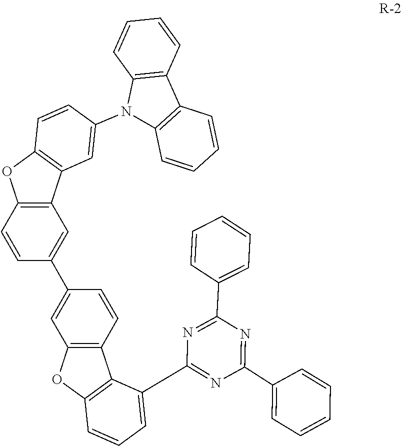 [SMILES: c1ccc(-c2nc(-c3ccccc3)nc(-c3cccc4oc5cc(-c6ccc7oc8ccc(-n9c%10ccccc%10c%10ccccc%109)cc8c7c6)ccc5c34)n2)cc1]

Comparative compound R-2 was synthesized with reference to Korean Patent KR2022-0013909.

HRMS (70 eV, EI+): m/z calcd for C51H30N4O2: 730.2369, found: 730.

Elemental Analysis: C, 84%; H, 4%

### Synthesis Example 48: Synthesis of Compound R-3

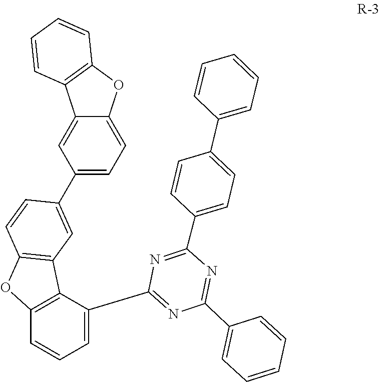 [SMILES: c1ccc(-c2ccc(-c3nc(-c4ccccc4)nc(-c4cccc5oc6ccc(-c7ccc8oc9ccccc9c8c7)cc6c45)n3)cc2)cc1]

Compound R-3 was synthesized with reference to Korean Patent KR2018-0068869.

HRMS (70 eV, EI+): m/z calcd for C45H27N3O2: 641.2103, found: 641.

Elemental Analysis: C, 84%; H, 4%

### Synthesis Example 49: Synthesis of Compound B-40

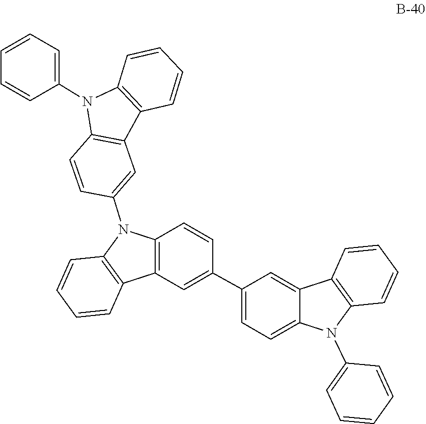 [SMILES: c1ccc(-n2c3ccccc3c3cc(-c4ccc5c(c4)c4ccccc4n5-c4ccc5c(c4)c4ccccc4n5-c4ccccc4)ccc32)cc1]

Compound B-40 was obtained by purchasing from Gemchem (http://www.ytgemchem.com).

HRMS (70 eV, EI+): m/z calcd for C48H31N3: 649.2518, found: 649.

Elemental Analysis: C, 89%; H, 5%

### Synthesis Example 50: Synthesis of Compound B-136

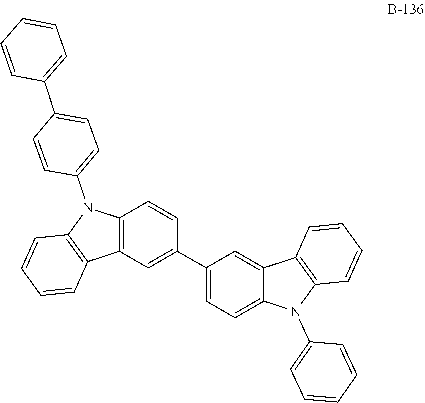 [SMILES: c1ccc(-c2ccc(-n3c4ccccc4c4cc(-c5ccc6c(c5)c5ccccc5n6-c5ccccc5)ccc43)cc2)cc1]

Compound B-136 was obtained by purchasing from Gemchem.

HRMS (70 eV, EI+): m/z calcd for C42H28N2: 560.2252, found: 560.

Elemental Analysis: C, 90%; H, 5%

### Synthesis Example 51: Synthesis of Compound C-4

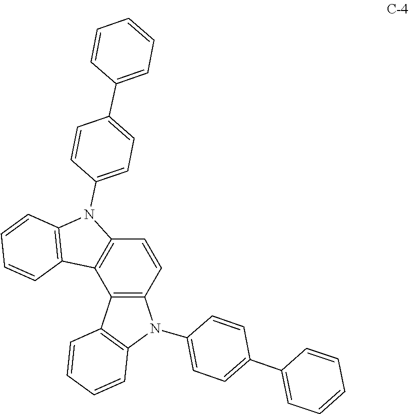 [SMILES: c1ccc(-c2ccc(-n3c4ccccc4c4c5c6ccccc6n(-c6ccc(-c7ccccc7)cc6)c5ccc43)cc2)cc1]

Compound C-4 was obtained by purchasing from Gemchem.

HRMS (70 eV, EI+): m/z calcd for C42H28N2: 560.2252, found: 560.

Elemental Analysis: C, 90%; H, 5%

### Example 1

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 ∈-thick hole injection layer, and Compound A was deposited on the hole injection layer to a thickness of 1,350 Å to form a hole transport layer. Compound B was deposited on the hole transport layer to a thickness of 350 Å to form a hole transport auxiliary layer, and on the hole transport auxiliary layer, Compound 1 (synthesized in Synthesis Example 13) was used as a host and doped with 7 wt % of PhGD as a dopant to form a 400 Å-thick light emitting layer by vacuum deposition. The ratios thereof are described separately in the following examples and comparative examples. Subsequently, Compound C was deposited on the light emitting layer to a thickness of 50 Å to form an electron transport auxiliary layer, and Compound D and LiQ were simultaneously vacuum-deposited at a weight ratio of 1:1 to form a 300 Å-thick electron transport layer. An organic light emitting diode was manufactured by sequentially vacuum depositing 15 Å of LiQ and 1,200 Å of Al on the electron transport layer to form a cathode.

ITO/Compound A (3% NDP-9 doping, 100 Å)/Compound A (1350 Å)/Compound B (350 Å)/EML [Compound 1 (93 wt %): PhGD (7 wt %)] (400 Å)/Compound C (50 Å)/Compound D: LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-Dibenzofuranyl)phenyl]-N-[4-(9-phenyl-9H-fluoren-9-yl)phenyl][1,1′-biphenyl]-4-amine
Compound C: 2,4-Diphenyl-6-(4′,5′,6′-triphenyl[1,1′:2′,1″:3″,1′″:3′″,1′″-quinquephenyl]-3″-yl)-1,3,5-triazine
Compound D: 2-(1,1′-Biphenyl-4-yl)-4-(9,9-diphenylfluoren-4-yl)-6-phenyl-1,3,5-triazine

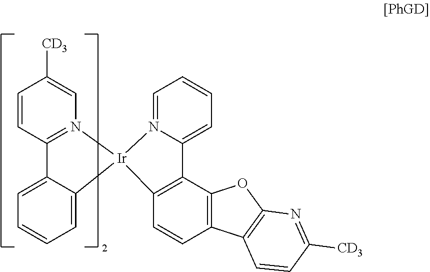

### Examples 2 to 9 and Comparative Examples 1 to 3

Organic light emitting diodes were manufactured in the same manner as Example 1, except that the compositions were changed to the one shown in Table 1.

### Example 10

A glass substrate coated with a thin film of ITO (indium tin oxide) was ultrasonically cleaned with distilled water. After washing with the distilled water, the glass substrate was washed ultrasonically with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1,350 Å to form a hole transport layer. Compound E was deposited on the hole transport layer to a thickness of 350 Å to form a hole transport auxiliary layer. On the hole transport auxiliary layer, Compound 1 synthesized in Synthesis Example 13 and Compound B-136 synthesized in Synthesis Example 50 were simultaneously used as hosts, and PhGD was doped at 10 wt % as a dopant to form a 400 Å-thick light emitting layer by vacuum deposition. Herein, Compound 1 and Compound B-136 were used in a weight ratio of 3:7. Then, Compound F was deposited on the light emitting layer to a thickness of 50 Å to form an electron transport auxiliary layer, and Compound G and LiQ were simultaneously vacuum-deposited in a weight ratio of 1:1 to form a 300 Å-thick electron transport layer. An organic light emitting diode was manufactured by sequentially vacuum depositing 15 Å of LiQ and 1,200 Å of Al on the electron transport layer to form a cathode.

ITO/Compound A (3% NDP-9 doping, 100 Å)/Compound A (1,350 Å)/Compound E (350 Å)/EML [Compound 1: Compound B-136: PhGD=27:63:10 wt %)](400 Å)/Compound F (50 Å)/Compound G: LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound E: N,N-bis(9,9-dimethyl-9H-fluoren-4-yl)-9,9-spirobi (fluorene)-2-amine
Compound F: 2-[3′-(9,9-Dimethyl-9H-fluoren-2-yl) [1,1′-biphenyl]-3-yl]-4,6-diphenyl-1,3,5-triazine
Compound G: 2-[4-[4-(4′-Cyano-1,1′-biphenyl-4-yl)-1-naphthyl]phenyl]-4,6-diphenyl-1,3,5-triazine

### Example 11 to 22 and Comparative Example 4 to 6

Organic light emitting diodes were manufactured in the same manner as Example 10, except that the compositions were changed to the one shown in Table 2.

### Evaluation

The driving voltage, luminous efficiency, and life-span characteristics of the organic light emitting diodes according to Examples 1 to 22 and Comparative Examples 1 to 6 were evaluated.

The measurement method was as follows, and the results are as shown in Tables 1 and 2.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Current efficiency (cd/A) at the same current density (10 mA/cm²) were calculated by using the luminance and current density from (1) and (2) above and voltage.

The luminous efficiency values of Examples 1 to 9 and Comparative Examples 1 to 3 were calculated as relative values based on Comparative Example 1 and are shown in Table 1.

The luminous efficiency values of Examples 10 to 22 and Comparative Examples 4 to 6 were calculated as relative values based on Comparative Example 4 and are shown in Table 2.

### (4) Measurement of Life-Span

Time when each current efficiency (cd/A) was reduced to 97%, while maintaining luminance (cd/m2) at 24,000 cd/m2, was measured as a life-span.

The life-span values of Examples 1 to 9 and Comparative Examples 1 to 3 were calculated as relative values based on Comparative Example 1 and are shown in Table 1.

The life-span values of Examples 10 to 22 and Comparative Examples 4 to 6 were calculated as relative values based on Comparative Example 4 and are shown in Table 2.

### (5) Measurement of Driving Voltage

A current-voltage meter (Keithley 2400) was used to measure a driving voltage of each device at 15 mA/cm².

The driving voltages of Examples 1 to 9 and Comparative Examples 1 to 3 were calculated as relative values based on Comparative Example 1 and are shown in Table 1.

The driving voltages of Examples 10 to 22 and Comparative Examples 4 to 6 were calculated as relative values based on Comparative Example 4 and are shown in Table 2.

TABLE 1

| | | | Color | Effi- | Life- |
|---|---|---|---|---|---|
| | Compound | Driving | (EL | ciency | span |
| No. | (wt %) | voltage (%) | color) | (%) | (%) |
| Example 1 | 1 (7) | 86 | Green | 145 | 193 |
| Example 2 | 2 (7) | 85 | Green | 149 | 233 |
| Example 3 | 9 (7) | 88 | Green | 133 | 260 |
| Example 4 | 10 (7) | 88 | Green | 130 | 233 |
| Example 5 | 11 (7) | 89 | Green | 122 | 267 |
| Example 6 | 17 (7) | 90 | Green | 112 | 200 |
| Example 7 | 18 (7) | 88 | Green | 109 | 273 |
| Example 8 | 61 (7) | 92 | Green | 102 | 320 |
| Example 9 | 101 (7) | 89 | Green | 118 | 367 |
| Comparative | R-1 (7) | 100 | Green | 100 | 100 |
| Example 1 | | | | | |
| Comparative | R-2 (7) | 102 | Green | 89 | 133 |
| Example 2 | | | | | |
| Comparative | R-3 (7) | 95 | Green | 84 | 167 |
| Example 3 | | | | | |

TABLE 2

| | | | Color | Effi- | Life- |
|---|---|---|---|---|---|
| | Compound | Driving | (EL | ciency | span |
| No. | (weight ratio) | voltage (%) | color) | (%) | (%) |
| Example 10 | 1/B-136 (3:7) | 86 | Green | 156 | 140 |
| Example 11 | 2/B-136 (3:7) | 85 | Green | 161 | 149 |
| Example 12 | 2/B-136 (4:6) | 83 | Green | 167 | 137 |
| Example 13 | 2/B-136 (5:5) | 82 | Green | 171 | 129 |
| Example 14 | 9/B-136 (3:7) | 88 | Green | 146 | 171 |
| Example 15 | 10/B-136 (3:7) | 87 | Green | 139 | 157 |
| Example 16 | 11/B-136 (3:7) | 88 | Green | 153 | 171 |
| Example 17 | 17/B-136 (3:7) | 86 | Green | 134 | 143 |
| Example 18 | 18/B-136 (3:7) | 86 | Green | 132 | 186 |
| Example 19 | 61/B-136 (3:7) | 89 | Green | 124 | 194 |
| Example 20 | 101/B-136 (3:7) | 87 | Green | 133 | 214 |
| Example 21 | 2/B-40 (4:6) | 85 | Green | 159 | 157 |
| Example 22 | 2/C-4 (4:6) | 80 | Green | 165 | 129 |
| Comparative | R-1/B-136 (4:6) | 100 | Green | 100 | 100 |
| Example 4 | | | | | |
| Comparative | R-2/B-136 (4:6) | 102 | Green | 89 | 114 |
| Example 5 | | | | | |
| Comparative | R-3/B-136 (4:6) | 93 | Green | 84 | 120 |
| Example 6 | | | | | |

Referring to Tables 1 and 2, the luminous efficiency and life-span characteristics of the organic light emitting diodes according to Examples 1 to 22 are significantly improved compared to the organic light emitting diodes according to Comparative Examples 1 to 6.

One or more embodiments may provide a compound for an organic optoelectronic device capable of lowering the driving voltage and realizing a high efficiency and long life-span organic optoelectronic device.

While lowering a driving voltage, it is possible to implement high-efficiency, long life-span organic optoelectronic devices.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 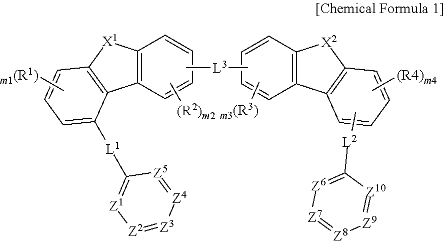
wherein, in Chemical Formula 1,
X¹ and X² are each independently O, S, CR^{a}R^{b}, SiR^{c}R^{d}, or S(O)₂,
Z¹ to Z¹⁰ are each independently N or CR^{e},
at least two of Z¹ to Z⁵ are N,
at least two of Z⁶ to Z¹⁰ are N,
R^{a}, R^{b}, R^{c}, R^{d}, R^{e}, and R¹ to R⁴ are each independently hydrogen, deuterium, a cyano group, a substituted or unsubstituted C1 to C20 alkyl group, a substituted or unsubstituted C6 to C20 aryl group, a substituted or unsubstituted C2 to C20 heterocyclic group, or a combination thereof,
L¹ to L³ are each independently a single bond, a substituted or unsubstituted C6 to C20 arylene group or a substituted or unsubstituted C2 to C30 heterocyclic group,
m1 to m4 are each independently an integer of 1 to 3,
when m1 to m4 are 2 or more, each of R¹ to R⁴ is the same or different, and R¹ to R⁴ are each separately present or adjacent groups are linked to each other to form a substituted or unsubstituted aromatic monocyclic ring, a substituted or unsubstituted aromatic polycyclic ring, a substituted or unsubstituted heteroaromatic monocyclic ring, or a substituted or unsubstituted heteroaromatic polycyclic ring, and
each R^{e} is the same or different, and each R^{e} is separately present or adjacent groups are linked to form a substituted or unsubstituted aromatic monocyclic ring, a substituted or unsubstituted aromatic polycyclic ring, a substituted or unsubstituted heteroaromatic monocyclic ring, or a substituted or unsubstituted heteroaromatic polycyclic ring.

2. The compound for an organic optoelectronic device as claimed in claim 1, wherein:
Chemical Formula 1 is represented by one of Chemical Formula 1-1 to Chemical Formula 1-4: 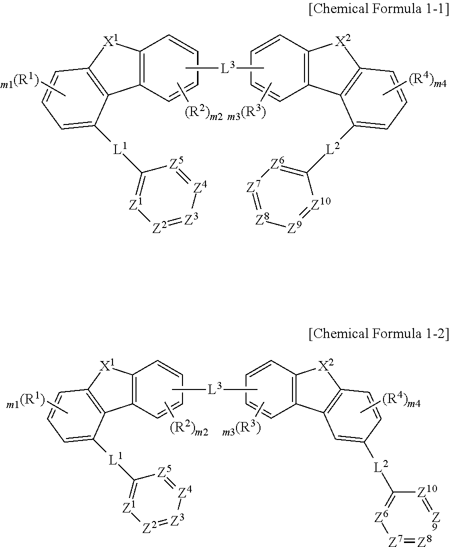 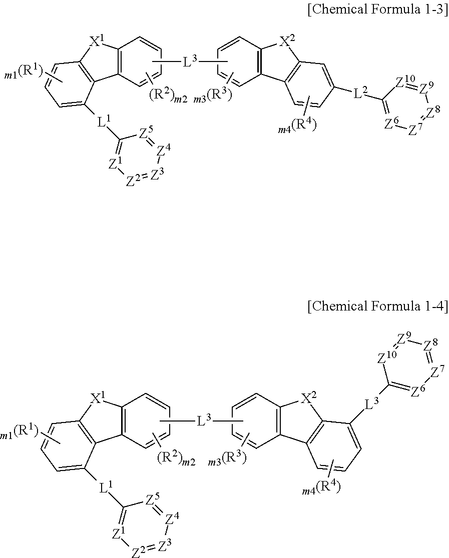
in Chemical Formula 1-1 to Chemical Formula 1-4, X¹ and X², Z¹ to Z¹⁰, R¹ to R⁴, L¹ to L³, and m1 to m4 are defined the same as those of Chemical Formula 1.

3. The compound for an organic optoelectronic device as claimed in claim 1, wherein:
Chemical Formula 1 is represented by one of Chemical Formula 1-5 to Chemical Formula 1-7: 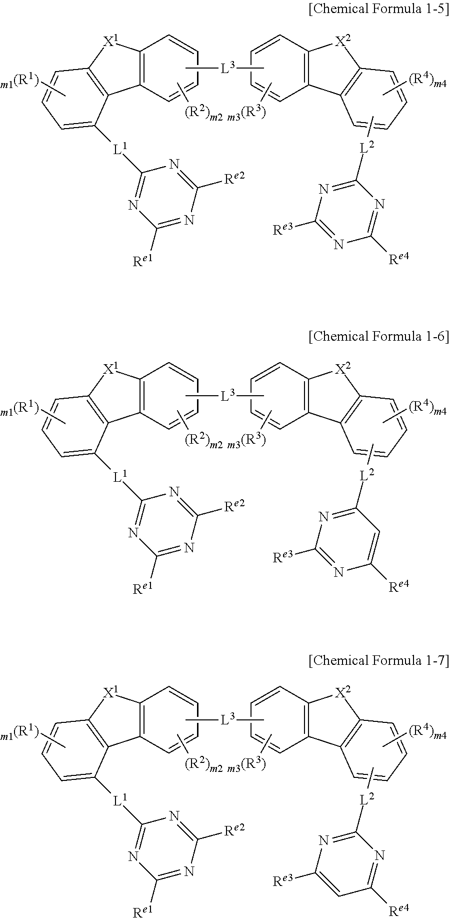
in Chemical Formula 1-5 to Chemical Formula 1-7,
X¹ and X², R¹ to R⁴, L¹ to L³, and m1 to m4 are defined the same as those of Chemical Formula 1, and
R^{e1} to R^{e4} are defined the same as R^{e} of Chemical Formula 1.

4. The compound for an organic optoelectronic device as claimed in claim 1, wherein:
X¹ and X² are each independently O, S, or SiR^{c}R^{d}, and
R^{c} and R^{d} are each independently hydrogen, deuterium, a cyano group, a substituted or unsubstituted C1 to C20 alkyl group, a substituted or unsubstituted C6 to C20 aryl group, or a combination thereof.

5. The compound for an organic optoelectronic device as claimed in claim 1, wherein each of L¹ to L³ is a single bond.

6. The compound for an organic optoelectronic device as claimed in claim 1, the compound being a compound of Group 1: 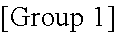 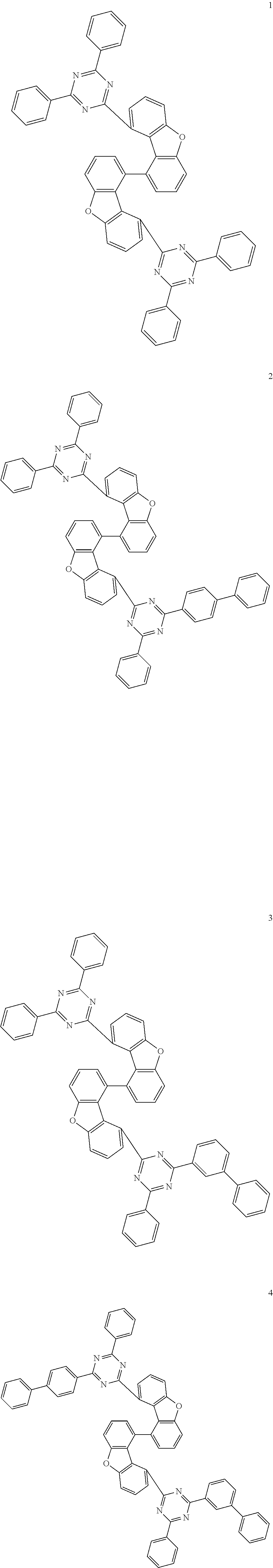 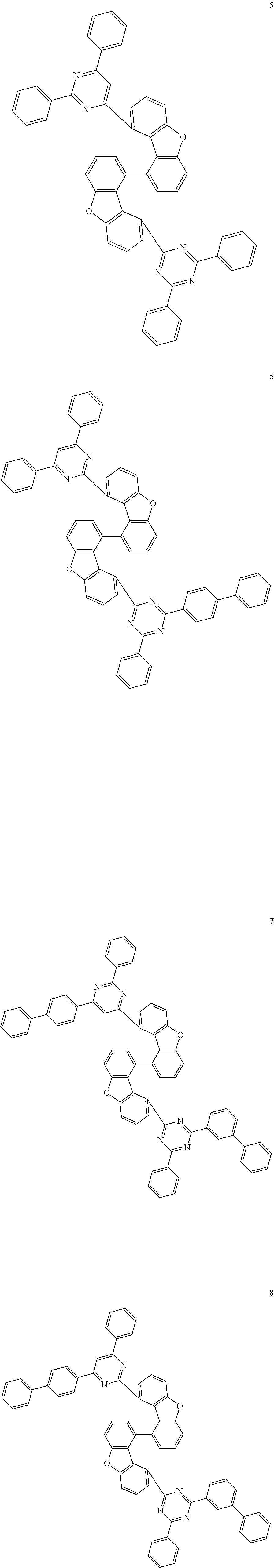 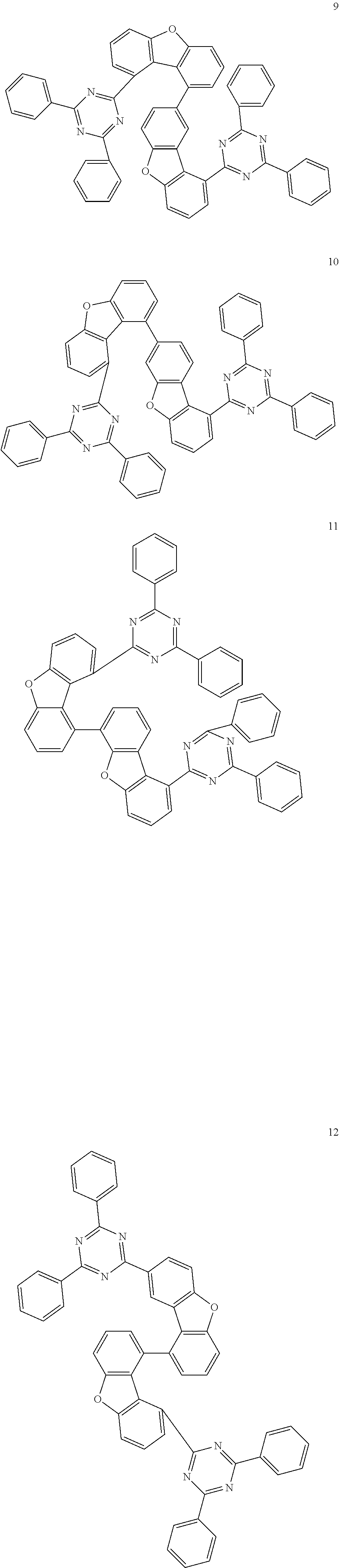 [SMILES: c1ccc(-c2nc(-c3ccccc3)nc(-c3cccc4oc5ccc(-c6cccc7oc8cccc(-c9nc(-c%10ccccc%10)nc(-c%10ccccc%10)n9)c8c67)cc5c34)n2)cc1 ; c1ccc(-c2nc(-c3ccccc3)nc(-c3cccc4oc5cc(-c6cccc7oc8cccc(-c9nc(-c%10ccccc%10)nc(-c%10ccccc%10)n9)c8c67)ccc5c34)n2)cc1 ; c1ccc(-c2nc(-c3ccccc3)nc(-c3cccc4oc5c(-c6cccc7oc8cccc(-c9nc(-c%10ccccc%10)nc(-c%10ccccc%10)n9)c8c67)cccc5c34)n2)cc1 ; c1ccc(-c2nc(-c3ccccc3)nc(-c3ccc4oc5cccc(-c6cccc7oc8cccc(-c9nc(-c%10ccccc%10)nc(-c%10ccccc%10)n9)c8c67)c5c4c3)n2)cc1] 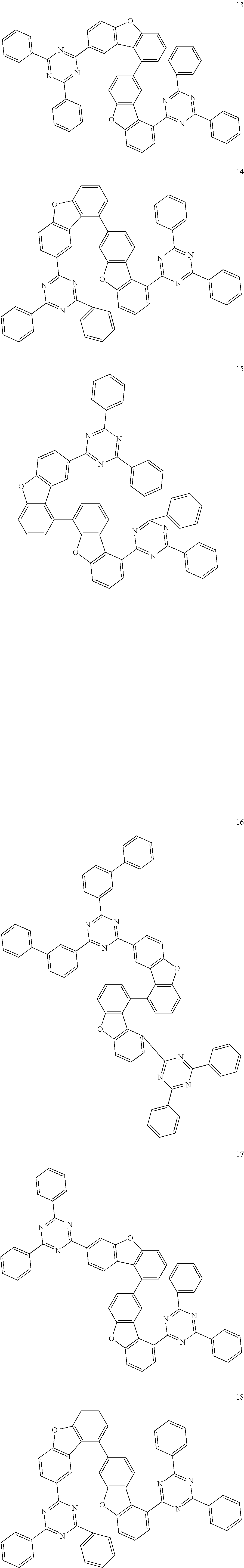 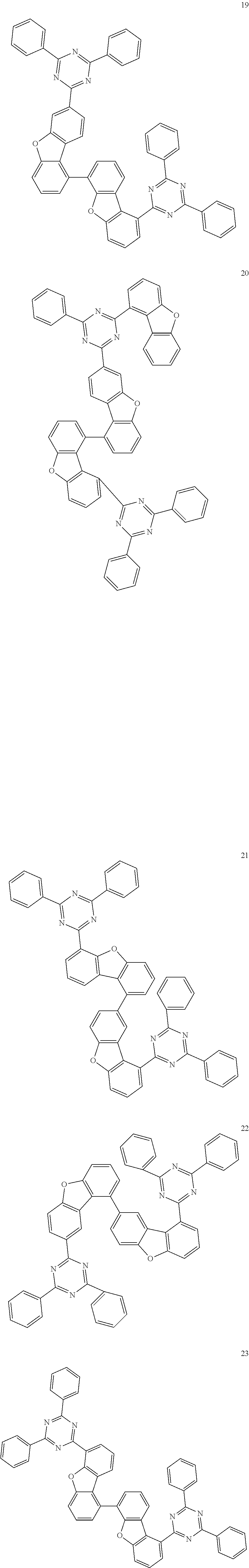 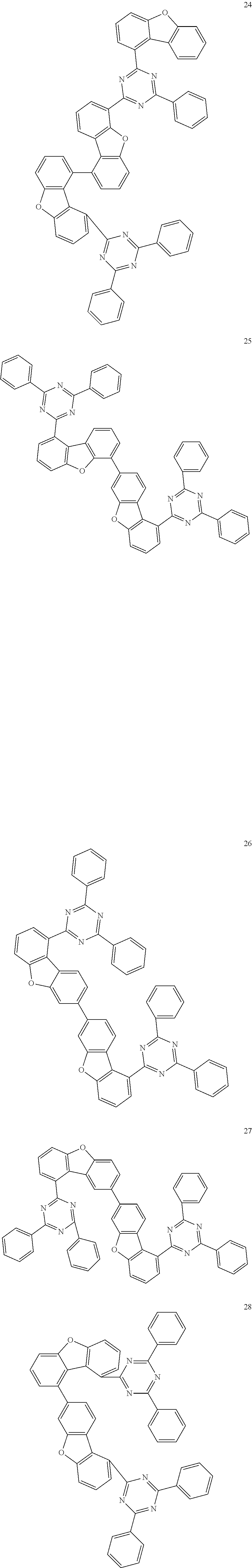 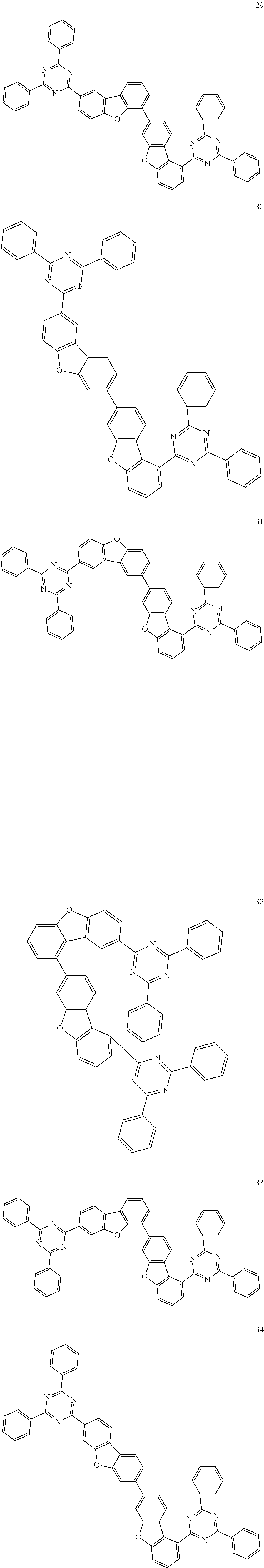 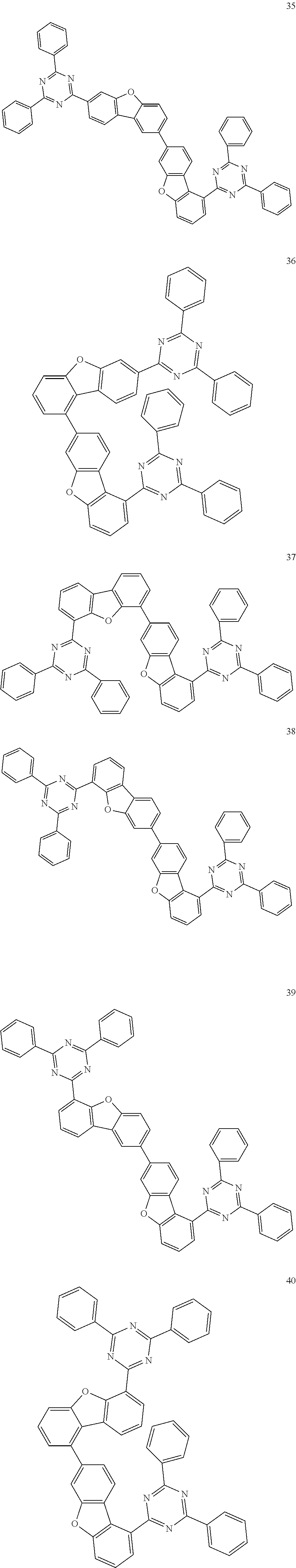 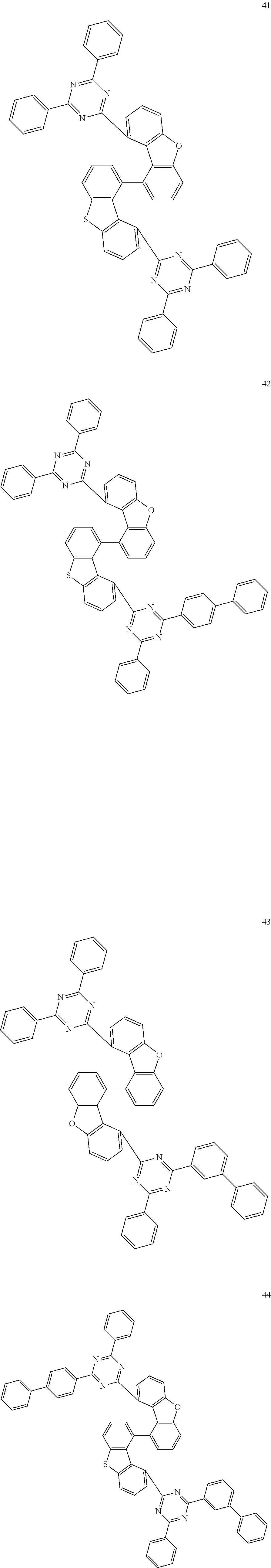 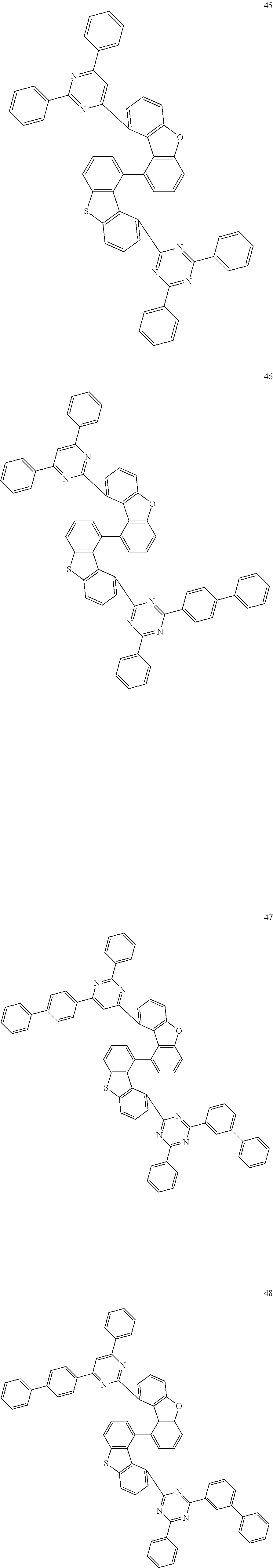 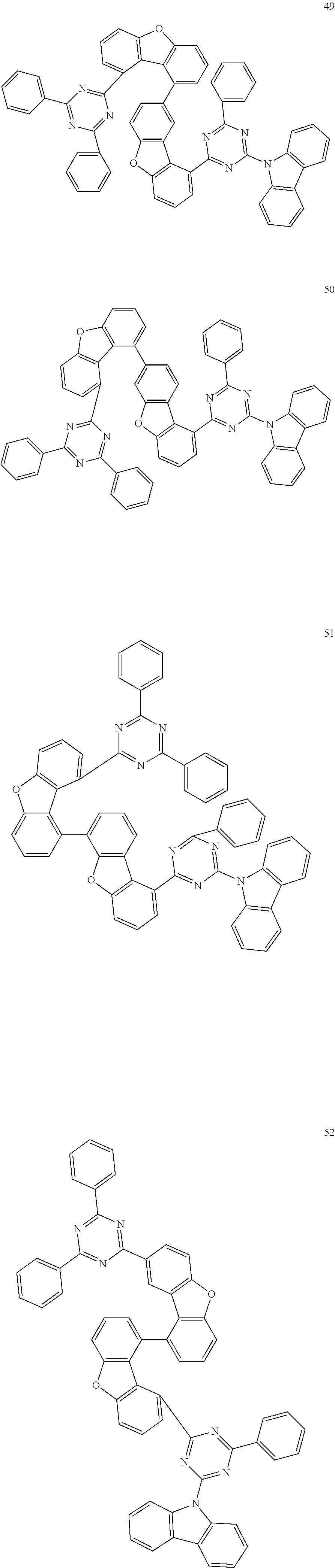 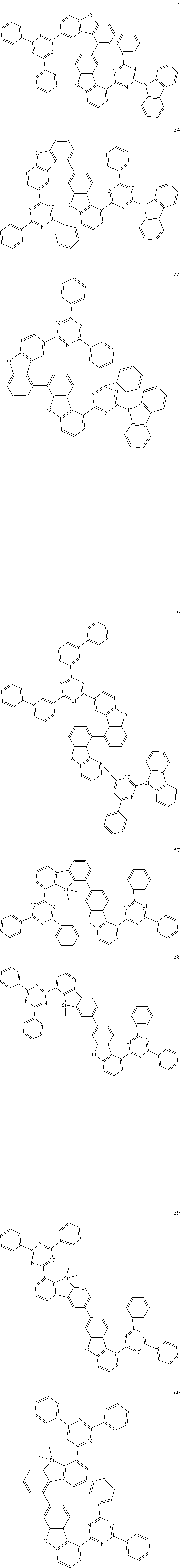 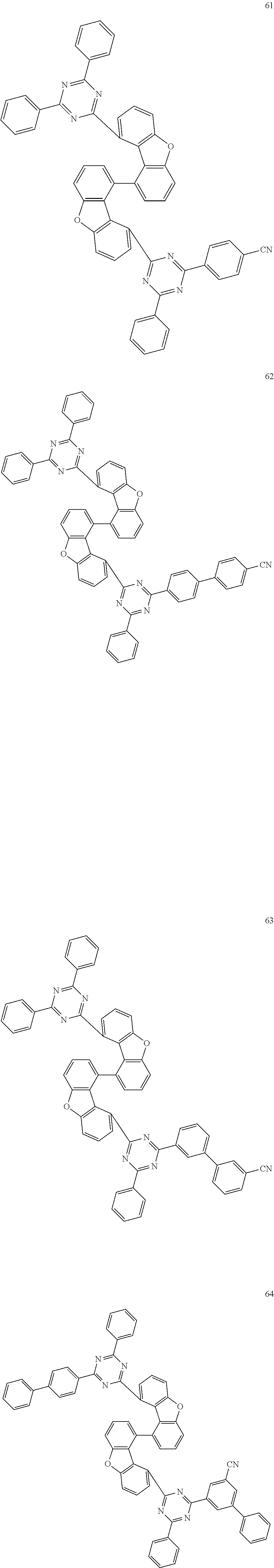 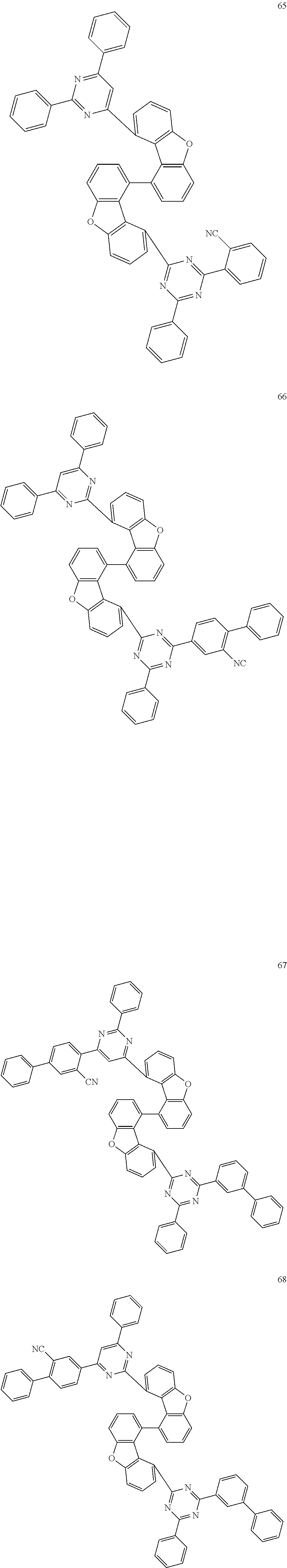 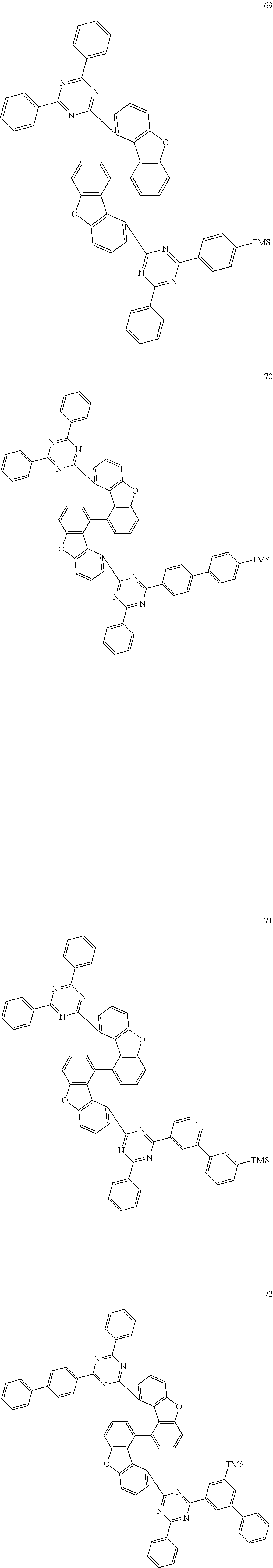 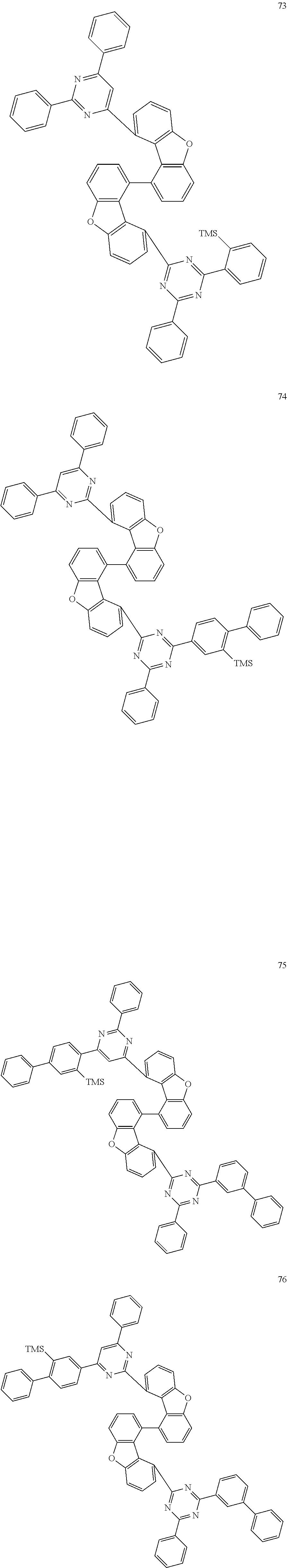 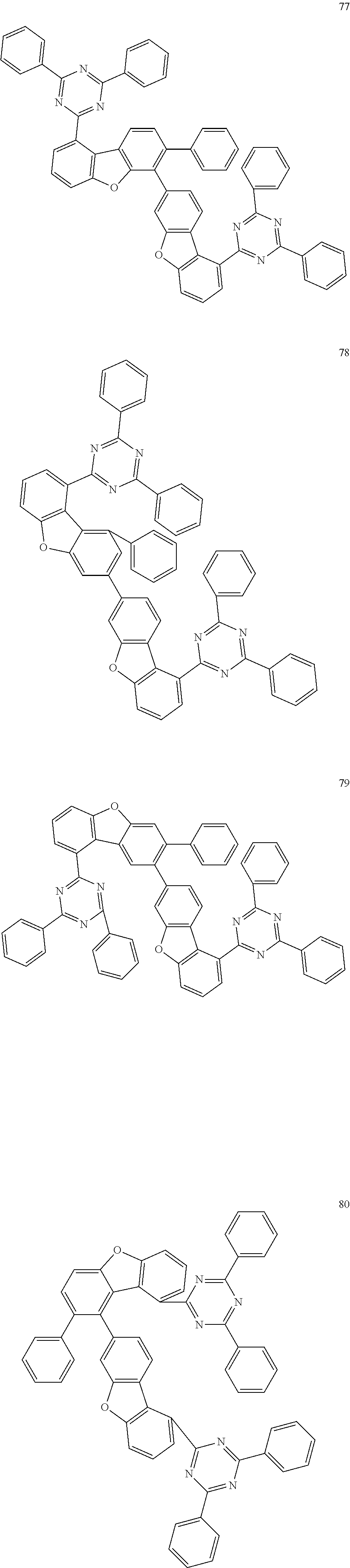 [SMILES: c1ccc(-c2nc(-c3ccccc3)nc(-c3cccc4oc5cc(-c6c(-c7ccccc7)ccc7c6oc6cccc(-c8nc(-c9ccccc9)nc(-c9ccccc9)n8)c67)ccc5c34)n2)cc1 ; c1ccc(-c2nc(-c3ccccc3)nc(-c3cccc4oc5cc(-c6cc(-c7ccccc7)c7c(c6)oc6cccc(-c8nc(-c9ccccc9)nc(-c9ccccc9)n8)c67)ccc5c34)n2)cc1 ; c1ccc(-c2nc(-c3ccccc3)nc(-c3cccc4oc5cc(-c6cc7c(cc6-c6ccccc6)oc6cccc(-c8nc(-c9ccccc9)nc(-c9ccccc9)n8)c67)ccc5c34)n2)cc1 ; c1ccc(-c2nc(-c3ccccc3)nc(-c3cccc4oc5cc(-c6c(-c7ccccc7)ccc7oc8cccc(-c9nc(-c%10ccccc%10)nc(-c%10ccccc%10)n9)c8c67)ccc5c34)n2)cc1] 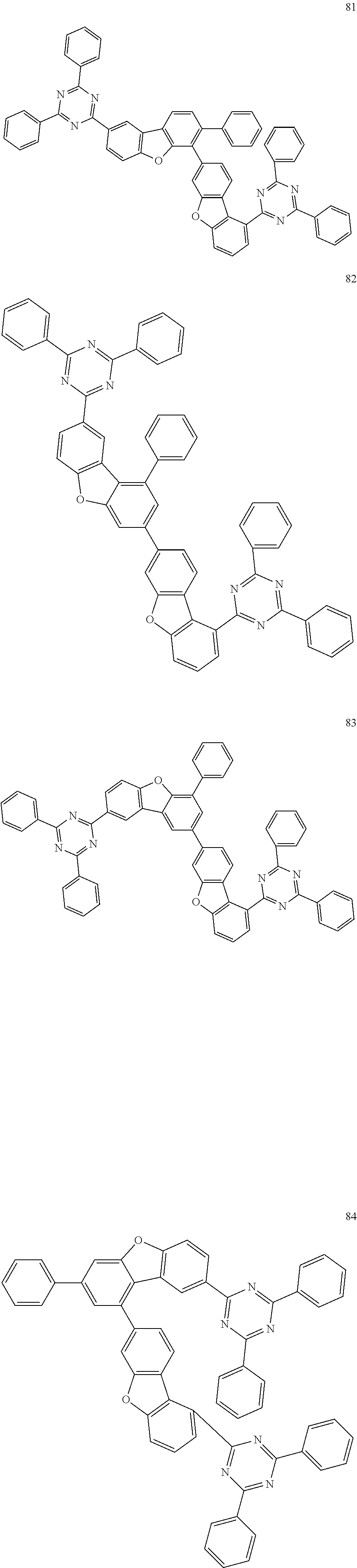 [SMILES: c1ccc(-c2nc(-c3ccccc3)nc(-c3ccc4oc5c(-c6ccc7c(c6)oc6cccc(-c8nc(-c9ccccc9)nc(-c9ccccc9)n8)c67)c(-c6ccccc6)ccc5c4c3)n2)cc1 ; c1ccc(-c2nc(-c3ccccc3)nc(-c3ccc4oc5cc(-c6ccc7c(c6)oc6cccc(-c8nc(-c9ccccc9)nc(-c9ccccc9)n8)c67)cc(-c6ccccc6)c5c4c3)n2)cc1 ; c1ccc(-c2nc(-c3ccccc3)nc(-c3ccc4oc5c(-c6ccccc6)cc(-c6ccc7c(c6)oc6cccc(-c8nc(-c9ccccc9)nc(-c9ccccc9)n8)c67)cc5c4c3)n2)cc1 ; c1ccc(-c2cc(-c3ccc4c(c3)oc3cccc(-c5nc(-c6ccccc6)nc(-c6ccccc6)n5)c34)c3c(c2)oc2ccc(-c4nc(-c5ccccc5)nc(-c5ccccc5)n4)cc23)cc1] 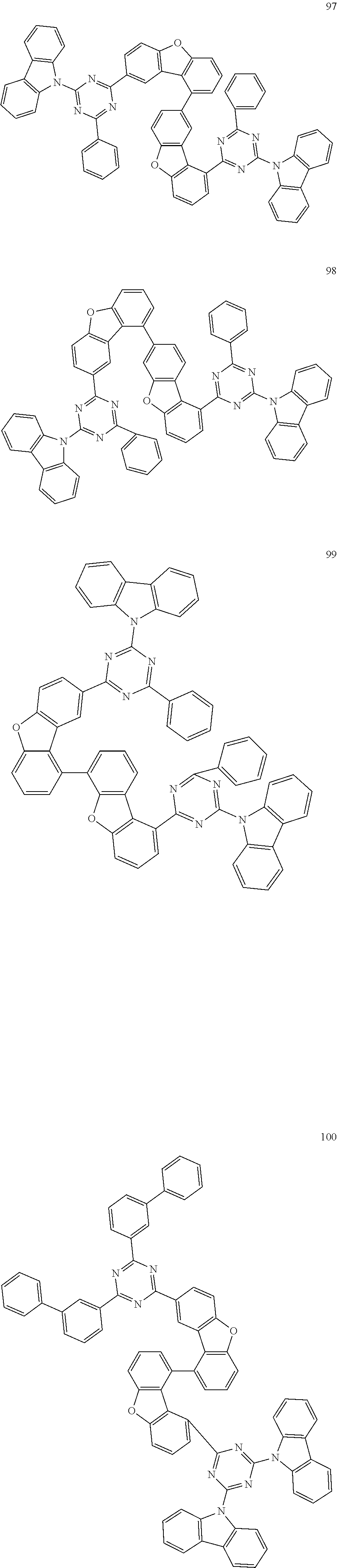 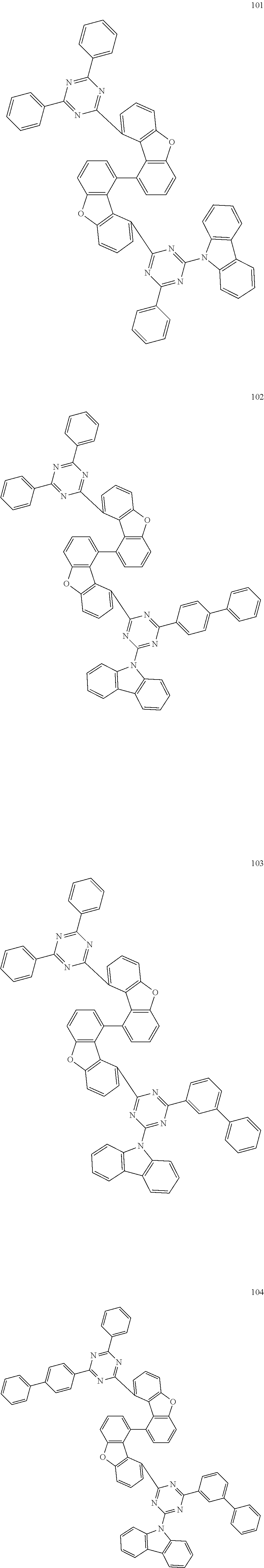
wherein TMS is —Si(CH₃)₃.

7. The compound for an organic optoelectronic device as claimed in claim 6, the compound being a compound of Group 1-1:

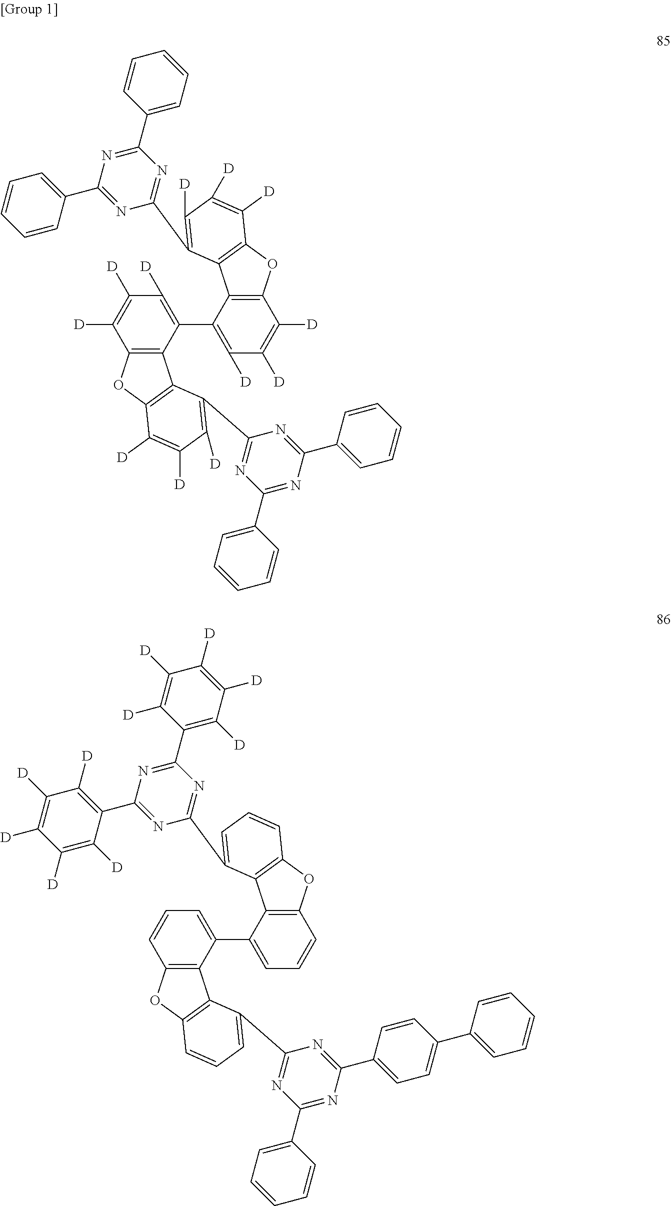 [SMILES: [2H]c1c([2H])c(-c2nc(-c3ccccc3)nc(-c3ccccc3)n2)c2c(oc3c([2H])c([2H])c([2H])c(-c4c([2H])c([2H])c([2H])c5oc6c([2H])c([2H])c([2H])c(-c7nc(-c8ccccc8)nc(-c8ccccc8)n7)c6c45)c32)c1[2H] ; [2H]c1c([2H])c([2H])c(-c2nc(-c3c([2H])c([2H])c([2H])c([2H])c3[2H])nc(-c3cccc4oc5cccc(-c6cccc7oc8cccc(-c9nc(-c%10ccccc%10)nc(-c%10ccc(-c%11ccccc%11)cc%10)n9)c8c67)c5c34)n2)c([2H])c1[2H]]

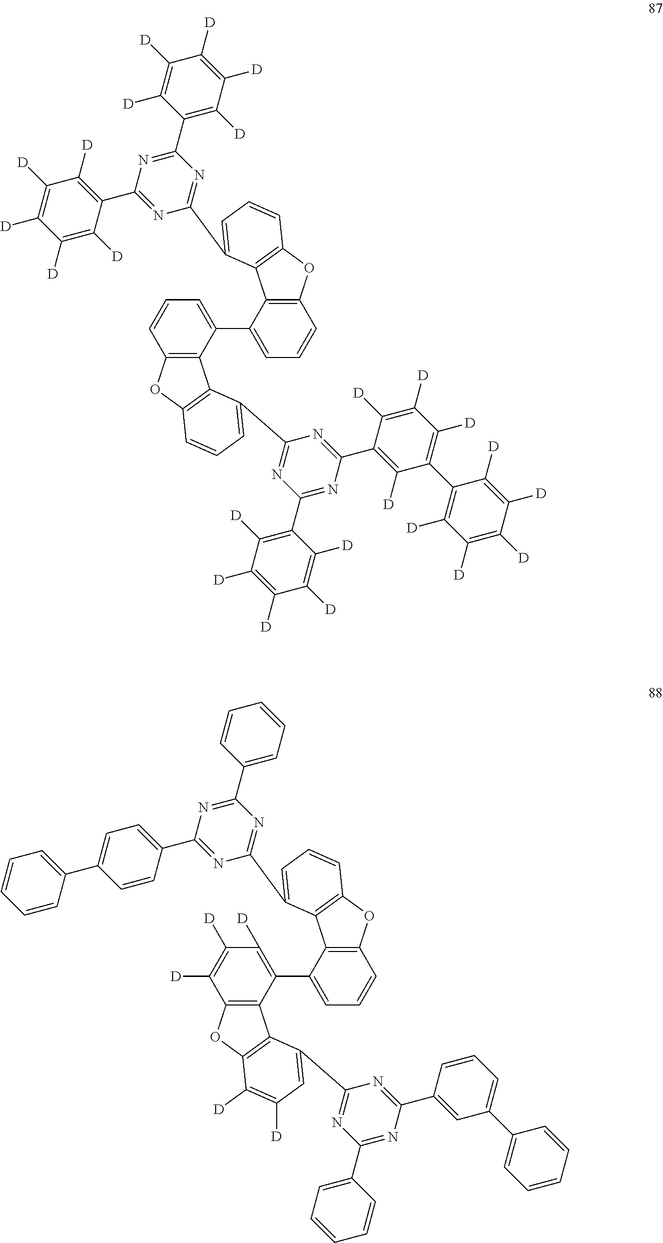 [SMILES: [2H]c1c([2H])c([2H])c(-c2nc(-c3c([2H])c([2H])c([2H])c([2H])c3[2H])nc(-c3cccc4oc5cccc(-c6cccc7oc8cccc(-c9nc(-c%10c([2H])c([2H])c([2H])c([2H])c%10[2H])nc(-c%10c([2H])c([2H])c([2H])c(-c%11c([2H])c([2H])c([2H])c([2H])c%11[2H])c%10[2H])n9)c8c67)c5c34)n2)c([2H])c1[2H] ; [2H]c1cc(-c2nc(-c3ccccc3)nc(-c3cccc(-c4ccccc4)c3)n2)c2c(oc3c([2H])c([2H])c([2H])c(-c4cccc5oc6cccc(-c7nc(-c8ccccc8)nc(-c8ccc(-c9ccccc9)cc8)n7)c6c45)c32)c1[2H]]

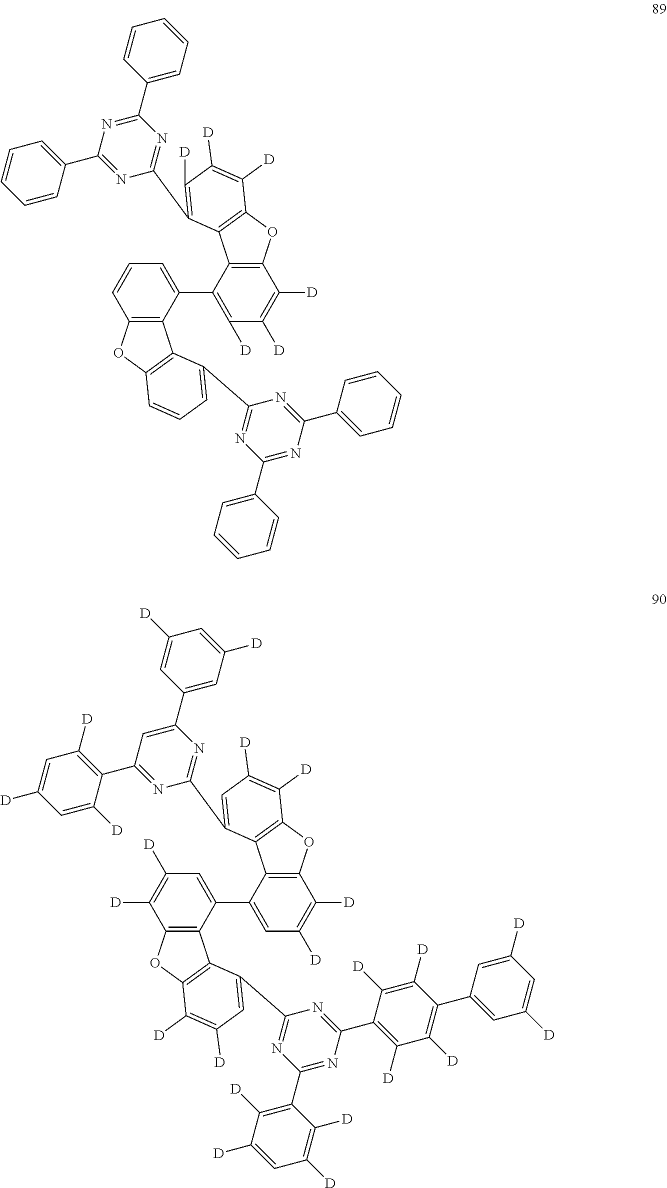 [SMILES: [2H]c1c([2H])c(-c2nc(-c3ccccc3)nc(-c3ccccc3)n2)c2c(oc3c([2H])c([2H])c([2H])c(-c4cccc5oc6cccc(-c7nc(-c8ccccc8)nc(-c8ccccc8)n7)c6c45)c32)c1[2H] ; [2H]c1cc([2H])cc(-c2cc(-c3c([2H])cc([2H])cc3[2H])nc(-c3cc([2H])c([2H])c4oc5c([2H])c([2H])cc(-c6cc([2H])c([2H])c7oc8c([2H])c([2H])cc(-c9nc(-c%10c([2H])c([2H])cc([2H])c%10[2H])nc(-c%10c([2H])c([2H])c(-c%11cc([2H])cc([2H])c%11)c([2H])c%10[2H])n9)c8c67)c5c34)n2)c1]

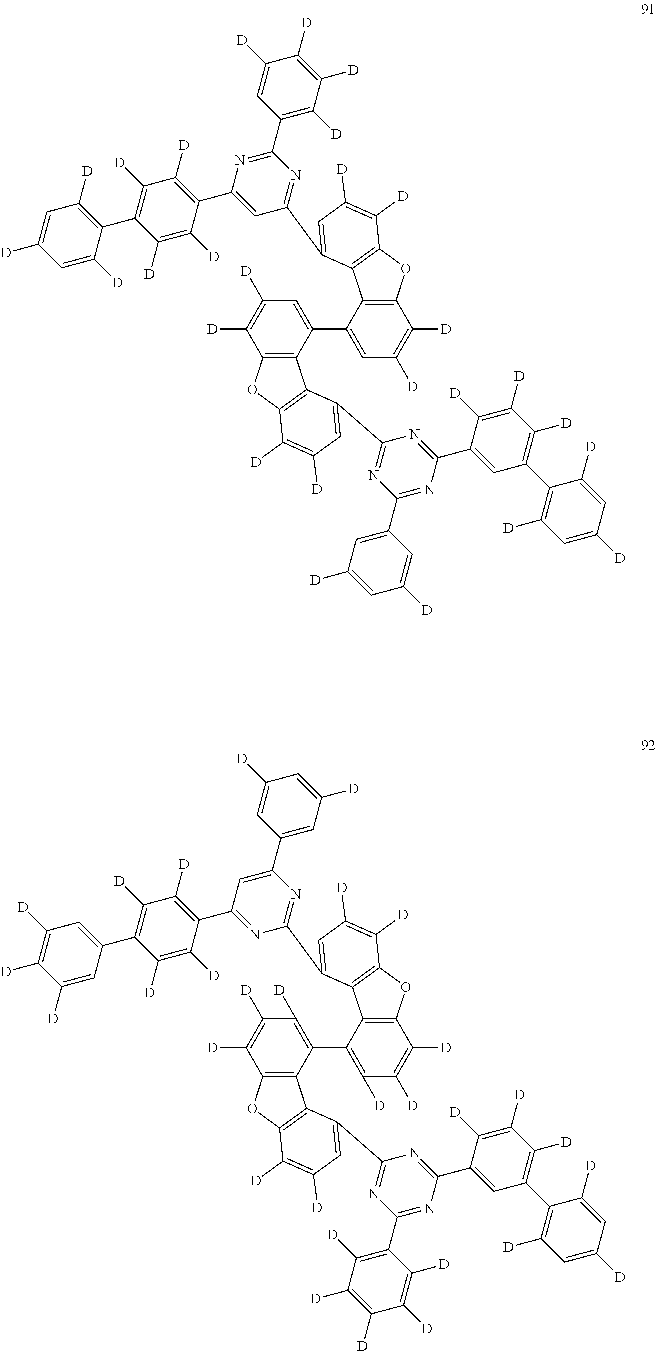

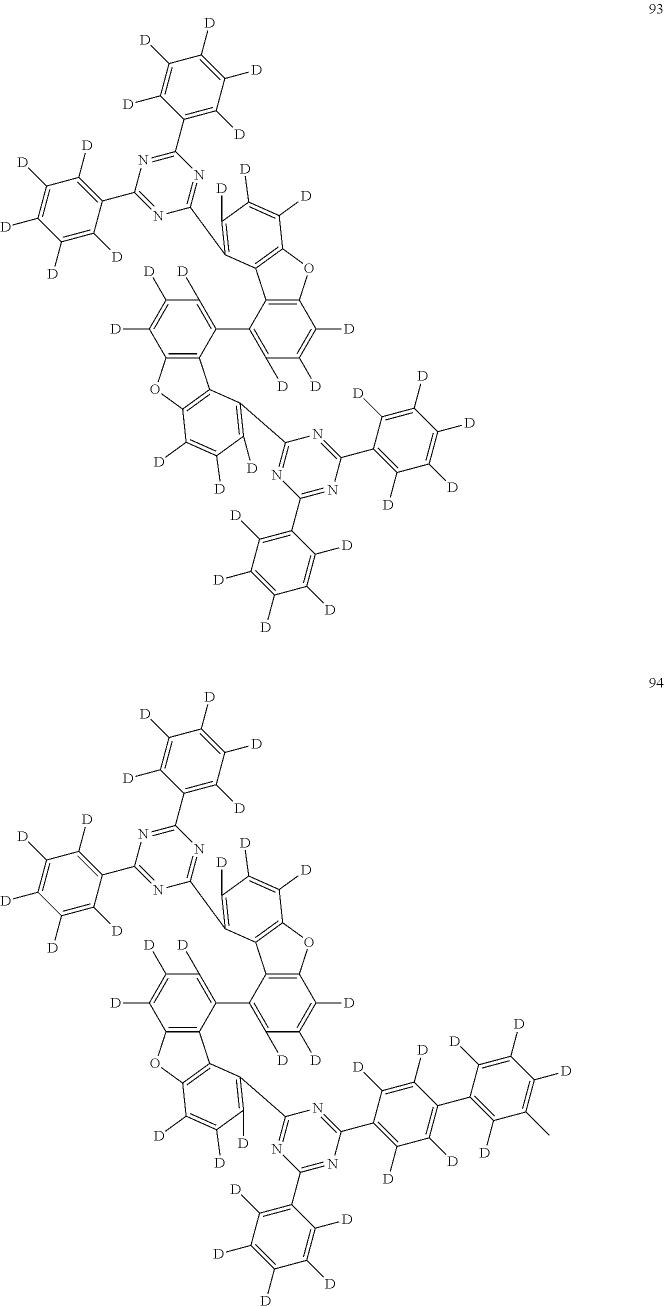

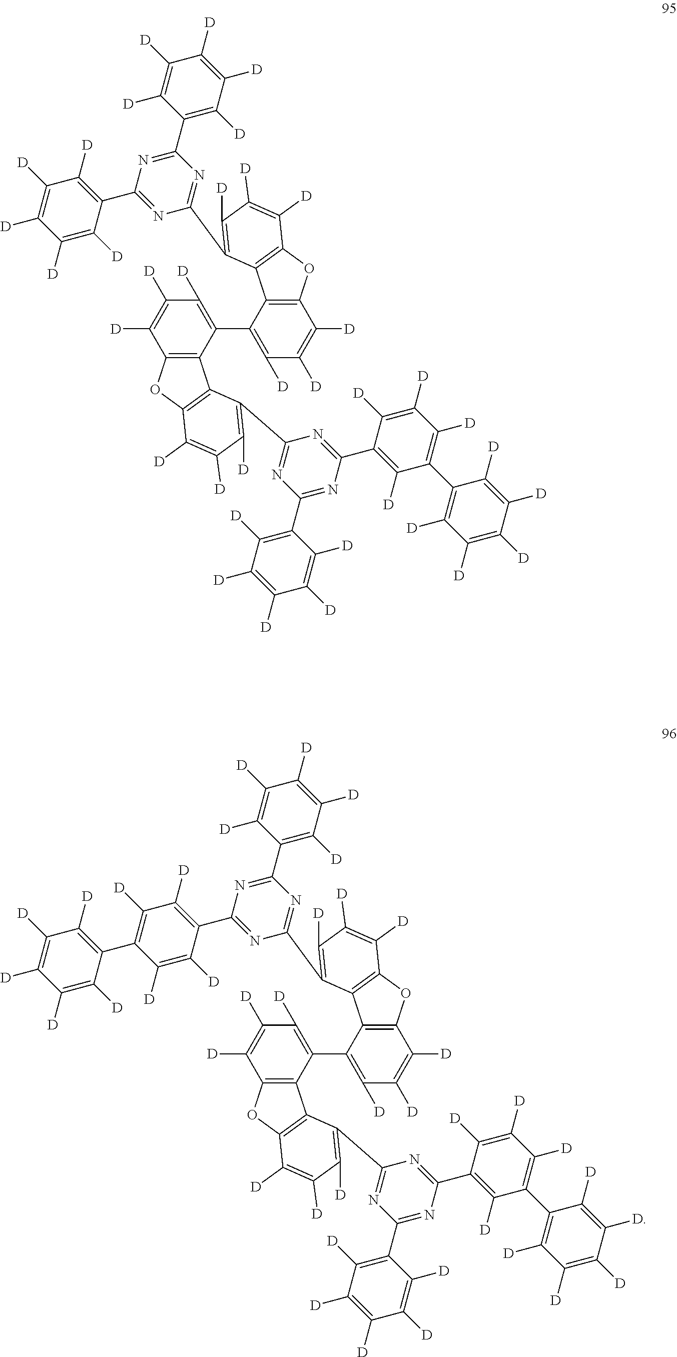

8. A composition for an organic optoelectronic device, the composition comprising:
a first compound; and
a second compound,
wherein:
the first compound is the compound for an organic optoelectronic device as claimed in claim 1, and
the second compound is represented by:
Chemical Formula 2,
a combination of Chemical Formula 3 and Chemical Formula 4, or
Chemical Formula 5: 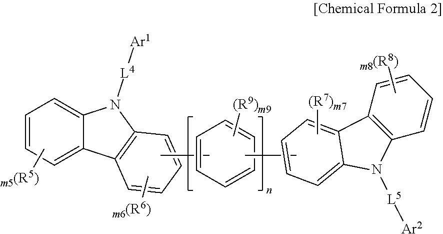
in Chemical Formula 2,
R⁵ to R⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar¹ and Ar² are each independently a substituted or unsubstituted C6 to C20 aryl group or a substituted or unsubstituted C2 to C30 heterocyclic group,
L⁴ and L⁵ are each independently a single bond or a substituted or unsubstituted C6 to C20 arylene group,
m5, m8, and m9 are each independently an integer of 1 to 4,
m6 and m7 are each independently an integer of 1 to 3,
when m5 is 2, 3, or 4, each R⁵ is the same or different from each other,
when m6 is 2 or 3, each R⁶ is the same or different from each other,
when m7 is 2 or 3, each R⁷ is the same or different from each other,
when m8 is 2, 3, or 4, each R⁸ is the same or different from each other,
when m9 is 2, 3, or 4, each R⁹ is the same or different from each other, and
n is an integer of 0 to 2; 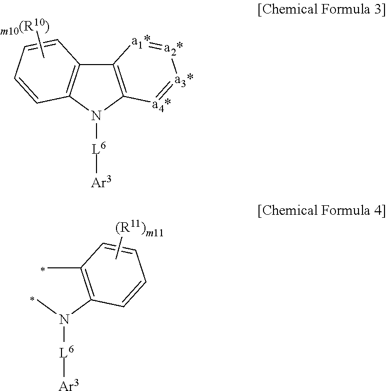
in Chemical Formula 3 and Chemical Formula 4,
two adjacent ones of a1* to a4* of Chemical Formula 3, are each linking carbons linked at * of Chemical Formula 4,
the remaining two of a1* to a4* of Chemical Formula 3, not linked at * of Chemical Formula 4, are independently C-L^{a}-R^{f},
L^{a}, L⁶, and L⁷ are each independently a single bond or a substituted or unsubstituted C6 to C20 arylene group,
R^{f}, R¹⁰, and R¹¹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar³ and Art are each independently a substituted or unsubstituted C6 to C20 aryl group or a substituted or unsubstituted C2 to C30 heterocyclic group,
m10 and m11 are each independently an integer of 1 to 4,
when m10 is 2, 3, or 4, each R¹⁰ is the same or different from each other, and
when m11 is 2, 3, or 4, each R¹¹ is the same or different from each other; 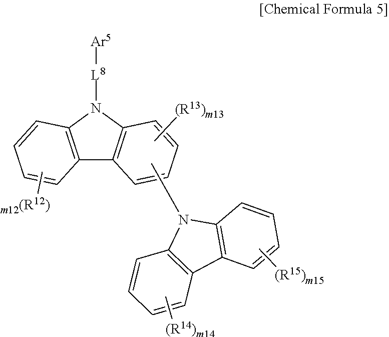
in Chemical Formula 5,
L⁸ is a single bond or a substituted or unsubstituted C6 to C20 arylene group,
R¹² to R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁵ is a substituted or unsubstituted C6 to C20 aryl group or a substituted or unsubstituted C2 to C30 heterocyclic group,
m12, m14, and m15 are each independently an integer of 1 to 4,
m13 is an integer of 1 to 3,
when m12 is 2, 3, or 4, each R¹² is the same or different from each other,
when m13 is 2 or 3, each R¹³ is the same or different from each other,
when m14 is 2, 3, or 4, each R¹⁴ is the same or different from each other, and
when m15 is 2, 3, or 4, each R¹⁵ is the same or different from each other.

9. The composition for an organic optoelectronic device as claimed in claim 8, wherein:
the second compound is represented by Chemical Formula 2, and
Chemical Formula 2 is represented by Chemical Formula 2-8: 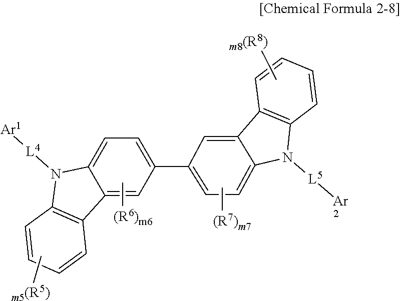
in Chemical Formula 2-8,
R⁵ to R⁸ are each independently hydrogen, deuterium, or a substituted or unsubstituted C6 to C12 aryl group,
m5 and m8 are each independently an integer of 1 to 4,
m6 and m7 are each independently an integer of 1 to 3,
moieties -L⁴-Ar¹ and -L⁵-Ar² are each independently a moiety of Group I, 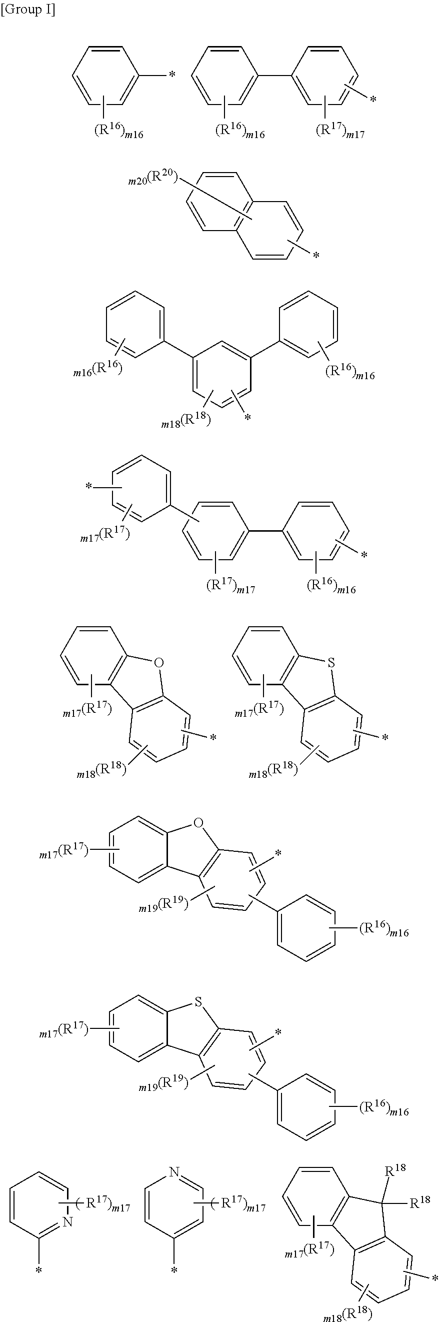 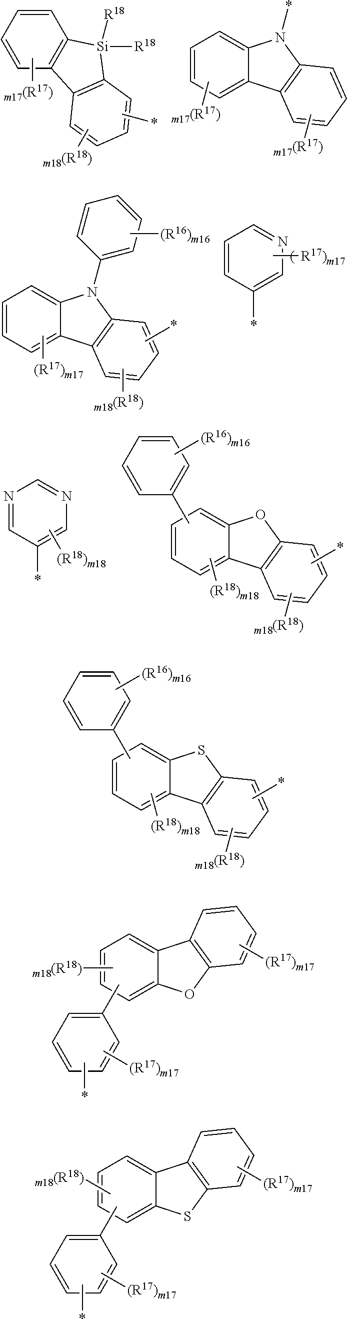 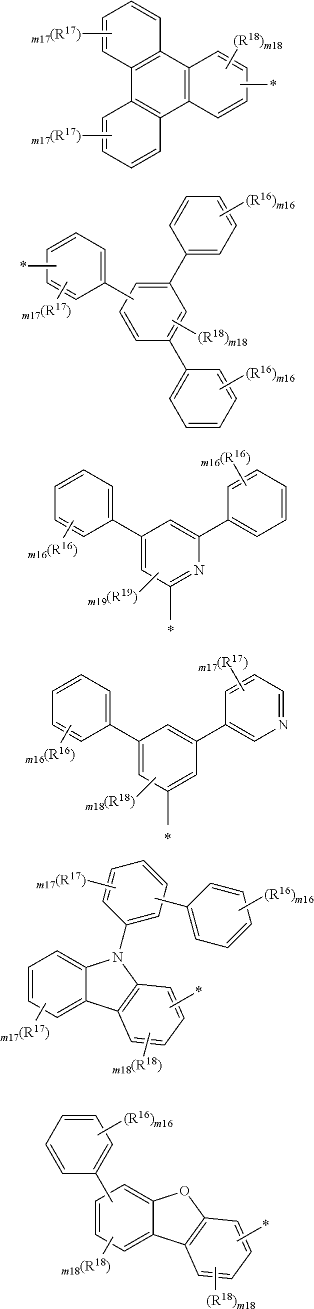 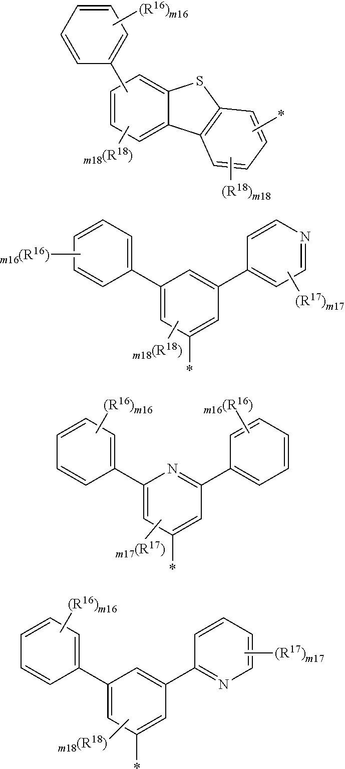
in Group I,
R¹⁶ to R²⁰ are each independently hydrogen, deuterium, a cyano group, a substituted or unsubstituted C1 to C10 alkyl group, or a substituted or unsubstituted C6 to C12 aryl group,
m16 is an integer of 1 to 5,
m17 is an integer of 1 to 4,
m18 is an integer of 1 to 3,
m19 is an integer of 1 or 2,
m20 is an integer of 1 to 7, and
* is a linking point.

10. The composition for an organic optoelectronic device as claimed in claim 8, wherein:
the second compound is represented by a combination of Chemical Formula 3 and Chemical Formula 4, and
the combination of Chemical Formula 3 and Chemical Formula 4 is represented by Chemical Formula 3C: 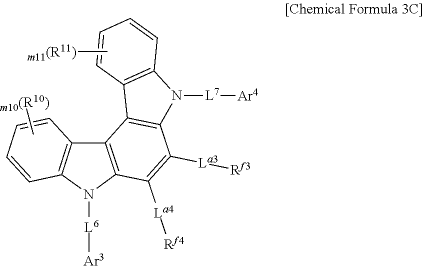
in Chemical Formula 3C,
L^{a3} and L^{a4} are each a single bond,
R¹⁰, R¹¹, R^{f3}, and R^{f4} are each independently hydrogen, deuterium, or a substituted or unsubstituted C6 to C12 aryl group,
m10 and m11 are each independently an integer of 1 to 4, and
moieties -L⁶-Ar³ and -L⁷-Ar⁴ are each independently a moiety of Group I, 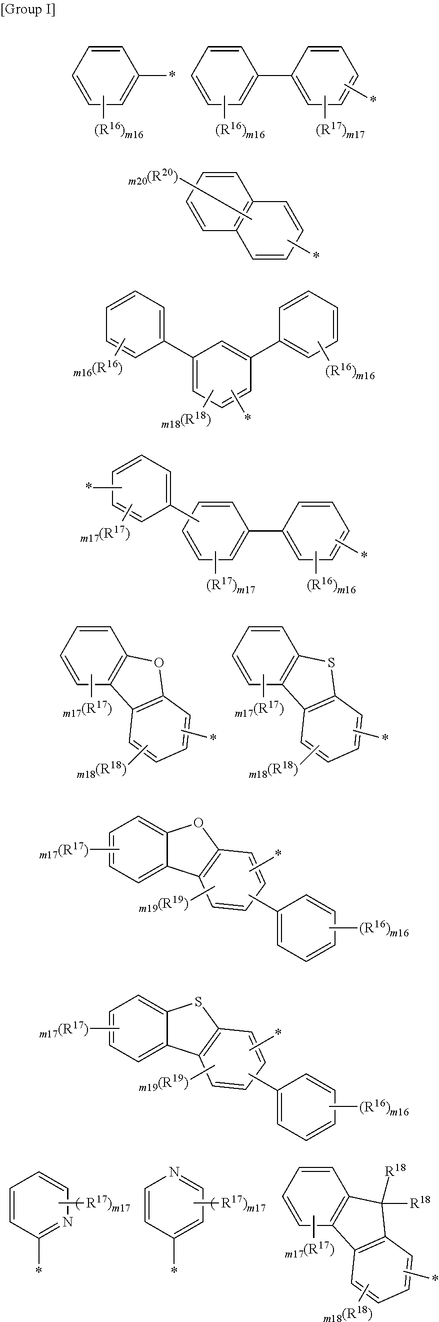 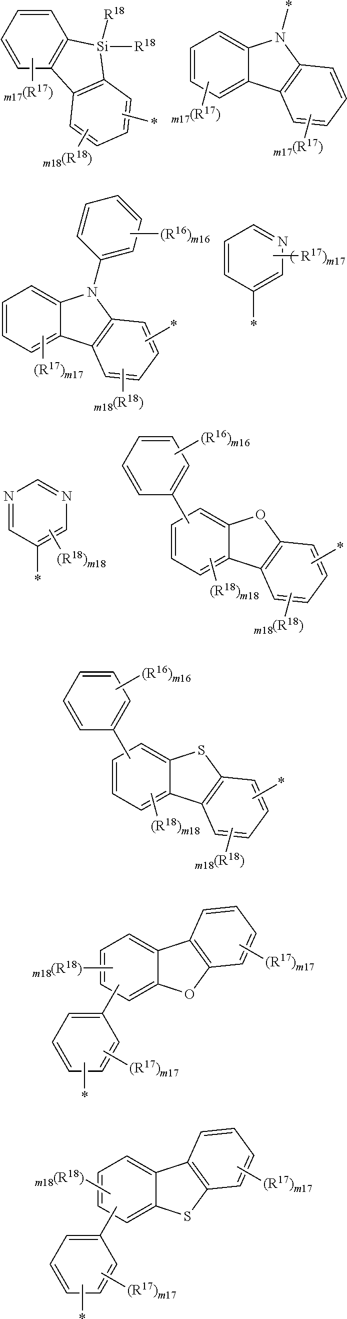 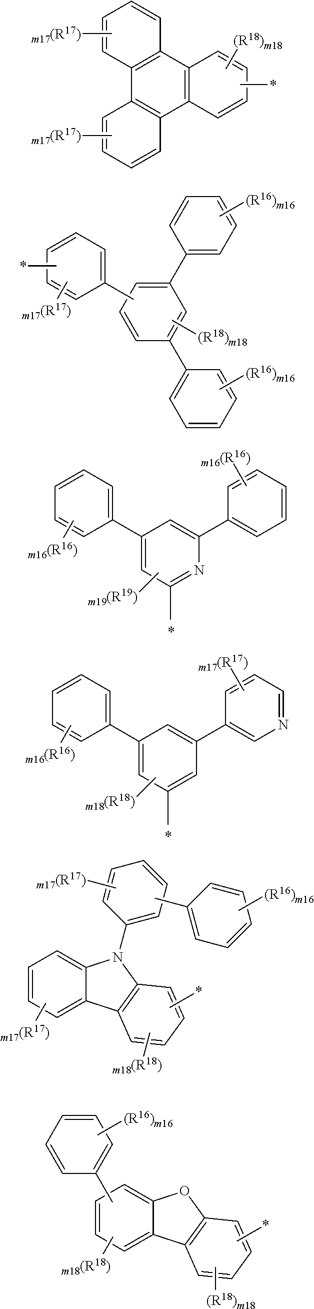 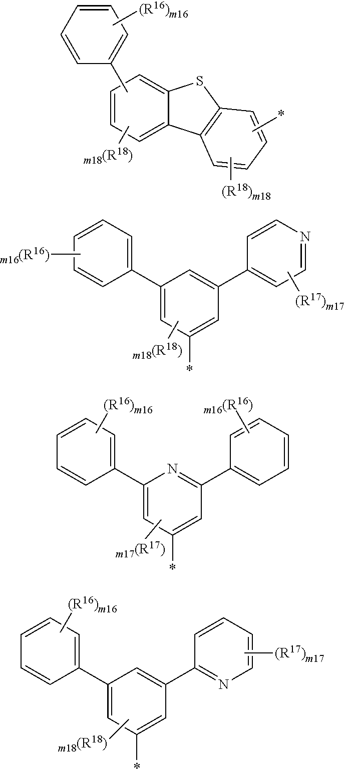
in Group I,
R¹⁶ to R²⁰ are each independently hydrogen, deuterium, a cyano group, a substituted or unsubstituted C1 to C10 alkyl group, or a substituted or unsubstituted C6 to C12 aryl group,
m16 is an integer of 1 to 5,
m17 is an integer of 1 to 4,
m18 is an integer of 1 to 3,
m19 is an integer of 1 or 2,
m20 is an integer of 1 to 7, and
* is a linking point.

11. An organic optoelectronic device, comprising:
an anode and a cathode facing each other, and
at least one organic layer between the anode and the cathode,
wherein the at least one organic layer includes the compound for the organic optoelectronic device of claim 1.

12. The organic optoelectronic device as claimed in claim 11, wherein:
the at least one organic layer includes a light emitting layer, and
the light emitting layer includes the compound.

13. A display device comprising the organic optoelectronic device as claimed in claim 11.

14. An organic optoelectronic device, comprising:
an anode and a cathode facing each other, and
at least one organic layer between the anode and the cathode,
wherein the at least one organic layer includes the composition for the organic optoelectronic device of claim 8.

15. The organic optoelectronic device as claimed in claim 14, wherein:
the at least one organic layer includes a light emitting layer, and
the light emitting layer includes the composition.

16. A display device comprising the organic optoelectronic device as claimed in claim 14.
